(19) United States
(12) Patent Application Publication
(10) Pub. No.: US 2026/0345503 A1
(43) Pub. Date: Nov. 5, 2026

(54) GENERATION OF SECRETOME-CONTAINING COMPOSITIONS, AND METHODS OF USING AND ANALYZING THE SAME

(71) Applicants: FUJIFILM CELLULAR DYNAMICS, INC., Madison, WI (US); Assistance Publique - Höpitaux de Paris, Paris (FR)

(72) Inventors: Nisa RENAULT, Middleton, WI (US); Michele HAMRICK, Madison, WI (US); Chad KOONCE, Cottage Grove, WI (US); Manon DESGRES, Bois le Roi (FR); Jacquelyn WONG, Phoenix, AZ (US); Guillaume CHURLAUD, Paris (FR); Camille HUMBERT, Paris (FR); Jéröme LARGHERO, Boulogne-Billancourt (FR); Philippe MENASCHÉ, Paris (FR); Valérie BELLAMY, L'Haÿs-les-Roses (FR); Chloé PEZZANA, Paris (FR)

(73) Assignees: FUJIFILM CELLULAR DYNAMICS, INC., Madison, WI (US); Assistance Publique - Hôpitaux de Paris, Paris (FR)

(21) Appl. No.: 19/122,408

(22) PCT Filed: Oct. 20, 2023
(86) PCT No.: PCT/US2023/035616

§ 371 Date: Apr. 18, 2025

Related U.S. Application Data
(60) Provisional application No. 63/417,887, filed on Oct. 20, 2022.
(60) Provisional application No. 63/469,359, filed on May 26, 2023.

(51) Int. Cl. A61K 9/50; A61K 31/7105; C12N 5/077

(52) U.S. Cl. CPC A61K 9/5068; A61K 9/5089; A61K 31/7105; C12N 5/0657; C12N 2500/02; C12N 2500/34; C12N 2501/115; C12N 2501/998; C12N 2506/45; C12N 2533/50; C12N 2533/52

(57) ABSTRACT

The present disclosure provides methods for generating and/or purifying secretomes, extracellular vesicles, and fractions thereof, from progenitor cells; and provides compositions containing such generated secretomes, extracellular vesicles, and fractions thereof. The present disclosure further provides methods for analyzing activities, and the functionality and potency, of such secretomes, extracellular vesicles, and fractions thereof. The present disclosure also relates to the therapeutic use of secretomes, extracellular vesicles, and fractions thereof. The present disclosure further relates to good manufacturing practices (GMP)-ready, scalable, culture protocol for the release of clinic-ready secretomes.

### CROSS REFERENCE TO RELATED APPLICATION

This application claims priority from U.S. Provisional Patent Application No. 63/417,887 filed Oct. 20, 2022, and U.S. Provisional Patent Application No. 63/469,359 filed May 26, 2023, the entire disclosures of which are incorporated herein by reference.

### INCORPORATION BY REFERENCE OF SEQUENCE LISTING

The content of the electronically submitted sequence listing, file name: F285249_Sequence listing as filed .xml; size: 13,305 bytes; date of creation: Oct. 20, 2023, filed herewith, is incorporated herein by reference in its entirety.

### FIELD OF THE INVENTION

The present disclosure relates generally to the generation, purification, isolation, and/or enrichment, of secretomes from cells (such as, but not limited to, progenitor cells); secretome-containing compositions containing such generated, purified, isolated, and/or enriched, secretomes; and to methods for analyzing one or more activities, properties, and/or characteristics, of such secretome-containing compositions. The present disclosure also relates to the therapeutic use of secretome-containing compositions containing secreted bioactive molecules, produced, purified, isolated, and/or enriched, by a method or methods disclosed herein. The present disclosure further relates to good manufacturing practices (GMP)-ready, scalable, culture protocols for the release, purification, isolation, and/or enrichment, of clinic-ready secretomes, compositions and use thereof.

### BACKGROUND

Cells, including those in in vitro or ex vivo culture, secrete a large variety of molecules and biological factors (collectively known as a secretome) into the extracellular space. See Vlassov et al. (Biochim Biophys Acta, 2012; 940-948). As part of the secretome, various bioactive molecules are secreted from cells within membrane-bound extracellular vesicles, such as exosomes. Extracellular vesicles are capable of altering the biology of other cells through signaling, or by the delivery of their cargo (including, for example, proteins, lipids, and nucleic acids). The cargo of extracellular vesicles is encased in a membrane which, amongst others, allows for specific targeting (e.g., to target cells) via specific markers on the membrane; and increased stability during transport in biological fluids, such as through the bloodstream or across the blood-brain-barrier (BBB).

Exosomes exert a broad array of important physiological functions, e.g., by acting as molecular messengers that traffic information between different cell types. For example, exosomes deliver proteins, lipids and soluble factors including RNA and microRNAs which, depending on their source, participate in signaling pathways that can influence apoptosis, metastasis, angiogenesis, tumor progression, thrombosis, immunity by directing T cells towards immune activation, immune suppression, growth, division, survival, differentiation, stress responses, apoptosis, and the like. See Vlassov et al. (Biochim Biophys Acta, 2012; 940-948). Extracellular vesicles may contain a combination of molecules that may act in concert to exert particular biological effects. Exosomes incorporate a wide range of cytosolic and membrane components that reflect the properties of the parent cell. Therefore, the terminology applied to the originating cell can in some instances be used as a simple reference for the secreted exosomes.

Progenitor cells have proliferative capacity and can differentiate into mature cells, making progenitor cells attractive for therapeutic applications such as regenerative medicine, e.g., in treating myocardial infarction and congestive heart failure. It has been reported that extracellular vesicles secreted by human embyonic stem cell-derived cardiovascular progenitor cells produce similar therapeutic effects to their secreting cells in a mouse model of chronic heart failure, see Kervadec et al. (J. Heart Lung Transplant, 2016; 35:795-807), suggesting that a significant mechanism of action of transplanted progenitor cells is in the release of biological factors following transplantation (e.g., which stimulate endogenous regeneration or repair pathways). This raises the possibility of effective, cell-free therapies (with benefits such as improved convenience, stability, and operator handling). However, there currently is a need for improved production methods for generating, purifying, isolating, and/or enriching, extracellular vesicles and compositions thereof for allogeneic or autologous human administration and use.

Established techniques for the generation of extracellular vesicles typically employ reagents and/or conditions that are not compatible with clinical or therapeutic use, or GMP standards. Furthermore, extracellular vesicles produced by one method would have different functionalities and properties from extracellular vesicles or secretomes produced by another similar method, see Théry et al. (J Extracell Vesicles. 2018 Nov. 23; 7(1):1535750).

Therefore, therapeutic administration, efficacy and safety of an extracellular vesicle containing composition can be method and process dependent. For instance, regulatory approval of production of drugs and biological substances requires strict adherence to laws and regulations that are promulgated with the goal of establishing safe and effective manufacturing facilities and products. As a non-limiting example, “Good Manufacturing Practices” (GMP) and “Good Laboratory Practices” (GLP) are established by regulation and implemented by the FDA (the U.S. Food and Drug Administration), CDER (Center for Drug Evaluation and Research), and CBER (Center for Biologics Evaluation and Research), with regard to drugs and biologics. Similar GMP and/or GLP laws are implemented worldwide, for instance in the EMEA.

For example, the use of serum in culturing protocols raises reliability- and biosafety-concerns, especially where serum obtained from an animal may be contaminated with, for example, infectious agents such as viruses or prions. Fetal bovine serum (FBS) is a widely used growth supplement for cell and tissue culture media; however, FBS is not well suited for clinical or therapeutic use for these reasons.

In contrast, the use of serum-free media confers many advantages, including consistency in formulations and safety. However, using only serum-free media can have disadvantageous effects on cell metabolism and growth, and there exists a need for good manufacturing practices (GMP)-ready compositions/methods for generating, purifying, isolating, and/or enriching, secretome compositions.

Additionally, there is a need for improved treatments for patients such as cancer survivors who have been treated with anthracyclines, and who are at risk of developing a left ventricular (LV) dysfunction (sometimes as late as even 10 to 20 years after the end of their cancer treatment). Several factors, like cumulative dose, age, and cardiovascular risk, increase the probability of developing anthracycline-induced cardiotoxicity, but in all cases, patients require careful monitoring, sometimes a reduction in dosing regimens, or the use of classical neuro-hormonal blockade preventive therapies (the benefit of which is still unclear).

Anthracycline treatment causes DNA damage, oxidative and energetic stress leading to inflammation, extracellular matrix remodeling, and defects in heart contractility (which, in the long term, lead to LV dysfunction).

In order to develop, optimize and release products for human therapeutic use, it is important to establish their safety and efficacy in appropriate models. A combined approach of testing products on in vitro human cells, together with animal studies will provide a strong data set describing product efficacy and safety, and predicting efficacy, safety, and use of the product in human subjects.

### SUMMARY OF THE INVENTION

The present disclosure addresses the above-described limitations in the art, by providing methods for generating, purifying, isolating, and/or enriching, secretomes using serum-free media, thereby permitting a GMP-ready, scalable, quality-controlled culture protocol for the release of clinic-ready secretomes.

The present disclosure also provides methods for generating, purifying, isolating, and/or enriching, secretomes, extracellular vesicles, and fractions thereof, from cells (such as, but not limited to, progenitor cells); and provides compositions containing such generated, purified, isolated, and/or enriched, secretomes, extracellular vesicles, and fractions thereof. The present disclosure further provides methods for analyzing one or more activities, properties, and/or characteristics, of such secretomes, extracellular vesicles, and fractions thereof, as well as the therapeutic use of secretomes, extracellular vesicles, and fractions thereof.

The present disclosure also provides assays for determining the effect of secretomes, extracellular vesicles, and fractions thereof, on the treatment of chemotherapy-induced cardiomyopathy. The present disclosure further provides compositions containing generated, purified, isolated, and/or enriched, secretomes, extracellular vesicles, and fractions thereof, for the treatment and/or prevention of chemotherapy-induced cardiomyopathy in a subject.

Non-limiting embodiments of the disclosure include as follows:
[1]A method for generating a secretome, said method comprising: (a) culturing one or more progenitor cells in a first serum-free culture medium, wherein said first serum-free culture medium comprises basal medium, human serum albumin, and one or more growth factors; (b) removing said first serum-free culture medium from said one or more progenitor cells; (c) culturing said one or more progenitor cells in a second serum-free culture medium, wherein said second serum-free culture medium comprises basal medium, but does not comprise human serum albumin or growth factors; and (d) recovering the second serum-free culture medium after the culturing of step (c), to thereby obtain conditioned medium comprising the secretome of the one or more progenitor cells.
[2] The method of [1], wherein one of said one or more growth factors is fibroblast growth factor 2 (FGF-2).
[3] The method of [1] or [2], wherein said first and second serum-free media are supplemented with a carbohydrate source.
[4] The method of [3], wherein said carbohydrate source is glucose.
[5] The method of any one of [1]-[4], wherein said first and second serum-free media are supplemented with an antibiotic.
[6] The method of [5], wherein said antibiotic is gentamicin.
[7] The method of any one of [1]-[6], wherein said first serum-free media further comprises one or more selected from the group consisting of: glutamine; biotin; DL alpha tocopherol acetate; DL alpha-tocopherol; vitamin A; catalase; insulin; transferrin; superoxide dismutase; corticosterone; D-galactose; ethanolamine, glutathione; L-carnitine; linoleic acid; progesterone; putrescine; sodium selenite; triodo-I-thyronine; an amino acid; sodium pyruvate; lipoic acid; vitamin B12; nucleosides; and ascorbic acid.
[8] The method of any one of [1]-[7], wherein said basal medium is a Minimum Essential Medium (MEM).
[9] The method of [8], wherein said MEM is α-MEM.
[10] The method of any one of [1]-[9], wherein the culturing of step (a) is for 6-96 hours.
[11] The method of [10], wherein the culturing of step (a) is for 12-96 hours.
[12] The method of [11], wherein the culturing of step (a) is for 36-84 hours.
[13] The method of [12], wherein the culturing of step (a) is for about 72 hours.
[14] The method of any one of [1]-[13], wherein the culturing of step (c) is for 6-96 hours.
[15] The method of [14], wherein the culturing of step (c) is for 12-72 hours.
[16] The method of [15], wherein the culturing of step (c) is for 36-60 hours.
[17] The method of [16], wherein the culturing of step (c) is for about 48 hours.
[18] The method of [14], wherein the last 12-36 hours of the culturing of step (c) is conducted under hypoxic conditions.
[19] The method of [18], wherein said culture conditions comprise culturing in an atmosphere having 1-21% oxygen.
[20] The method of any one of [1]-[19], wherein after step (b), but before step (c), said one or more progenitor cells are washed.
[21] The method of any one of [1]-[20], wherein said one or more progenitor cells comprise progenitor cells selected from the group consisting of cardiomyocyte progenitor cells, cardiac progenitor cells, and cardiovascular progenitor cells.
[22] The method of any one of [1]-[21], wherein said one or more progenitor cells are obtained from induced pluripotent stem cells (iPSCs).
[23] The method of any one of [1]-[4] and [7]-[22], wherein said first and second serum-free media do not contain an antibiotic.
[24] The method of any one of [1]-[23], wherein the culturing in one or more of steps (a) and (c) is two-dimensional cell culture.
[25] The method of [24], wherein said two-dimensional cell culture comprises culturing said one or more progenitor cells on a surface of a culture vessel.
[26] The method of [25], wherein said culture vessel surface is coated with a substance to promote cell adhesion.
[27] The method of [26], wherein said substance to promote cell adhesion is vitronectin or fibronectin.
[28] The method of any one of [1]-[23], wherein the culturing in one or more of steps (a) and (c) is three-dimensional cell culture.
[29] The method of [28], wherein the three-dimensional cell culture comprises culturing cell aggregates in suspension in a bioreactor, spinner flask, or stirred culture vessel, or comprises culturing cells in a microcarrier culture system.
[30] The method of any one of [1]-[29], wherein said method further comprises pre-clearing the medium recovered in step (d) by centrifugation, filtration, or a combination of centrifugation and filtration.
[31] The method of any one of [1]-[30], wherein said method further comprises freezing the medium recovered in step (d).
[32] The method of any one of [1]-[31], wherein said one or more progenitor cells cultured in step (a) have previously been frozen.
[33] The method of any one of [1]-[32], wherein said method further comprises concentrating, and/or enriching for a small extracellular vesicle-enriched fraction (sEV) from the medium recovered in step (d).
[34] The method of [33], wherein said sEV is concentrated, and/or enriched, from the recovered medium by at least one process selected from the group consisting of ultracentrifugation, filtration, ultrafiltration, tangential flow filtration, size exclusion chromatography, and affinity capture.
[35] The method of [33], wherein said enriching enriches for extracellular vesicles that have one or more of the following characteristics: (a) are CD63⁺, CD81⁺ and/or CD9+; (b) are between 50-200 nm in diameter; (c) are positive for one or more of CD49e, ROR1 (Receptor Tyrosine Kinase Like Orphan Receptor 1), SSEA-4 (Stage-specific embryonic antigen 4), MSCP (Mesenchymal stem cell-like protein), CD146, CD41b, CD24, CD44, CD236, CD133/1, CD29 and CD142; and/or (d) are negative for one or more of CD19, CD4, CD209, HLA-ABC (human leukocyte antigen-ABC), CD62P, CD42a and CD69.
[36] The method of [33], wherein said sEV comprises one or more of exosomes, microparticles, extracellular vesicles and secreted peptides/proteins.
[37]A secretome-containing composition obtained by the method of any one of [1]-[32].
[[38] An sEV-containing composition obtained by the method of any one of [33]-[36].
[39]A method for producing a therapeutic composition suitable for administration to a patient, said method comprising producing a secretome-containing composition according to the method of any one of [1]-[32].
[40] The method of [39], wherein said method further comprises purifying, concentrating, isolating, and/or enriching, said secretome-containing composition by one or more purification, concentrating, isolation, and/or enrichment, steps.
[41] The method of [39], wherein said method further comprises adding a pharmaceutically acceptable excipient or carrier to the secretome-containing composition.
[42]A method for producing a therapeutic composition suitable for administration to a patient, said method comprising producing an sEV-containing composition according to the method of any one of [33]-[36].
[43] The method of [42], wherein said method further comprises purifying, concentrating, isolating, and/or enriching, said sEV-containing composition by one or more purification, concentration, isolation, and/or enrichment, steps.
[44] The method of [42], wherein said method further comprises adding a pharmaceutically acceptable excipient or carrier to the sEV-containing composition.
[45]A therapeutic composition comprising the secretome-containing composition of [37], and a pharmaceutically acceptable excipient or carrier.
[46]A therapeutic composition comprising the sEV-containing composition of [38], and a pharmaceutically acceptable excipient or carrier.
[47]A secretome-containing composition obtained by the method of [1], wherein said one or more progenitor cells comprise progenitor cells selected from the group consisting of cardiomyocyte progenitor cells, cardiac progenitor cells, and cardiovascular progenitor cells.
[48] An sEV-containing composition obtained by the method of [33], wherein said one or more progenitor cells comprise progenitor cells selected from the group consisting of cardiomyocyte progenitor cells, cardiac progenitor cells, and cardiovascular progenitor cells.
[49]A therapeutic composition comprising the composition of [47], and a pharmaceutically acceptable excipient or carrier.
[50]A therapeutic composition comprising the composition of [48], and a pharmaceutically acceptable excipient or carrier.
[51]A method for treating acute myocardial infarction, heart failure, myocarditis, ischemic cardiomyopathy, cardiomyopathy, ventricular dysfunction, atrial dysfunction, or arrhythmia in a subject in need thereof comprising administering to the subject the therapeutic composition of [49] or [50].
[52]A method for improving angiogenesis, comprising administering to a subject in need thereof the therapeutic composition of [49] or [50].
[53]A method for improving cardiac performance, comprising administering to a subject in need thereof the therapeutic composition of [49] or [50].
[54] The method of [11], wherein the culturing of step (a) is for 60-84 hours.
[55] The method of [14], wherein the last 12-36 hours of the culturing of step (c) is conducted under normoxic conditions.
[56] The method of [55], wherein said normoxic conditions comprise culturing in an atmosphere containing 20-21% oxygen.
[57] The method of [29], wherein the bioreactor is a vertical wheel bioreactor.
[58] The method of [39], wherein said method further comprises cryopreserving, freezing, or lyophilizing, said secretome-containing composition.
[59] The method of [42], wherein said method further comprises cryopreserving, freezing, or lyophilizing, said sEV-containing composition.
[60] The method of [2], wherein said first serum-free media comprises 0.1-10 g/mL FGF-2.
[61] The method of [60], wherein said first serum-free media comprises 0.5-5 μg/mL FGF-2.
[62] The method of [61], wherein said first serum-free media comprises 0.5-2.5 μg/mL FGF-2.
[63] The method of [62], wherein said first serum-free media comprises about 1 μg/mL FGF-2.
[64] The method of any of [1]-[36], [39]-[44] and [54]-[63], wherein said method is Good Manufacturing Practices (GMP)-ready.
[65] The secretome-containing composition of [37], wherein said composition is GMP-ready.
[66] The sEV-containing composition of [38], wherein said composition is GMP-ready.
[67] The method of [14], wherein the last 12-36 hours of the culturing of step (c) is conducted under normoxic conditions.
[68] The method of [67], wherein said normoxic conditions comprises culturing in an atmosphere containing between 20-21% of oxygen.
[69] The method of [30], wherein said pre-clearing comprises at least three filtration steps.
[70] The method of [34], wherein the separation of said sEV from the recovered medium comprises tangential flow filtration.
[71] The secretome-containing composition of [37] comprising trehalose and L-histidine.
[72] The sEV-containing composition of [38] comprising trehalose and L-histidine.
[73] The secretome-containing composition of [37] or [65], wherein said composition is able to promote wound scratch healing in an in vitro wound scratch healing assay, and/or is able to promote cardiomyocyte viability in an in vitro cardiomyocyte viability assay.
[74] The sEV-containing composition of [38] or [66], wherein said composition is able to promote wound scratch healing in an in vitro wound scratch healing assay, and/or is able to promote cardiomyocyte viability in an in vitro cardiomyocyte viability assay.
[75] The secretome-containing composition of [37] or [65], wherein said composition is at least one of the following: a composition that has been enriched for extracellular vesicles having a diameter of between about 50-200 nm or between 50-200 nm, preferably having a diameter of between about 50-150 nm or between 50-150 nm; a composition that is substantially free or free of whole cells; and/or a composition that is substantially free of one or more culture medium components.
[76] The sEV-containing composition of [38] or [66], wherein said composition is at least one of the following: a composition that has been enriched for extracellular vesicles having a diameter of between about 50-200 nm or between 50-200 nm, preferably having a diameter of between about 50-150 nm or between 50-150 nm; a composition that is substantially free or free of whole cells; and/or a composition that is substantially free of one or more culture medium components.
[77] The method of [51], wherein the heart failure is acute heart failure, chronic heart failure, ischemic heart failure, non-ischemic heart failure, heart failure with ventricular dilation, heart failure without ventricular dilation, heart failure with reduced left ventricular ejection fraction, or heart failure with preserved left ventricular ejection fraction.
[78] The method of [77], wherein the heart failure is selected from the group consisting of ischemic heart disease, cardiomyopathy, myocarditis, hypertrophic cardiomyopathy, diastolic hypertrophic cardiomyopathy, dilated cardiomyopathy, and post-chemotherapy induced heart failure.
[79] The secretome-containing composition of [37] or [65], wherein said composition is able to promote cardiomyocyte viability in an in vitro chemotherapy-induced cardiomyopathy viability assay.
[80] The sEV-containing composition of [38] or [66], wherein said composition is able to promote cardiomyocyte viability in an in vitro chemotherapy-induced cardiomyopathy viability assay.
[81] The secretome-containing composition of [79], wherein in said in vitro chemotherapy-induced cardiomyopathy viability assay, said chemotherapy is an anthracycline.
[82] The secretome-containing composition of [81], wherein said anthracycline is doxorubicin.
[83] The sEV-containing composition of [80], wherein in said in vitro chemotherapy-induced cardiomyopathy viability assay, said chemotherapy is an anthracycline.
[84] The sEV-containing composition of [83], wherein said anthracycline is doxorubicin.
[85] The method according to [51], wherein said chemotherapy-induced cardiomyopathy is caused by an anthracycline.
[86] The method according to [85], wherein said anthracycline is doxorubicin.
[87]A method of maintaining physiological heart volume in a subject by administering to said subject the therapeutic composition of any one of [45], [46], [49] and [50].
[88] The method of any one of [51]-[53] and [87], wherein said method maintains Left Ventricular End Systolic Volume (LVESV) within 15% of the pre-treatment LVESV.
[89] The method of any one of [51]-[53] and [87], wherein said method maintains LVEDV within 2% of the pre-treatment volume.
[90] The method of any one of [51]-[53] and [87], wherein the method prevents progressive post-ischemic heart failure.
[91] The method of any one of [51]-[53] and [87], wherein the method improves endothelial cell survival, health and function in said subject.
[92] The method of any one of [51]-[53] and [87], wherein said method reduces fibrosis in stimulated cardiac fibroblasts.
[93] The method of [92], wherein said method reduces the expression of the pro-fibrotic marker, POSTN, in TGF-β1-stimulated cardiac fibroblasts to level prior to stimulation with TGF-β1 or below.
[94] The method of any one of [51]-[53] and [87], wherein the method does not induce an allogeneic inflammatory response in a subject.
[95] The method of any one of [51]-[53] and [87], wherein said method does not induce an allogeneic peripheral blood mononuclear cell (PBMC) activation.
[96] The method of any one of [51]-[53] and [87], wherein said method does not induce a significant increase in the percentage of IFNg or IL-2 expressing PBMCs.
[97] The method of any one of [51]-[53] and [87], wherein the method does not induce allogeneic natural killer (NK) cell degranulation.
[98] The method of any one of [51]-[53] and [87], wherein the method does not induce a significant increase in the percentage of CD107 expressing NK cells.
[99]A method of improving heart function in a patient experiencing heart failure by administering the therapeutic composition of any one of [45], [46], [49] and [50].
[100] The method of [99], wherein the method improves survival of stressed cardiomyocyte cells.
[101] The method of [99], wherein the method improves one or more of the seeding, survival, viability and proliferation of stressed endothelial cells in vitro.
[102] The method of [99], wherein the method improves cell migration and/or wound healing capabilities in stressed endothelial cells.
[103] The method of any one of [99]-[102], wherein the method improves wound healing in said subject.
[104] The method of any of one of [99]-[103], wherein the method reduces signs of fibrosis in fibroblast cells of said subject.
[105] The method of [104], wherein the fibroblasts are activated with TGF-β1.
[106] The method of any one of [99]-[105], wherein the method does not stimulate allogeneic human PBMC activation.
[107] The method of any of [99]-[106], wherein the method does not induce NK degranulation of allogeneic human NK cells.
[108] The method of any of [99]-[107], wherein the composition is non-toxic in mice and rats at a dose of 4×10¹¹ particles/kg.
[109] The method of any of [99]-[108], wherein the composition is not tumorigenic in mice at dose of 4×10¹¹ particles/kg.
[110] The method of any of [99]-[109], wherein the composition does not contain DNA fragments ranging from 179 to 742 pb, at concentrations in the μg/mL range.
[111] The method of any of [51]-[53] and [87]-[110], wherein said therapeutic composition is administered as an intravenous infusion, direct cardiac injection or is administered intra-arterially.
[112] The method of [111], wherein the therapeutic composition is administered at a dose containing secretome obtained from 0.1 to 10 million cells per kg weight of said subject per administration.
[113] The method of [111], wherein the therapeutic composition is administered at a dose containing secretome obtained from 0.5 to 5 million cells per kg weight of said subject per administration.
[114] The method of [111], wherein the therapeutic composition is administered at a dose containing secretome obtained from 1 to 3 million cells per kg weight of said subject.
[115] The method of [111], wherein the therapeutic composition is administered at a dose containing secretome obtained from 1 to 2 million cells per kg weight of said subject.
[116] The method of [111], wherein said therapeutic composition is administered at a dose containing from 1×10⁹ to 60×10⁹ particles, as measured by Nanoparticle Tracking Analysis (NTA), per kg weight of the subject.
[117] The method of [111], wherein said therapeutic composition is administered at a dose containing from 10×10⁹ to 60×10⁹ particles, as measured by NTA, per kg weight of the subject.
[118] The method of [111], wherein said therapeutic composition is administered at a dose containing from 10×10⁹ to 40×10⁹ particles, as measured by NTA, per kg weight of the subject.
[119] The method of [111], wherein said therapeutic composition is administered at a dose containing from 20×10⁹ to 40×10⁹ particles, as measured by NTA, per kg weight of the subject.
[120] The method of [111], wherein said therapeutic composition is administered at a cumulative daily dose containing from 20×10⁹ to 200×10⁹ particles, as measured by NTA, per kg weight of the subject.
[121] The method of [111], wherein said therapeutic composition is administered at a cumulative daily dose containing from 30×10⁹ to 100×10⁹ particles, as measured by NTA, per kg weight of the subject.
[122] The method of [111], wherein said therapeutic composition is administered at a cumulative daily dose containing 60×10⁹ particles, as measured by NTA, per kg weight of the subject.
[123] The method of [111], wherein said therapeutic composition is administered at a cumulative daily dose containing 40×10⁹ particles, as measured by NTA, per kg weight of the subject.
[124] The method of any one of [111]-[123], wherein said composition is administered from 1 to 10 times per day.
[125] The method of any one of [111]-[123], wherein said composition is administered from 3 to 6 times per day.
[126] The method of any one of [111]-[123], wherein said composition is administered from 1 to 5 times per day.
[127] The method of any one of [111]-[123], wherein said composition is administered 3 times per day.
[128] The method of any one of [111]-[123], wherein said composition is administered 2 times per day.
[129] The method of any one of [51]-[53] and [87]-[128], wherein the duration of treatment is 60 days or less.
[130] The method of [129], wherein the duration of treatment is from 5 to 50 days.
[131] The method of [129], wherein the duration of treatment is from 10 to 50 days.
[132] The method of [129], wherein the duration of treatment is from 20 to 45 days.
[133] The method of [129], wherein the duration of treatment is 42 days.
[134] The method of any one of [111]-[133], wherein said therapeutic composition is administered every day.
[135] The method of any one of [111]-[133], wherein said therapeutic composition is administered every other day.
[136] The method of any one of [111]-[133], wherein said therapeutic composition is administered at a frequency of from every day to every 30 days.
[137] The method of any one of [111]-[133], wherein said therapeutic composition is administered at a frequency of from every 7 days to every 21 days.
[138] The method of any one of [111]-[133], wherein said therapeutic composition is administered every 21 days.
[139]. The method of any one of [111]-[138], wherein said therapeutic composition is
formulated in a solution comprising one or more pharmaceutically acceptable excipient. [140]A secretome-containing composition obtained by the method of any one of [1]-[32], wherein said secretome-containing composition comprises extracellular vesicles secreted from said progenitor cells.
[141] The secretome-containing composition of [140], wherein said extracellular vesicles comprise one or more miR selected from hsa-miR-302a-5p, hsa-miR-16-5p, hsa-miR-93-5p, hsa-miR-126-3p, hsa-miR-148a-3p, hsa-miR-21-5p, hsa-miR-20a-5p, hsa-miR-143-3p, hsa-miR-335-5p, hsa-miR-218-5p, hsa-miR-101-3p, hsa-miR-302d-3p, hsa-miR-25-3p, hsa-miR-126-5p, hsa-miR-423-5p, hsa-miR-532-5p, hsa-miR-1246, hsa-miR-302a-3p, hsa-miR-20b-5p, hsa-miR-148b-3p, hsa-miR-34a-5p, hsa-miR-1-3p, hsa-miR-191-5p, hsa-miR-26b-5p, hsa-miR-151a-3p, hsa-miR-103a-3p/107, hsa-miR-660-5p, hsa-miR-320a-3p/320b/320c/320d/320e, hsa-miR-130a-3p, hsa-miR-19b-3p, hsa-miR-27a-3p/27b-3p, hsa-miR-186-5p, hsa-miR-26a-5p, hsa-miR-125b-5p, hsa-miR-7-5p, hsa-miR-24-3, hsa-miR-483-5p, hsa-miR-99b-5p, hsa-miR-205-5p, and hsa-miR-302b-3p.
[142] The secretome-containing composition of [140], wherein said extracellular vesicles comprise one or more miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p.
[143] The secretome-containing composition of [140], wherein said extracellular vesicles comprise at least five miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p.
[144] The secretome-containing composition of any one of [141]-[143], wherein an expression level of the one or more miR ranges from −5 to +5 units.
[145]A secretome-containing composition comprising a secretome from progenitor cells, said secretome comprising extracellular vesicles secreted from said progenitor cells.
[146] The secretome-containing composition of [145], wherein said progenitor cells are cardiovascular progenitor cells.
[147] The secretome-containing composition of [145] or [146], wherein said extracellular vesicles comprise one or more miR selected from hsa-miR-302a-5p, hsa-miR-16-5p, hsa-miR-93-5p, hsa-miR-126-3p, hsa-miR-148a-3p, hsa-miR-21-5p, hsa-miR-20a-5p, hsa-miR-143-3p, hsa-miR-335-5p, hsa-miR-218-5p, hsa-miR-101-3p, hsa-miR-302d-3p, hsa-miR-25-3p, hsa-miR-126-5p, hsa-miR-423-5p, hsa-miR-532-5p, hsa-miR-1246, hsa-miR-302a-3p, hsa-miR-20b-5p, hsa-miR-148b-3p, hsa-miR-34a-5p, hsa-miR-1-3p, hsa-miR-191-5p, hsa-miR-26b-5p, hsa-miR-151a-3p, hsa-miR-103a-3p/107, hsa-miR-660-5p, hsa-miR-320a-3p/320b/320c/320d/320e, hsa-miR-130a-3p, hsa-miR-19b-3p, hsa-miR-27a-3p/27b-3p, hsa-miR-186-5p, hsa-miR-26a-5p, hsa-miR-125b-5p, hsa-miR-7-5p, hsa-miR-24-3, hsa-miR-483-5p, hsa-miR-99b-5p, hsa-miR-205-5p, and hsa-miR-302b-3p.
[148] The secretome-containing composition of [145] or [146], wherein said extracellular vesicles comprise one or more miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p. [149] The secretome-containing composition of [145] or [146], wherein said extracellular vesicles comprise at least five miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p.
[150] The secretome-containing composition of any one of [147]-[149], wherein an expression level of the one or more miR ranges from −5 to +5 units.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an iPSC to CPC process flow diagram, illustrating the generation of cardiovascular progenitor cells from hiPSCs (steps 1-4). After CPC generation, cells were maintained as fresh aggregates (5a) or dissociated to single cells (step 5b) for the vesiculation process. Single cells were plated fresh or cryo-preserved and plated post-thaw (steps 6-7) for the vesiculation process.

FIG. 2A and FIG. 2B depict flowcharts showing the material generated in Example 1. As shown in FIG. 2A and FIG. 2B, two batches of CPCs (CPC1, CPC2) were generated and each were divided into three vesiculation conditions: aggregate vesiculation, fresh CPC plated vesiculation, and thawed CPC plated vesiculation. The conditioned media from each condition were collected, pre-cleared, and frozen (MC1-6). The cells at the end of four days of the vesiculation process (day+4) were also collected and analyzed (C+4 #1-6). Conditioned media were subjected to ultracentrifugation (UC) to isolate the small vesicular fraction (sEV 1-6). For MC5, three separate rounds of UC were performed on separate aliquots of MC5. In parallel, vessels containing media but no cells were incubated in the same conditions as the cell-containing vessels as described above. The media generated by this process, referred to as virgin media, were collected (virgin media 1-3). Subsequently, mock EV (also called MV) controls were generated from the virgin media via the same UC protocol as described above (MV1.1-3).

FIG. 3A and FIG. 3B depict heatmaps of the gene expression of 48 relevant genes to CPC differentiation and potential off targets. Data were generated using a custom Fluidigm qPCR panel. (A) Heatmap generated using the “SINGuLAR Analysis Toolset” package in R3.1.1 by calculating the global z-score. (B) Heatmap generated by calculating the gene Z-Score followed by hierarchical clustering in JMP software version 17 (ward method, unstandardized). The Ct values are presented in TABLE 1. Data from CPCs at the end of the differentiation process (CPC), as well as four days into the vesiculation process (C+4), are shown in addition to iPSC and cardiomyocyte (CM) controls. Under these conditions, CPC are clustered and separate from C+4 cells, which are more mature than CPCs but less mature than CM. Fourth vesiculation day aggregates (Agg+4) are distinct from fourth day hyperflask plated cells (HF+4). Both conditions show increased cTNT (cardiac Troponin T) and alpha-MHC (alpha-myosin heavy chain) expression compared to CPC. This supports the idea that CPC in the vesiculation process remain on the cardiac differentiation lineage, but do not attain the CM differentiation state, as shown by the persistence of CPC marker expression such as PDGFRa, ISL-1 and KDR.

FIG. 4 depicts a process flow diagram for the generation of conditioned media and virgin media controls.

FIG. 5 depicts a process flow diagram for the isolation of sEV or mock (virgin media) control samples.

FIG. 6 depicts representative size distribution curves from two sEVs and two control MV samples. Suspension culture yielded higher concentrations of particles than plated culture, and both were much higher than controls. Mode particle sizes for sEV 1 and sEV 2 (74 nm, 99 nm respectively) are consistent with exosomes or small microparticles.

FIG. 7 depicts ELISA results for the detection of CD-63. Bars numbered one through nine from left to right. sEVs (bars one, four, five, six, and seven) and MV controls (bars two, three, eight, and nine) were analyzed by FUJIFILM Wako Elisa kit for the detection of CD-63, a protein found on the surface of EV, especially exosomes. The results show that for a given protein input, MVs contain no CD-63 signal, whereas sEVs from both aggregate and plated cultures do. Aggregate sEV (bar one) produced more CD-63/protein signal than sEV from plated vesiculation protocols (bars four through seven). Replicate preparations of sEV from the same MC (5.1, 5.2 and 5.3, bars five, six, and seven) yielded similar CD63 signals. Furthermore, sEV isolated from different MCs generated from separate lots also yielded similar CD-63/g protein (sEV 2 (bar four) vs sEV 5.1/.2/.3). Protein signal is given as (absorbance 450−absorbance 620) minus the result for the blank (“abs 450-620, blank adj).

FIG. 8 depicts relative scratch wound closure in a HUVEC scratch wound healing assay. Bars numbered one through seven from left to right. sEVs from suspension and plated vesiculation processes (bars four and six) as well as their corresponding mock EV controls (MV) (bars five and seven) were tested in a HUVEC scratch wound healing assay. Controls were complete HUVEC media (positive control, “Positive”, bar one), poor HUVEC media (no supplements, negative control, “Negative”, bar two), and poor media+the sEV isolated from fetal bovine serum by UC (“FBS-EV”, additional positive control, bar three). sEV from suspension (bar four) and plated (bar six) vesiculation processes showed improved wound healing compared to Negative and MV controls.

FIG. 9 depicts the results of an H9c2 viability assay. Bars are labeled one through seven from left to right. The results of the H9c2 cell viability assay show that the sEVs from suspension (bar three) and plated (bars five and seven) cultures improve H9c2 survival in a serum deprivation assay. MVs (bars four and six) showed minimal to no positive effect in this assay. sEV generated from the suspension vesiculation method showed an improvement in fold change over negative control over the positive control, suggesting increased cell proliferation in addition to sustained survival.

FIG. 10 depicts a time course of cardiomyocyte survival in a staurosporine-induced cardiotoxicity assay. Condition lines labeled A through F from top to bottom, according to last data point. sEV from plated (line C) and aggregate (line B) cultures improve CM survival in this staurosporine assay. Aggregate cultures are suspension cultures in this experiment. MVs (lines D and F) showed little to no effect on CM survival. Arrows link each sEV with its corresponding MV control. The 18-hour data points from FIG. 10 are given in TABLE 2.

FIGS. 11A and 11B depict flowcharts illustrating the stages of production (vesiculation, conditioned media clarification, and TFF for Test Example 20, FIG. 11A; followed by final formulation, FIG. 11B) in a first GMP-compatible process, described in Example 5 and Example 6. The final formulation in this example was produced with and without trehalose addition prior to sterilizing filtration. The different stages at which samples were taken for in-process testing and quality control testing was undertaken are indicated with a “*” (e.g., *1, *2, *3, etc.).

FIG. 12 depicts the results of flow cytometry experiments to analyze the cell marker expression profile of CPCs at different times during the vesiculation process (D+0, D+3 and D+5). iPSCs and cardiomyocytes (CM) were used as control cells and were analyzed separately. The values shown are average values.

FIG. 13 depicts the results of transcriptome analysis of CPCs at different times during the vesiculation process (D+0, D+3 and D+5). RNA was extracted from CPCs at D+0, and from cells at D+3 and D+5 of the vesiculation process. RNA was also extracted from iPSCs (pluripotent cell controls), and from iPSC-derived cardiomyocytes (differentiated cardiomyocyte controls; CM). Total RNA was sequenced on the Illumina NovaSeq 6000 platform, and differential gene expression was determined on normalized data. The FIG. 13 and FIG. 13B heatmaps were generated based on hierarchical clustering analysis using the UPGMA clustering method, with correlation distance metric in TIBCO Spotfire software v11.2.0. The FIG. 13 heatmap has a blue to red color scale where dark blue represents low expression and dark red represents high expression. The FIG. 13B heatmap is in grey scale where white represents low expression and dark grey/black represents high expression. The data (log₂FPKM) used to generate both heatmaps are given in TABLE 3.

FIG. 14 depicts the morphology of CPCs during the vesiculation process, as observed under light microscopy. Cell morphology was analyzed in cells within both T75 and selected CS10 flasks. The left image is a representative image showing the typical D+3 morphology observed in all vessels analyzed at D+3. The right image is a representative image showing the typical D+5 morphology observed in all vessels analyzed at D+5, T75 flasks were used for image capture for clarity.

FIGS. 15A and 15B depict the results of an analysis of particle concentration and size distribution of EVs. FIG. 15A depicts the particle concentration and size distribution of EVs in clarified conditioned media before tangential flow filtration (TFF) (*5(Test 20)), and in final formulations without trehalose (*7, samples a (Test 20)) and with trehalose (*7, sample b (Test 20)), using nanoparticle tracking analysis. FIG. 15B depicts the particle concentration and size distribution of EVs in clarified conditioned media before tangential flow filtration (TFF) (*5(Test 20)), and in stored retentate samples without trehalose or histidine (*6, sample a (Test 20)), with trehalose (*6, sample b (Test 20)) or with histidine (*6, sample c (Test 20)) which were not filter sterilized. As FIGS. 15A and 15B show, TFF increased the particle concentration by about 32-fold.

FIGS. 16A-16D depict the results of MACSPlex analysis. FIGS. 16A and 16B depict the results of analysis of small EV-enriched secretome final formulations with and without trehalose, for expression of extracellular vesicle tetraspanins often expressed on the surface of extracellular vesicles (CD9, CD81 and CD63) (FIG. 16A); and for various additional markers, which exhibited little or no expression (FIG. 16B). FIGS. 16C and 16D depict the results of analysis of stored retentate samples (with and without trehalose or histidine) which were not filter sterilized [see FIG. 11B; *6, sample a (Test 20); *6, sample b (Test 20); *6, samples c (Test 20)], for expression of extracellular vesicle tetraspanins often expressed on the surface of extracellular vesicles (CD9, CD81 and CD63) (FIG. 16C); and for various additional markers, which exhibited little or no expression (FIG. 16D).

FIGS. 17A and 17B depict the results of analysis of samples *7, sample a (Test 20); *7, sample b (Test 20); (*6, sample 1 (Test 20); *6, sample b (Test 20); *6, sample c (Test 20) for the presence of cardiac-related markers. FIG. 17A depicts the results for small EV-enriched secretome final formulations with and without trehalose, for expression of cardiac-related markers. FIG. 17B depicts the results for stored retentate samples (with and without trehalose or histidine) which were not filter sterilized, for expression of cardiac-related markers. For all samples depicted in FIG. 17A and FIG. 17B, the interrogated markers were found to be present.

FIG. 18 depicts relative scratch wound healing in a HUVEC scratch wound healing assay. Bars labeled one through seven from left to right. Small EV-enriched secretome final formulations with (bars six and seven) and without (bars four and five) trehalose, were tested in a HUVEC scratch wound healing assay. The positive control (“+ve”, bar 1) consisted of culturing the scratched well in complete HUVEC cell medium (“Comp”) plus PBS “treatment,” and the negative control (“−ve”, bar 2) consisted of culturing the scratched wells in basal medium (“Poor”) plus PBS “treatment.” FBS-derived EV served as an EV control (“EV Ctl”, bar three). A 1× treatment equals the secretome derived from 150,000 cells. Values are baseline (negative control) subtracted and normalized to the positive control.

FIG. 19 depicts cardiomyocyte survival in a staurosporine-induced cardiotoxicity assay. Bars labeled one to seven from left to right. Small EV-enriched secretome final formulations with (bars six and seven) and without (bars four and five) trehalose, were tested in a cardiomyocyte survival assay. A 1× treatment equals the secretome derived from 150,000 cells. PBS controls with (bar two) and without (bar one) staurosporine served as negative (“−ve”) and positive (“+ve”) controls, respectively. Mesenchymal Stem Cell (MSC)-derived EV served as an EV control (“EV Ctl”, bar three). Plated cells were either stressed with staurosporine for 4 hours prior to treatment (“+”) or were not stressed with staurosporine (“−”).

FIGS. 24A and 24B depict flowcharts illustrating the stages of production (vesiculation, conditioned media clarification, and TFF, FIG. 24A; and final formulation, FIG. 24B) in a second GMP-compatible process, described in Example 12 and Example 13, i.e., for Test Example 22. The final formulation in this example was produced with and without trehalose addition prior to sterilizing filtration. The different samples which underwent in-process and quality control testing are indicated with a “*” (e.g., *6, *7, etc.).

FIG. 25 depicts the results of flow cytometry experiments to analyze the cell marker expression profile of CPCs at different times during the vesiculation process (D+O, D+3 and D+5). iPSCs and cardiomyocytes (CM) were used as control cells and were analyzed separately. The values shown are average values.

FIG. 26 depicts the morphology of CPCs during the vesiculation process, as observed under light microscopy. Cell morphology was analyzed in cells within both T75 and selected CS 10 flasks. The left image is a representative image showing the typical D+3 morphology observed in all vessels analyzed at D+3 The right image is a representative image showing the typical D+15 morphology observed in all vessels analyzed at D-+5. 175 flasks were used for image capture for clarity.

FIGS. 27A and 27B depict the results of an analysis of particle concentration and size distribution of EVs. FIG. 27A depicts the particle concentration and size distribution of EVs in conditioned media, before clarification, conditioned media after clarification, in the final formulation (i.e., after TFF) and in the final formulation with trehalose using nanoparticle tracking analysis. Sample naming is depicted in FIG. 24A and FIG. 24B. FIG. 27B depicts the concentration and size distribution of particles detected by NTA in *6, sample a (Test 22); *7, sample c (Test 22); and *7, sample d (Test 22).

FIGS. 28A-28B depict the MACSPlex results of analysis of small EV-enriched secretome final formulations with and without trehalose, for expression of extracellular vesicle tetraspanins often expressed on the surface of extracellular vesicles (CD9, CD81 and CD63) (FIG. 28A); and for various other markers, which exhibited little or no expression (FIG. 28B).

FIG. 29 depicts the MACSPlex results for small EV-enriched secretome final formulations with and without trehalose, for expression of cardiac-related markers. For all samples depicted in FIG. 29, the markers depicted in FIG. 29 were found to be expressed.

FIGS. 30A and 30B depict relative scratch wound healing in a HUVEC scratch wound healing assay. Bars labeled 1 through 15 from left to right in 30A and 16 though 30 from left to right in 30B. The results for samples *7, sample a (Test 22) (bars 4 through 9) and *7, sample b (Test 22) (bars 10 through 15) (depicted in FIG. 2411) are shown in FIG. 30A. The results for samples *7, sample c (Test 22) (bars 19 through 24) and *7, sample d (Test 22) (bars 25 through 30) (depicted in FIG. 24B) are shown in FIG. 30B. The positive control (“+ve”, bars 1 and 16) consisted of culturing the scratched well in complete HUVEC cell medium (“Comp”) plus PBS “treatment”, and the negative control (“−ve”, bars 2 and 17) consisted of culturing the scratched wells in basal medium (Poor) plus PBS “treatment”. FBS-derived EV served as an EV control (EV Ctl, bars 3 and 18). A 1× treatment equals the secretome derived from 150,000 cells. Values are baseline subtracted (negative control) and normalized to the positive control. Samples *7, sample a (Test 22); *7, sample b (Test 22); *7, sample c (Test 22)”; and “*7 sample d (Test 22), whose preparation is described in detail in Example 12 and Example 13 were tested in a scratch wound healing assay as described in Example 17. These four samples are derived from the same TFF retentate but differ in their method of final formulation. These four variations are to use fresh retentate and filter sterilize with Sterivex-GP, 0.22 μm filter (resulting in sample a); to use fresh retentate, supplement with trehalose, and filter sterilize with a Sterivex-GP, 0.22 μm filter (resulting in sample b); freezing a retentate, thawing it, and then filter sterilizing with a Sterivex-GP, 0.22 μm filter (resulting in sample c); or freezing a retentate, thawing it, and then filter sterilizing with a Sartopore 2; 0.45+0.2 μm filter (resulting in sample d).

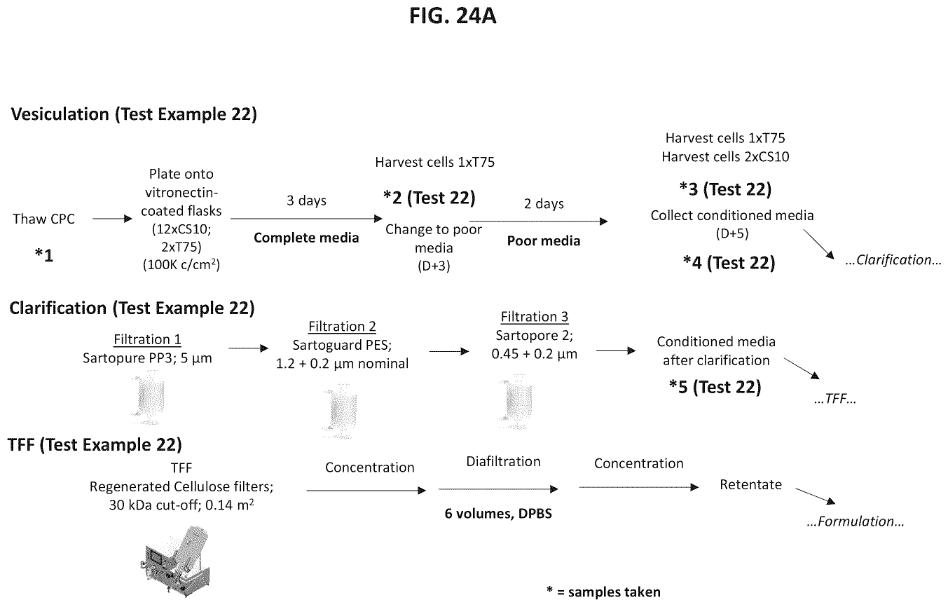

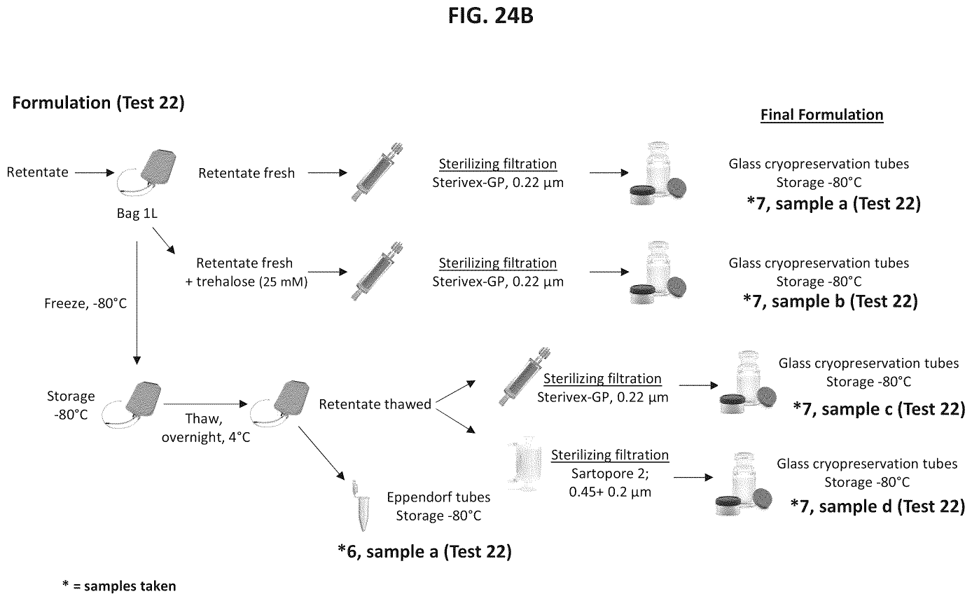

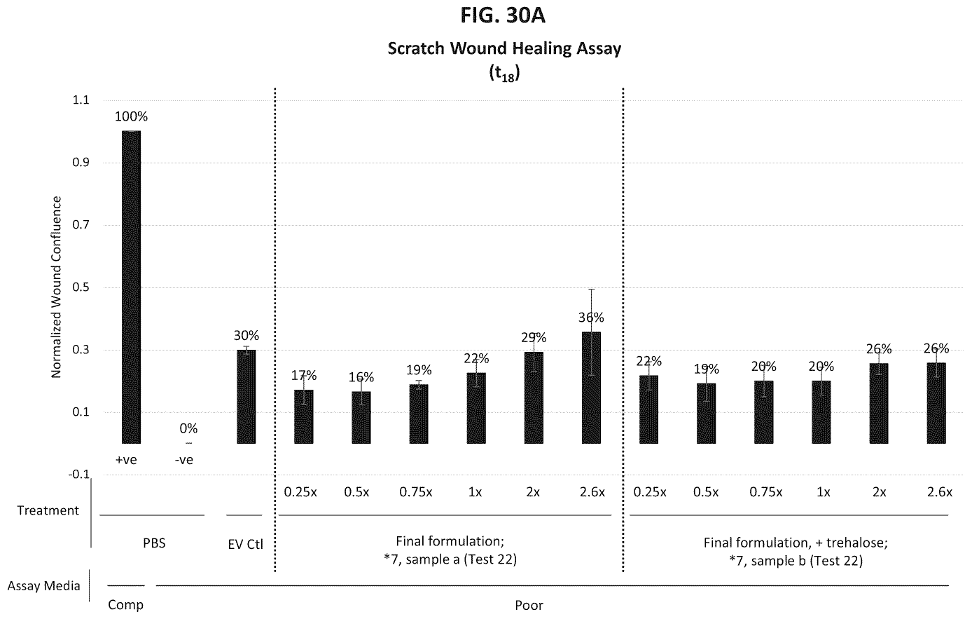

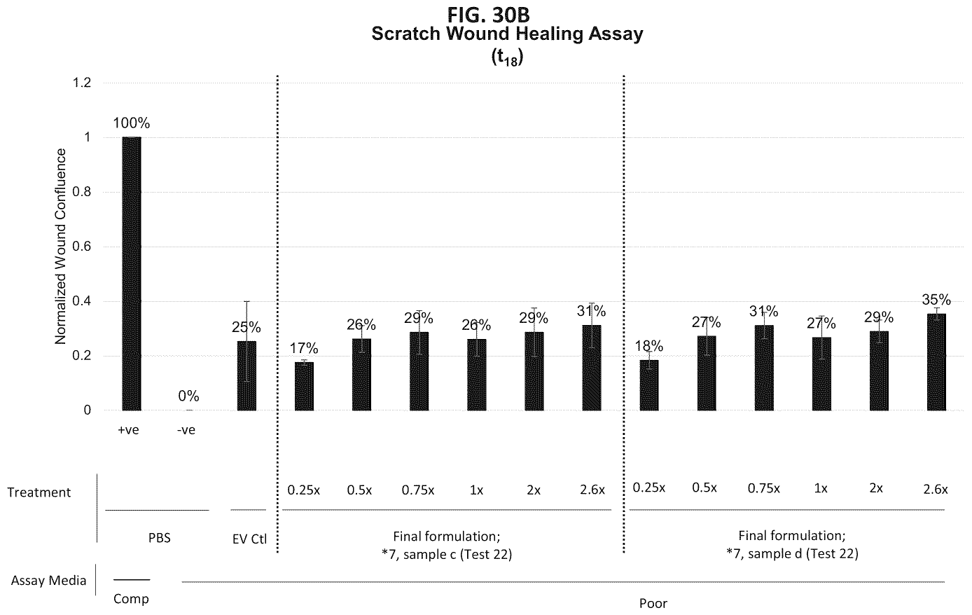

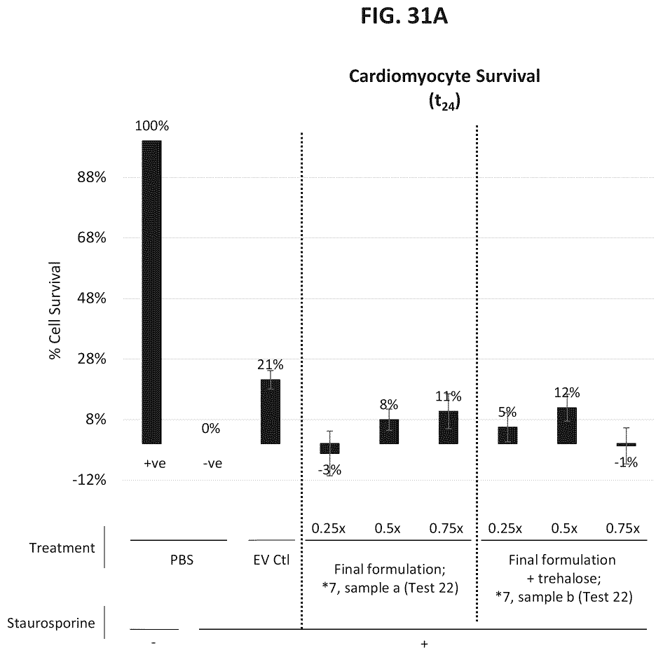

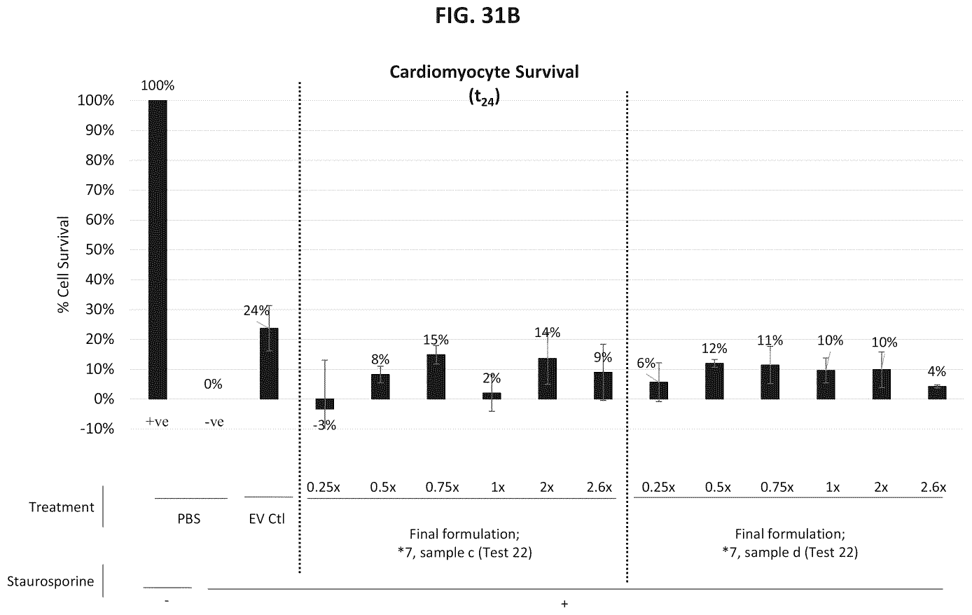

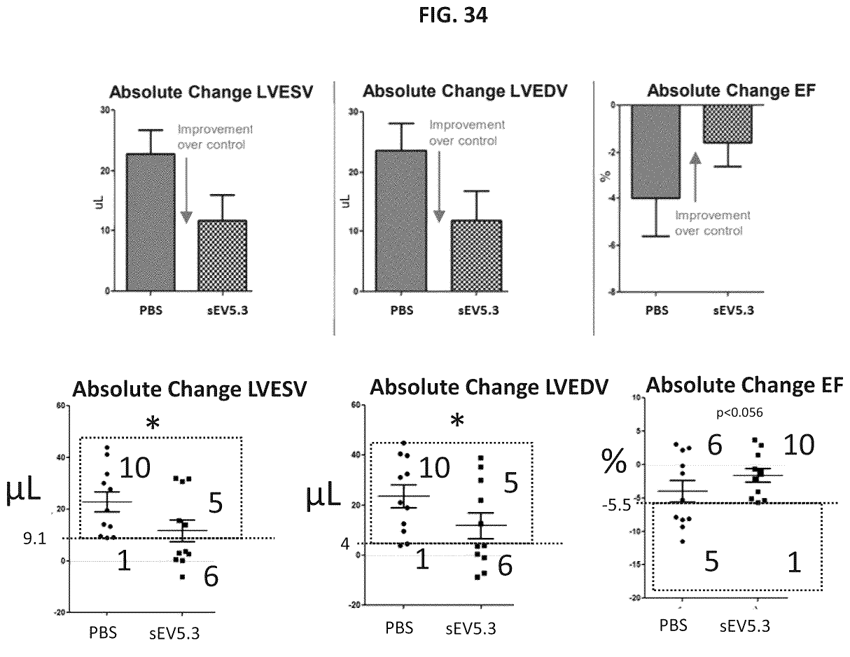

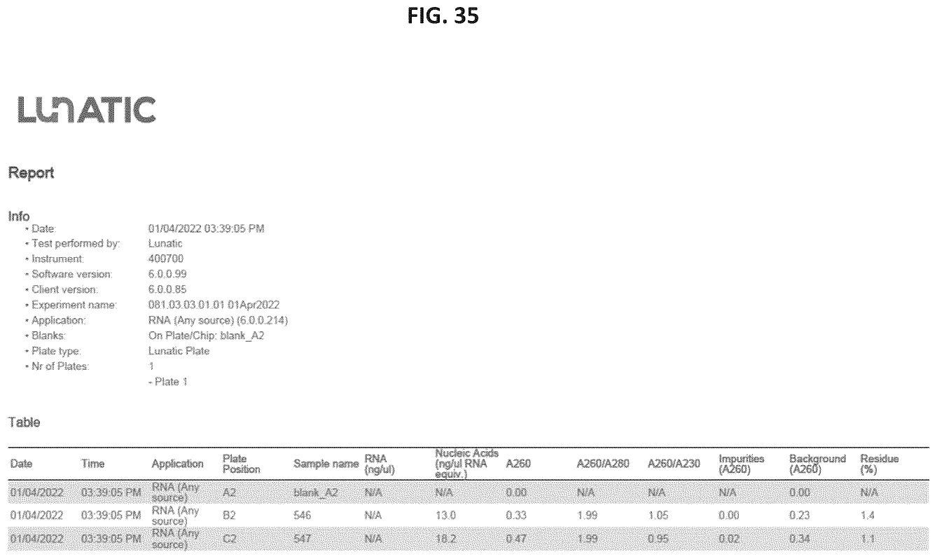

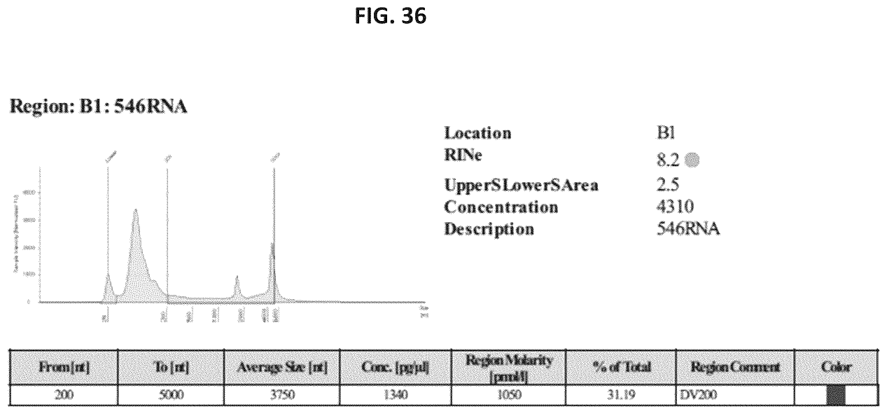

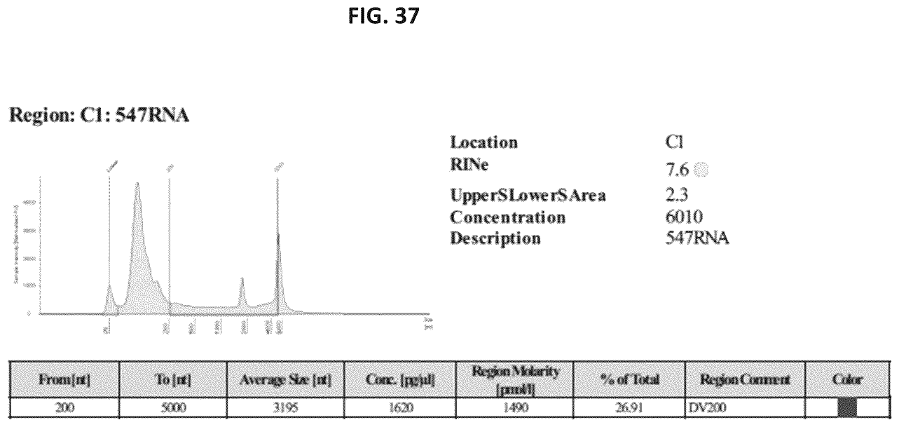

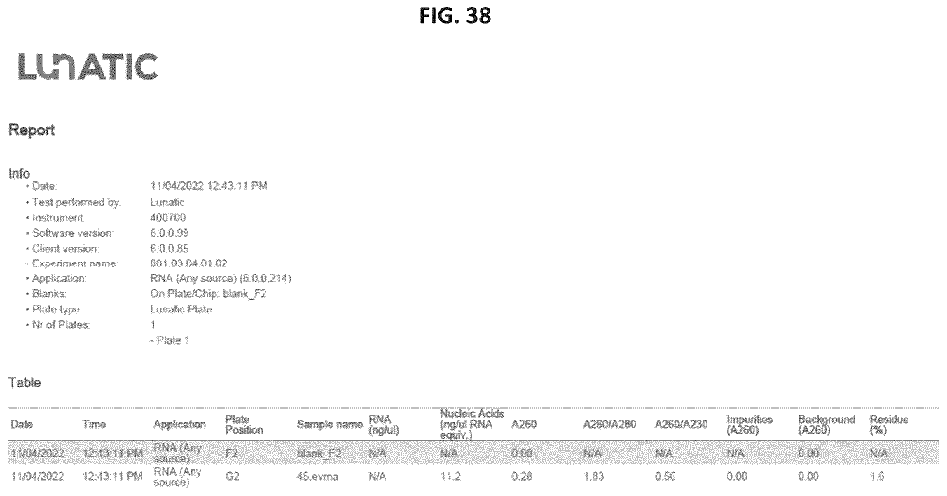

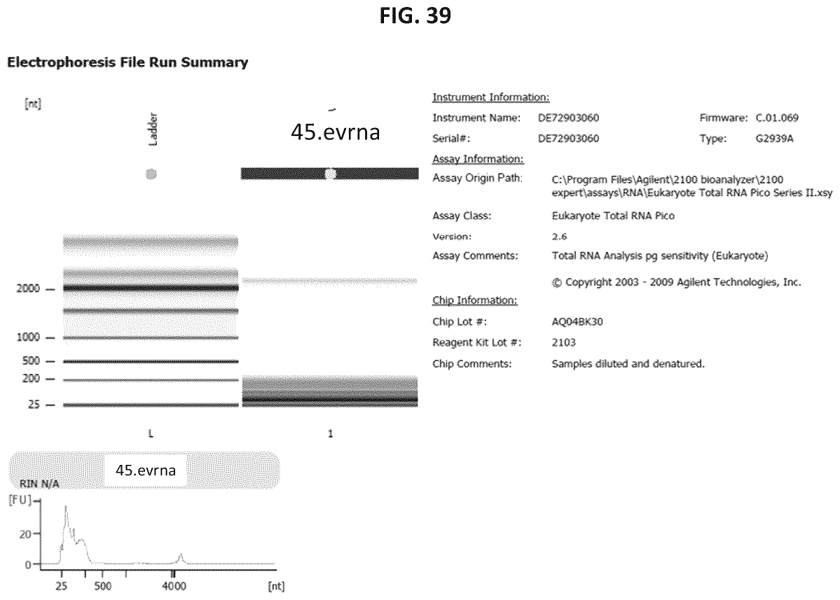

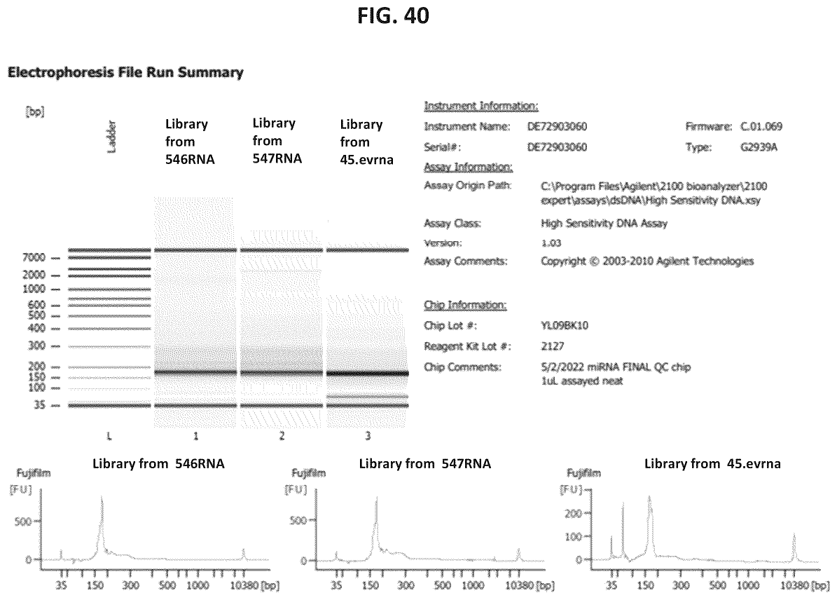

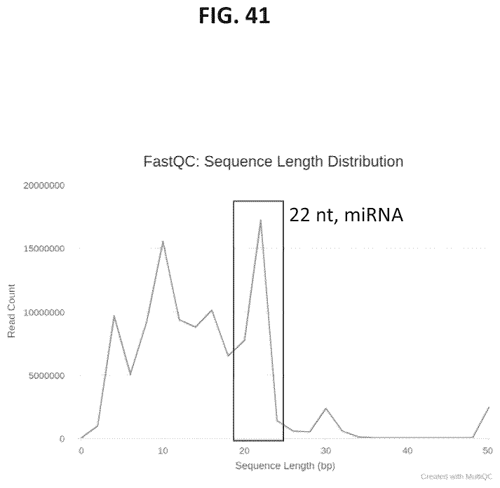

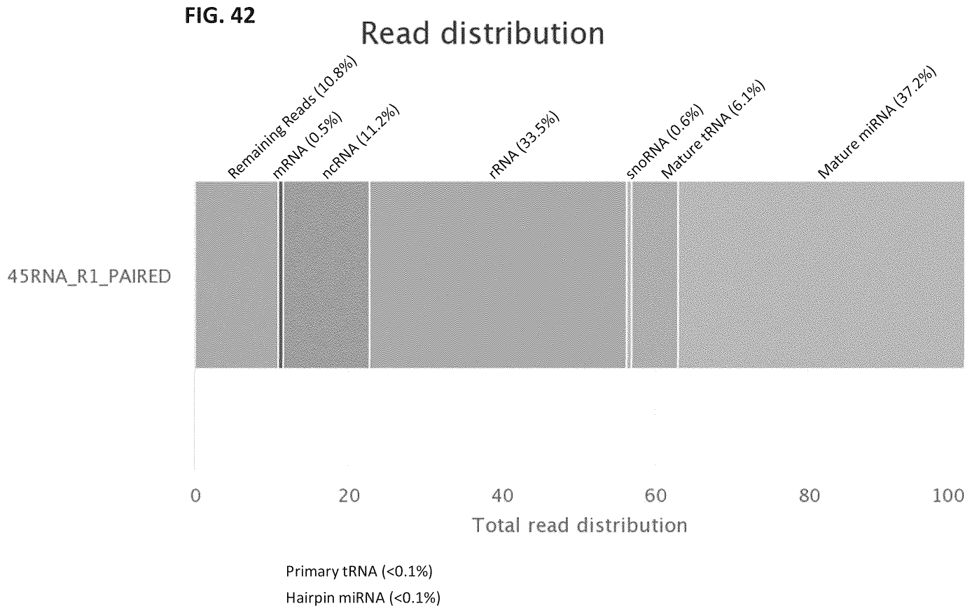

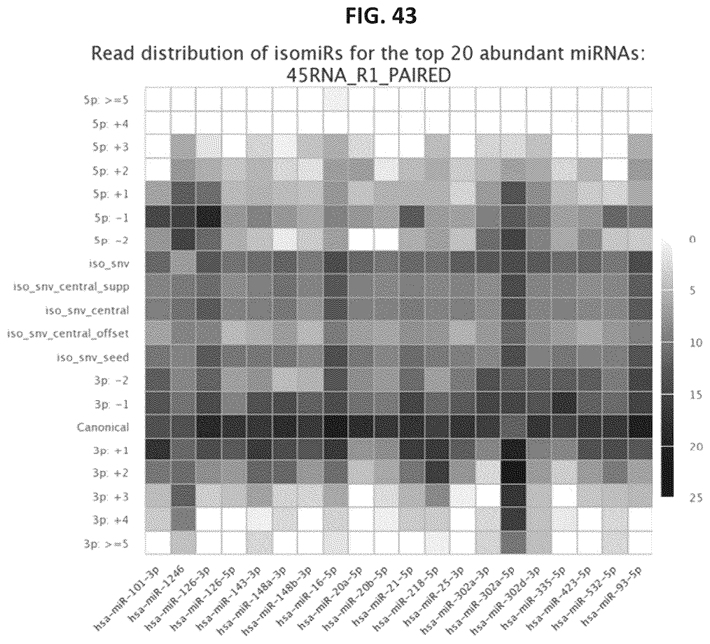

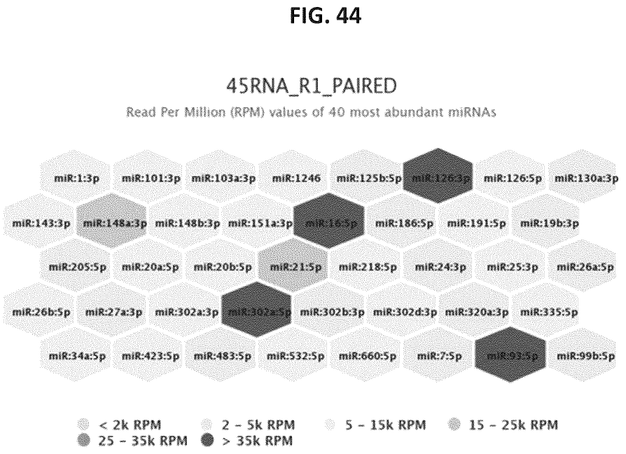

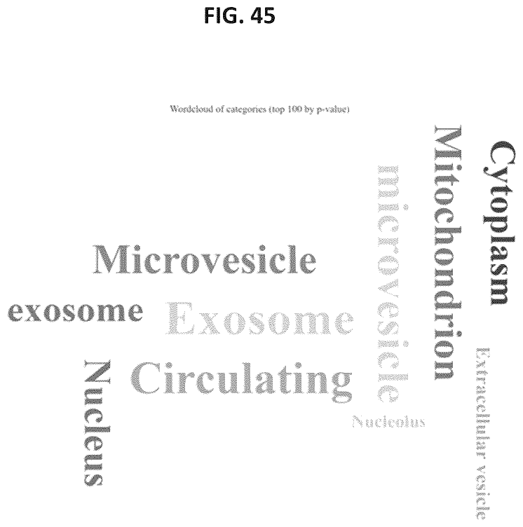

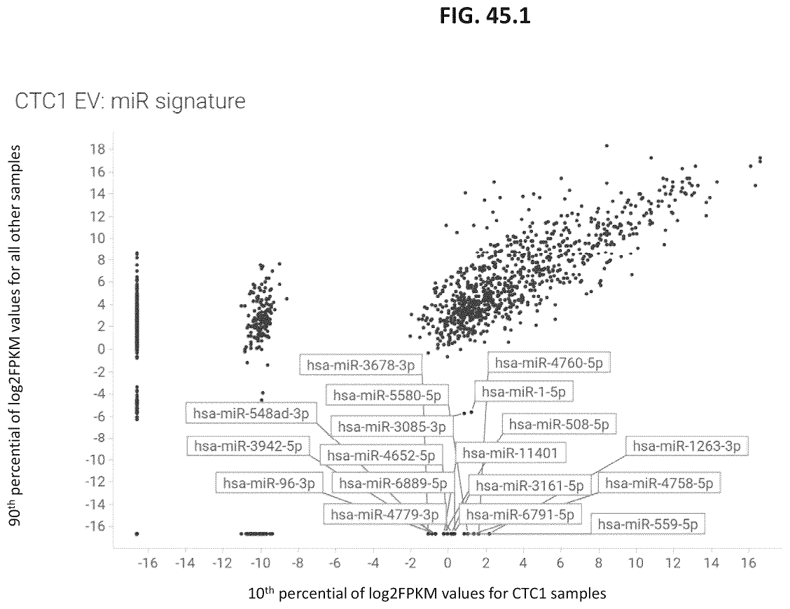

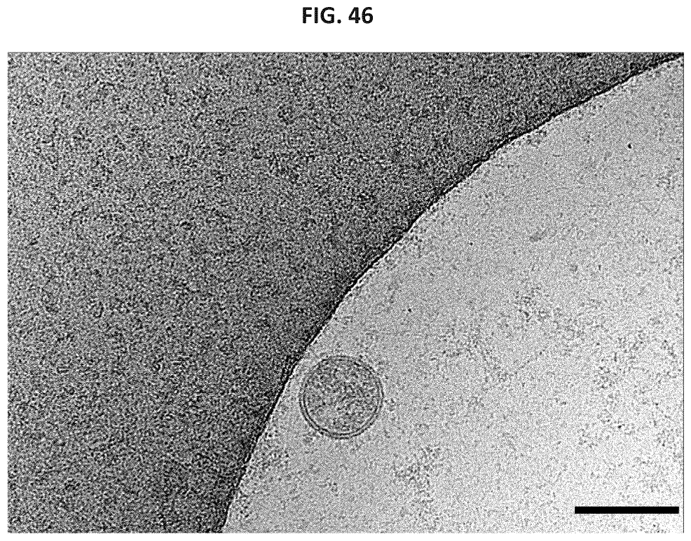

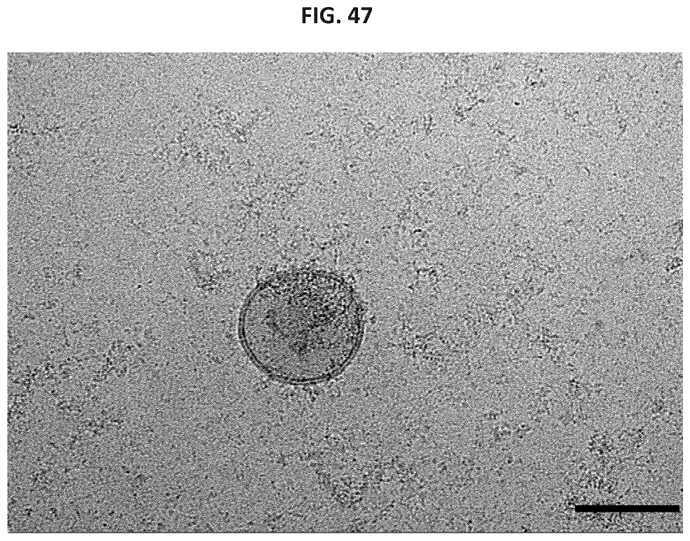

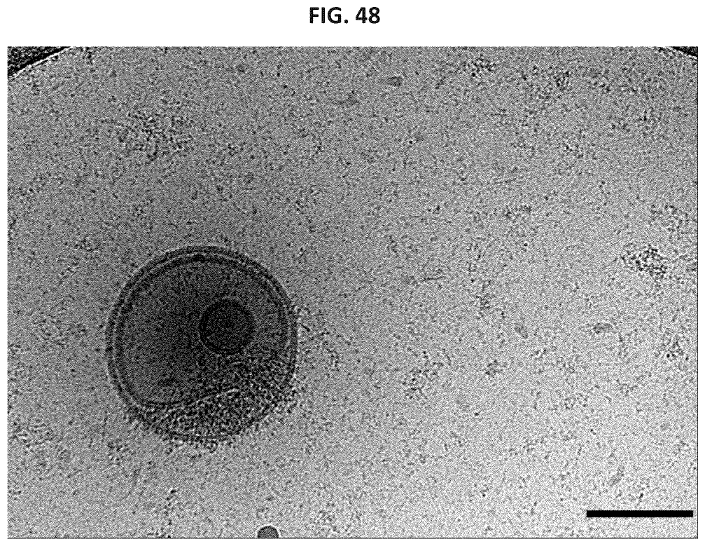

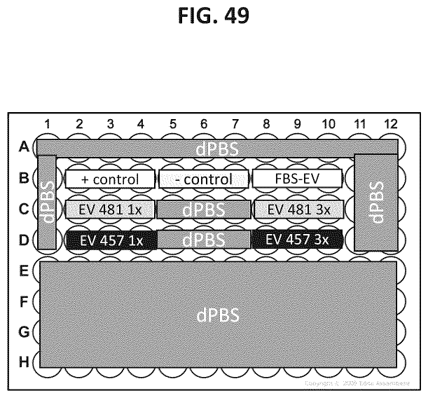

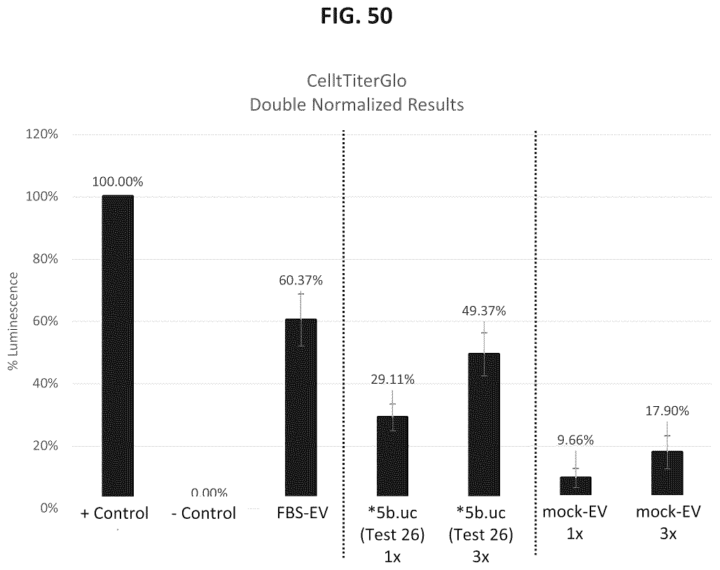

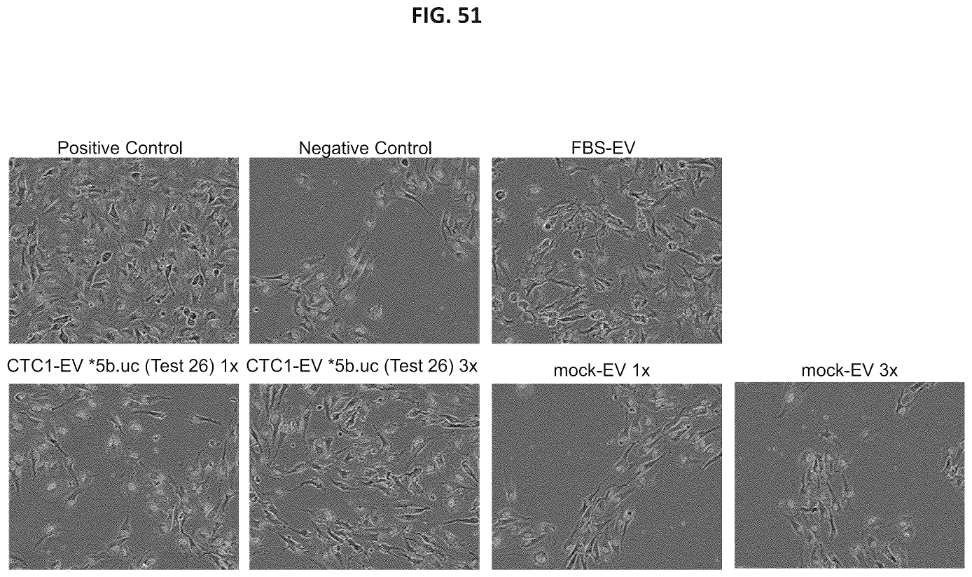

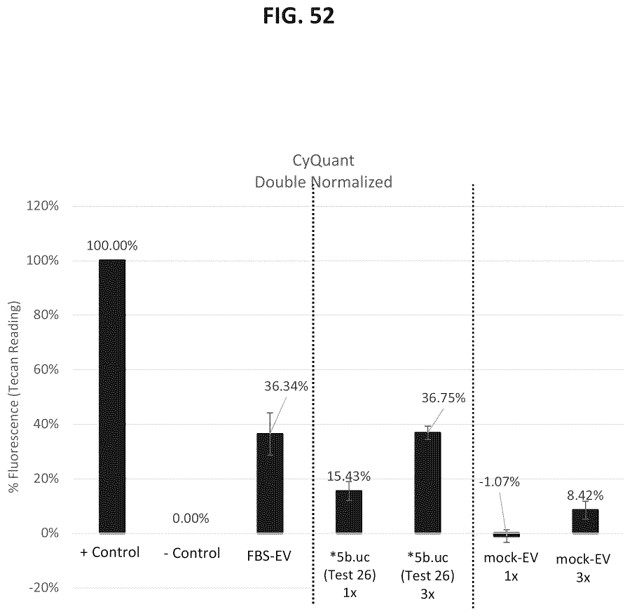

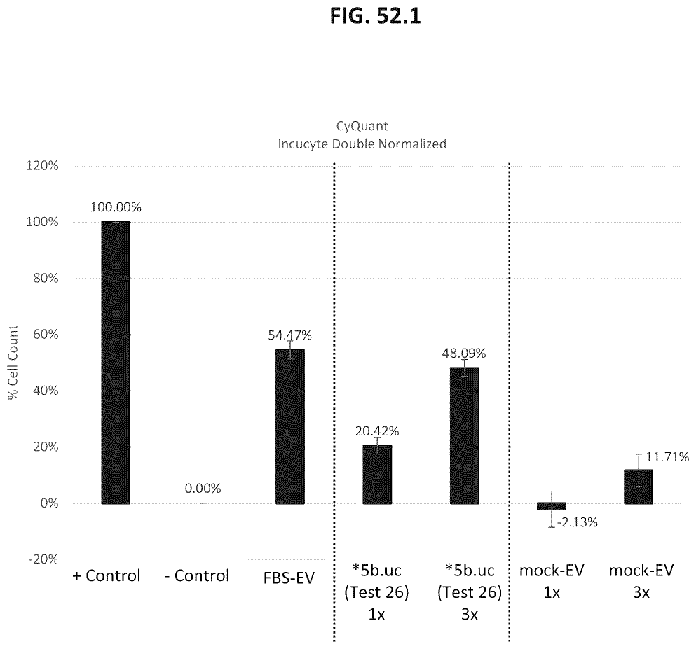

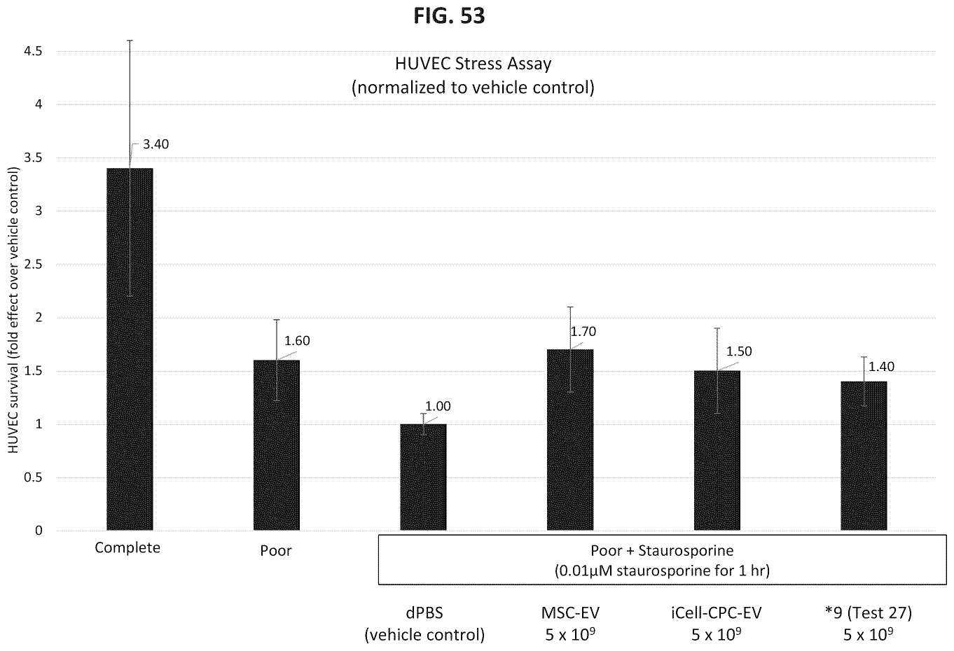

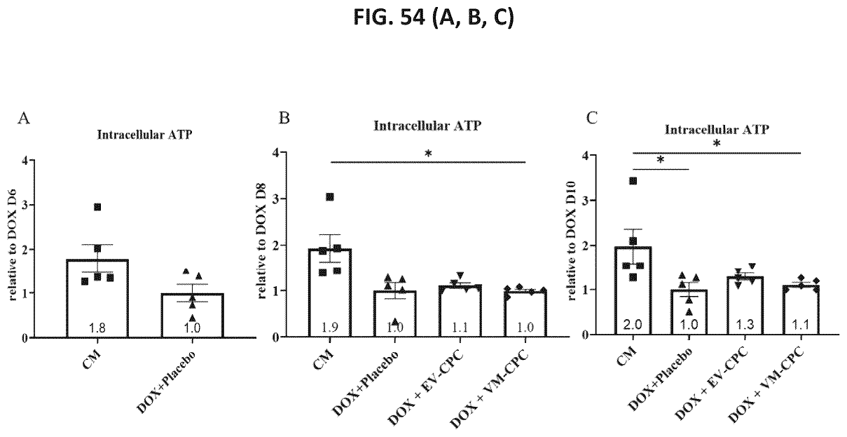

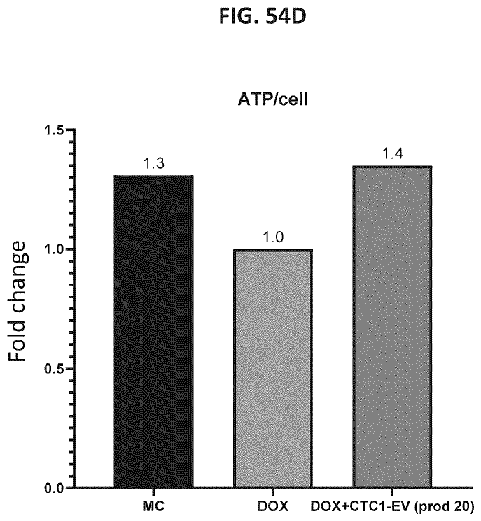

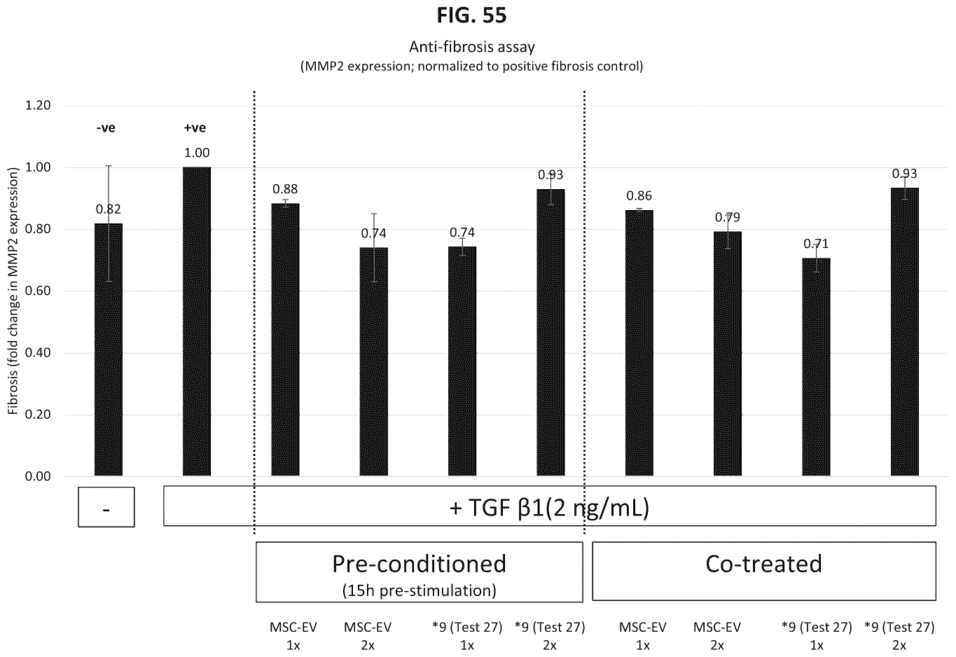

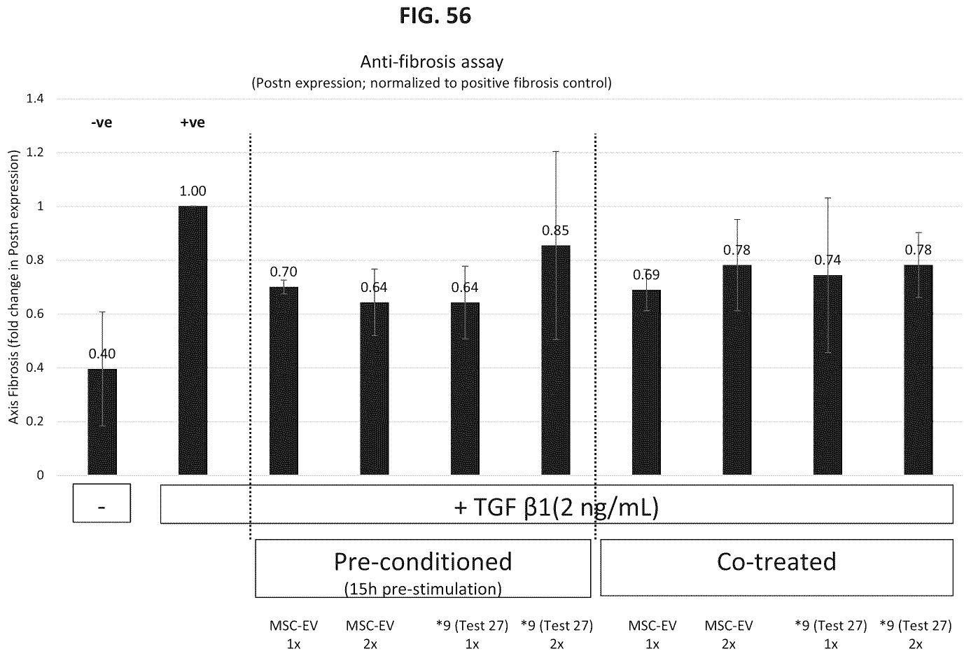

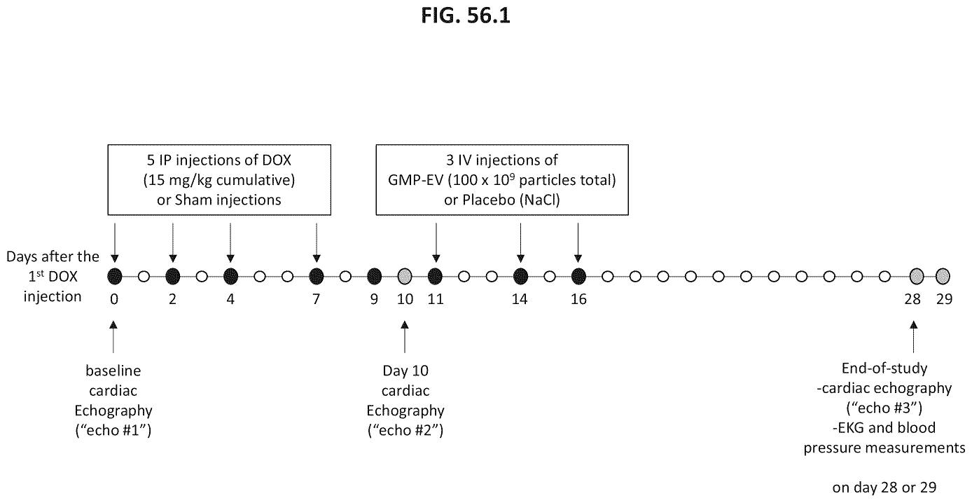

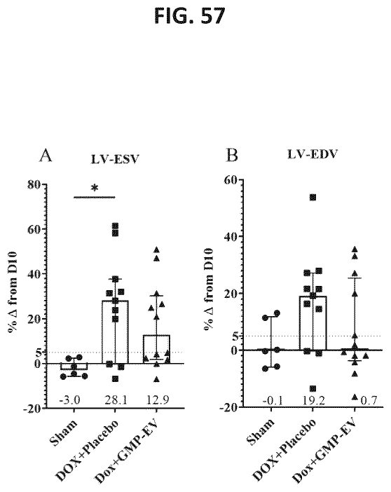

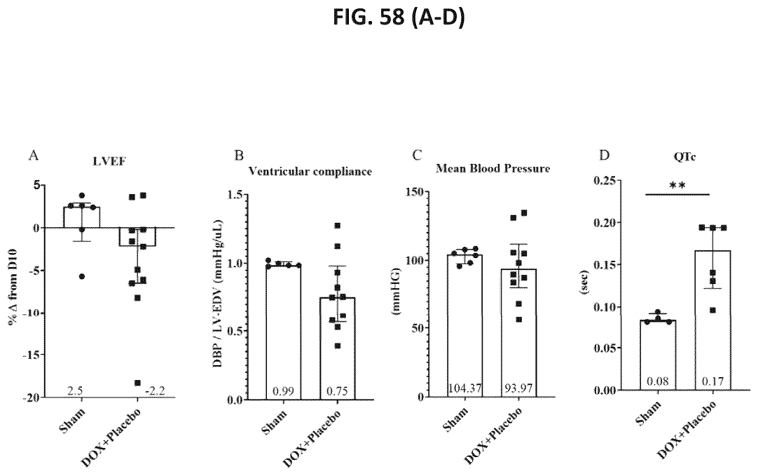

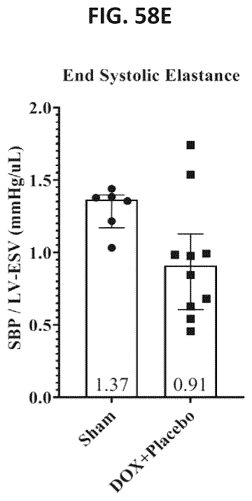

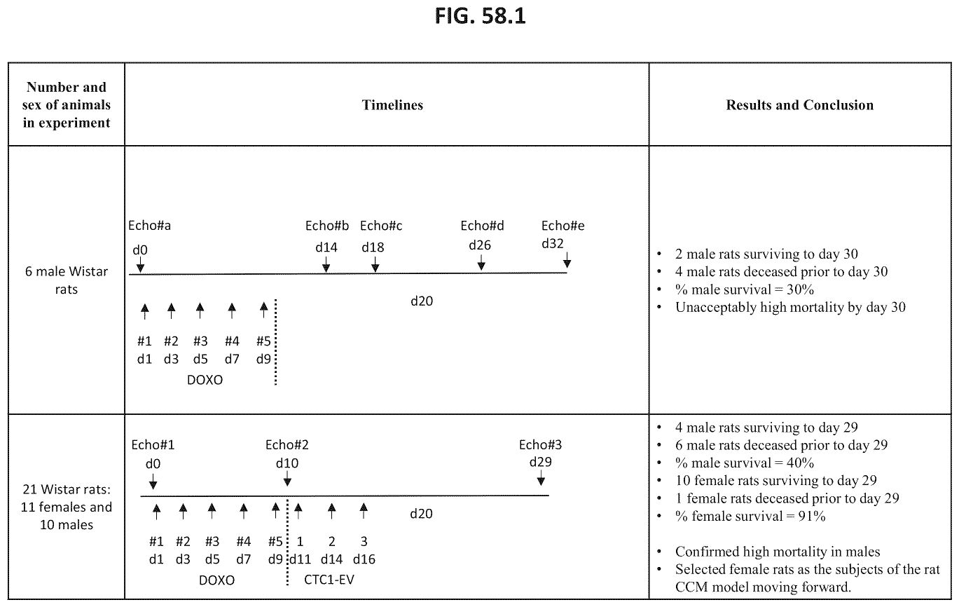

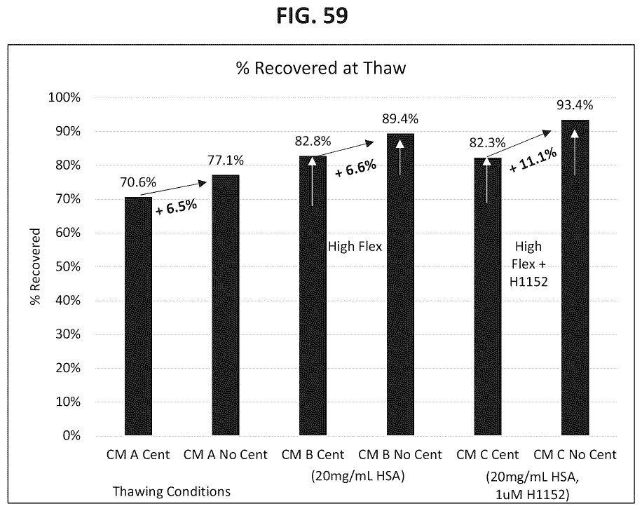

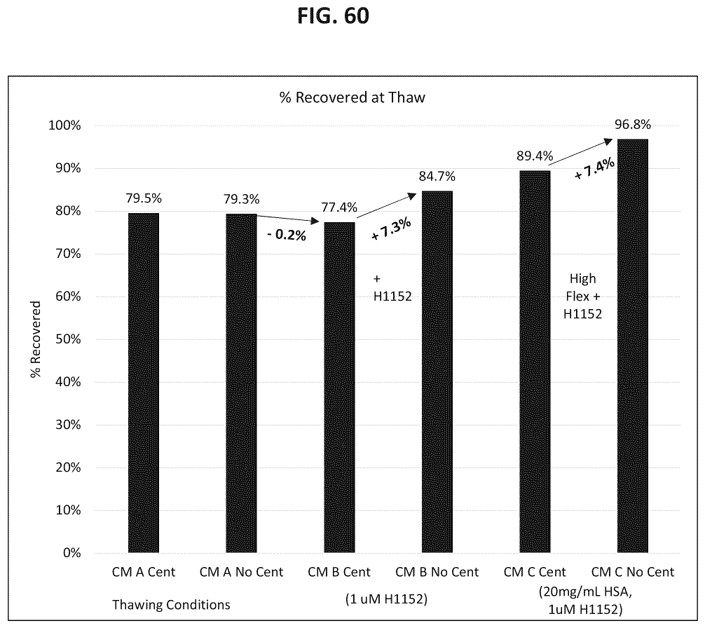

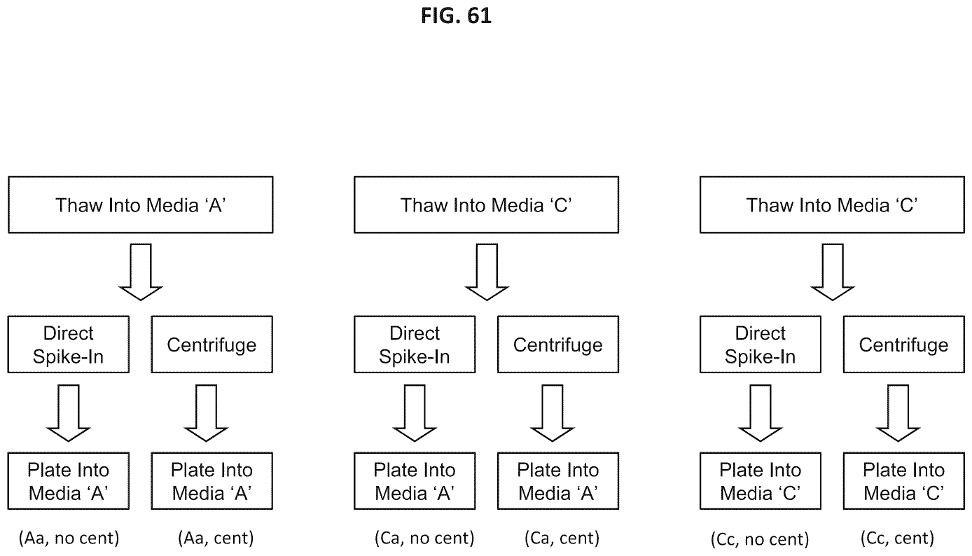

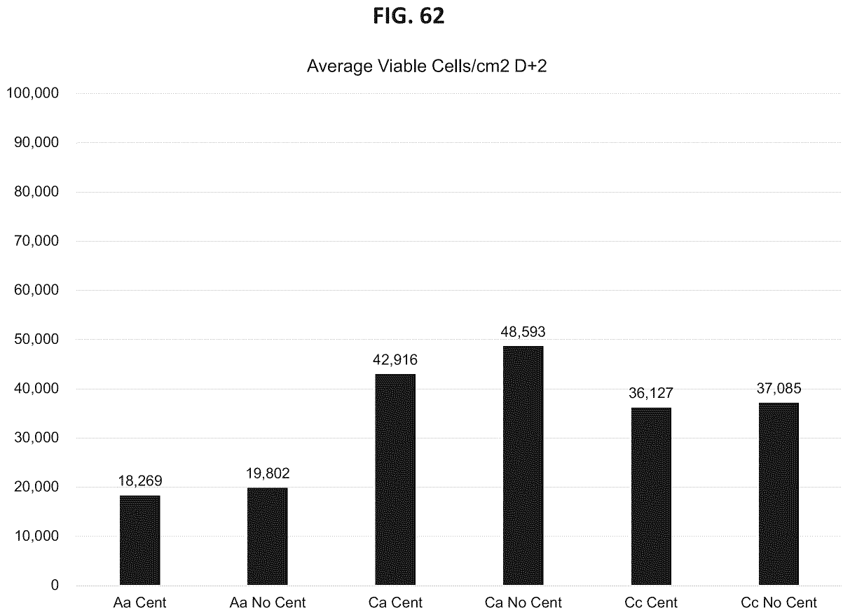

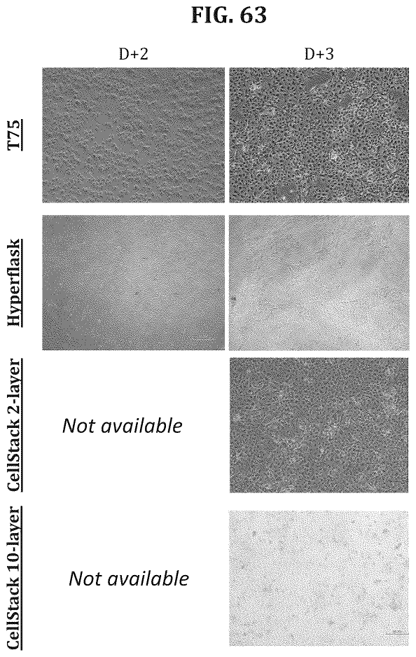

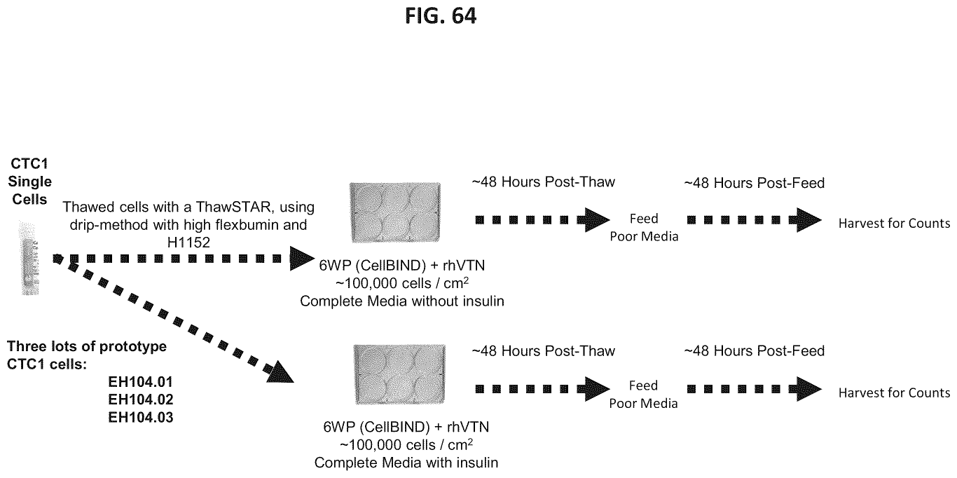

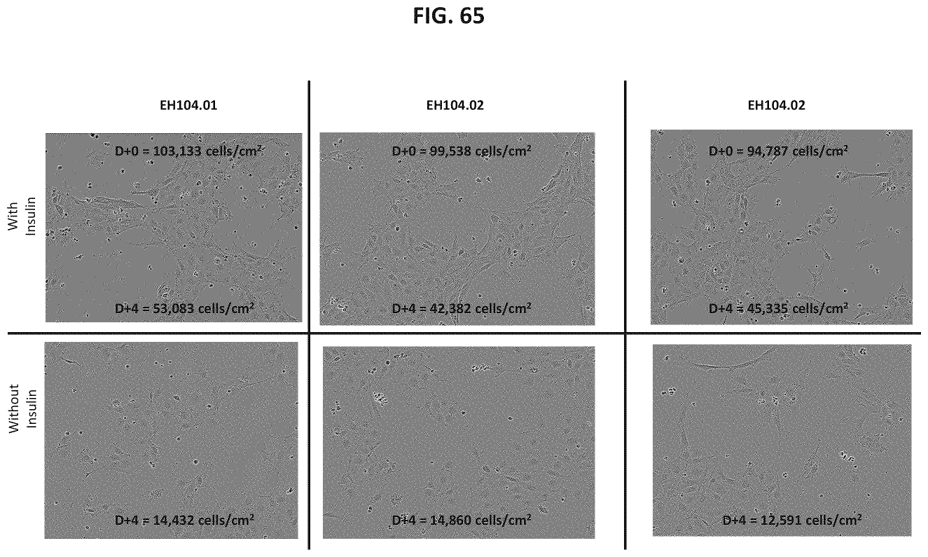

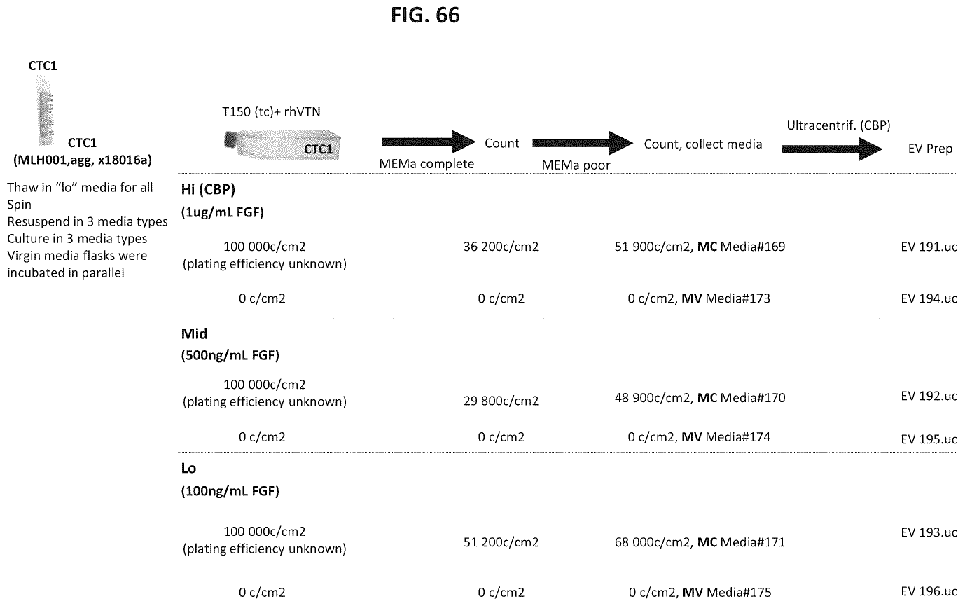

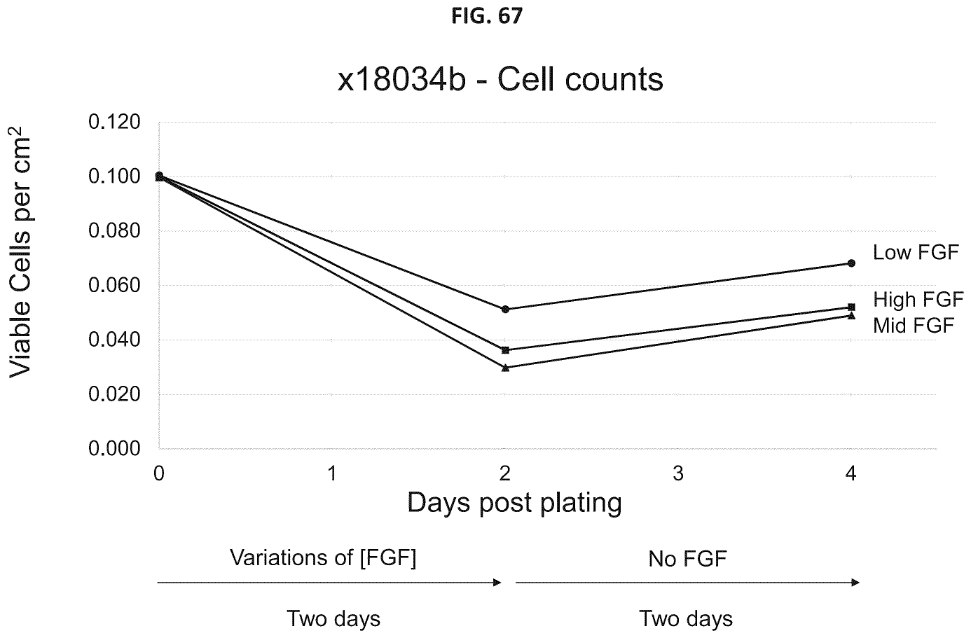

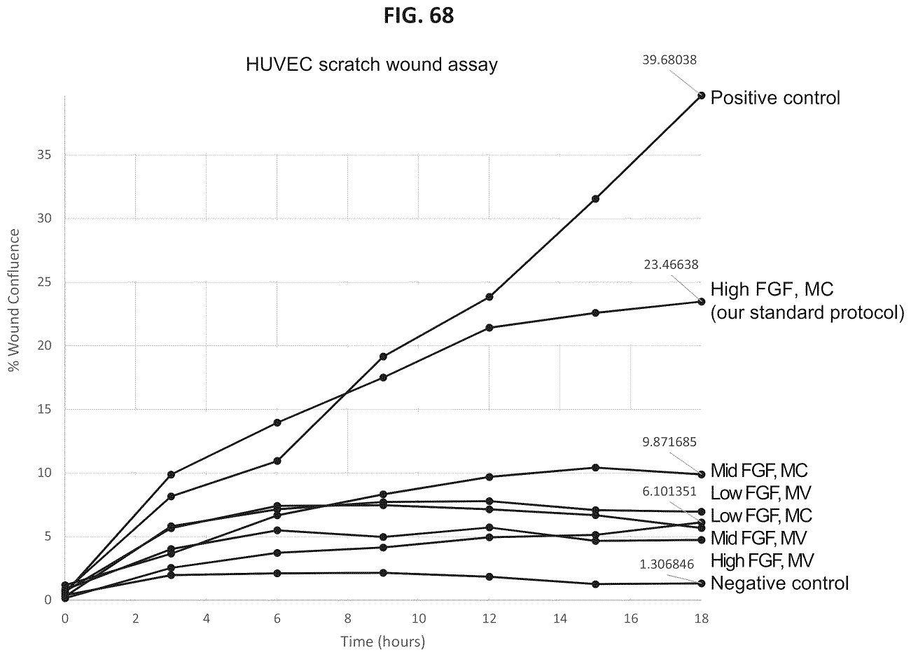

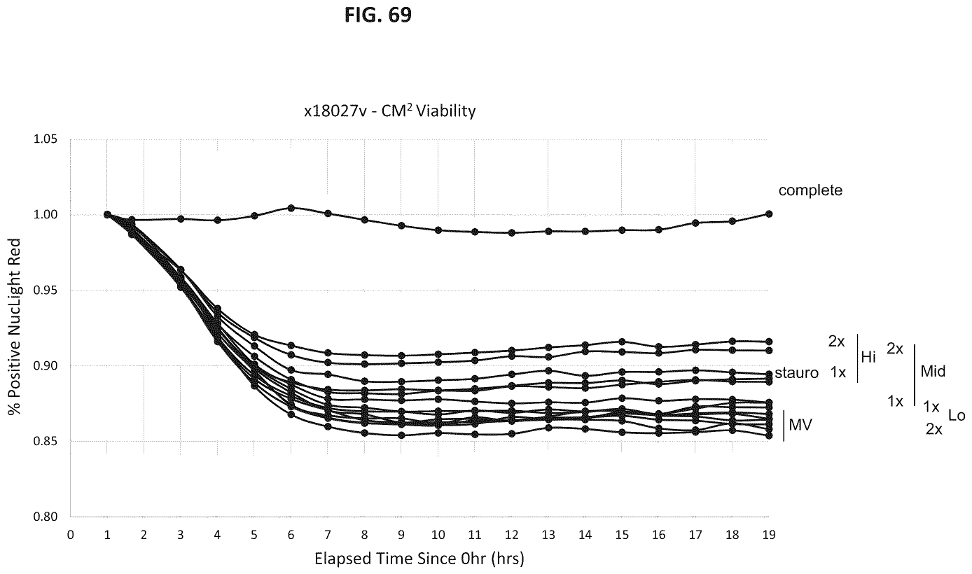

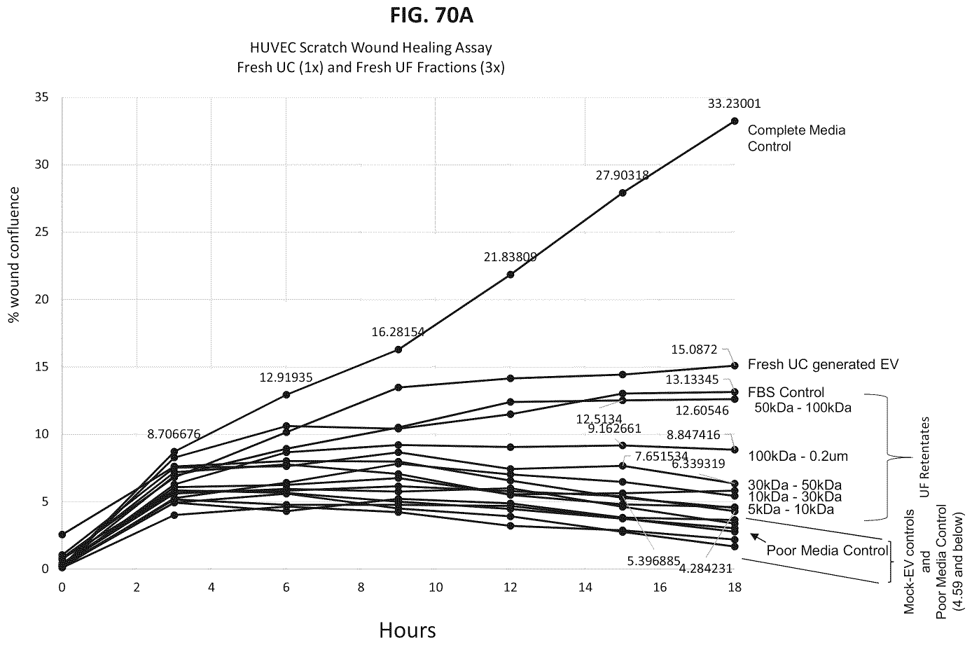

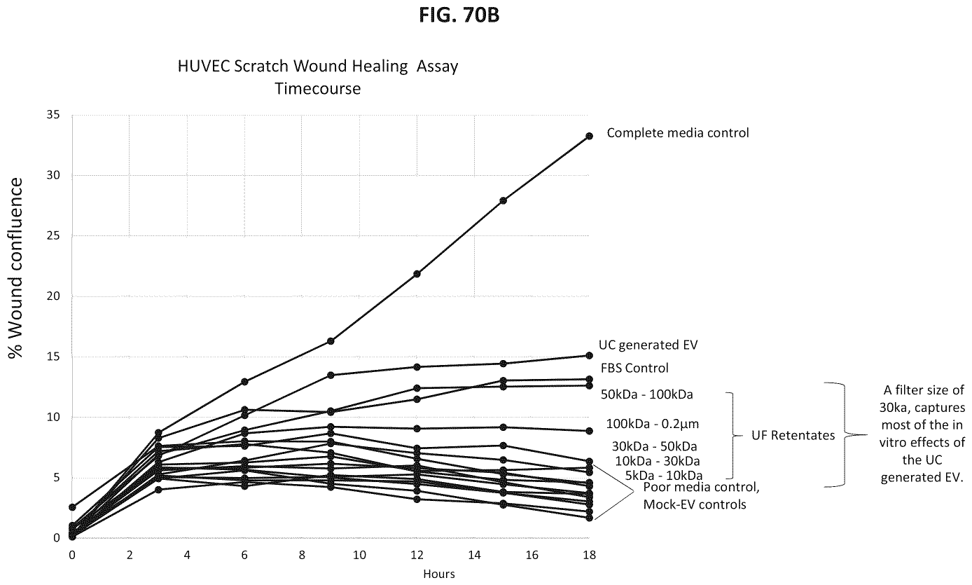

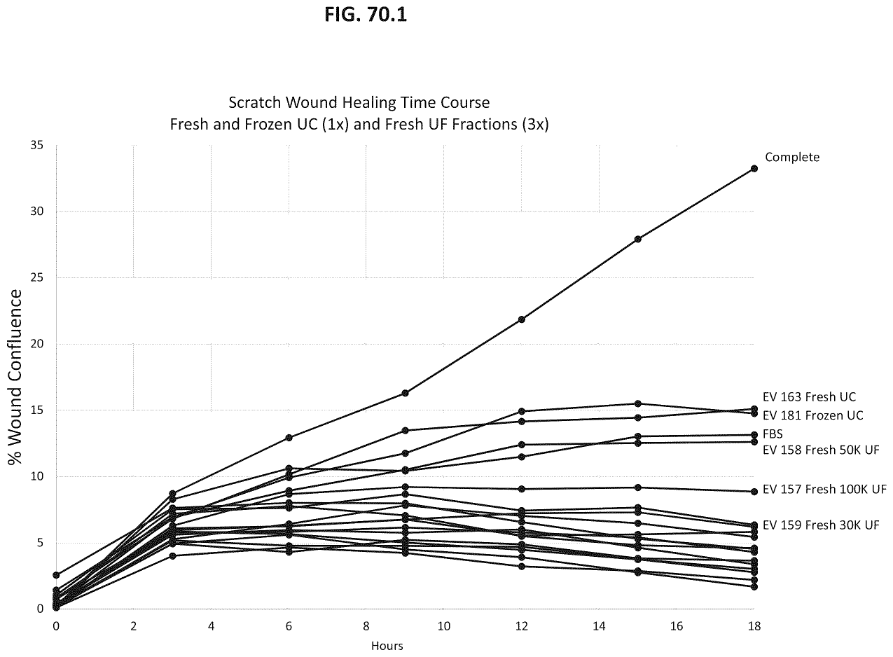

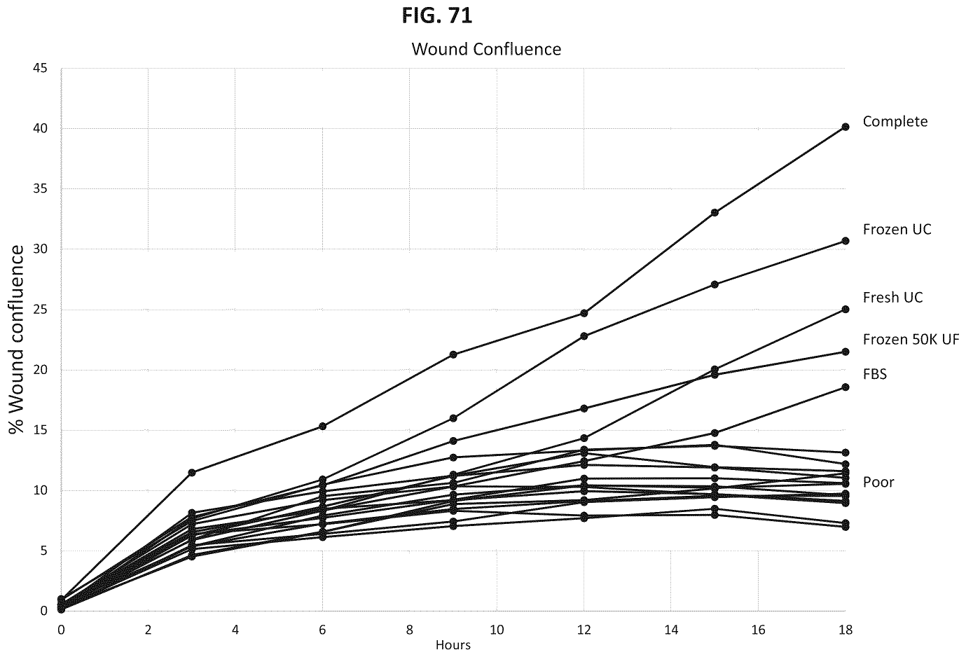

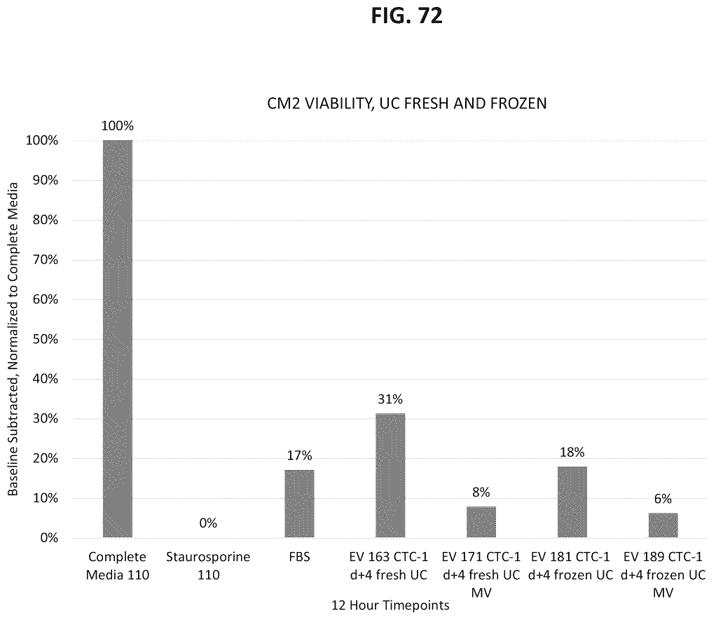

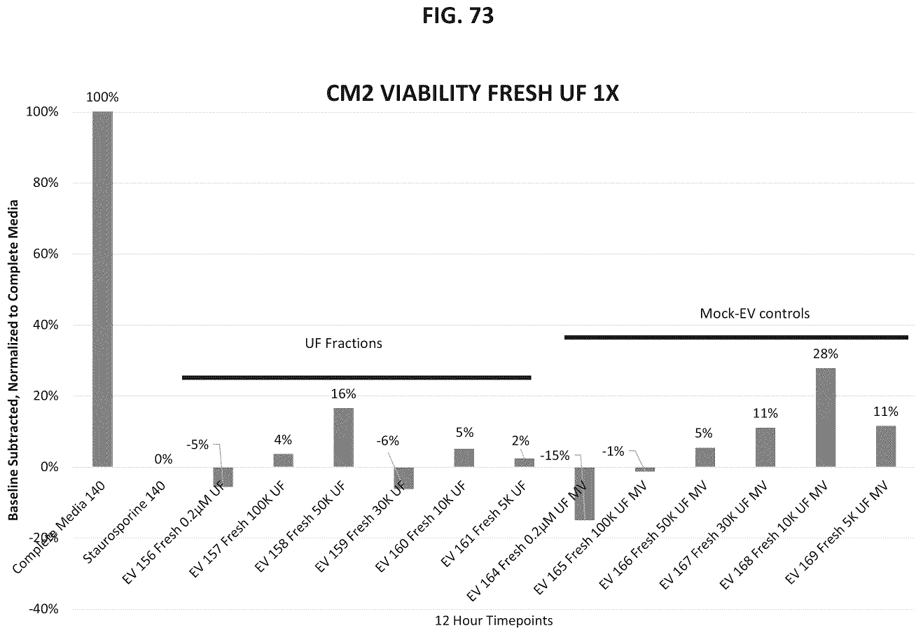

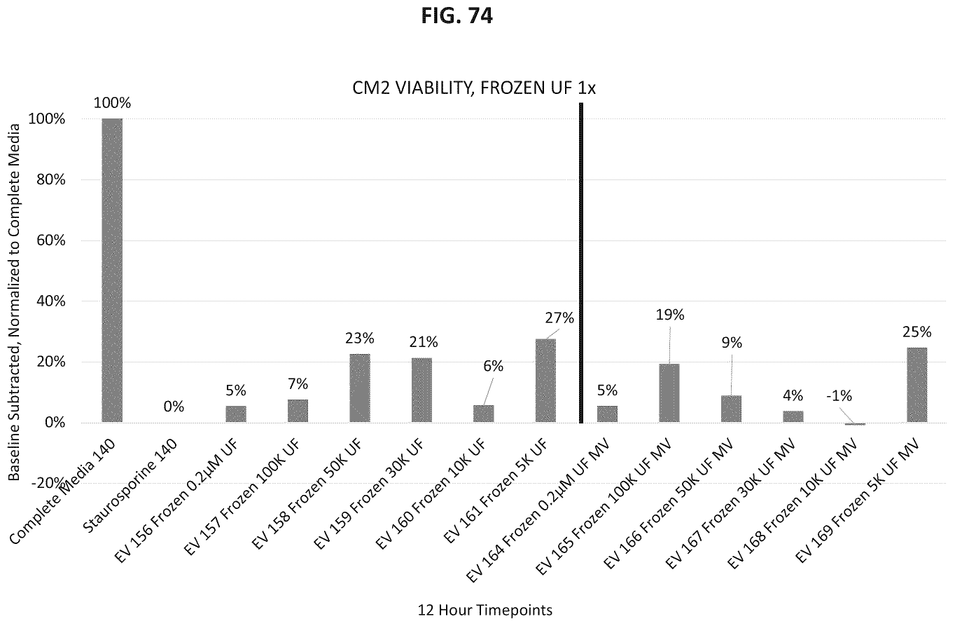

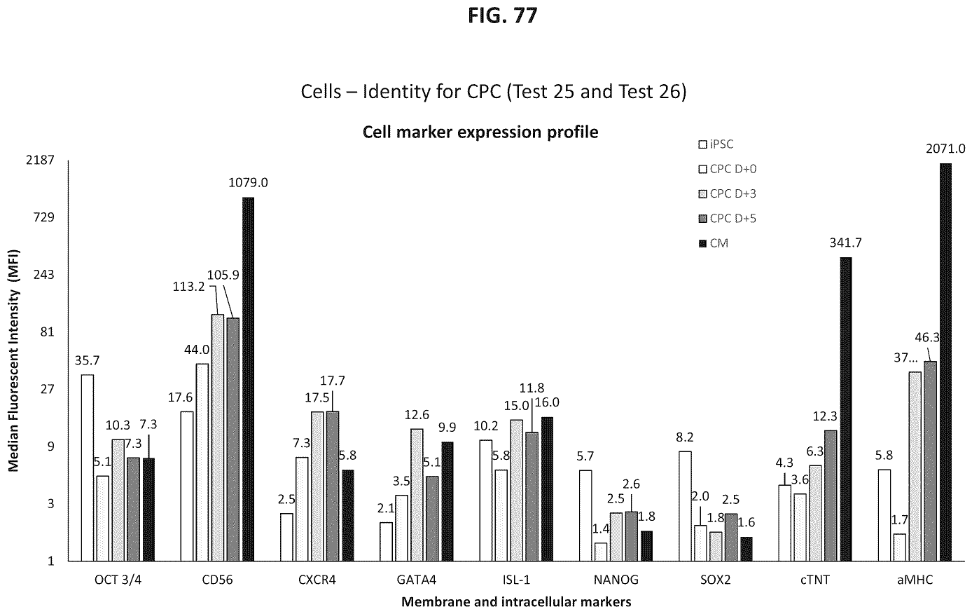

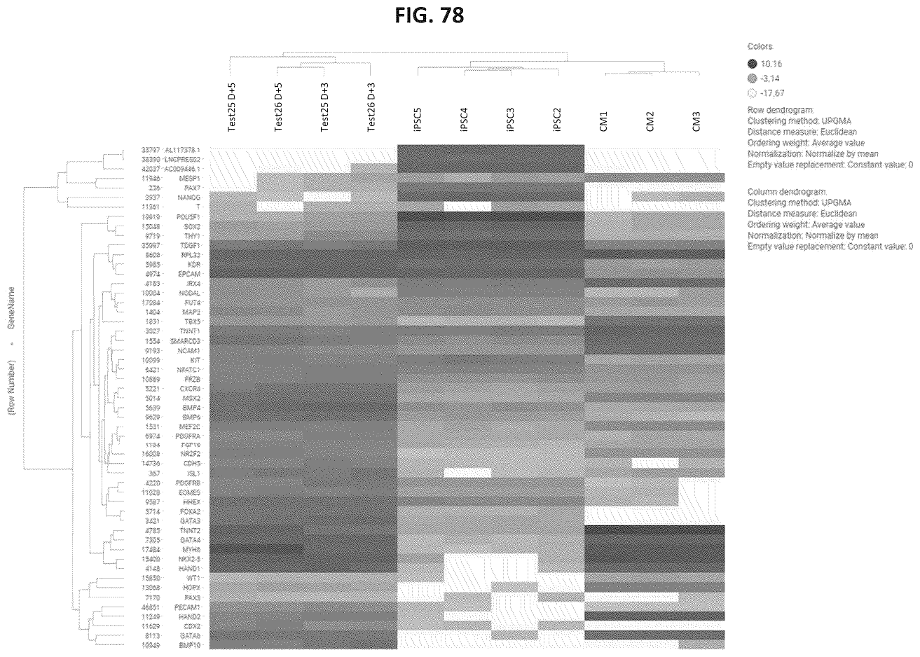

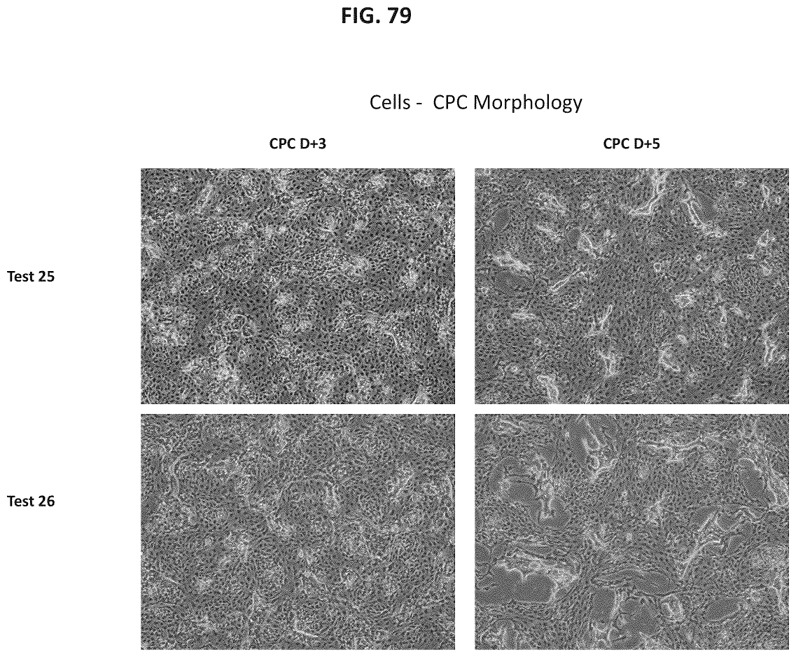

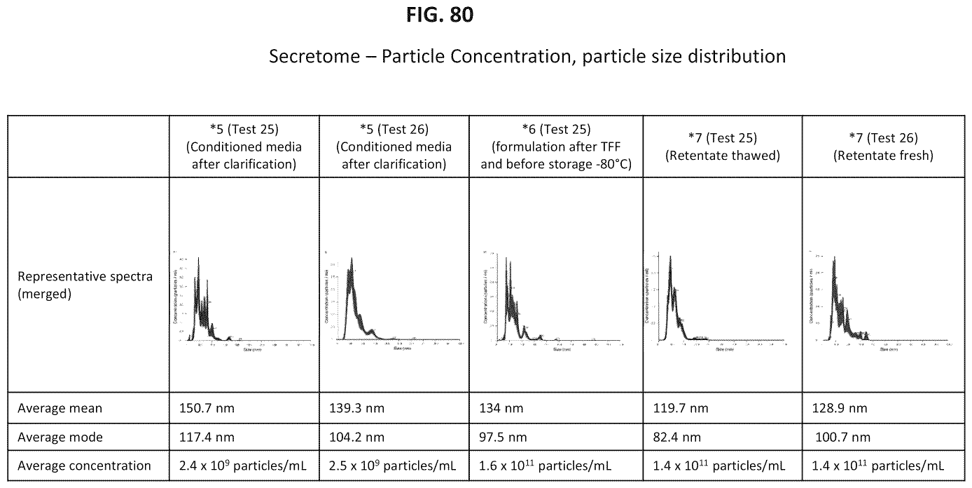

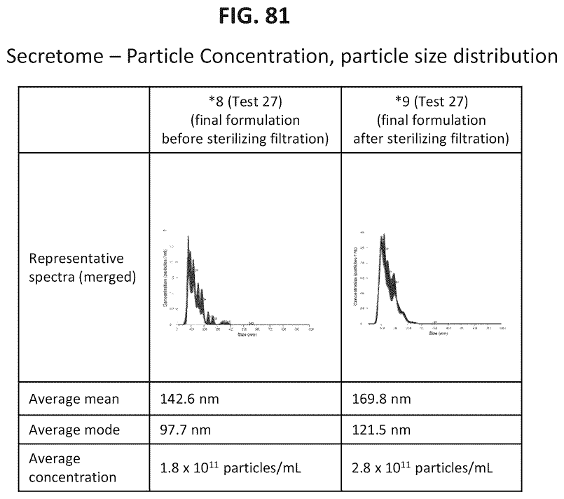

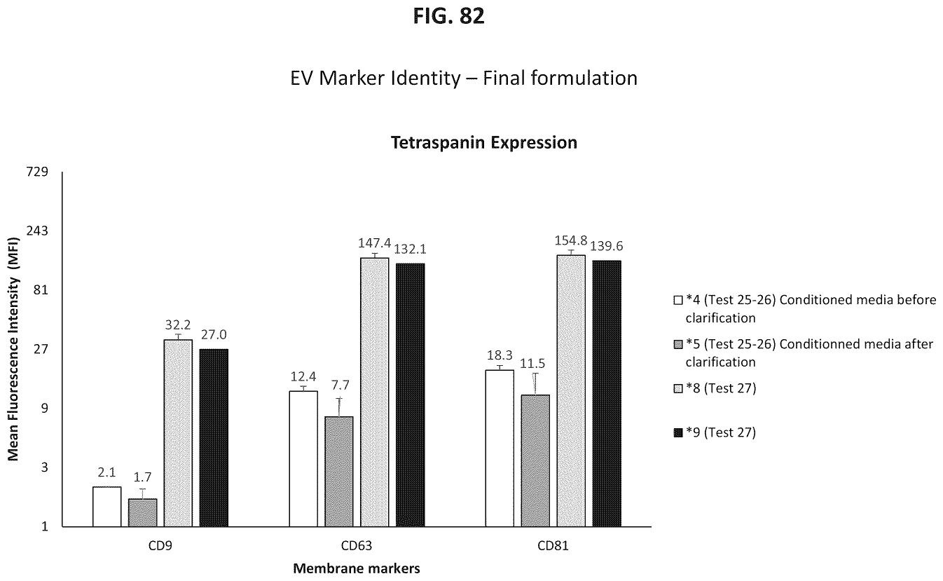

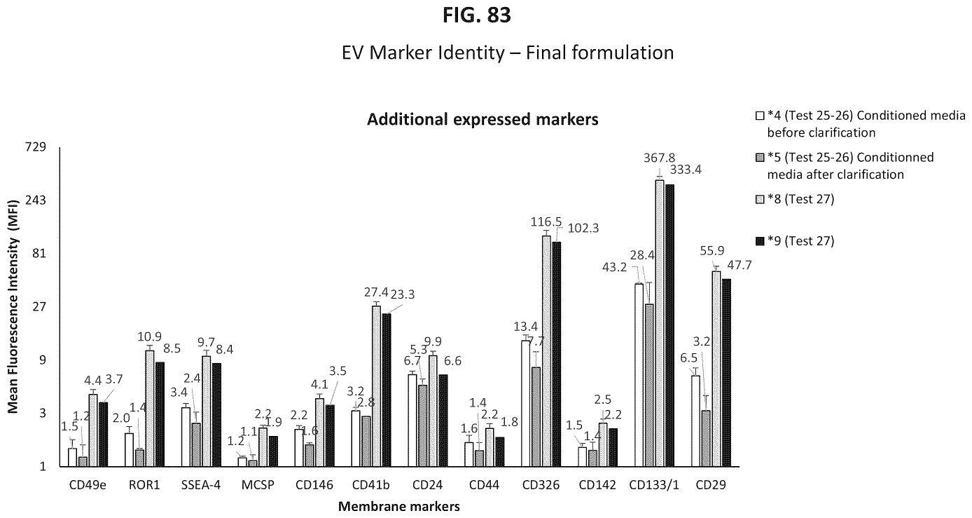

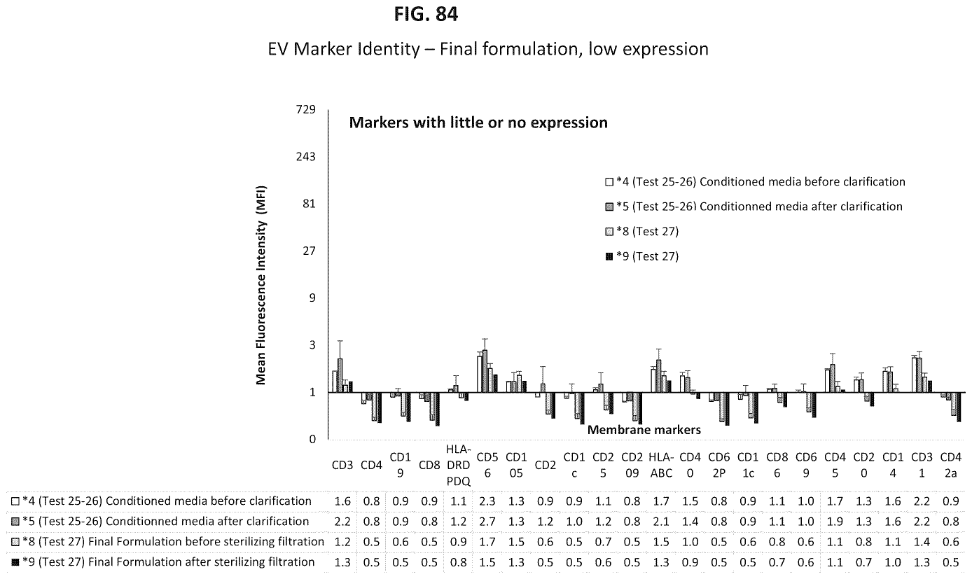

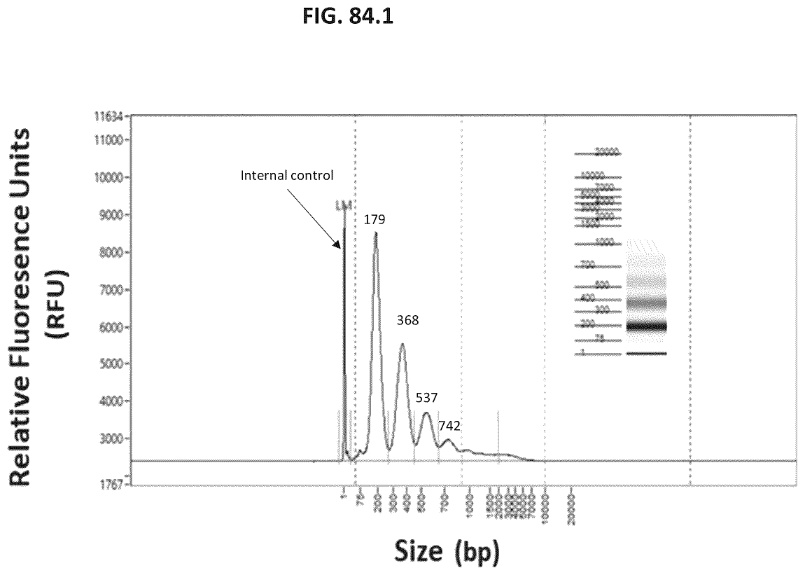

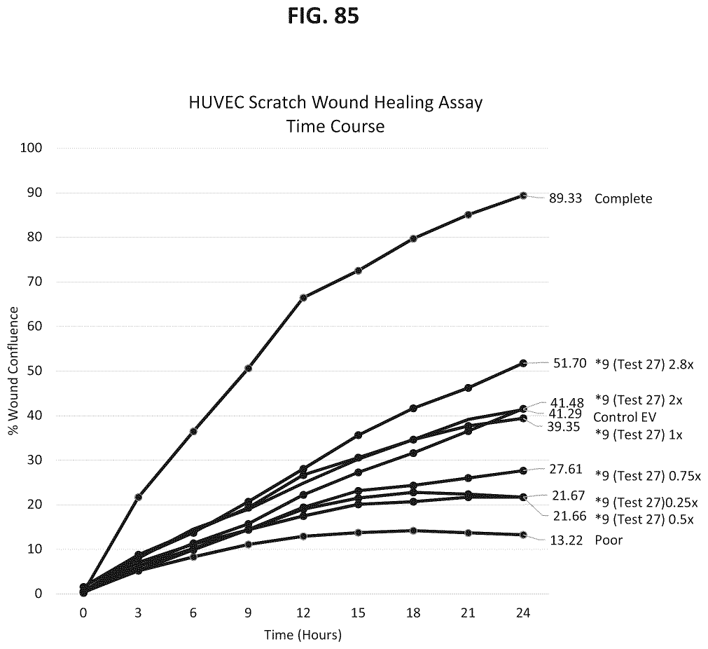

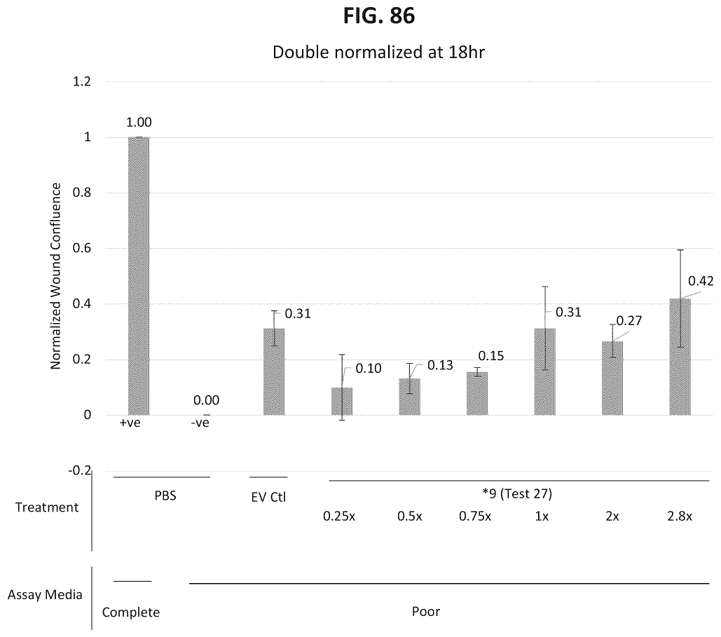

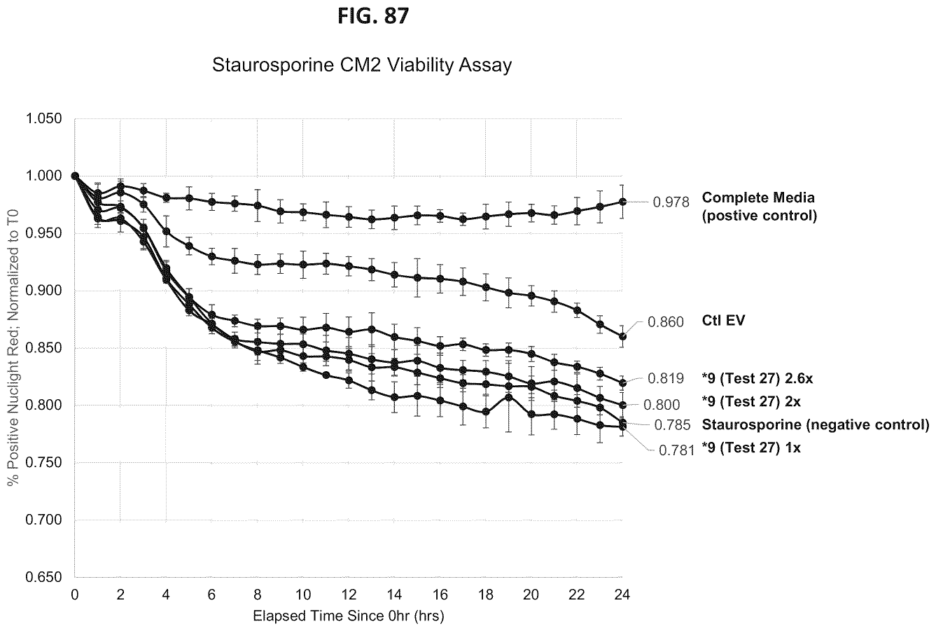

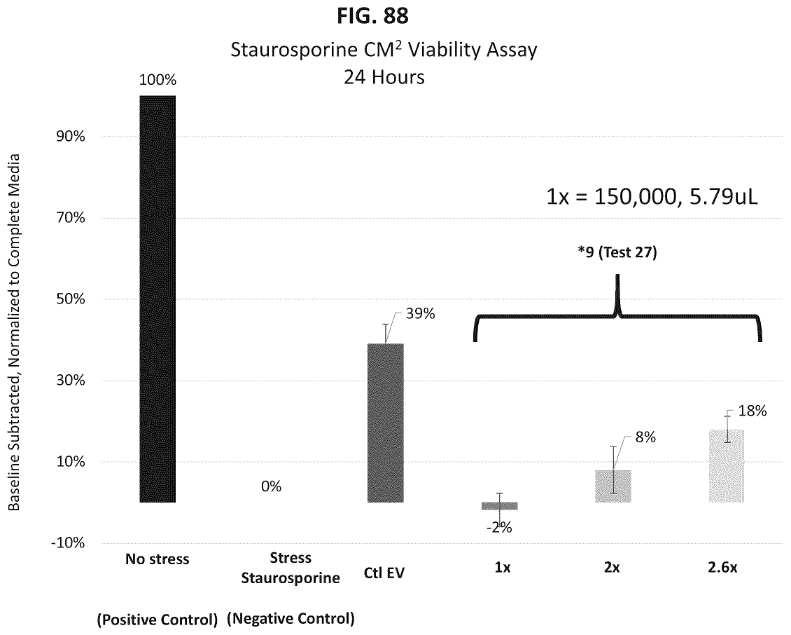

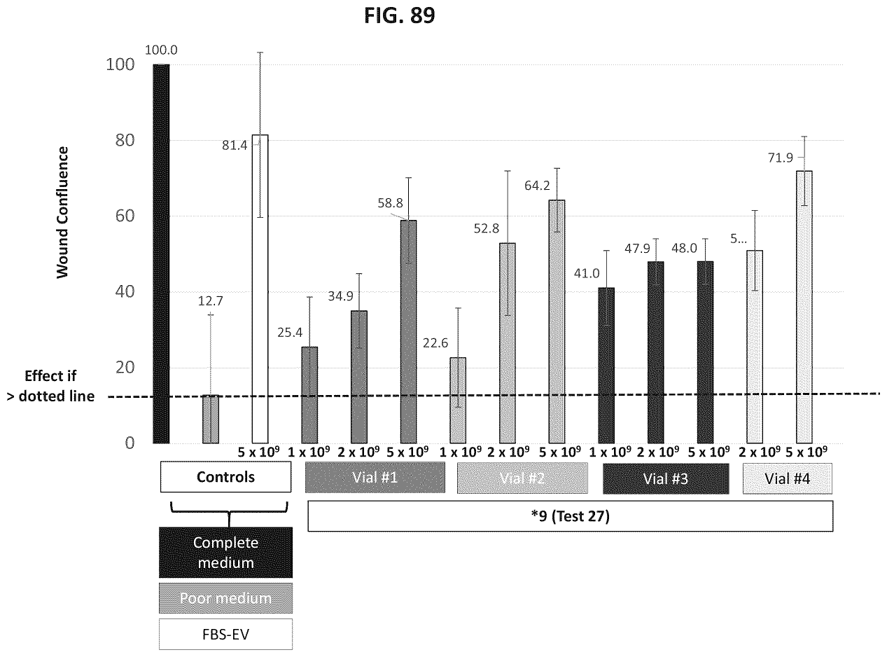

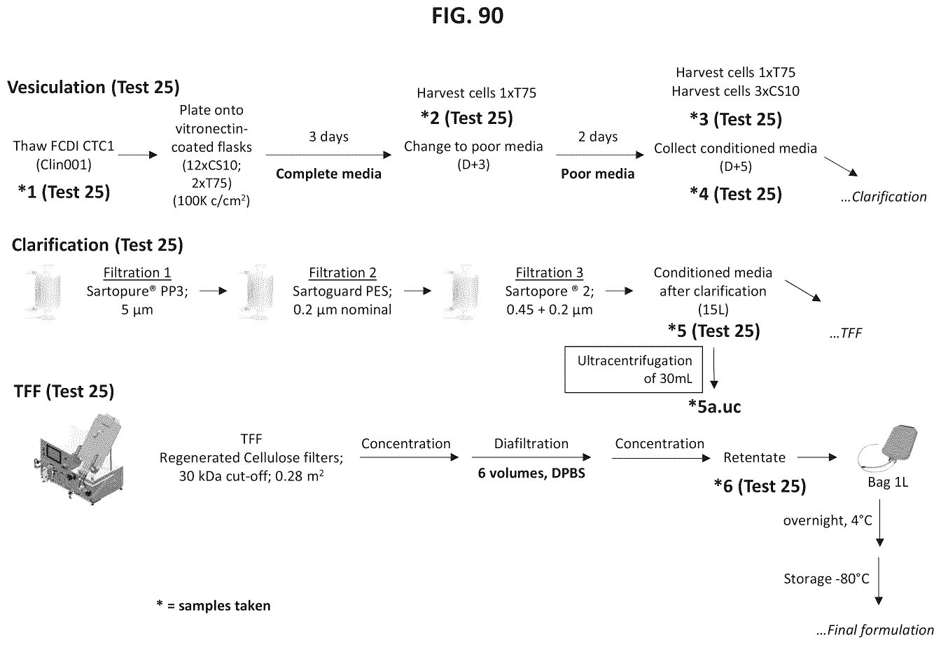

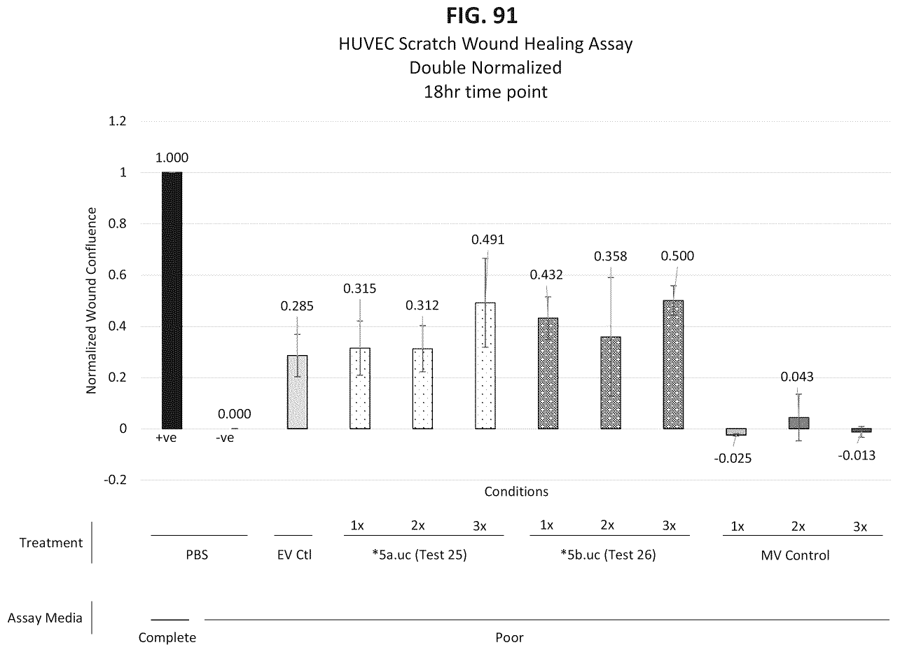

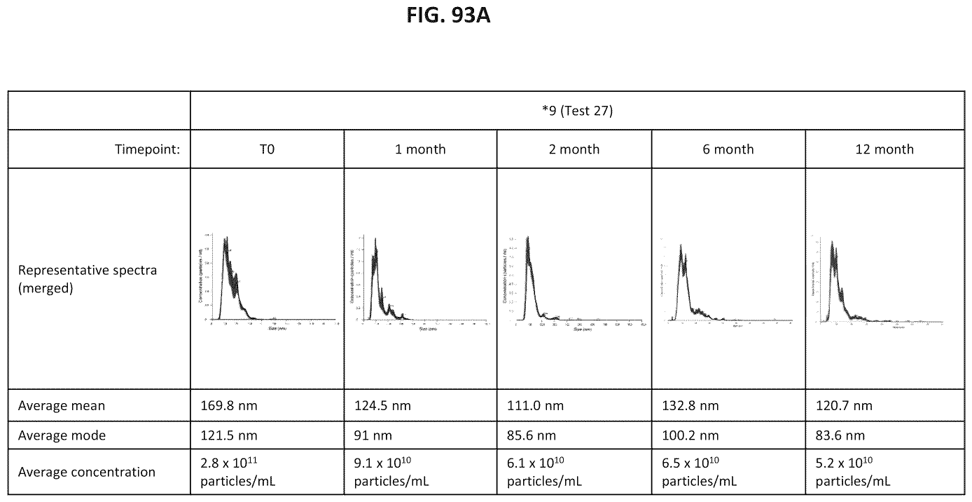

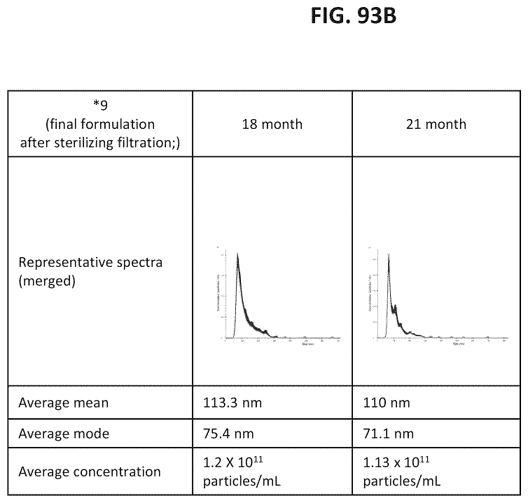

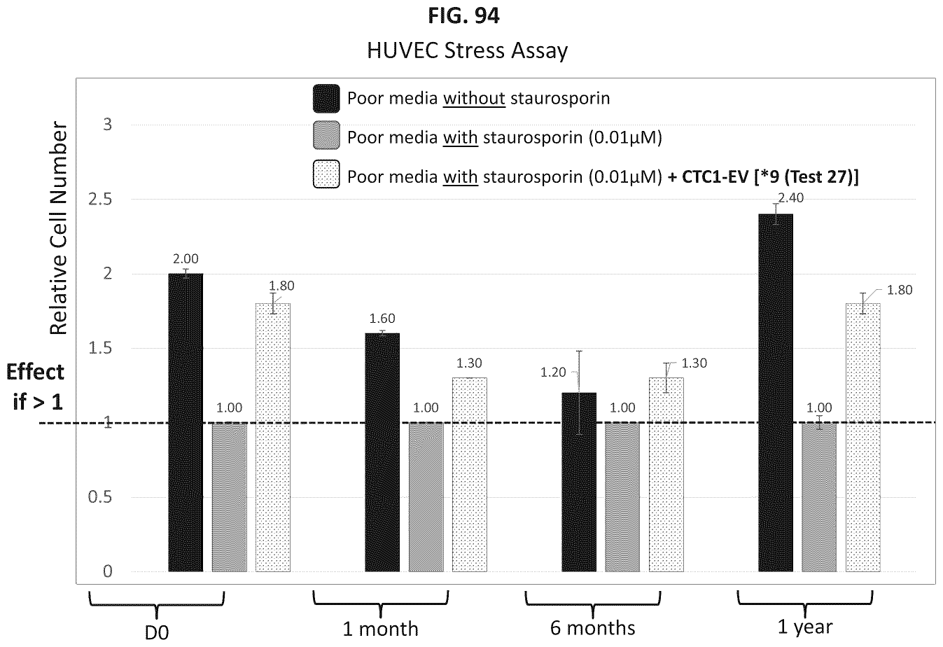

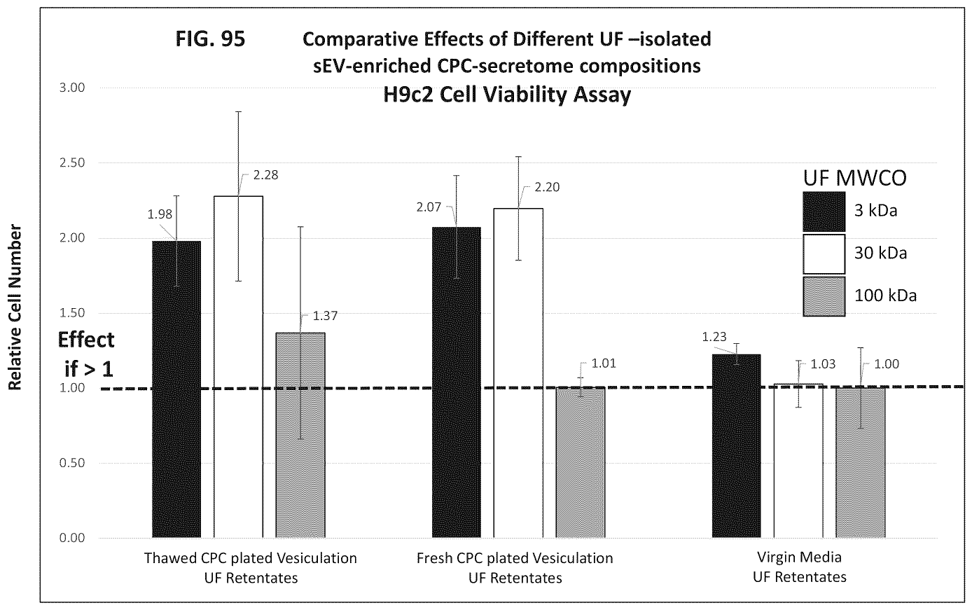

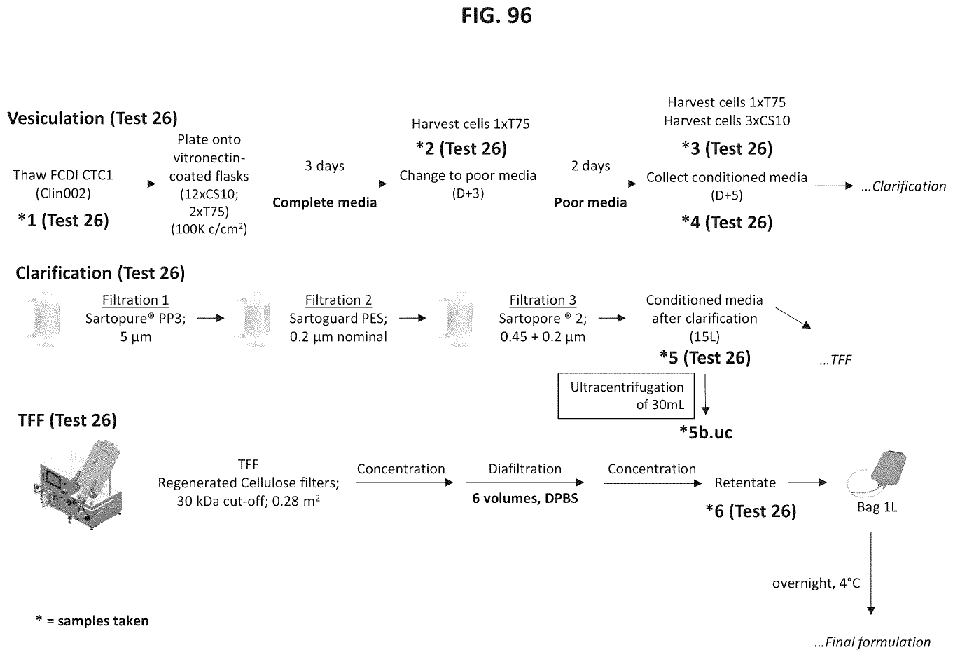

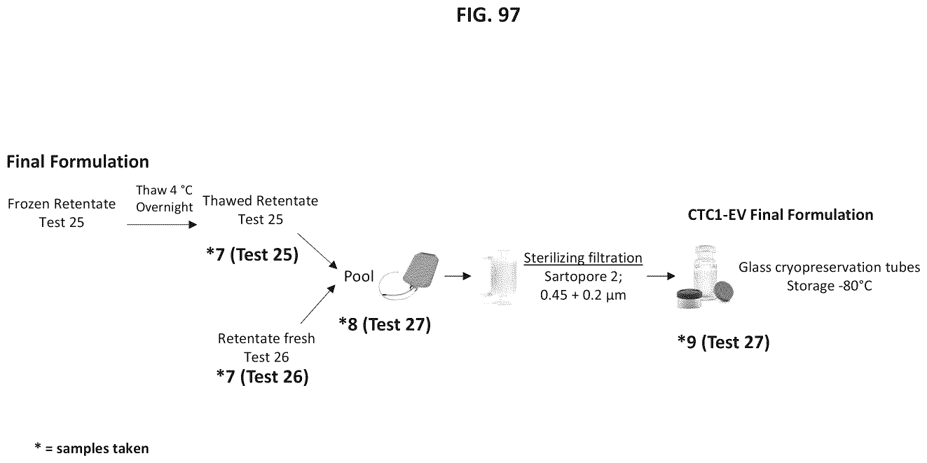

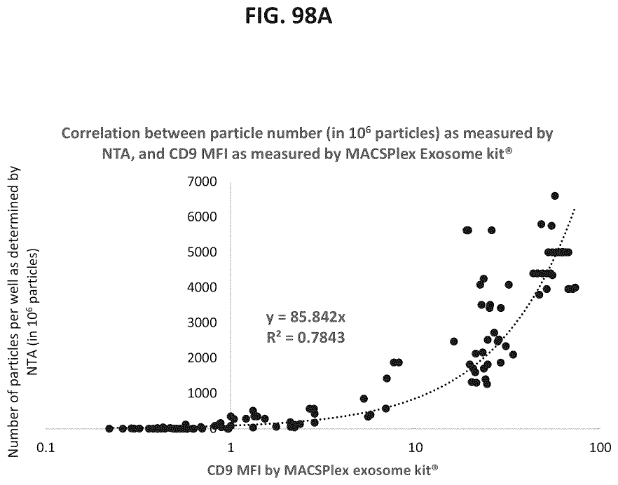

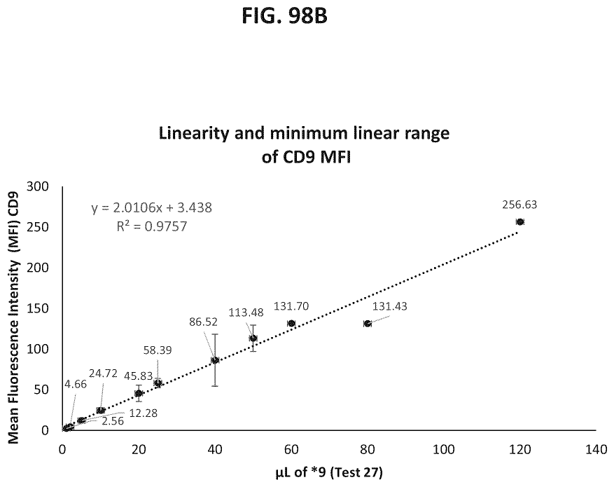

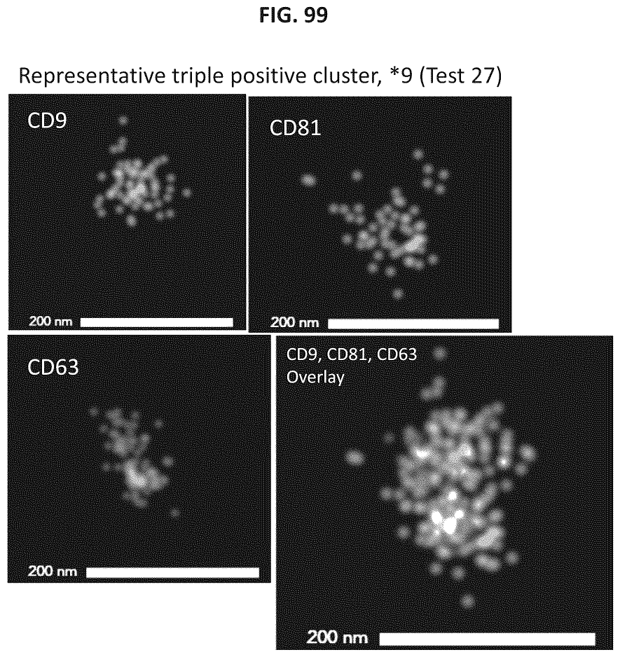

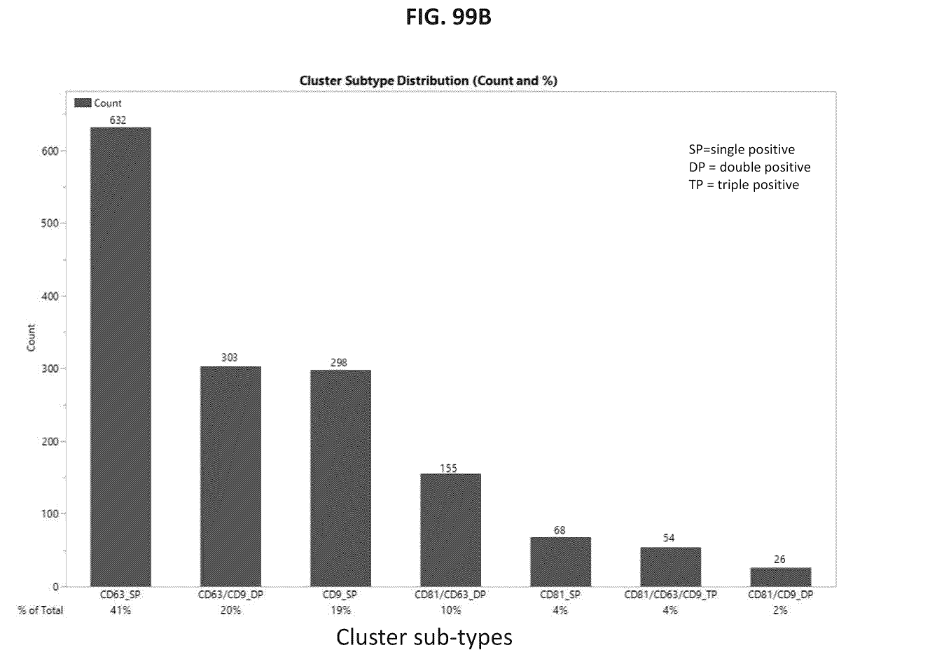

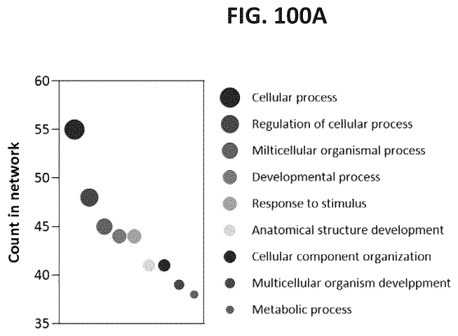

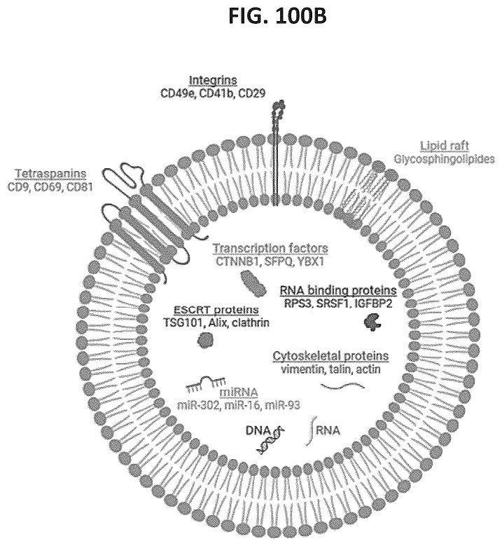

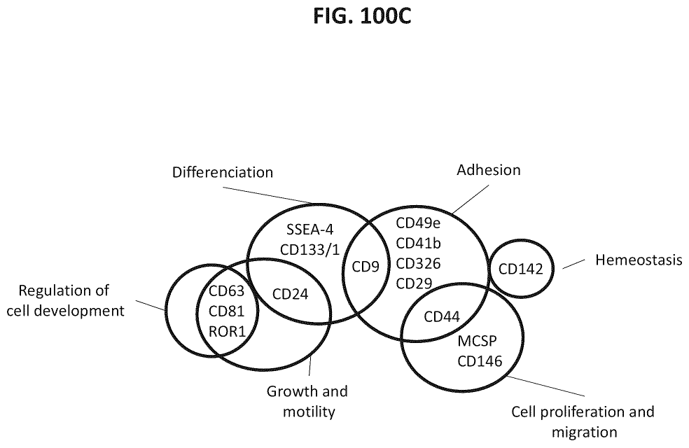

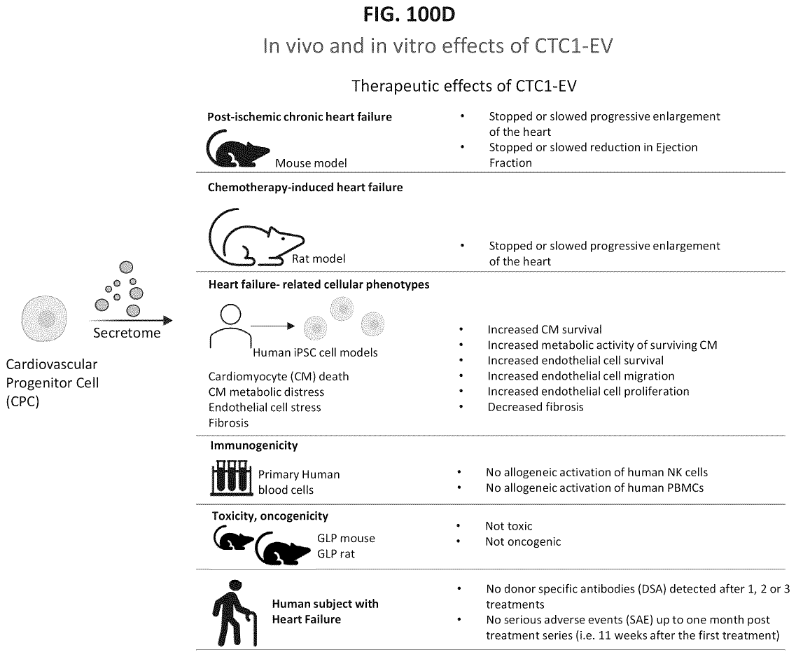

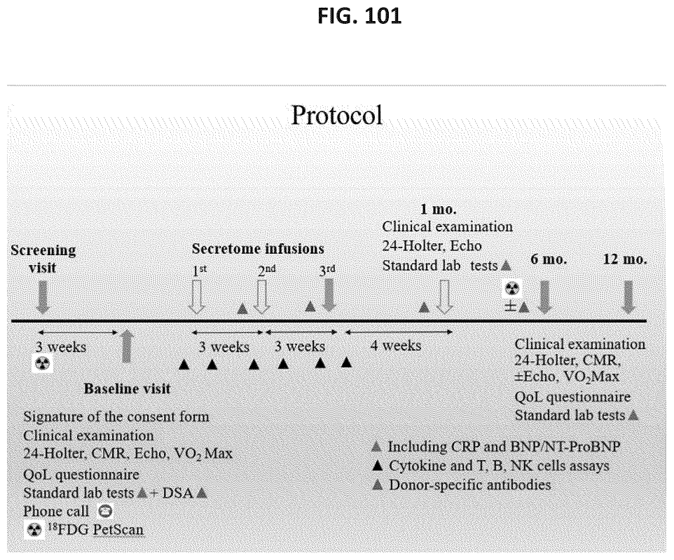

In FIG. 30A, the +ve control result is at 100% (first bar on the left). The −ve control is at 0% (second bar from the left). The EV Ctl is 29.8% (third bar from the left). The Final Formulations *7, sample a (Test 22) and *7, sample b (Test 22) gave similar results. Both materials improved scratch wound healing, with indications of a dose-response from the doses ranging from 0.25× to 2.6×. The lowest dose tested, which was 0.25× gave at least a 17% increase in wound healing capacity over the −ve control for both samples. At a dose of 2.6×, both samples improved scratch wound healing by greater than 25% over the negative control. The *7, sample a (Test 22) improved scratch wound healing by 35.6% at the 2.6× dose. Taken together, the data here indicate that both Sterivex-GP, 0.22 μm filtered and Sartopore 2; 0.45+0.2 μm filtered *7 Final formulations are equally potent in the scratch wound healing assay. This indicates that both filtration devices are equally suitable filtration devices for maintaining scratch wound healing potency of the CPC-EV-enriched secretomes prepared by the GMP-Compatible process for producing small extracellular vesicle-enriched fraction (sEV) formulations described as in Example 12 and 13. In terms of potency in a scratch wound healing assay, all four variations of final formulation method depicted in FIG. 24B and described in Example 13 are equally suitable.

In FIG. 30B, the +ve control result is at 100% (first bar on the left). The −ve control is at 0% (second bar from the left). The EV Ctl is a 25.2% (third bar from the left). The Final Formulations *7, sample c (Test 22) and *7, sample d (Test 22) gave similar results. Both materials improved scratch wound healing, with indications of a dose-response from the doses ranging from 0.25× to 2.6×. The lowest dose tested, which was 0.25× gave at least a 17% increase in wound healing capacity over the −ve control for both samples. At a dose of 2.6×, both samples improved scratch wound healing by greater than 30% over the negative control. Taken together, the data here indicate that both Sterivex-GP, 0.22 μm filtered and Sartopore 2; 0.45+0.2 pam filtered *7 Final formulations are equally potent in the scratch wound healing assay. This indicates that both filtration devices are equally suitable filtration devices for maintaining scratch wound healing potency of the CPC-EV-enriched secretomes prepared by the GMP-Compatible process for producing small extracellular vesicle-enriched fraction (sEV) formulations described as in Example 12 and 13. In terms of potency in a scratch wound healing assay, all four variations of final formulation method depicted in FIG. 24B and described in Example 13 are equally suitable.

FIGS. 31A and 31B depict cardiomyocyte survival in a staurosporine-induced cardiotoxicity assay. The results for samples *7, sample a (Test 22) and *7, sample b (Test 22) (depicted in FIG. 24B) are shown in FIG. 31A, bars referred to as bars one through nine from left to right. The results for samples *7, sample c (Test 22) and *7, sample d (Test 22) (depicted in FIG. 24B) are shown in FIG. 31B, bars referred to as bars 1 through 15 from left to right. 1× equals the secretome derived from 150,000 cells. PBS controls with and without staurosporine served as negative control (“−ve”, bar two in both figures) and positive control (“+ve”, bar one in both figures), respectively. Mesenchymal Stem Cell (MSC)-derived EV served as an EV control (“EV Ctl”, bar three in both figures). Plated cells were either stressed with staurosporine for 4 hours prior to treatment (“+”, bars two through nine in FIG. 31A and bars two through 15 in FIG. 31B), or were not stressed with staurosporine (“−“, bar one in both figures). *7, sample a (Test 22); *7, sample b (Test 22); *7, sample c (Test 22); *7, sample d (Test 22) (depicted in FIG. 24B) whose preparation is described in detail in Example 12 and Example 13 were tested in a cardiomyocyte survival assay as described in Example 17. These four samples are derived from the same TFF retentate but differ in their method of final formulation. These four variations are to use fresh retentate and filter sterilize with Sterivex-GP, 0.22 μm filter (resulting in *7, sample a (Test 22)); to use fresh retentate, supplement with trehalose, and filter sterilize with a Sterivex-GP, 0.22 μm filter (resulting in *7, sample b (Test 22)); to freeze retentate, thaw it, and then filter sterilize with a Sterivex-GP, 0.22 μm filter (resulting in *7, sample c (Test 22)); or freeze retentate, thaw it, and then filter sterilize with a Sartopore 2; 0.45+0. μm filter (resulting in *7, sample d (Test 22)).

In FIG. 31A, at the 24-hour time point illustrated in the figure, the greatest effect seen for *7, sample a (Test 22) was at the 0.75× dose (bar six), which corresponded to an increase (improvement) in cell survival of 10.64% more than the −ve control. At the 24-hour time point illustrated in the figure, the greatest effect seen for *7, sample b (Test 22) was at the 0.5× dose (bar eight), which corresponded to an increase (improvement) in cell survival of 11.85% improvement over the −ve control.

In FIG. 31B, at the 24-hour time point illustrated in the figure, the greatest effect seen for *7, sample c (Test 22) was at the 0.75× dose (bar six), which corresponded to an increase (improvement) in cell survival of 14.82% more than the −ve control. At the 24-hour time point illustrated in the figure, the greatest effect seen for *7, sample d (Test 22) was at the 0.5× dose (bar 11), which corresponded to an increase (improvement) in cell survival of 11.90% improvement over the −ve control.

FIG. 34 depicts echocardiography results of mice with induced chronic heart failure following administration of CPC EVs (“sEV5.3”), or PBS (as a control). The data depicts the absolute changes in Left Ventricular End Systolic Volume (LVESV); Left Ventricular End Diastolic Volume (LVEDV); and ejection fraction (EF). The bottom three graphs show the absolute change in each animal as as individual point with an overlayed quantile plot for the group. For illustrative purposes, a dotted horizontal line is added to each graph to indicate the approximate location of the threshold used to define severely progressive heart failure for each of the three parameters. For illustrative purposes, a dotted-line-box has been added to the figure to identify the animals considered to have severely progressive heart failure in each graph. The actual number of animals with and without severely progressive heart failure is noted in large font on each graph. The graph on the left shows a threshold of 9.1 μL. Animals at or above this threshold have severely progressive heart failure. The middle graph shows a threshold of 4 μL. Animals at or above this threshold have severely progressive heart failure. The graph on the right shows a threshold of −5.5%. Animals at or below this threshold have severely progressive heart failure, with severely decreasing EF. In all three graphs, less of the sEV treated animals have severely progressive heart failure than in the PBS group. For the Absolute Change in LVESV, the sEV5.3 group had significantly less animals with severely progressive heart failure than PBS controls (5 of 11 animals versus 10 of 11 for PBS controls, p<0.05). For the Absolute Change in LVEDV, the sEV5.3 group had significantly less animals with severely progressive heart failure than PBS controls (5 of 11 animals versus 10 of 11 for PBS controls, p<0.05). For the Absolute Change in EV, the sEV5.3 group had less animals with severely progressive heart failure than PBS controls, approaching significance (1 of 11 animals versus 5 of 11 for PBS controls, p<0.05). Taken together, these results indicate that cardiac therapy candidate-1-extracellular vesicle enriched secretome (CTC1-EV) improves heart failure outcomes in animals with chronic heart failure by limiting the progression of that heart failure.

FIG. 35 depicts the results of Lunatic analysis for cellular RNA extracted from *3 (Test 25) as depicted in FIG. 90. The RNA extracted from *3 (Test 25) is labeled as sample “546” in the figure. FIG. 35 also depicts the results of Lunatic analysis for cellular RNA extracted from *3 (Test 26) as depicted in FIG. 96. The RNA extracted from *3 (Test 26) is labeled as sample “547” in the figure. The preparation of samples *3 (Test 25) and *3 (Test 26) is described in detail in Example 19.

FIG. 36 depicts the results for quality control (QC) testing of cellular RNA that was extracted from *3 (Test 25). This RNA is described as “546RNA” in the figure. This analysis was completed to assess the quality of the extracted RNA.

FIG. 37 depicts the results for quality control (QC) testing of cellular RNA that was extracted from*3 (Test 26). This RNA is described as “547RNA” in the figure. This analysis was completed to assess the quality of the extracted RNA.

FIG. 38 depicts the results of Lunatic analysis for CTC1-EV RNA extracted from *9 (Test 27). This RNA is described as “45.evrna” in the figure. This analysis was completed to assess the quality of the extracted RNA.

FIG. 39 depicts the results for quality control (QC) testing of CTC1-EV RNA extracted from *9 (Test 27). This RNA is described as “45.evrna” in the figure. This analysis was completed to assess the quality of the extracted RNA.

FIG. 40 depicts the results for quality control (QC) testing of the cDNA libraries produced from three different RNA samples. The “Library from 546RNA” is the cDNA library generated from the RNA extracted from *3 (Test 25). The “Library from 547RNA” is the cDNA library generated from the RNA extracted from *3 (Test 26). The “Library from 45.evrna” is the cDNA library from the RNA extracted from *9 (Test 27). These assessments were performed to assess the quality of the cDNA libraries.

FIG. 41 depicts the results of the analysis of the sequencing read lengths for the small RNA sequencing analysis of CTC1-EV, which is *9 (Test 27) in this experiment.

FIG. 42 depicts the prevalence (read distribution) of different RNA biotypes in CTC1-EV, which is *9 (Test 27) in this experiment. The RNA biotypes illustrated here were determined by sequence mapping. Results for this sample are identified as “45RNA” in the figure.

FIG. 43 depicts the results of the analysis of read distributions for isomirs of the top 20 miRs identified in CTC1-EV, which is *9 (Test 27) in this experiment. Results for this sample are identified as “45RNA” in the figure.

FIG. 44 depicts the top 40 most abundant miRNA identified in CTC1-EV, which is *9 (Test 27) in this experiment. The data are displayed as a honeycomb representation. The results for this sample are labeled as “45RNA” in this figure. The data which was used to generate FIG. 44 are tabulated in TABLE 9.

FIG. 45 shows a wordcloud indicating the top localization terms associated with the RNA sequences identified in CTC1-EV, which is *9 (Test 27) in this experiment.

FIG. 45.1 shows a scatterplot identifying an miRNAs signature in CTC1-EV as compared to extracellular vesicles from other cell types included in this study (astrocyte, cardiac fibroblast, cardiomyocyte, neurons (GABAergic, Glutamatergic, Dopaminergic, Motor Neurons, and induced Neurons by forward reprogramming), endothelial, hematopoietic progenitor cells, hepatocyte, induced pluripotent stem cell, microglia, macrophage, mesenchymal stem cells, pericytes, and retinal pigment epithelial). The CTC1 EV miR signature was extracted by calculating the gene-wise 10^{th} percential of log 2FPKM values of CTC1-EV sample replicates and 90^{th} percentile of all the other samples in the study.

FIG. 46 and FIG. 47 depict cryo-electron micrographs of extracellular vesicles identified in CTC1-EV, which is *9 (Test 27) in this experiment. Scale bar=100 nm.

FIG. 48 depicts a cryo-electron micrograph of a large bilipid membrane vesicle (identified in CTC1-EV, which is *9 (Test 27) in this experiment) of approximately 200 nm in diameter, which contains therein a second bilipid membrane vesicle of a similar diameter as well as a third, smaller (approximately 50 nm in diameter) bilipid membrane. Scale bar=100 nm.

FIG. 49 depicts a 96-well platemap for the analysis of the effects of CTC1-EV in a HUVEC plating assay as described in Example 23. CTC1-EV in this experiment is *5b.uc (Test 26). This sample is labeled “EV 481” in the figure. A mock-EV control is also included (labeled “EV 457” in the figure).

FIG. 50 depicts the effects of CTC1-EV in the HUVEC plating assay, as measured by Tecan for Life Science® plate reader. Bars referred to as bars one through seven from left to right. CTC1-EV is *5b.uc (Test 26) in this experiment (results depicted in bars four and five) was analyzed in a HUVEC plating assay. In this assay, the number of HUVEC cells in each well are determined by measuring the amount of intracellular ATP in the well, which is a surrogate for the number of cells. The amount of ATP is determined using the Cell Titer Glo kit as described in Example 23. The readout is luminescence. The higher the luminescence, the more ATP was present in the well, which means more cells were present in the cell. The higher luminescence therefore means the better the HUVEC plating. In this assay, the positive control (“+ Control”, bar one) is HUVEC cells plated in their complete media as described in Example 23. The negative control (“−Control”, bar two) is the HUVEC cells plated in poor media as described in Example 23. For the remaining conditions, the HUVEC cells are plated in poor media supplemented with FBS-EV (bar three), *5b.uc (Test 26) (bars four and five), or matched mock-EV controls (“mock-EV”, bars six and seven) as described in Example 23. The results are double normalized such that the negative control is set to 0% and the positive control is set to 100%. The results for the positive control are in the first bar on the left (100%). The results for the negative control are in the second bar from the left (0%). The FBS-EV condition gave a 60.37% result. The *5b.uc (Test 26) resulted in²⁹0.11% luminescence of the positive control when dosed at first dose (“1×”). The *5b.uc (Test 26) resulted in 49.37% luminescence of the positive control when dosed at a three times higher dose than the first dose (“3×”). The matched mock-EV controls were also dosed at 1× and 3× doses, resulting in 9.66% and 17.90% luminescence of the positive control. The greater the % luminescence in this assay, the greater the improvement the material tested has on HUVEC cell plating. Both the 1× and 3× doses of the CTC1-EV tested here improve HUVEC plating in this assay as compared to the negative control, and as compared to their matched mock-EV controls. The improvement in HUVEC seeding in this assay is more than twice the improvement seen from the matched mock-EV controls.

FIG. 51 depicts the effects of CTC1-EV (which is sample *5b.uc (Test 26) in this experiment) in a HUVEC plating assay, as measured by visual inspection (the nuclei of living cells are labeled in green, which resembles a bright light grey in black and white rendering). This sample is labeled “CTC1-EV *5b.uc (Test 26)” in the figure. The mock-EV control is labeled “mock-EV” in this figure.

FIG. 52 depicts the effects of CTC1-EV, which is sample *5b.uc (Test 26) in this experiment, in the HUVEC plating assay, as determined by CyQuant nucleic acid stain (bars referred to as one through seven from left to right). Sample *5b.uc was analyzed in a HUVEC plating assay as described in Example 23. In this assay, the number of HUVEC cells in each well is determined by measuring the amount of fluorescence in each well. The fluorescence comes from the CyQuant Green dye, which is fluorescent inside cells. The higher the fluorescence signal at the end of the assay, the more cells are present in the well. The greater the number of cells present in the well, the better the tested material is at improving HUVEC cell plating. In this assay, the positive control (“+Control”, bar one) is HUVEC cells plated in their complete media as described in Example 23. The negative control (“− Control”, bar two) is the HUVEC cells plated in poor media as described in Example 23. For the remaining conditions, the HUVEC cells are plated in poor media supplemented with FBS-EV (bar three), *5b.uc (bars four and five), or matched mock-EV controls (“mock-EV”, bars six and seven) as described in Example 23. The results are double normalized such that the negative control is set to 0% and the positive control is set to 100%. The results for the positive control are in the first bar on the left (100%). The results for the negative control are in the second bar from the left (0%). The FBS-EV condition gave a 36.34% result. The *5b.uc (Test 26) resulted in 15.43% of the positive control when dosed at a first dose (“1×”). The *5b.uc (Test 26) resulted in 36.75% of the positive control when dosed at a three times higher dose than the first dose (“3×”). The matched mock-EV controls were also dosed at 1× and 3× doses, resulting in −1.07% and 8.42% of the positive control. Both the 1× and 3× doses of the CTC1-EV tested here [*5b.uc (Test 26)] improve HUVEC plating in this assay as compared to the negative control, and as compared to their matched mock-EV controls. The improvement in HUVEC seeding in this assay by the CTC1-EV tested here [*5b.uc (Test 26)] is more than four times any improvement seen from the matched mock-EV controls.

FIG. 52.1 depicts the effects of CTC1-EV, which is sample *5b.uc (Test 26) in this experiment, in the HUVEC plating assay, as determined by CyQuant nucleic acid stain (bars referred to as one through seven from left to right). Sample *5b.uc (Test 26) (bars four and five) was analysed in a HUVEC plating assay as described in Example 23. In this assay, the number of HUVEC cells in each well are determined by analysing microscope images where the cells are easily identified by CyQuant green straining. The greater the number of cells present in the well, the better the tested material is at improving HUVEC cell plating. In this assay, the positive control (“+Control”, bar one) is HUVEC cells plated in their complete media as described in Example 23. The negative control (“− Control”, bar two) is the HUVEC cells plated in poor media as described in Example 23. For the remaining conditions, the HUVEC cells are plated in poor media supplemented with FBS-EV (bar three), Sample *5b.uc (Test 26) (bars four and five), or matched mock-EV controls (“mock-EV”, bars six and seven) as described in Example 23. The results are double normalized such that the negative control is set to 0% and the positive control is set to 100%. The results for the positive control are in the first bar on the left (100%). The results for the negative control are in the second bar from the left (0%). The FBS-EV condition gave a 54.47% result. The *5b.uc (Test 26) resulted in 20.42% of the positive control when dosed at a first dose (“1×”). The *5b.uc (Test 26) resulted in 48.09% of the positive control when dosed at a three times higher dose than the first dose (“3×”). The matched mock-EV controls were also dosed at 1× and 3× doses, resulting in −2.13% and 11.71% of the positive control. Both the 1× and 3× doses of the CTC1-EV tested here [*5b.uc (Test 26)] improve HUVEC plating in this assay as compared to the negative control, and as compared to their matched mock-EV controls. The improvement in HUVEC seeding in this assay by the CTC1-EV tested here [*5b.uc (Test 26)] is more than four times any improvement seen from the matched mock-EV controls.

FIG. 53 depicts the results of an analysis of CTC1-EV in a HUVEC stress assay, in which HUVECs were stressed with staurosporine as described in Example 24. Bars are referred to as bars one through six from left to right. Three different EV types were tested in a HUVEC Stress Assay. In this assay, HUVEC cells in culture are not stressed (“Complete”; positive control; bar one), stressed by culturing in serum-free media (“Poor”; bar two) or stressed by culturing in serum-free media containing staurosporine (“Poor+Staurosporine” conditions; bars three through six. For the “Poor+Staurosporine” conditions, either vehicle control was added to the culture media (“dPBS”, bar three) or a dose of 5×10⁹ particles was added from one of three different EV-enriched secretome preparations. These EV-enriched secretome preparations were isolated from MSC conditioned media (“MSC-EV”, bar four), from iCell CPC conditioned media (“iCell-CPC-EV”, bar five) or from CTC1 conditioned media (“CTC1-EV”, bar six). In this example, the CTC1-EV (bar six) is *9 (Test 27). The number of HUVEC cells remaining in culture at the end of the assay period was determined. The result for each condition was normalized to the “dPBS” (bar three) vehicle control condition. The CTC1-EV sample tested here [*9 (Test 27)] improved HUVEC cell survival by 40% in this experiment.

FIG. 54 depicts the results of an analysis of EV-CPC in an in-vitro chemotherapy-induced cardiomyopathy assay, as determined by measuring intracellular ATP concentration (A) at day 6, (B) at day 8, and (C) at day 10, in doxorubicin-stressed cardiomyocytes (and non-stressed control cardiomyocytes) as described in Example 25. The results were normalized to the control (“DOX+Placebo”) at the day of the measurements. The results are from five separate experiments, with each sample within each experiment being performed in triplicate. The bars show the mean+/−SEM. *p<0.05 (Kruskal-Wallis with Dunn's multiple comparisons test). CM: complete maintenance cardiomyocyte medium; DOX: doxorubicin, EV-CPC: extracellular vesicles derived from cardiac progenitor cells; VM-CPC: CPC-virgin medium; ATP: Adenosine Triphosphate.

As can be seen from FIG. 54D, CTC1-EV sample (which is *7, sample a (Test 20) in this experiment; labeled “CTC1-EV (prod 20)” in the figure), improved (increased) the amount of intracellular ATP per cell in the doxorubicin stressed cardiomyocytes by 40% over the stressed control. This result shows that CTC1-EV [*7, sample a (Test 20)] was able to promote cardiomyocyte metabolic health in surviving cells. The results of the positive control are shown in bar 1 (as numbered left to right, 1 through 3). The results of the negative control, which is doxorubicin stressed cells, are shown in bar 2. The results of CTC1-EV treatment of doxorubicin stressed cells are shown in bar 3.

FIG. 55 and FIG. 56 depict the results of an anti-fibrosis assay in which HCF cells were stimulated with TGF-β1, and the effects of MSC-EV and CTC1-EV [*9 (Test 27) in this experiment] on various fibrosis-associated markers were then analyzed by quantitative reverse transcription polymerase chain reaction (RT-qPCR) as described in Example 26 (bars referred to as one through ten in both figures from left to right). The results for the MMP2 expression analysis are shown in FIG. 55. The results for the Periostin (“Postn”) expression analysis are shown in FIG. 56. In this example, the CTC1-EV is *9 (Test 27) (bars five, six, nine, and ten in each figure).

FIG. 56.1 depicts the experimental schedule for the experiment described in Example 27. A timeline is presented illustrating the five days on which rats received IP injections of doxorubicin (where applicable), the three days on which rats were evaluated by echocardiography, and the three days on which rats received IV injection of placebo (NaCl) or CTC1-EV where applicable, where CTC1-EV is *7, sample a (Test 20) in this experiment. In this figure, *7, sample a (Test 20) is labeled as “GMP-EV”.

FIG. 57A and FIG. 57B depict the effects of CTC1-EV (which is *7, sample a (Test 20) in this experiment), on cardiac function in a rat chemotherapy (doxorubicin)-induced cardiomyopathy (CCM) model as described in Example 27. In this figure, *7, sample a (Test 20) is labeled as “GMP-EV”. FIG. 57A depicts the % change in LV-ESV since D10. FIG. 57B depicts the % change in LV-EDV since day 10. Results were measured by echocardiography and expressed as a percent change (Median+/−IQR) from day 10 (post-DOX administration). There were 6 animals that did not receive doxorubicin (Sham: n=6). There were 11 animals that received the doxorubicin stress and then NaCl injections (“DOX+Placebo”; n=11) and there were 12 animals that received the doxorubicin stress and then *7, sample a (Test 20) injections (“DOX+GMP-EV”; n=12). In this figure, the symbol ‘*’ on the figure indicates p≤0.05, Kruskal Wallis with Dunn's correction test. LV-ESV/LV-EDV: left ventricular systolic/diastolic function.

Cardiac function is related to heart volumes. Two types of heart volumes are examined here: the left ventricular end systolic volume (LVESV, or LV-ESV) and the left ventricular end diastolic volume (LVEDV, or LV-EDV). During heart failure, these two volumes increase. The more they increase, the worse the heart failure has progressed. The two volumes are measured by echocardiography (echo). The two volumes for each animal are measured once before doxorubicin injection (or before sham injections for “Sham” animals) (baseline echo, echo #1), then on the tenth day after the first doxorubicin administration/sham injection (which is before CTC1-EV treatment or placebo administration; echo #2), and finally at the end of the study period on or around 28 or 29 days after the first doxorubicin injection/sham injection (echo #3). The CTC1-EV in this experiment was *7, sample a (Test 27). The group of rats receiving this material is referred to as “Dox+GMP-EV” in FIG. 57A and FIG. 57B. The Placebo group received isotonic buffer, NaCl 0.9%, “Placebo”; this group of animals is referred to as “DOX+Placebo” in FIG. 57A and FIG. 57B). CTC1-EV or Placebo were administered 11, 14 and 16 days after the first doxorubicin injection as depicted in FIG. 56.1. The more the heart volumes increase between the echo #2 and echo #3, the more the heart failure has progressed in that animal during that time period. The “Sham” animals are not in heart failure; they show no markers of failing hearts. The Sham animals were not administered any doxorubicin or CTC1-EV.

In the experiment depicted in FIG. 57A and FIG. 57B, the results are expressed as a percent change (Median+/−IQR) from day 10 post-DOX administration (echo #2) to the end of the study period (echo #3). There were 6 Sham animals, 11 Placebo injected animals, and 12 animals injected with CTC1-EV. (Sham: n=6; DOX+Placebo: n=11, Dox+GMP-EV: n=12).

For the LV-ESV results (depicted in FIG. 57A), the Sham group, on average, had a −3.0% change in volume; the DOX+Placebo group, on average, increased LVESV by 28.1%; the Dox+GMP-EV increased LVESV by 12.9%, which means that their heart failure progressed less than 1/2 as much as the placebo group as determined by LV-ESV change, which is a 2.2-times improvement in outcome.

For the LV-EDV results (depicted in FIG. 57B), the Sham group, on average, had a −0.1% change in volume; the DOX+Placebo group, on average, increased LVEDV by 19.2%; the Dox+GMP-EV increased LVEDV by 0.7%, which means that their heart failure progressed less than 4-tenths (0.7/19.2) as much as the placebo group as determined by LV-ESV change, which is a 27-times improvement in outcome.

While LV-ESV volumes were significantly increased in placebo-injected hearts compared with Sham (p=0.033), they were preserved by GMP-EV injections (effect size Hedges' g index of 0.4). Likewise, the percentages of responder rats which did not increase their LV-EDV volumes by more than 5% from their post-DOX pre-treatment values were 58% (7 out of 12) vs. 28% (3 out of 11) in the Dox+GMP-EV and DOX+Placebo rat hearts, respectively (effect size Hedges' g index of 0.5, OR=3.7).

FIG. 58A, FIG. 58B, FIG. 58C, FIG. 58D, FIG. 58E depict the results of experiments validating the rat model of doxorubicin-induced cardiomyopathy as described in Example 27. FIG. 58A depicts LVEF as a percent change (Mean+/−SEM) from day 10 (post-DOX administration). FIG. 58B depicts the end-study ratio of diastolic blood pressure to LV-EDV taken as a surrogate marker for ventricular compliance. FIG. 58C depicts mean blood pressure. FIG. 58D depicts the QT interval corrected for heart rate. **p<0.005; (Mann Whitney test). LVEF: left ventricular ejection fraction (%); QTc: length of QT segment corrected to heart rate (HR) (in seconds, “sec”), DBP: diastolic blood pressure; LV-EDV: left ventricular end diastolic volume.

FIG. 58E depicts the results of experiments further validating the rat model of doxorubicin-induced cardiomyopathy as described in Example 27. The figure depicts the end-of-study ratio of systolic blood pressure to LV-ESV. This ratio is taken as a surrogate marker for ventricular contractility. This ratio is termed the “End Systolic Elastance”. LV-EDV: left ventricular end-diastolic volume, SBP: systolic blood pressure. That data show that the average SBP/LV-ESV decreased in doxorubicin (DOX) treated animals (“DOX+Placebo” group) by 0.46 mmHg/uL (which is a decrease of 34%) from the animals that did not receive DOX (“Sham” group).

FIG. 58.1 depicts the experimental design of two of the experiments used to establish a novel chemotherapy-induced cardiomyopathy model in rats. The first experiment, in which 6 male rats were injected with doxorubicin resulted in an unacceptable mortality rate over the 30 or 32 day long procedure. 70% of these male rats died prior to completing the study period. A second experiment is depicted in which male and female rats were included. The female rats had a survival rate much greater than the males (91% vs 40%, respectively). d=day; DOXO=doxorubicin injection; echo=echocardiographic measurements.

FIG. 59 and FIG. 60 depict the results of experiments analyzing the post-thaw viability of CTC1 cells under different conditions as described in Example 28 (referred to as bars one through six from left to right in each figure respectively).

To improve CTC1 post-thaw survival, plating and expansion, three modifications to the process were tested. Details of this experiment are given in Example 28. The starting process thawed cells in the same media that was used for plating and expansion (referred to as Complete Media A, “CM A”, bars one and two in both FIG. 59 and FIG. 60). The starting process used a gentle centrifugation step to pellet cells after thaw, enabling the removal of the cryopreservation media (use of the centrifugation step is referred to as the centrifuged condition, “Cent”, bars one, three, and five in both FIG. 59 and FIG. 60). The starting process used a thaw media containing 2 mg/mL human serum albumin. The starting process used a thaw media that did not contain a ROC inhibitor. The starting process is referred to as “CM A Cent” in FIG. 59 and FIG. 60. The number of viable cells which were placed into each vial at the cryopreservation stage was known. The number of viable cells recovered after the thaw process was noted. The percentage of recovered cells at thaw (“% Recovered”) was calculated by taking the number of viable cells per vial after the thaw process divided by the number of viable cells placed into each vial prior to cryopreservation, times 100%. The greater the percentage recovered at thaw, the more successful the thaw process was deemed. The % Recovered was calculated for six conditions illustrated in FIG. 59 and six conditions illustrated in FIG. 60, which varied by the thaw media compositions used and whether or not a centrifugation step was included in the process. The recipes for the various thaw media used are detailed in Example 28.

In FIG. 59, the results of the starting process (CM A with a Centrifugation step; “CM A Cent”) are given in the bar one. The results of the modified process using the starting thaw media but omitting the centrifugation step (“CM A No Cent”) are shown in bar two. The results of the modified process using the thaw media containing higher albumin concentration (20 mg/mL HSA) with and without a centrifugation step (“CM B Cent”, “CM B No Cent”, respectively) are shown in bar three and bar four respectively. The results of the modified process using the thaw media containing higher albumin concentration (20 mg/mL) and 1 μM H1152 ROC inhibitor with and without a centrifugation step (“CM C Cent”, “CM C No Cent”, respectively) are shown in bar five and bar six, respectively.

FIG. 60 depicts the results of post-thaw cell viability assays conducted under different conditions.

In FIG. 60, the results of the starting process (CM A with a Centrifugation step; “CM A Cent”) are given in bar one. The results of the modified process using the starting thaw media but omitting the centrifugation step (“CM A No Cent”) are shown in bar two. The results of the modified process using the thaw media containing 1 μM H1152 with and without a centrifugation step (“CM B Cent”, “CM B No Cent”, respectively) are shown in bar three and bar four, respectively. Note that this is a different set of conditions from the “CM B” conditions illustrated in FIG. 59. The results of the modified process using the thaw media containing higher albumin concentration (20 mg/mL) and 1 M H1152 ROC inhibitor with and without a centrifugation step (“CM C Cent”, “CM C No Cent”, respectively) are shown in bars five and six, respectively from the left.

Taken together, the results FIGS. 59 and 60 show that when the centrifugation step used to remove the cryopreservation media components is omitted, the % Recovery at Thaw increases by an average of 6.45 percentage points over the matched centrifuged conditions. (6.45 is the average of −0.2, +11.1, +6.6, +6.5, +7.4, and +7.3)

FIG. 61 depicts an experimental design for analyzing post-thaw platability of CTC1 cells as described in Example 28.

FIG. 62 depicts the results of experiments analyzing cell densities (cells/cm²) after thawing and plating CTC1 cells under different conditions as described in Example 28.

FIG. 63 depicts the results of experiments optimizing the CTC1 cell culture vessel for plating as described in Example 29.

FIG. 64 depicts an experimental design for analyzing the effect of insulin concentration on CTC1 cell yield throughout vesiculation as described in Example 30.

FIG. 65 depicts the results of experiments analyzing the effect of insulin concentration on CTC1 cell yield throughout vesiculation as described in Example 30.

FIG. 66 depicts an experimental design for analyzing the effect of FGF concentration on CTC1 cells as described in Example 31.

FIG. 67 depicts the results of experiments analyzing CTC1 cell counts after incubation at different FGF concentrations as described in Example 31.

FIG. 68 depicts the results of scratch wound healing experiments using EV/secretome from CTC1 cells incubated with different FGF concentrations as described in Example 31 (lines referred to as A through H from highest to lowest at the 18-hour timepoint). Conditions included in this assay include positive control (line A); High FGF, MC (labeled as “our standard protocol”, line B); Mid FGF, MC (line C); Low FGF, MV (line D); Low FGF, MC (line E), Mid FGF, MV (line F); High FGF, MV (line G); and negative control (line H). The results at the 18-hour time point are also summarized in TABLE 22.

FIG. 69 depicts the results of cardiomyocyte survival assay experiments using EV/secretome from CTC1 cells incubated with different FGF concentrations as described in Example 31. The results at the 19 hour time point are also summarized in TABLE 23.

FIG. 70A and FIG. 70B depict the results of a time course monitoring scratch wound healing, for fresh media samples. The y-axis is the % wound confluence, and the x-axis is hours since the start of the assay. Lines referred to as A through P from top to bottom at the 18-hour time point, UF retentates were produced from freshly collected and freshly clarified CTC1 conditioned media as described in Example 32 (lines D through H). UF retentates from freshly collected and clarified CPC virgin media controls were prepared as described in Example 32 (“Mock-EV controls”, lines I, K through P). Fresh conditioned media was also subjected to ultracentrifugation as described in Example 32 (“Fresh UC generated EV”, line B). An EV-enriched secretome was also prepared from FBS by ultracentrifugation (“FBS Control”, line C). These compositions were tested for the ability to stimulate HUVEC scratch wound healing in a HUVEC Scratch Wound Healing Assay, as described in Example 32. The positive control is the condition in which HUVEC are maintained in complete media (“Complete Media Control”, line A). The positive control attained 33.2% wound confluence at the 18-hour time point. The negative control is the condition in which HUVEC are cultured in serum-free, or poor, media (“Poor Media Control”, line J). The negative control attained for this HUVEC scratch wound healing assay, retentate samples were dosed into the assay at a 3× dose (where 1× doses were the secretome/EV/secretome fraction produced by 150,000 mother cells). Mock-EV controls were volume matched to the corresponding condition. The Fresh UC generated EV sample was dosed at 1×. The figure depicts the results of a time course monitoring scratch wound healing, for fresh media samples. At the 18-hour time point, the Fresh UC generated EV sample (line B) had the strongest effect out of all test conditions. Fresh UF retentate fractions containing components with molecular weight in the 5 kDa-10 kDa (line H), 10 kDa −30 kDa (line G), 30 kDa −50 kDa, (line F) 50 kDa−100 kDa (line D) and 100 kDa−0.2 μm (line E) ranges all had a greater effect on HUVEC scratch wound healing than the Mock-EV and Poor media controls. This indicates that the retentates have a positive effect on endothelial cell migration into the scratch wound. The strongest effects are caused by components of the conditioned media having molecular weights between 30 kDa and 0.2 μm. At the 15-hour timepoint, the 30 kDa-50 kDa retentate (line F) has a 1.4-fold greater % wound confluence than the negative control (line J). At the 15-hour timepoint, the 50 kDa-100 kDa retentate (line D) has a 1.7-fold greater % wound confluence than the negative control (line J). At the 15-hour timepoint, the 100 kDa-2 μm retentate (line D) has a 2.3-fold greater % wound confluence than the negative control (line J). The 18-hour time point results depicted in FIG. 70A are given in TABLE 24. The 18-hour time point results depicted in FIG. 70B are given in TABLE 25.

FIG. 70.1 is an alternative depiction of the data presented in FIG. 70A. In addition, it depicts the results of a CTC1-EV secretome composition prepared by ultracentrifugation of previously frozen CTC1 conditioned medium (labeled “EV 181 Frozen UC” in the figure) and its mock-EV control (labeled “EV 189 Frozen UC MV” in the figure). The 18-hour time point results depicted in FIG. 70.1 are given in TABLE 26.

FIG. 71 depicts the results of a time course monitoring scratch wound healing, for the CTC1-EV compositions isolated from fresh or frozen/thawed media samples as described in Example 32. The y-axis is the % wound confluence, and the x-axis is hours since the start of the assay. The 18 hour timepoint results depicted in FIG. 71 are given in TABLE 27.

FIGS. 72-74 depict histograms of double normalized data from cardiomyocyte survival assays as described in Example 32. Bars referred to as one through seven from left to right in FIG. 72. Bars referred to as one through fourteen from left to right in FIG. 73. Bars referred to as one through fourteen from left to right in FIG. 74.

FIG. 77 depicts the results of the flow cytometry analysis described in Example 35. Samples preparation is described in detail in Example 19. The data are expressed as a Mean Fluorescence Intensity (MFI) of technical and biological replicates. The MFI for the iPSC is shown by the white bars (the “iPSC” series, which is the first series of bars, starting from the left). The average MFI for “CPC D+0” (the second series of bars, starting from the left) was calculated by averaging the results from *1 (Test 25) and from *l (Test 26). The average MFI for “CPC D+3” (the third series of bars, starting from the left), was calculated by averaging the results from *2 (Test 25) and from *2 (Test 26). The average MFI for “CPC D+5” (the fourth series of bars, starting from the left) was calculated by averaging sample *3 (Test 25) and from *3 (Test 26). The average MFI for CM samples is shown by the black bars (the “CM” series, the fifth series of bars, starting from the left).

FIG. 78 depicts the results of the transcriptomic analysis for cells at day+3 (“D+3”) and day+5 (“D+5”) as described in Example 35. Sample preparation is described in Example 19. The transcriptome analysis shown in the figure shows that the cells collected from Test Example 25 on day+3, from Test Example 25 on day+5, Test example 26 on day+3, and from Test Example 26 on day+5 have mRNA contents consistent with cardiovascular progenitor cells (CPC). Their mRNA profiles of the CPC are similar between the four cell samples. The profiles of the mRNA from the CPC are distinct from both the iPSC and CM controls. The heatmap was generated based on hierarchical clustering analysis using the UPGMA clustering method, with correlation distance metric in TIBCO Spotfire software v11.2.0. The data (log₂FPKM) used to generate the heatmap depicted in FIG. 78 are presented in TABLE 36.

FIG. 79 depicts the results of cell morphology analysis for cells at day+3 (labeled as “CPC D+3” in figure) and day+5 (labeled as “CPC D+5” in figure) as described in Example 35. Sample preparation is described in Example 19. The microscope images shown in the figure show that the cell morphology is similar between *2 (Test 25) and *2 (Test 26) on day+3. The microscope images shown in the figure show that the cell morphology is similar between Test *3 (Test 25) and *3(Test 26) on day+5.

FIGS. 80 and 81 depict particle concentration, mean and mode for samples *5 (Test 25), *6 (Test 25), *7 (Test 25) and *5 (Test 26), *6 (Test 26), *7 (Test 26), and samples *8 (Test 27) and *9 (Test 27) as described in Example 35. Samples preparation is described in Example 19. The particle concentration increased 67-fold between sample *5 (Test 25) to sample *6 (Test 25). The particle concentration increased 58-fold between sample *5 (Test 25) to sample *7 (Test 25). The particles concentration increased by 56-fold between sample *5 (Test 26) to sample *7 (Test 26), a very similar factor to the fold change in Test Example 25. There is no significant change in particle concentration between sample *8 (Test 27) to sample *9 (Test 27). The final concentration of particles in the *9 (Test 27) is 2.8×10¹¹ particles/mL by Nanosight measurement. Throughout Test Examples 25, 26 and 27, the average mean particle size remained relatively constant, ranging from 119.7 to 169.8 nm. Throughout Test Examples 25, 26, and 27, the average mode particle size also remained relatively constant, ranging from 82.4 to 121.5 nm.

FIGS. 82-84 depict CTC1-EV surface marker expression evaluated as described in Example 10 and Example 35. All results are normalized to 13 μL of sample. In these figures, the average of the results for *4 (Test 25) and *4 (Test 26) are noted as “*4 (Tests 25-26)”. In these figures, the average of the results for *5 (Test 25) and *5 (Test 26) are noted as “*5 (Test 25-26)”. In these figures, several technical replicates were evaluated for *8 (Test 27) and the average is shown. In these figures the single result obtained for *9 (Test 27) is shown.

FIG. 82 shows that the MFI for the tetraspanin markers CD9, CD81 and CD63 are greatly increased from the media sample *4 (Test 25-26) to the Final Formulation *9 (Test 27) by greater than 12×, 10× and 7× for CD9, CD63 and CD81, respectively.

The associated MFI of the three canonical tetraspanin EV markers (CD9, CD63, and CD81) for *9 (Test 27) are within the top six highest MFI response of all of the proteins investigated in this assay. Using 13 μL of sample, CD9 had the 6th highest MFI (27.0), CD63 had the third highest MFI (132.1), and CD81 had the second highest MFI (139.6) of the proteins investigated in this assay. Taken together, the strong presence of the three tetraspanin markers indicate the presence of extracellular vesicles. Using 13 μL of sample, other markers with high MFI are CD326 (102.3), CD133/1 (333.4), and CD29 (47.7). Using 13 μL of sample, markers with MFI greater than 1.5 (=expressed markers on *9(Test 27)) are shown in FIG. 82 and FIG. 83. Markers with MFI less than 1.5 as measured in the Final Formulation (*9 (Test 27)) (=little or no expression markers on *9 (Test 27)) are in FIG. 84.

FIG. 84.1 depicts the results of fragments size obtained for *9 (Test 27). The peaks have a size of 179, 368, 537 and 742 base pairs.

FIG. 85 depicts the results of the HUVEC scratch wound healing assay described in Example 37.1 (lines referred to as A through I from top to bottom based on their position at the 24-hour timepoint). The Complete Media positive control (labeled “Complete (dotted line)” in figure, line A), attained 89.33% wound confluence after 24 hours. The Poor Media negative control (labeled “Poor (dashed line)” in the figure, line I), attained 13.22% wound confluence after 24 hours. The Control EV sample, which is the pelleted material obtained after ultracentrifugation of FBS (labeled “Control EV (dash-dot line)” in the figure, line D), obtained 41.29% wound confluence after 24 hours. *9 (Test 27) was tested in this assay at 0.25× (line G), 0.5× (line H), 0.75× (line F), 1× (line E), 2× (line C), and 2.8× (line B), where 1× is 5.79 μL of material and is the secretome collected from 150,000 cells. The results are shown as colored circles, connected by solid lines. The 24 hour timepoint results depicted in FIG. 85 are given in TABLE 51.

FIG. 86 depicts the results of the HUVEC scratch wound healing assay at the 18-hour timepoint described in Example 37.1 (bars referred to as one through nine from left to right). The assay included a Complete Media positive control (labeled “+ve” in figure, bar one), a Poor Media negative control (labeled “−ve” in figure, bar two). The Control EV sample (the pelleted material obtained after FBS ultracentrifugation, labeled “EV Ctl” in figure, bar three), obtained 31% normalized wound confluence after 18 hours. *9 (Test 27), bars four through nine) was tested in this assay at 0.25× (bar four), 0.5× (bar five), 0.75× (bar six), 1× (bar seven), 2× (bar eight), and 2.8× (bar nine), where 1× is 5.79 μL of material and is the secretome prepared from 150,000 cells.

FIG. 87 depicts the results of the in vitro analysis of the potency of *9 (Test 27) whose preparation is described in detail in Example 19 in a cardiomyocyte survival assay described in Example 37.2 (lines labeled A through F from top to bottom based on position at 24-hour elapsed timepoint). Percent of positive NucLight Red cells normalized to T₀ is shown at the indicated timepoint. The 24 hour data points as depicted in FIG. 87 are given in TABLE 52.

FIG. 88 depicts the results of the in vitro analysis of the potency of *9 (Test 27) in a cardiomyocyte survival assay described in Example 37.2 at the 24-hour timepoint (bars referred to as one through six from left to right). The results were baseline (negative control) subtracted and normalized to the positive control.

FIG. 89 depicts the results of the in vitro analysis of the potency of *9 (Test 27) in a Scratch Wound Healing Assay described in Example 38 (bars referred to as one through fourteen from left to right). “Complete medium” positive control (bar one) and the “FBS-EV” control (bar three) were used as positive controls. The negative control was the “Poor medium” control (bar two). The Complete medium (bar one) control consists of HUVEC cultured in Complete Medium [Endothelial Cell Basal Media (PromoCell; ref: C-22210), supplemented with the Endothelial Cell Growth Medium Supplement Pack (PromoCell, ref: C-39210)]. The Poor medium control (bar two) consists of HUVEC cultured in Poor medium alone [Endothelial Cell Basal Media (PromoCell; ref: C-22210]. The “FBS-EV” control (bar three) consists of HUVEC cultured in Poor medium supplemented with 5×10⁹ particles of FBS-EV (FBS-EV is the EV-enriched secretome produced by ultracentrifugation of fetal bovine serum). Four separate vials of *9 (Test 27) were tested (bars 4 through 14) at doses ranging from 1×10⁹ to 5×10⁹ particles per well, as measured by NTA. The particle dose per well is noted under the corresponding data bar.

FIG. 90 illustrates the process used to generate Test Example 25 as described in Example 19.

FIG. 91 depicts the results of the HUVEC Scratch Wound Healing Assay described in Example 41 (bars referred to as one through twelve from left to right). The positive control (labeled “+ve” in the figure, bar one) which is a HUVEC scratch wound healing assay performed in the presence of complete assay media (labeled “Complete” in figure) and treated with vehicle control, which is 0.1 pin filtered PBS (labeled “PBS” in figure). The negative control (labeled “−ve” in the figure, bar two) which is a HUVEC scratch wound healing assay performed in Poor Assay Media (labeled “Poor” in figure) and treated with vehicle control, which is 0.1 μm filtered PBS (labeled “PBS” in figure). The EV control (labeled “EV Ctl” in figure, bar three) is the pellet collected after FBS ultracentrifugation. The results of the HUVEC scratch wound healing assay for 1× (bar four), 2× (bar five), and 3× (bar six) doses of *5a.uc (Test 25), whose preparation is described in detail in Example 19 are shown. The results of the HUVEC scratch wound healing assay for 1× (bar seven), 2× (bar eight), and 3× (bar nine) doses of Test Example 26 CTC1-EV (sample *5b.uc), whose preparation is described in detail in Example 19 are shown. The results of the HUVEC scratch wound healing assay for 1× (bar ten), 2× (bar eleven), and 3× (bar twelve) doses of a mock-EV control (labeled “MV Control” in figure) are shown. The results of this assay are double normalized such that the “−ve” control (bar two) is at 0% wound confluence, and the “+ve “control (bar one) is at 100% wound confluence at the 18 hour timepoint.

FIGS. 93A and 93B depicts the results of the stability testing (described in Example 43) for *9 (Test 27) by NTA.

FIG. 94 depicts the results of the HUVEC Survival Assay at various timepoints as described in Example 44. Sample is *9 (Test 27), (labeled “Poor media with staurospoin (0.01 μM)+EV [Test 27 (*9)]” in figure, results shown in third bar of each cluster of three bars). Data are shown as fold change over the negative control (labeled “Poor media with staurosporine (0.01 μM)” in figure, second bar in each cluster of three bars). Positive control results also shown as fold-change over the negative control (labeled “Poor media without staurosporin” in figure, results shown in first bar of each cluster of three bars).

FIG. 95 depicts the results of the H9c2 cell viability assay described in Example 45. The results of the technical replicates were averaged and normalized to the Virgin Media 100 kDa test condition.

FIG. 96 illustrates the process used to generate Test Example 26 as described in Example 19.

FIG. 97 illustrates the process used to generate Test Example 27 as described in Example 19.

FIG. 98A illustrates the correlation found between particle number (in 10⁶ particles) as measured by NTA, and CD9 MFI as measured by MACSPlex Exosome Kit®.

FIG. 98B illustrates the linearity and minimum linear range of CD9 MFI (which is the Mean Fluorescence Intensity determined by flow cytometry using the MASCPlex Exosome kit human (Miltenyi Ref: 130-108-813) probing for CD9) versus the input volume of *9 (Test 27) for inputs ranging from 1 and 120 μL.

FIG. 99 shows a representative cluster from *9 (Test 27) visualized by ONi that is CD81/CD63/CD9_TP. The CD9 signal, CD81 signal and CD63 signal are shown individually and as an overlay, confirming the presence of each of the three markers in a single cluster. FIG. 99B indicates the relative abundance of each cluster sub-type in the *9 (Test 27) as detected by ONi super-resolution microscopy (shown as a % under each bar). The absolute counts are graphed in the figure and given above each bar.

FIG. 100A depicts the results of a GO (Gene Ontology) enrichment analysis in terms of biological Process. Analysed using String Prot.

FIG. 100B depicts some of the molecular components identified in CTC1-EV. Image created with BioRender.com.

FIG. 100C depicts some of the protein components identified in CTC1-EV and the biological processes in which those components are implicated. The varied processes depicted in the figure highlight the potential for CTC1-EV to have multiple beneficial effects on multiple cell types, culminating in improved physiological outcomes.

FIG. 100D summarizes some of the important biological effects of CTC1-EV treatment as described in this specification. Importantly, mouse, rat, human cell, and human patient data agree that CTC1-EV can impart molecular, cellular and physiological effects on target cells and tissues which are beneficial to stressed human cells and mammals with impaired ventricular function, including humans with heart failure.

FIG. 101 Summary illustration of the clinical trial design.

### DETAILED DESCRIPTION OF THE INVENTION

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a cell” includes one or more cells.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

As used herein, “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females. In some embodiments, cells (for example, stem cells, including pluripotent stem cells, progenitor cells, or tissue-specific cells) are derived from a subject. In some embodiments, the subject is a non-human subject.

As used herein, “differentiation” refers to processes by which unspecialized cells (such as pluripotent stem cells, or other stem cells), or multipotent or oligopotent cells, for example, acquire specialized structural and/or functional features characteristic of more mature, or fully mature, cells. “Transdifferentiation” is a process of transforming one differentiated cell type into another differentiated cell type or to a certain fate.

As used herein, “embryoid bodies” refers to three-dimensional aggregates of pluripotent stem cells. These cells can undergo differentiation into cells of the three germ layers, the endoderm, mesoderm and ectoderm. The three-dimensional structure, including the establishment of complex cell adhesions and paracrine signaling within the embryoid body microenvironment, enables differentiation and morphogenesis.

As used herein, “stem cell” refers to a cell that has the capacity for self-renewal, i.e., the ability to go through numerous cycles of cell division while maintaining their non-terminally-differentiated state. Stem cells can be totipotent, pluripotent, multipotent, oligopotent, or unipotent. Stem cells may be, for example, embryonic, fetal, amniotic, adult, or induced pluripotent stem cells.

As used herein, “pluripotent stem cell” (PSC) refers to a cell that has the ability to reproduce itself indefinitely, and to differentiate into any other cell type of an adult organism. Generally, pluripotent stem cells are stem cells that are capable of inducing teratomas when transplanted in immunodeficient (SCID) mice; are capable of differentiating into cell types of all three germ layers (e.g., can differentiate into ectodermal, mesodermal, and endodermal, cell types); and express one or more markers characteristic of PSCs. Examples of such markers expressed by PSCs, such as embryonic stem cells (ESCs) and iPSCs, include Oct 4, alkaline phosphatase, SSEA-3 surface antigen, SSEA-4 surface antigen, nanog, TRA-1-60, TRA-1-81, SOX2, and REX1.

As used herein, “induced pluripotent stem cell” (iPSC) refers to a type of pluripotent stem cell that is artificially derived from a non-pluripotent cell, typically a somatic cell. In some embodiments, the somatic cell is a human somatic cell. Examples of somatic cells include, but are not limited to, dermal fibroblasts, bone marrow-derived mesenchymal cells, cardiac muscle cells, keratinocytes, liver cells, stomach cells, neural stem cells, lung cells, kidney cells, spleen cells, and pancreatic cells. Additional examples of somatic cells include cells of the immune system, including, but not limited to, B-cells, dendritic cells, granulocytes, innate lymphoid cells, megakaryocytes, monocytes/macrophages, myeloid-derived suppressor cells, natural killer (NK) cells, T cells, thymocytes, and hematopoietic stem cells.

iPSCs may be generated by reprogramming a somatic cell, by expressing or inducing expression of one or a combination of factors (herein referred to as reprogramming factors) in the somatic cell. iPSCs can be generated using fetal, postnatal, newborn, juvenile, or adult somatic cells. In some instances, factors that can be used to reprogram somatic cells to pluripotent stem cells include, for example, OCT4 (OCT3/4), SOX2, c-MYC, and KLF4, NANOG, and LIN28. In some instances, somatic cells may be reprogrammed by expressing at least two reprogramming factors, at least three reprogramming factors, or at least four reprogramming factors, to reprogram a somatic cell to a pluripotent stem cell. The cells may be reprogrammed by introducing reprogramming factors using vectors, including, for example, lentivirus, retrovirus, adenovirus, and Sendai virus vectors. Alternatively, non-viral techniques for introducing reprogramming factors include, for example, mRNA transfection, miRNA infection/transfection, PiggyBac, minicircle vectors, and episomal plasmids. iPSCs may also be generated by, for example, using CRISPR-Cas9-based techniques, to introduce reprogramming factors, or to activate endogenous programming genes.

As used herein, “embryonic stem cells” are embryonic cells derived from embryo tissue, preferably the inner cell mass of blastocysts or morulae, optionally that have been serially passaged as cell lines. The term includes cells isolated from one or more blastomeres of an embryo, preferably without destroying the remainder of the embryo. The term also includes cells produced by somatic cell nuclear transfer. ESCs can be produced or derived from a zygote, blastomere, or blastocyst-staged mammalian embryo produced by the fusion of a sperm and egg cell, nuclear transfer, or parthenogenesis, for example. Human ESCs include, without limitation, MAO1, MAO9, ACT-4, No. 3, H1, H7, H9, H14 and ACT30 embryonic stem cells. Exemplary pluripotent stem cells include embryonic stem cells derived from the inner cell mass (ICM) of blastocyst stage embryos, as well as embryonic stem cells derived from one or more blastomeres of a cleavage stage or morula stage embryo. These embryonic stem cells can be generated from embryonic material produced by fertilization or by asexual means, including somatic cell nuclear transfer (SCNT), parthenogenesis, and androgenesis. PSCs alone cannot develop into a fetal or adult animal when transplanted in utero because they lack the potential to contribute to all extraembryonic tissue (e.g., placenta in vivo or trophoblast in vitro).

As used herein, the term “progenitor cell” refers to a descendant of a stem cell which is capable of further differentiation into one or more kinds of specialized cells, but which cannot divide and reproduce indefinitely. That is, unlike stem cells (which possess an unlimited capacity for self-renewal), progenitor cells possess only a limited capacity for self-renewal. Progenitor cells may be multipotent, oligopotent, or unipotent, and are typically classified according to the types of specialized cells they can differentiate into. For instance, a “cardiomyocyte progenitor cell” is a progenitor cell derived from a stem cell that has the capacity to differentiate into a cardiomyocyte. Similarly, “cardiac progenitor cells” may differentiate into multiple specialized cells constituting cardiac tissue, including, for example, cardiomyocytes, smooth muscle cells, and endothelial cells. Additionally, a “cardiovascular progenitor cell” has the capacity to differentiate into, for example, cells of cardiac and vascular lineages.

As used herein, “expand” or “proliferate” may refer to a process by which the number of cells in a cell culture is increased due to cell division.

“Multipotent” implies that a cell is capable, through its progeny, of giving rise to several different cell types found in an adult animal.

“Pluripotent” implies that a cell is capable, through its progeny, of giving rise to all the cell types that comprise the adult animal, including the germ cells. Embryonic stem cells, induced pluripotent stem cells, and embryonic germ cells are pluripotent cells under this definition.

The term “autologous cells” as used herein refers to donor cells that are genetically identical with the recipient.

As used herein, the term “allogeneic cells” refers to cells derived from a different, genetically non-identical, individual of the same species.

The term “totipotent” as used herein can refer to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps.

As used herein, the term “extracellular vesicles” collectively refers to biological nanoparticles derived from cells, and examples thereof include exosomes, ectosomes, exovesicles, microparticles, microvesicles, nanovesicles, blebbing vesicles, budding vesicles, exosome-like vesicles, matrix vesicles, membrane vesicles, shedding vesicles, membrane particles, shedding microvesicles, oncosomes, exomeres, and apoptotic bodies, but are not limited thereto.

Extracellular vesicles can be categorized, for example, according to size. For instance, as used herein, the term “small extracellular vesicle” refers to extracellular vesicles having a diameter of between about 50-200 nm. In contrast, extracellular vesicles having a diameter of more than about 200 nm, but less than 400 nm, may be referred to as “medium extracellular vesicles,” and extracellular vesicles having a diameter of more than about 400 nm may be referred to as “large extracellular vesicles.” As used herein, the term “small extracellular vesicle fraction” (“sEV”) refers to a part, extract, or fraction, of secretome or conditioned medium, that is concentrated and/or enriched for small extracellular vesicles having a diameter of between about 50-200 nm. Such concentration and/or enrichment may be obtained using one or more of the purification, isolation, concentration, and/or enrichment, techniques disclosed herein. In some alternative embodiments herein, enrichment may not be performed, may not be achieved, or may not be possible.

The term “exosome” as used herein refers to an extracellular vesicle that is released from a cell upon fusion of the multivesicular body (MVB) (an intermediate endocytic compartment) with the plasma membrane.

“Exosome-like vesicles,” which have a common origin with exosomes, are typically described as having size and sedimentation properties that distinguish them from exosomes and, particularly, as lacking lipid raft microdomains. “Ectosomes,” as used herein, are typically neutrophil- or monocyte-derived microvesicles.

“Microparticles” as used herein are typically about 50-1000 nm in diameter and originate from the plasma membrane. “Extracellular membranous structures” also include linear or folded membrane fragments, e.g., from necrotic death, as well as membranous structures from other cellular sources, including secreted lysosomes and nanotubes.

As used herein, “apoptotic blebs or bodies” are typically about 1 to 5 μm in diameter and are released as blebs of cells undergoing apoptosis, i.e., diseased, unwanted and/or aberrant cells.

Within the class of extracellular vesicles, important components are “exosomes” themselves, which may be between about 40 to 50 nm and about 200 nm in diameter and being membranous vesicles, i.e., vesicles surrounded by a phospholipid bilayer, of endocytic origin, which result from exocytic fusion, or “exocytosis” of multivesicular bodies (MVBs). In some cases, exosomes can be between about 40 to 50 nm up to about 200 nm in diameter, such as being from 60 nm to 180 nm.

As used herein, the terms “secretome” and “secretome composition” interchangeably refer to one or more molecules and/or biological factors that are secreted by cells into the extracellular space (such as into a culture medium). A secretome or secretome composition may include, without limitation, extracellular vesicles (e.g., exosomes, microparticles, etc.), proteins, nucleic acids, cytokines, and/or other molecules secreted by cells into the extracellular space (such as into a culture medium). A secretome or secretome composition may be left unpurified or further processed (for example, components of a secretome or secretome composition may be present within culture medium, such as in a conditioned medium; or alternatively, components of a secretome or secretome composition may be purified, isolated, and/or enriched, from a culture medium or extract, part, or fraction thereof). A secretome or secretome composition may further comprise one or more substances that are not secreted from a cell but includes media elements (e.g., culture media, additives, nutrients, etc.). Alternatively, a secretome or secretome composition does not comprise media elements (e.g., culture media, additives, nutrients, etc.).

As used herein, the term “conditioned medium” refers to a culture medium (or extract, part, or fraction thereof) in which one or more cells of interest have been cultured. Preferably, conditioned medium is separated from the cultured cells before use and/or further processing. The culturing of cells in culture medium may result in the secretion and/or accumulation of one or more molecules and/or biological factors (which may include, without limitation, extracellular vesicles (e.g., exosomes, microparticles, etc.), proteins, nucleic acids, cytokines, and/or other molecules secreted by cells into the extracellular space); the medium containing the one or more molecules and/or biological factors is a conditioned medium. Examples of methods of preparing conditioned media have been described in, for example, U.S. Pat. No. 6,372,494, which is incorporated by reference herein in its entirety.

As used herein, the term “cell culture” refers to cells grown under controlled condition(s) outside the natural environment of the cells. For instance, cells can be propagated completely outside of their natural environment (in vitro) or can be removed from their natural environment and the cultured (ex vivo). During cell culture, cells may survive in a non-replicative state, or may replicate and grow in number, depending on, for example, the specific culture media, the culture conditions, and the type of cells. An in vitro environment can be any medium known in the art that is suitable for maintaining cells in vitro, such as suitable liquid media or agar, for example.

The term “cell line” as used herein can refer to cultured cells that can be passaged at least one time without terminating.

The term “suspension” as used herein can refer to cell culture conditions in which cells are not attached to a solid support. Cells proliferating in suspension can be stirred while proliferating using an apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel that has been supplemented with medium suitable for further cell proliferation.

As used herein, the terms “culture medium,” “growth medium” or “medium” are used interchangeably and refer to a composition that is intended to support the growth and survival of organisms. While culture media is often in liquid form, other physical forms may be used, such as, for example, a solid, semi-solid, gel, suspension, and the like.

As used herein, the term “serum-free,” in the context of a culture medium or growth medium, refers to a culture or growth medium in which serum is absent. Serum typically refers to the liquid component of clotted blood, after the clotting factors (e.g., fibrinogen and prothrombin) have been removed by clot formation. Serum, such as fetal bovine serum, is routinely used in the art as a component of cell culture media, as the various proteins and growth factors therein are particularly useful for the survival, growth, and division of cells.

As used herein, the term “basal medium” refers to an unsupplemented synthetic medium that may contain buffers, one or more carbon sources, amino acids, and salts. Depending on the application, basal medium may be supplemented with growth factors and supplements, including, but not limited to, additional buffering agents, amino acids, antibiotics, proteins, and growth factors useful, for instance, for promoting growth, or maintaining or changing differentiation status, of particular cell types (e.g., fibroblast growth factor-basic (bFGF), also known as fibroblast growth factor 2 (FGF-2)).

As used herein, the terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

As used herein, the term “isolated” refers to material removed from its original environment, and is thus altered “by the hand of man” from its natural state.

As used herein, the term “enriched” means to selectively concentrate or increase the amount of one or more components in a composition, with respect to one or more other components. For instance, enrichment may include reducing or decreasing the amount of (e.g., removing or eliminating) unwanted materials; and/or may include specifically selecting or isolating desirable materials from a composition.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

As used herein, the terms “nucleic acid sequence,” “nucleotide sequence,” and “oligonucleotide” all refer to polymeric forms of nucleotides. As used herein, the term “polynucleotide” refers to a polymeric form of nucleotides that, when in linear form, has one 5′ end and one 3′ end, and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Inc., Woburn, MA) nucleosides, glycol nucleic acid, bridged nucleic acids, and morpholino structures. Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA. Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., Exonerate, BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is often between about 90% identity and 100% identity over the length of the reference polynucleotide or polypeptide or query sequence, for example, about 90% identity or higher, about 91% identity or higher, about 92% identity or higher, about 93% identity or higher, about 94% identity or higher, about 95% identity or higher, about 96% identity or higher, about 97% identity or higher, about 98% identity or higher, or about 99% identity or higher, over the length of the reference polynucleotide or polypeptide or query sequence. Sequence identity can also be calculated for the overlapping region of two sequences where only a portion of the two sequences can be aligned.

A moderate degree of sequence identity between two polynucleotides or two polypeptides is often between about 80% identity to about 90% identity over the length of the reference polynucleotide or polypeptide or query sequence, for example, about 80% identity or higher, about 81% identity or higher, about 82% identity or higher, about 83% identity or higher, about 84% identity or higher, about 85% identity or higher, about 86% identity or higher, about 87% identity or higher, about 88% identity or higher, or about 89% identity or higher, but less than 90%, over the length of the reference polynucleotide or polypeptide or query sequence.

A low degree of sequence identity between two polynucleotides or two polypeptides is often between about 50% identity and 75% identity over the length of the reference polynucleotide or polypeptide or query sequence, for example, about 50% identity or higher, about 60% identity or higher, about 70% identity or higher, but less than 75% identity, over the length of the reference polynucleotide or polypeptide or query sequence.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

“Gene” as used herein refers to a polynucleotide sequence comprising exons and related regulatory sequences. A gene may further comprise introns and/or untranslated regions (UTRs).

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, a “different” or “altered” level of, for example, a characteristic or property, is a difference that is measurably different, and preferably, statistically significant (for example, not attributable to the standard error of the assay). In some embodiments, a difference, e.g., as compared to a control or reference sample, may be, for example, a greater than 10% difference, a greater than 20% difference, a greater than 30% difference, a greater than 40% difference, a greater than 50% difference, a greater than 60% difference, a greater than 70% difference, a greater than 80% difference, a greater than 90% difference, a greater than 2-fold difference; a greater than 5-fold difference; a greater than 10-fold difference; a greater than 20-fold difference; a greater than 50-fold difference; a greater than 75-fold difference; a greater than 100-fold difference; a greater than 250-fold difference; a greater than 500-fold difference; a greater than 750-fold difference; or a greater than 1,000-fold difference, for example.

As used herein, the term “between” is inclusive of end values in a given range (e.g., between about 1 and about 50 nucleotides in length includes 1 nucleotide and 50 nucleotides).

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description.

The terms “effective amount” or “therapeutically effective amount” of a composition or agent, such as a therapeutic composition as provided herein, refers to a sufficient amount of the composition or agent to provide the desired response. Such responses will depend on the particular disease in question and related conditions.

“Transformation” as used herein refers to the insertion of an exogenous polynucleotide into a host cell, irrespective of the method used for insertion. For example, transformation can be by direct uptake, transfection, infection, and the like. The exogenous polynucleotide may be maintained as a nonintegrated vector, for example, an episome, or, alternatively, may be integrated into the host genome.

As used herein, the term “hypoxia” or “hypoxic” refers to a condition where the oxygen (O₂) concentration is below atmospheric O₂ concentration (typically 20-21%). In some embodiments, hypoxia refers to a condition with an O₂ concentration that is between 0% and 19%, between 2% and 18%, between 3% and 17%, between 4% and 16%, between 5% and 15%, between 5% and 10%, or less than 10%, less than 9%, less than 8%, less than 7%, less than 6%, less than 5%, less than 4%, less than 3%, less than 2%, or less than 1%.

As used herein, the term “normoxia” refers to a normal atmospheric concentration of oxygen, typically around 20% to 21% 02.

Generation of Progenitor Cells from Stem Cells

The present disclosure relates, in part, to methods for generating a secretome containing extracellular vesicles (EVs) from progenitor cells. In certain embodiments herein, progenitor cells may be isolated from a subject or tissue and used in the methods of the present disclosure. In other embodiments, progenitor cells may be generated from pluripotent stem cells, such as from embryonic stem (ES) cells or induced pluripotent stem cells (iPSCs).

Generation of iPSC Cells

iPSC cells may be obtained from, for example, somatic cells, including human somatic cells. The somatic cell may be derived from a human or non-human animal, including, for example, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like.

In some embodiments, the somatic cell is selected from keratinizing epithelial cells, mucosal epithelial cells, exocrine gland epithelial cells, endocrine cells, liver cells, epithelial cells, endothelial cells, fibroblasts, muscle cells, cells of the blood and the immune system, cells of the nervous system including nerve cells and glial cells, pigment cells, and progenitor cells, including hematopoietic stem cells. The somatic cell may be fully differentiated (specialized) or may be less than fully differentiated. For instance, undifferentiated progenitor cells that are not PSCs, including somatic stem cells, and finally differentiated mature cells, can be used. The somatic cell may be from an animal of any age, including adult and fetal cells.

The somatic cell may be of mammalian origin. Allogeneic or autologous stem cells can be used, if for example, the secretome (or extracellular vesicles) from a progenitor cell thereof is used for administration in vivo. In some embodiments, iPSCs are not MHC-/HLA-matched to a subject. In some embodiments, iPSCs are MHC-/HLA-matched to a subject. In embodiments, for example, where iPSCs are to be used to produce PSC-derived progenitor cells (to obtain a secretome, or extracellular vesicles, for therapeutic use in a subject), somatic cells may be obtained from the subject to be treated, or from another subject with the same or substantially the same HLA type as that of the subject. Somatic cells can be cultured before nuclear reprogramming, or can be reprogrammed without culturing after isolation, for example.

To introduce reprogramming factors into somatic cells, for example, viral vectors may be used, including, e.g., vectors from viruses such as SV40, adenovirus, vaccinia virus, adeno-associated virus, herpes viruses including HSV and EBV, Sindbis viruses, alphaviruses, human herpesvirus vectors (HHV) such as HHV-6 and HHV-7, and retroviruses. Lentiviruses include, but are not limited to, Human Immunodeficiency Virus type 1 (HIV-1), Human Immunodeficiency Virus type 2 (HIV-2), Simian Immunodeficiency Virus (SIV), Feline Immunodeficiency Virus (FIV), Equine Infectious Anaemia Virus (EIAV), Bovine Immunodeficiency Virus (BIV), Visna Virus of sheep (VISNA) and Caprine Arthritis-Encephalitis Virus (CAEV). Lentiviral vectors are capable of infecting non-dividing cells and can be used for both in vivo and in vitro gene transfer and expression of nucleic acid sequences. A viral vector can be targeted to a specific cell type by linkage of a viral protein, such as an envelope protein, to a binding agent, such as an antibody, or a particular ligand (for targeting to, for instance, a receptor or protein on or within a particular cell type).

In some embodiments, a viral vector, such as a lentiviral vector, can integrate into the genome of the host cell. The genetic material thus transferred is then transcribed and possibly translated into proteins inside the host cell. In other embodiments, viral vectors are used that do not integrate into the genome of a host cell.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector, for example.

Somatic cells can be reprogrammed to produce induced pluripotent stem cells (iPSCs) using methods known to one of skill in the art. One of skill in the art can readily produce induced pluripotent stem cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; and 8,268,620, all of which are incorporated herein by reference.

Generally, reprogramming factors which can be used to create induced pluripotent stem cells, either singly, in combination, or as fusions with transactivation domains, include, but are not limited to, one or more of the following genes: Oct4 (Oct3/4, Pou5f1), Sox (e.g., Sox1, Sox2, Sox3, Sox18, or Sox15), Klf (e.g., Klf4, Klf1, Klf3, Klf2 or Klf5), Myc (e.g., c-myc, N-myc or L-myc), nanog, or LIN28. As examples of sequences for these genes and proteins, the following accession numbers are provided: Mouse MyoD: M84918, NM_010866; Mouse Oct4 (POU5F1): NM_013633; Mouse Sox2: NM_011443; Mouse Klf4: NM_010637; Mouse c-Myc: NM_001177352, NM_001177353, NM_001177354 Mouse Nanog: NM_028016; Mouse Lin28: NM_145833: Human MyoD: NM_002478; Human Oct4 (POU5F1): NM_002701, NM 203289, NM_001173531; Human Sox2: NM_003106; Human Klf4: NM_004235; Human c-Myc: NM_002467; Human Nanog: NM_024865; and/or Human Lin28: NM_024674. Also contemplated are sequences similar thereto, including those having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity. In some embodiments, at least three, or at least four of Klf4, c-Myc, Oct3/4, Sox2, Nanog, and Lin28 are utilized. In other embodiments, Oct3/4, Sox2, c-Myc and Klf4 are utilized.

Exemplary reprogramming factors for the production of iPSCs include (1) Oct3/4, Klf4, Sox2, L-Myc (Sox2 can be replaced with Sox1, Sox3, Sox15, Sox17 or Sox18; Klf4 is replaceable with Klf1, Klf2 or Klf5); (2) Oct3/4, Klf4, Sox2, L-Myc, TERT, SV40 Large T antigen (SV40LT), (3) Oct3/4, Klf4, Sox2, L-Myc, TERT, human papilloma virus (HPV)16 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, Bmi1; (7) Oct3/4, Klf4, Sox2, L-Myc, Lin28; (8) Oct3/4, Klf4, Sox2, L-Myc, Lin28, SV40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb is replaceable with Esrrg); (12) Oct3/4, Klf4, Sox2; (13) Oct3/4, Klf4, Sox2, TERT, SV40LT; (14) Oct3/4, Klf4, Sox2, TERT, HPV16 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, Bmi1; (18) Oct3/4, Klf4, Sox2, Lin28 (19) Oct3/4, Klf4, Sox2, Lin28, SV40LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb is replaceable with Esrrg).

iPSCs typically display the characteristic morphology of human embryonic stem cells (hESCs), and express the pluripotency factor, NANOG. Embryonic stem cell specific surface antigens (SSEA-3, SSEA-4, TRA1-60, TRA1-81) may also be used to identify fully reprogrammed human cells. Additionally, at a functional level, PSCs, such as ESCs and iPSCs, also demonstrate the ability to differentiate into lineages from all three embryonic germ layers, and form teratomas in vivo (e.g., in SCID mice).

### Differentiating PSCs to Generate Progenitor Cells

The present disclosure further contemplates differentiating PSCs, including ESCs and iPSCs, into progenitor cells. Such progenitor cells can then be used to produce a secretome (and extracellular vesicles) of the present disclosure.

Progenitor cells of the present disclosure include, for example, hematopoietic progenitor cells, myeloid progenitor cells, neural progenitor cells; pancreatic progenitor cells, cardiac progenitor cells, cardiomyocyte progenitor cells, cardiovascular progenitor cells, renal progenitor cells, skeletal myoblasts, satellite cells, intermediate progenitor cells formed in the subventricular zone, radial glial cells, bone marrow stromal cells, periosteum cells, endothelial progenitor cells, blast cells, boundary caop cells, and mesenchymal stem cells. Methods for differentiating pluripotent stem cells to progenitor cells, and for culturing and maintaining progenitor cells are described in for example, U.S. patent application Ser. No. 17/931,669, entitled “Methods for the Production of Committed Cardiac Progenitor Cells,” which is incorporated by reference herein in its entirety.

### Production of Secretome/Extracellular Vesicles

The present disclosure encompasses the culturing of progenitor cells for secretome/extracellular vesicle production under GMP-ready and/or GMP-compatible conditions, to produce, e.g., GMP-ready and/or GMP-compatible products. The present disclosure also encompasses the culturing of progenitor cells for secretome/extracellular vesicle production under non-GMP-ready and/or non-GMP-compatible conditions, to produce, e.g., non-GMP-ready and/or non-GMP-compatible products.

In methods for generating secretomes or extracellular vesicles of the present disclosure, progenitor cells are typically subjected to two or more culturing steps in a serum-free culture medium.

In a first culturing step, one or more progenitor cells are cultured in a first serum-free culture medium that comprises basal medium, human serum albumin, and one or more growth factors. This first serum-free culture medium is then replaced with a second serum-free culture medium that comprises basal medium but does not comprise human serum albumin or the one or more growth factors. In a second culturing step, the one or more progenitor cells are then cultured in the second serum-free culture medium. Following the second culturing step, the second serum-free culture medium is recovered, to thereby obtain conditioned medium containing the secretome of the one or more progenitor cells.

The one or more progenitor cells can be, for example, progenitor cells that have recently been isolated or differentiated (e.g., from stem cells). Alternatively, in some embodiments, progenitor cells that have previously been refrigerated, frozen, and/or cryopreserved, may be used in the culturing methods of the present disclosure. In some embodiments, progenitor cells are thawed from a cryopreserved state (e.g., −80 °C or colder) before use. In some embodiments thereof, the cells are thawed in a thawing medium. In some embodiments, the thawing medium may comprise a liquid medium (e.g., alpha-MEM, STEMdiff™ Cardiomyocyte Support Medium (StemCell, Ref. 05027)) containing one or more supplements. In some embodiments, the supplement in the thawing medium may be one or more of a carbon source (e.g., glucose), an albumin, B-27, insulin, FGF-2, FGF, and an antibiotic (e.g., gentamicin). In some embodiments, the cells may be thawed in a thawing device, such as, for example, a water bath or a water-free thawing system (e.g., ThawSTAR™ Automated Thawing System, Biolife Solutions®). Cells may be thawed, for example, within a tube or bottle (e.g., plastic, glass), or bag (e.g., an Ethyl Vinyl Acetate (EVA) bag), such as a 500-1000 mL volume bag (e.g., Corning, Refs: 91-200-41, 91-200-42).

The one or more growth factors may be selected based on the type of progenitor cell, for example. In some embodiments, the one or more growth factors may be selected from Adrenomedullin, Angiopoietin, Autocrine motility factor, Bone morphogenetic proteins (BMPs), Ciliary neurotrophic factor (CNTF), Leukemia inhibitory factor (LIF), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1 (FGF-1), Fibroblast growth factor 2 (FGF-2), Fibroblast growth factor 3 (FGF-3), Fibroblast growth factor 4 (FGF-4), Fibroblast growth factor 5 (FGF-5), Fibroblast growth factor 6 (FGF-6), Fibroblast growth factor 7 (FGF-7), Fibroblast growth factor 8 (FGF-8), Fibroblast growth factor 9 (FGF-9), Fibroblast growth factor 10 (FGF-10), Fibroblast growth factor 11 (FGF-11), Fibroblast growth factor 12 (FGF-12), Fibroblast growth factor 13 (FGF-13), Fibroblast growth factor 14 (FGF-14), Fibroblast growth factor 15 (FGF-15), Fibroblast growth factor 16 (FGF-16), Fibroblast growth factor 17 (FGF-17), Fibroblast growth factor 18 (FGF-18), Fibroblast growth factor 19 (FGF-19), Fibroblast growth factor 20 (FGF-20), Fibroblast growth factor 21 (FGF-21), Fibroblast growth factor 22 (FG-F22), Fibroblast growth factor 23 (FGF-23), Foetal Bovine Somatotrophin (FBS), Glial cell line-derived neurotrophic factor (GDNF), Neurturin, Persephin, Artemin, Growth differentiation factor-9 (GDF-9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-3), Tumor necrosis factor-alpha (TNF-α), and Vascular endothelial growth factor (VEGF).

The amount of growth factor may be adjusted depending on the desired culture conditions and/or need. In some embodiments, the one or more growth factors may each independently be present in an amount from 0.001 μg/mL-1000 μg/mL, in an amount from 0.01 μg/mL-100 g/mL, in an amount from 0.1 g/mL-10 g/mL, in an amount from 0.05 μg/mL-5 g/mL, in an amount from 0.5 μg/mL-2.5 g/mL, or in an amount of about 0.5 g/mL, about 1 g/mL, about 2 μg/mL, about 3 μg/mL, about 4 μg/mL or about 5 μg/mL.

In some embodiments, the one or more growth factors comprise FGF-2. In some embodiments, the one or more growth factors consist of FGF-2.

The basal medium may be any basal culture medium suitable for the cell type to be cultured, including, for example, Dulbecco's Modified Eagle's Medium (DMEM), DMEM F12 medium, Eagle's Minimum Essential Medium (MEM), a-MEM, F-12K medium, Iscove's Modified Dulbecco's Medium, Knockout DMEM, or RPMI-1640 medium, or variants, combinations, or modifications thereof.

Additional supplements can also be added to the basal medium to supply the cells with trace elements for optimal growth and expansion. Such supplements include, for example, insulin, transferrin, sodium selenium, Hanks' Balanced Salt Solution, Earle's Salt Solution, antioxidant supplements, MCDB-201, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids, and combinations thereof. Such amino acids include, but are not limited to, L-alanine, L-arginine, L-aspartic acid, L-asparagine, L-cysteine, L-cysteine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

Optionally, hormones can also be used in cell culture and include, but are not limited to, D-aldosterone, diethylstilbestrol (DES), dexamethasone, beta-estradiol, hydrocortisone, insulin, prolactin, progesterone, somatostatin/human growth hormone (HGH), thyrotropin, thyroxine, and L-thyronine. Beta-mercaptoethanol can also be supplemented in cell culture media.

Lipids and lipid carriers can also be used to supplement cell culture media, depending on the type of cell. Such lipids and carriers can include, but are not limited to, cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

In certain embodiments, an albumin, such as human serum albumin, is present in the first serum-free culture medium. The albumin, including human serum albumin, may be, for example, isolated, synthetic, recombinant, and/or modified. The amount of albumin may be adjusted depending on the desired culture conditions and/or need. In some embodiments, the albumin may be present in an amount from 0.1 g/mL-50 mg/mL, in an amount from 1 g/mL-25 mg/mL, in an amount from 10 μg/mL-20 mg/mL, in an amount from 100 μg/mL-10 mg/mL, in an amount from 0.5 mg/mL-5 mg/mL, in an amount from 1 mg/mL-3 mg/mL, or in an amount of about 0.5 mg/mL, 1 mg/mL, 2 mg/mL, 3 mg/mL, 4 mg/mL or 5 mg/mL.

In some embodiments, the serum-free media further comprises one or more selected from the group consisting of: glutamine; biotin; DL alpha tocopherol acetate; DL alpha-tocopherol, vitamin A; catalase; insulin; transferrin; superoxide dismutase; corticosterone; D-galactose; ethanolamine, glutathione; L-carnitine; linoleic acid; progesterone; putrescine, sodium selenite; triodo-I-thyronine; an amino acid; sodium pyruvate; lipoic acid; vitamin B12; nucleosides; and ascorbic acid.

The basal medium may also be supplemented with one or more carbon sources. The one or more carbon sources may be selected from, for example, carbon sources such as glycerol, glucose, galactose, sucrose, fructose, mannose, lactose, or maltose. In some embodiments, a carbon source, such as glucose, may be present in an amount of at least 0.01 g/mL, 0.05 g/mL, 0.1 g/mL, 0.5 g/mL, 1 g/mL, 1.5 g/mL, 2 g/mL, 2.5 g/mL, 3 g/mL, 4 g/mL, or 5 g/mL.

A rock inhibitor may also be included in a culture medium, such as, for example, the rock inhibitor H1152.

The first and second culturing steps may be performed for differing lengths of time. For instance, the first and second culturing steps may each independently be performed for a period of 6-96 hours, 12-72 hours, 36-60 hours, 42-56 hours, or for about 12 hours, about 18 hours, about 24 hours, about 30 hours, about 36 hours, about 42 hours, about 48 hours, about 54 hours, about 60 hours, about 66 hours, about 72 hours, about 78 hours, about 84 hours, about 90 hours, or about 96 hours.

In some embodiments, the first culturing step is performed for a period of 42-56 hours, such as about 48 hours. In some embodiments, the second culturing step is performed for a period of 42-56 hours, such as about 48 hours.

In some embodiments, the first culturing step is performed for a period of 42-96 hours, such as about 72 hours. In some embodiments, the second culturing step is performed for a period of 42-56 hours, such as about 48 hours.

In some embodiments, all or a part of the first and/or second culturing step is performed under hypoxic conditions. In some embodiments, all or a part of the second culturing step is performed under hypoxic conditions. In some embodiments, the last 6-72 hours, the last 10-48 hours, or the last 12-36 hours, of the second culturing step is performed under hypoxic conditions. In some embodiments, the hypoxic condition is an O₂ concentration that is between 0% and 15%, between 0% and 10%, or less than 10%, less than 9%, less than 8%, less than 7%, less than 6%, less than 5%, less than 4%, less than 3%, less than 2%, or less than 1%.

In some embodiments, all or a part of the first and/or second culturing step is performed under normoxic conditions. In some embodiments, all or a part of the second culturing step is performed under normoxic conditions. In some embodiments, at least the last 6-72 hours, the last 10-48 hours, or the last 12-36 hours, of the second culturing step is performed under normoxic conditions. In some embodiments, the normoxic condition is an 02 concentration that is between 20% and 21%.

In some embodiments, all or a part of the first and/or second culturing step is performed in the presence of insulin. In some embodiments, all or a part of the first culturing step is performed in the presence of insulin. In some embodiments, the first culturing step comprises culturing in the presence of insulin for at least 24 hours, at least 48 hours, or at least 72 hours. In some embodiments, all or a part of the second culturing step is performed in the presence of insulin. In some embodiments, the second culturing step comprises culturing in the presence of insulin for at least 24 hours, at least 48 hours, or at least 72 hours.

In some embodiments, the one or more progenitor cells are washed, using one or more washing steps, between the first and second culturing steps. In some embodiments, the washing medium may comprise a liquid medium (e.g., alpha-MEM, DMEM) optionally containing one or more supplements. In some embodiments, the supplement is a carbon source (e.g., glucose). In some embodiments, the one or more progenitor cells are not washed between the first and second culturing steps (for instance, the first culture medium is removed and the second culture medium is then added).

The first and/or second culturing steps can be performed in suspension or attached to a solid support. The culturing may be two-dimensional or three-dimensional cell culturing.

For instance, in some embodiments, the culture vessel used for culturing may be a flask, flask for tissue culture (e.g., T25, T75), hyperflask (e.g., CellBind surface HYPERFlask®; Corning, Ref: 10024) or hyperstack (e.g., 12 or 36 chamber, HYPERStacks®, Corning, Refs: 10012, 10036, 10013, 10037), dish, petri dish, dish for tissue culture, multi dish, micro plate, micro-well plate, multi plate, multi-well plate, micro slide, chamber slide, tube, tray, CellSTACK® Chambers (e.g., 1ST, 2ST, 5ST, 10ST; Corning, Refs: 3268, 3269, 3313, 3319), culture bag, roller bottle, bioreactor, stirred culture vessel, spinner flask, microcarrier, or a vertical wheel bioreactor, for example. The one or more progenitor cells may be cultured in a volume of at least or about 0.2, 0.5, 1, 2, 5, 10, 15, 20, 30, 40, 50 mL, 100 mL, 150 mL, 200 mL, 250 mL, 300 mL, 350 mL, 400 mL, 450 mL, 500 mL, 550 mL, 600 mL, 800 mL, 1000 mL, 1500 mL, 1 L, 5 L, 10 L, 50 L, 100 L, 1000 L, 5000 L, or 10,000 L, for example.

In embodiments in which culturing comprises two-dimensional cell culture, such as on the surface of a culture vessel, the culture surface (to which the cells are intended to adhere) may be coated with one or more substances that promote cell adhesion. Such substances useful for enhancing attachment to a solid support include, for example, type I, type II, and type IV collagen, concanavalin A, chondroitin sulfate, fibronectin, fibronectin-like polymers, gelatin, laminin, poly-D and poly-L-lysine, Matrigel, thrombospondin, osteopontin, poly-D-lysine, human extracellular matrix, Corning® Cell-Tak™ Cell and Tissue Adhesive, Corning PuraMatrix® Peptide Hydrogel, and/or vitronectin.

In some embodiments, where culturing of cells is performed as adherent culture, e.g., where cells are adhered to a solid support, cells may be seeded at an amount of 25,000-250,000 cells per cm²; 50,000-200,000 cells per cm²; 75,000-175,000 cells per cm²; or between 100,000-150,000 cells per cm².

In some embodiments, where culturing of cells is performed as adherent culture, e.g., where cells are adhered to a solid support, cells may be seeded to the solid support under gravitational force. In other embodiments, the cells may be seeded to the solid support under centrifugation.

In some embodiments, following the second culturing step, the second serum-free culture medium used in the second culturing step is recovered to obtain a conditioned medium containing the secretome of the one or more progenitor cells.

The recovered, conditioned medium may in some embodiments be subjected to one or more further processing steps. Following the second culturing step, the second serum-free culture medium used in the second culturing step may be removed, analyzed, recovered, concentrated, enriched, isolated, purified, refrigerated, frozen, cryopreserved, lyophilized, sterilized, etc.

In some embodiments, the recovered, conditioned medium may be pre-cleared or clarified to remove particulates of greater than a certain size. For instance, the recovered, conditioned medium may be pre-cleared or clarified by one or more centrifugation and/or filtration techniques. In some embodiments, in-line filters may be used, gradually stepping down the pore size to minimize clogging and loss of material. In some embodiments, such as where TFF is performed, pore sizes as low as 0.2 μm may be used to avoid clogging/high pressures at the TFF stage.

In some embodiments, the recovered, conditioned medium is further processed to obtain a particular extract or fraction of the recovered, conditioned medium. For instance, the recovered, conditioned medium may be further processed to separate a small extracellular vesicle-enriched fraction (sEV) therefrom. An sEV fraction may be separated from the recovered, conditioned medium (or from a previously processed extract or fraction thereof) by one or more techniques such as centrifugation, ultracentrifugation, filtration, ultrafiltration, gravity, sonication, density-gradient ultracentrifugation, tangential flow filtration, size-exclusion chromatography, ion-exchange chromatography, affinity capture, polymer-based precipitation, or organic solvent precipitation, for example.

In some embodiments, conditioned medium is subjected to clarification by one or more filtration steps. In some embodiments thereof, one or more of the filtration steps utilizes a filter membrane having a particular pore size. In some embodiments thereof, a filter is used having a pore size of between 0.1 μm and 500 μm, or between 0.2 μm and 200 μm; or having a pore size less than or equal to 500 μm, 400 μm, 300 μm, 200 μm, 100 μm, 50 μm, 40 μm, 30 μm, 20 μm, 15 μm, 10 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm or 0.1 μm.

In some embodiments, the clarification comprises at least 1, at least 2, at least 3, at least 4, at least 5, at least 6, or at least 7, filtration steps. In some embodiments, the clarification comprises 4 filtration steps. In some embodiments, successive filtration steps utilize filters having increasingly smaller pores.

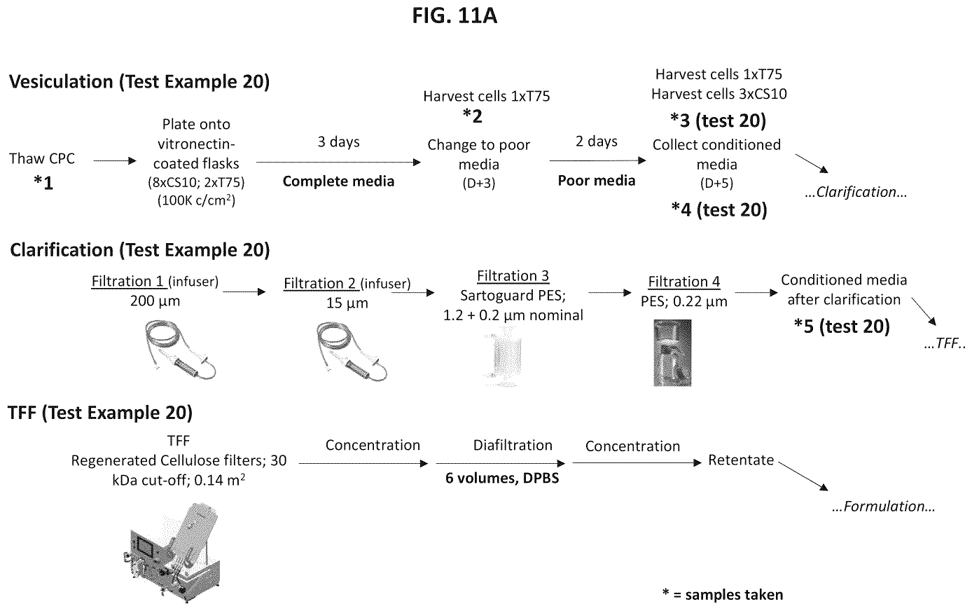

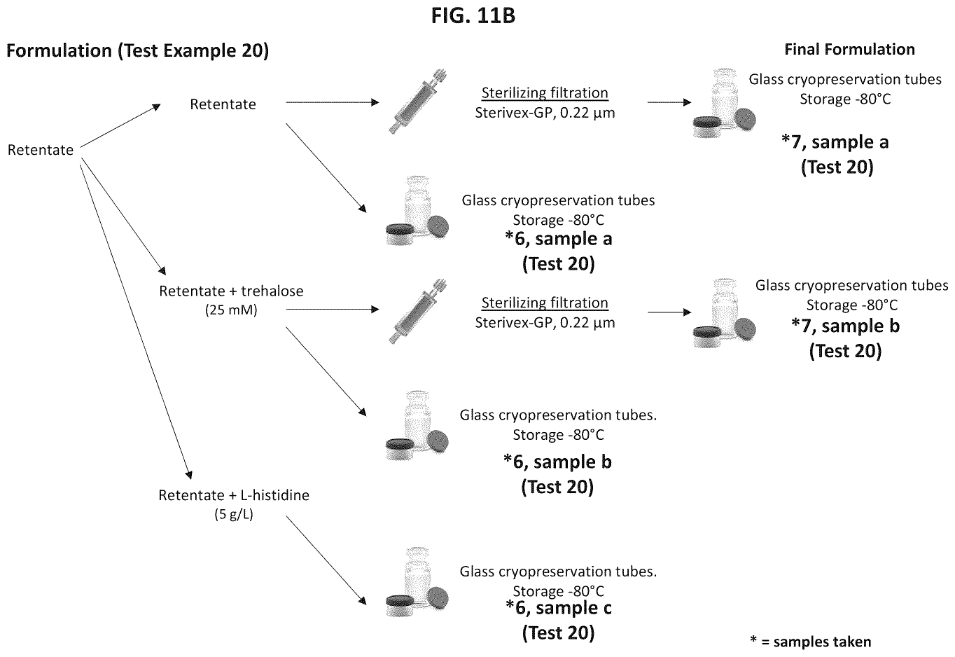

In some embodiments thereof, a first filtration step comprises use of an approximately 200 μm filter (e.g., a 200 μm drip chamber filter; Gravity Blood set, BD careFusion, Ref: VH-22-EGA); a second filtration step comprises use of an approximately 15 μm filter (e.g., DIDACTIC, Ref: PER1FL25), a third filtration step comprises use of an approximately 0.2 μm filter, optionally containing a pre-filter, for example, an approximately 1.2 μm pre-filter (e.g., Sartoguard PES XLG MidiCaps, pore sizes: 1.2 μm+0.2 μm, Sartorius, Ref: 5475307F7-OO-A); and a fourth filtration step comprises use of an approximately 0.22 μm filter (e.g., Vacuum Filter/Storage Bottle System, 0.22 μm pore, 33.2 cm² PES Membrane, Corning, Ref: 431097), as illustrated in Example 5 and in FIG. 11A.

In other embodiments thereof, a first filtration step comprises use of an approximately 5 μm filter (e.g., Sartopure PP3 MidiCaps, pore size: 5 μm, Sartorius, Ref: 5055342P9-OO-A), a second filtration step comprises use of an approximately 0.2 μm filter, optionally containing a pre-filter, for example, an approximately 1.2 μm pre-filter (e.g., Sartoguard PES MidiCaps, pore sizes: 1.2 μm+0.2 μm, Sartorius, Ref: 5475307F9-OO-A, and a third filtration step comprises use of an approximately 0.2 μm filter, optionally containing a pre-filter, for example, an approximately 0.45 μm pre-filter (e.g., Sartopure 2 MidiCaps, pore sizes: 0.45 μm+0.2 μm, Sartorius, Ref: 5445307H8-OO-A), as illustrated in Example 12 and in FIG. 24A.

In some embodiments, conditioned medium may be subjected to clarification by one or more centrifugation steps. In some embodiments, conditioned medium may be subjected to clarification by a combination of centrifugation and filtration step(s).

In some embodiments, one or more additives are added to the conditioned medium, such as before clarification, and/or after clarification. In some embodiments, an additive is added that reduces aggregation. In some embodiments thereof, the additive is one or more selected from trehalose, histidine (e.g., L-histidine), arginine (e.g., L-arginine), citrate-dextrose solution, a Dnase (e.g., Dnase I), ferric citrate, or Anti-Clumping Agent (Gibco/Life technologies, Ref: 01-0057; Lonza, Ref: BE02-058E).

In some embodiments, conditioned medium or sEV may be subjected to isolation, enrichment, and/or concentration step(s) using tangential flow filtration (TFF). In some embodiments, the conditioned medium or sEV is subjected to TFF after clarification that employed one or more clarification steps (e.g., such as after one or more filtration and/or centrifugation steps). TFF is a rapid and efficient method for separating, enriching and purifying biomolecules. In some embodiments, TFF can be used, e.g., for concentrating (e.g., concentrating small extracellular vesicles from conditioned media); for diafiltration; and for concentrating and diafiltration. Diafiltration is a type of ultrafiltration process in which the retentate (the fraction that does not pass through the membrane) is diluted with buffer and re-ultrafiltered, to reduce the concentration of soluble permeate components and increase further the concentration of retained components.

In some embodiments, TFF is used for enriching, concentrating and diafiltration of conditioned medium or sEV (e.g., for concentration and diafiltration of EV secretome). In some embodiments, TFF is first used to concentrate conditioned medium or sEV, and is subsequently used for diafiltration. In some embodiments, a TFF process may comprise a further step of concentrating after diafiltration. In some embodiments, TFF is used for diafiltration but not concentrating. In some embodiments, TFF is used for concentrating but not diafiltration.

In some embodiments, the TFF membrane has a cut-off value of or less than 10 kDa, of or less than 20 kDa, of or less than 30 kDa, of or less than 40 kDa, of or less than 50 kDa, of or less than 60 kDa, of or less than 70 kDa, of or less than 80 kDa, of or less than 90 kDa, of or less than 100 kDa, or of or less than 150 kDa. In some embodiments, the TFF membrane has a cut-off value of about 10 kDa, about 30 kDa, about 100 kDa, or about 500 kDa. In some embodiments, the TFF membrane has a cut-off value of 30 kDa or about 30 kDa.

In some embodiments, the TFF membrane comprises cellulose. In some embodiments, the TFF membrane comprises regenerated cellulose. In some embodiments, the TFF membrane comprises a polyethersulfone (PES) membrane.

In some embodiments, a TFF pressure of less than 0.1 bar, 0.5 bar, 1 bar, 1.5 bar, 2 bar, 2.5 bar, 3 bar, 3.5 bar, 4 bar, 4.5 bar, or 5 bar, may be used. In some embodiments, a TFF pressure of less than or equal to 3.5 bar is used, to address filter clogging and slow filtration rates (e.g., when using large scale (>5 L) media processing of spent medias using a low cut-off, such as 30 kDa).

In some embodiments, conditioned media or sEV subjected to TFF can be further purified, isolated, and/or enriched (after TFF) using one or more purification, isolation, and/or enrichment, techniques. For instance, the resulting product from TFF can be subjected to a chromatography step, such as an ion exchange chromatography step or a steric exclusion chromatography step, to even further purify small extracellular vesicles. In some embodiments, conditioned media subjected to TFF, with or without further purification, isolation, and/or enrichment, may be further concentrated, such as by ultracentrifugation.

Any of the above-described processing techniques can be performed on recovered, conditioned medium (or a previously processed extract or fraction thereof) that is fresh, or has previously been frozen and/or refrigerated, for example.

In some embodiments, secretome-, extracellular vesicle-, and sEV-containing compositions produced by the methods herein may have added thereto at least one additive to prevent aggregation. The additive may be one or more selected from trehalose, histidine (e.g., L-histidine), arginine (e.g., L-arginine), citrate-dextrose solution, a Dnase (e.g., Dnase I), ferric citrate, or Anti-Clumping Agent (Gibco/Life technologies, Ref: 01-0057; Lonza, Ref: BE02-058E). In some embodiments, trehalose is added. In some embodiments, trehalose or L-histidine is added.

In some embodiments, the sEV fraction is CD63⁺, CD81⁺, and/or CD9⁺. The sEV fraction may contain one or more extracellular vesicle types, such as, for example, one or more of exosomes, microparticles, and extracellular vesicles. The sEV fraction may also contain secreted proteins (enveloped and/or unenveloped). Extracellular vesicles within conditioned media or sEV fractions of the present disclosure may contain, for example, one or more components selected from tetraspanins (e.g., CD9, CD63 and CD81), ceramide, MHC class I, MHC class II, integrins, adhesion molecules, phosphatidylserine, sphingomyelin, cholesterol, cytoskeletal proteins (e.g., actin, gelsolin, myosin, tubulin), enzymes (e.g., catalase, GAPDH, nitric oxide synthase, LT synthases), nucleic acids (e.g., RNA, miRNA), heat shock proteins (e.g., HSP70 and HSP90), exosome biogenesis proteins (ALIX, Tsg101), LT, prostaglandins, and S100 proteins.

In some embodiments, the presence of desired extracellular vesicle types in a fraction can be determined, for example, by nanoparticle tracking analysis (to determine the sizes of particles in the fraction); and/or by confirming the presence of one or more markers associated with a desired extracellular vesicle type. For instance, a fraction of recovered, conditioned media can be analyzed for the presence of desired extracellular vesicle types by detecting the presence of one or more markers in the fraction, such as, for example, CD9, CD63 and/or CD81.

In some embodiments, an sEV formulation or composition is positive for CD9, CD63 and CD81 (canonical EV markers), and is positive for the cardiac-related markers CD49e, ROR1, SSEA-4, MSCP, CD146, CD41b, CD24, CD44, CD236, CD133/1, CD29 and CD142. In some embodiments, an sEV formulation or composition contains a lesser amount of one or more markers selected from the group consisting of CD3, CD4, CD8, HLA-DRDPDQ, CD56, CD105, CD2, CD1c, CD25, CD40, CD11c, CD86, CD31, CD20, CD19, CD209, HLA-ABC, CD62P, CD42a and CD69, as compared to the amount of CD9, CD63 and/or CD81 in the sEV formulation or composition. In some embodiments, an sEV formulation or composition contains an undetectable amount of (e.g., by MACSPlex assay, by immunoassay, etc.), or is negative for, one or more markers selected from the group consisting of CD19, CD209, HLA-ABC, CD62P, CD42a and CD69.

In some embodiments, the sEV formulation or composition is at least one of the following: an sEV formulation or composition that has been enriched for extracellular vesicles having a diameter of between about 50-200 nm or between 50-200 nm; an sEV formulation or composition that has been enriched for extracellular vesicles having a diameter of between about 50-150 nm or between 50-150 nm; an sEV formulation or composition that is substantially free or free of whole cells; and an sEV formulation or composition that is substantially free of one or more culture medium components (e.g., phenol-red).

In some embodiments, such as, for example, some GMP-compatible processes, testing panels are conducted to analyze and/or determine one or more properties of the processes, products thereof, or intermediate products, etc.

For instance, during the vesiculation stage (including, e.g., thawing, plating, culturing and/or harvesting steps), one or more properties of the cells may be examined (including, for example: the number of viable cells, the percentage viability of the cells; morphologies of the cells; identity of the cells; karyotype of the cells; and/or transcriptome of the cells).

Additionally, or alternatively, one or more properties of a secretome and/or extracellular vesicle-containing fraction, extract, or composition can be analyzed using one or more tests (including, e.g., particle concentration and/or particle size distribution; protein concentration; protein profile concentration; RNA profile; potency; marker identity; host cell protein assessment; residual DNA quantification and/or characterization; sterility; mycoplasma; endotoxin; appearance; pH; osmolarity; extractable volume; hemolytic activity; complement activation; platelet activation; and/or genotoxicity), to determine one or more properties of the secretome/extracellular vesicles. In some embodiments, an EV composition, formulation, fraction, or secretome, etc., may be analyzed by electron microscopy.

In some embodiments, RNA may be extracted from EVs to analyze the RNA transcriptome of an EV composition, formulation, fraction, or secretome, etc. In some embodiments thereof, microRNA is analyzed, such as, for example, by generating a cDNA library from extracted RNA; and sequencing all or a part of the library. In some embodiments, the sequence analysis comprises sorting the sequenced RNAs into different biotypes. In some embodiments, an EV composition, formulation, fraction, or secretome, etc., contains all or some of the miRNAs depicted in FIG. 43 or TABLE 9. In some embodiments, an EV composition, formulation, fraction, or secretome, etc., contains at least 1, at least 2, at least 3, at least 4, at least 5, at least 6, at least 7, at least 8, at least 9, or at least 10 of the miRNAs depicted in FIG. 43 or TABLE 9. In some embodiments, an EV composition, formulation, fraction, or secretome, etc., contains all or some of the miRNAs listed in TABLE 80. In some embodiments, an EV composition, formulation, fraction, or secretome, etc., contains at least 1, at least 2, at least 3, at least 4, at least 5, at least 6, at least 7, at least 8, at least 9, or at least 10 of the miRNAs listed in TABLE 80. In some embodiments, an EV composition, formulation, or fraction, etc., contains at least one of miR-302, miR-16, miR-126 and miR-93.

In some embodiments, proteomic analysis of an EV composition, formulation, fraction, or secretome, etc., may be conducted. In some embodiments, proteins may be isolated from an EV composition, formulation, fraction, or secretome, etc., and analyzed by mass spectrometry, such as, for example, nano-LC-MS/MS and HPLC-MS/MS analysis. In some embodiments, an EV composition, formulation, fraction, or secretome, etc., contains at least 1, at least 2, at least 3, at least 4, at least 5, at least 6, at least 7, at least 8, at least 9, or at least 10, at least 15, at least 20, or at least 25 of the proteins listed in TABLE 81.

Additionally, one or more of the above properties can be assessed on conditioned media before clarification; on conditioned media after clarification; on isolated and/or concentrated secretome/extracellular vesicles; and/or on final formulations.

In some embodiments, final formulations may be tested immediately after production and/or 1-week, 2-weeks, 1-month, 2-months, 3-months, 6-months, 1-year, 18 months and several years, after being formulated.

An exemplary process/product testing panel is shown in TABLE 49. This exemplary process is in addition to the description above. This panel was developed to characterize our process and ensure reproducibility, which further led to the development of CTC1-EV.

TABLE 49
An exemplary secretome/extracellular vesicle process/product testing panel

| | | Test Panel (samples tested) | | | | | |
|---|---|---|---|---|---|---|---|
| | | *1 | | | | | |
| | | (D + 0 cells, | | | | *5 | |
| | | final pooled | | | *4 | (Condi- | |
| | | cell | | | (D + 5 | tioned | *7 |
| | | suspension | *2 | *3 | Conditioned | media | (Final |
| | | prior | (D + 3 | (D + 5 | media before | after | formu- |
| Test | Method | to seeding) | material) | cells) | clarification) | clarification) | lation) |
| Cells-Number of | NucleoCounter NC-200 | Y | Y | Y | | | |
| Viable Cells | (DAPI/AO staining) | | (harvested | (harvested | | | |
| | (PhE 2.7.29) | | from T75) | from CS10s | | | |
| | | | | and T75) | | | |
| Cells-% Viability | NucleoCounter NC-200 | Y | Y | Y | | | |
| | (DAPI/AO staining) | | (harvested | (harvested | | | |
| | (PhE 2.7.29) | | from T75) | from CS10s | | | |
| | | | | and T75) | | | |
| Cells-Morphology | Microscopy | | Y | Y | | | |
| | | | (as viewed | (as viewed | | | |
| | | | on T75 and | on T75 and | | | |
| | | | CS10) | CS10) | | | |
| Cells-Identity | Flow Cytometry | Y | Y | Y | | | |
| | (PhE 2.7.24) | | (harvested | (harvested | | | |
| | | | from T75) | from CS10s) | | | |
| Cells-Karyotype | Conventional | | | Y | | | |
| | Cytogenetics | | | (harvested | | | |
| | | | | from flasks) | | | |
| Cells- | RNA sequencing | | Y | Y | | | |
| Transcriptome | | | (harvested | (harvested | | | |
| | | | from T75) | from CS10s) | | | |
| Secretome-Particle | Nanoparticle Tracking | | | | Y | Y | Y |
| concentration, | Analysis (NanoSight) | | | | | | |
| particle size | | | | | | | |
| distribution | | | | | | | |
| Secretome-Protein | BCA assay | | | | Y | Y | Y |
| concentration | (PhE 2.5.33) | | | | | | |
| Secretome-Protein | Mass Spectrometry | | | | | | Y |
| profile content | | | | | | | |
| Secretome-RNA | RNA sequencing | | | | | | Y |
| profile | | | | | | | |
| Secretome-Potency | Scratch Wound Healing | | | | | | Y |
| | Assay | | | | | | |
| | CM Survival Assay | | | | | | |
| Secretome-EV | Flow Cytometry: CD9/ | | | | | Y | Y |
| marker identity | CD63/CD81 and other EV | | | | | | |
| | surface markers by | | | | | | |
| | Macsplex ® Miltenyi | | | | | | |
| | (PhE 2.7.24) | | | | | | |
| Secretome-Host | GRP94 and HLA Class I | | | | | Y | Y |
| cell protein | ELISA assays | | | | | | |
| assessment | (PhE 2.6.34) | | | | | | |
| Secretome- | Immunoassay | | Y | | Y | | |
| Antibiotics | | | | | | | |
| Secretome- | Qubit, spectrophotometry, | | | | | | Y |
| Residual DNA, | qPCR (PhE 2.6.35) | | | | | | |
| quantification | | | | | | | |
| Secretome- | Exome sequencing | | | | | | Y |
| Residual DNA, | | | | | | | |
| characterisation | | | | | | | |
| Adventitious agent | Bovine, porcine virus | | | Y | Y | | |
| testing | detection; retrovirus testing | | | | | | |
| Sterility | Aerobic + anaerobic | Y | Y | | Y | | Y |
| | bacterial cultures | | (spent | | | | |
| | (PhE 2.6.1/2.6.27) | | media, | | | | |
| | | | CS10) | | | | |
| Mycoplasma | qPCR | | Y | | Y | | Y |
| | (PhE 2.6.7) | | (spent | | | | |
| | | | media, | | | | |
| | | | CS10) | | | | |
| Endotoxin | Kinetic chromogenic LAL | | Y | | Y | | Y |
| | (PhE 2.6.14) | | (spent | | | | |
| | | | media, | | | | |
| | | | CS10) | | | | |
| Secretome- | Clarity and degree of | | | | | | Y |
| Appearance | opalescence of liquids | | | | | | |
| | (PhE 2.2.1) | | | | | | |
| | Degree of coloration of | | | | | | |
| | liquids | | | | | | |
| | (PhE 2.2.2) | | | | | | |
| Secretome-pH | Potentiometric | | | | | | Y |
| | determination | | | | | | |
| | (PhE 2.2.3) | | | | | | |
| Secretome- | Osmometer | | | | | | Y |
| Osmolarity | (PhE 2.2.35) | | | | | | |
| Secretome- | PhE 2.9.17 | | | | | | Y |
| Extractable volume | | | | | | | |
| Secretome- | PhE 2.9.40 and 2.9.6 | | | | | | Y |
| Uniformity | | | | | | | |
| Secretome- | PhE 2.9.19 | | | | | | Y |
| Particulate | | | | | | | |
| contamination: sub- | | | | | | | |
| visible particles | | | | | | | |
| Secretome- | PhE 2.9.20 | | | | | | Y |
| Particulate | | | | | | | |
| contamination: | | | | | | | |
| visible particles | | | | | | | |
| Secretome-in vivo | GLP animal studies | | | | | | Y |

Therapeutic Compositions and Applications The present disclosure contemplates the generation of secretome-, extracellular vesicle-, and sEV-containing compositions useful as therapeutic agents. In some embodiments, the methods of the present disclosure comprise administering an effective amount of a secretome-, extracellular vesicle-, and/or sEV-containing composition to a subject in need thereof.

Tissues treated according to the methods of the present disclosure include, without limitation, cardiac tissue, brain or other neural tissue, skeletal muscle tissue, pulmonary tissue, arterial tissue, capillary tissue, renal tissue, hepatic tissue, tissue of the gastrointestinal tract, epithelial tissue, connective tissue, tissue of the urinary tract, etc. The tissue to be treated may be damaged or fully or partly non-functional due to an injury, age-related degeneration, acute or chronic disease, cancer, or infection, for example. Such tissues may be treated, for example, by intravenous administration of a secretome-, extracellular vesicle-, and/or sEV-containing composition.

In some embodiments, compositions of the present disclosure may be used to treat diseases such as myocardial infarction, stroke, heart failure, and critical limb ischemia, for example. In some embodiments, compositions of the present disclosure may be used to treat heart failure which has one or more of the following characteristics: is acute, chronic, ischemic, non-ischemic, with ventricular dilation, without ventricular dilation, with reduced left ventricular ejection fraction, or with preserved left ventricular ejection fraction. In some embodiments, compositions of the present disclosure may be used to treat heart failure selected from the group consisting of ischemic heart disease, cardiomyopathy, myocarditis, hypertrophic cardiomyopathy, diastolic hypertrophic cardiomyopathy, dilated cardiomyopathy, and post-chemotherapy induced heart failure. In some embodiments, compositions of the present disclosure may be used to treat diseases such as congestive heart failure, heart disease, ischemic heart disease, valvular heart disease, connective tissue diseases, viral or bacterial infection, myopathy, dystrophinopathy, liver disease, renal disease, sickle cell disease, diabetes, ocular diseases, and neurological diseases. In some embodiments, compositions of the present disclosure may be used to treat chemotherapy-induced cardiomyopathy (e.g., caused by anthracycline administration). It will be recognized that a suitable progenitor cell type(s) may be selected depending on the disease to be treated, or the tissue to be targeted.

For example, in some embodiments, a subject with a cardiac disease, such as acute myocardial infarction, chemotherapy-induced cardiomyopathy, or heart failure, can be treated with a secretome-, extracellular vesicle-, and/or sEV-containing composition, produced from cardiomyocyte progenitor cells, cardiac progenitor cells, and/or cardiovascular progenitor cells.

Additionally, a secretome-, extracellular vesicle-, and/or sEV-containing composition produced from an appropriate progenitor cell type can also be used to improve the functioning or performance of a tissue. For instance, an improvement in angiogenesis, or an improvement in cardiac performance, may be effected by delivering a secretome-, extracellular vesicle-, and/or sEV-containing composition, produced from cardiomyocyte progenitor cells, cardiac progenitor cells, and/or cardiovascular progenitor cells, to a subject in need thereof.

In some embodiments, the administration comprises administration at a tissue or organ site that is the same as the target tissue. In some embodiments, the administration comprises administration at a tissue or organ site that is different from the target tissue. Such administration may include, for example, intravenous administration.

A secretome-, extracellular vesicle-, and/or sEV-containing composition may contain, or be administered with, a pharmaceutically-acceptable diluent, carrier, or excipient. Such a composition may also contain, in some embodiments, pharmaceutically acceptable concentrations of one or more of a salt, buffering agent, preservative, or other therapeutic agent. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations. For instance, in some embodiments, a secretome-, extracellular vesicle-, and/or sEV-containing composition, may be formulated with a biomaterial, such as an injectable biomaterial. Exemplary injectable biomaterials are described, for example, in WO 2018/046870, incorporated by reference herein in its entirety.

The secretome-, extracellular vesicle-, and/or sEV-containing compositions of the present disclosure may be administered in effective amounts, such as therapeutically effective amounts, depending on the purpose. An effective amount will depend upon a variety of factors, including the material selected for administration, whether the administration is in single or multiple doses, and individual patient parameters including age, physical condition, size, weight, and the stage of disease. These factors are well known to those of ordinary skill in the art.

Any appropriate route of administration may be employed, for example, administration may be parenteral, intravenous, intra-arterial, subcutaneous, intratumoral, intramuscular, intracranial, intraorbital, ophthalmic, intraventricular, intrahepatic, intracapsular, intrathecal, intracisternal, intraperitoneal, intranasal, intramyocardial, intra-coronary, aerosol, suppository, epicardial patch, oral administration, or by perfusion. For instance, therapeutic compositions for parenteral administration may be in the form of liquid solutions or suspensions; for oral administration, formulations may be in the form of tablets or capsules; and for intranasal formulations, in the form of powders, nasal drops, or aerosols. For instance, in some embodiments, a subject with a cardiac disease, such as acute myocardial infarction or heart failure, can be treated with a secretome-, extracellular vesicle-, and/or sEV-containing composition, produced from cardiomyocyte progenitor cells, cardiac progenitor cells, and/or cardiovascular progenitor cells, wherein the composition is administered intravenously.

In some embodiments, a single dose of a secretome-, extracellular vesicle-, and/or sEV-containing composition may be administered. In other embodiments, multiple doses, spanning one or more doses per day, week, or month, are administered to the subject. In some embodiments, single or repeated administration of a secretome-, extracellular vesicle-, and/or sEV-containing composition, including two, three, four, five or more administrations, may be made. In some embodiments, the secretome-, extracellular vesicle-, and/or sEV-containing composition may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) or months, depending on the nature and/or severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ. As an example, if symptoms worsen, or do not improve, the secretome-, extracellular vesicle-, and/or sEV-containing composition, may be administered more frequently. Contrarily, if symptoms stabilize or diminish, the secretome-, extracellular vesicle-, and/or sEV-containing composition may be administered less frequently.

In some embodiments, a secretome-, extracellular vesicle-, and/or sEV-containing composition is administered in several doses, for example three, on or about several days, weeks, or months apart, for example two weeks apart, by intravenous administration. In some embodiments thereof, the composition may be diluted with, formulated with, and/or administered together with, a carrier, diluent, or suitable material (e.g., saline).

Assays for Determining Secretome and Extracellular Vesicle Activity, Functionality, and/or Potency The present disclosure also encompasses methods for analyzing the activity, functionality, and/or potency, of conditioned media; or of a secretome-, extracellular vesicle-, and/or sEV-containing composition.

The activity, functionality, and/or potency, of conditioned media; or of a secretome-, extracellular vesicle-, and/or sEV-containing composition, can be assessed by various techniques, depending on, for example, the type of progenitor cells used to produce the conditioned media or composition, and the desired use of the conditioned media or composition.

For instance, the activity, functionality, and/or potency, of conditioned media; or of a secretome-, extracellular vesicle-, and/or sEV-containing composition, can be assessed by administering the conditioned media, secretome-, extracellular vesicle-, and/or sEV-containing composition, to target cells in vitro, ex vivo, or in vivo. One or more properties of the target cells can then be analyzed, such as, for example, cell viability, hypertrophy, cell health, cell adhesion, cell physiology, ATP content, cell number, and cell morphology, to determine the activity, functionality, and/or potency, of conditioned media; or of a secretome-, extracellular vesicle-, and/or sEV-containing composition.

In some embodiments, assays known in the art may be used to determine the activity, functionality, and/or potency, of conditioned media; or of a secretome-, extracellular vesicle-, and/or sEV-containing composition.

For instance, for conditioned media; or for a secretome-, extracellular vesicle-, and/or sEV-containing composition, obtained from cardiovascular progenitor cells or cardiomyocyte progenitor cells, the activity, functionality, and/or potency, thereof may be measured using a known cardiomyocyte viability assay, such as described in El Harane et al. (Eur. Heart J., 2018, 39(20): 1835-1847).

Specifically, serum-deprived cardiac myoblasts (e.g., H9c2 cells) may be contacted with conditioned media; or a secretome-, extracellular vesicle-, and/or sEV-containing composition, and the viability of the cells measured thereafter. In some embodiments of this assay, the cells are deprived of serum before administering the conditioned media or the secretome-, extracellular vesicle-, and/or sEV-containing composition. In other embodiments, the cells are deprived of serum after administering the conditioned media or the secretome-, extracellular vesicle-, and/or sEV-containing composition. In some embodiments, the cells are deprived of serum before and after administering the conditioned media or the secretome-, extracellular vesicle-, and/or sEV-containing composition.

In other embodiments, the angiogenic activity of a conditioned media or a secretome-, extracellular vesicle-, and/or sEV-containing composition, can be measured, for example, using a HUVEC scratch wound healing assay. In HUVEC scratch wound healing assays, HUVEC cells are cultured on a culture surface, and the cultured cell layer(s) is then scratched; angiogenic activity of a conditioned media or a secretome-, extracellular vesicle-, and/or sEV-containing composition, can then be determined by the capacity of the conditioned media or the secretome-, extracellular vesicle-, and/or sEV-containing composition, to produce closure of the wound under serum-free conditions.

In some embodiments, the activity, functionality, and/or potency, of conditioned media; or of a secretome-, extracellular vesicle-, and/or sEV-containing composition, may be analyzed using a HUVEC (Human umbilical vein endothelial cells) plating assay. In some embodiments thereof, HUVEC cells are cultured in basal medium in the presence of conditioned media; or a secretome-, extracellular vesicle-, and/or sEV-containing composition, to analyze the effect of the conditioned media; or of a secretome-, extracellular vesicle-, and/or sEV-containing composition, on HUVEC viability. In some embodiments thereof, the conditioned media; secretome-, extracellular vesicle-, and/or sEV-containing composition, improves cell seeding, survival, and/or proliferation, of the HUVEC cells.

Cell viability (in cell viability assays) may be measured using, for example, a DNA-labeling dye or a nuclear-staining dye. The dye may be used with live cell imaging. Cell viability may also be measured by microscopy, such as fluorescence microscopy, using such a DNA-labeling dye or a nuclear-staining dye. Cell viability may also be measured, in a HUVEC plating assay for example, by analyzing ATP content.

In some embodiments, a conditioned media; or a secretome-, extracellular vesicle-, and/or sEV-containing composition, may be analyzed in an anti-fibrosis assay, to determine the effect of the conditioned media; or the secretome-, extracellular vesicle-, and/or sEV-containing composition, on treating or reducing fibrosis. In some embodiments thereof, cells are stimulated to induce a fibrotic state. In some embodiments, the cells induced to a fibrotic state are fibroblasts, such as, for example, cardiac fibroblasts. In some embodiments, the cells are stimulated with at least one stimulating agent to induce a fibrotic state. In some embodiments, the stimulating agent is a TGF-β (e.g., TGF-β1, TGF-β2 and/or TGF-β3), and/or bleomycin. In some embodiments, the induction, treatment, and/or reduction, of fibrosis is determined by analyzing one or more markers of fibrosis. In some embodiments, the one or more markers of fibrosis are analyzed by quantifying the amount of transcript encoding a marker of fibrosis. In some embodiments, the amount of transcript is quantified by quantitative reverse transcription polymerase chain reaction. In some embodiments, the amount of transcript is quantified using an array or next generation sequencing. In some embodiments, the expression of at least one of NMMP2 and Periostin are analyzed.

An activity, functionality, and/or potency, of conditioned media; or of a secretome-, extracellular vesicle-, and/or sEV-containing composition, may also be determined with reference to one or more control samples. For instance, control cells may be one or more of: serum-deprived control cells which are not administered the conditioned media or the secretome-, extracellular vesicle-, and/or sEV-containing composition; control cells which are not serum-deprived; or serum-deprived control cells which are administered a mock conditioned media or mock secretome-, extracellular vesicle-, and/or sEV-containing composition.

In some methods of the present disclosure, an activity, functionality, and/or potency, of conditioned media; or of a secretome-, extracellular vesicle-, and/or sEV-containing composition, can be assessed by a method comprising administering the conditioned media or the secretome-, extracellular vesicle-, and/or sEV-containing composition, to target cells cultured under at least one stress-inducing condition, and analyzing at least one property of the cells. The one or more properties of the target cells that may be analyzed can be selected from, for instance, cell migration, cell survival, cell viability, hypertrophy, cell health, cell adhesion, cell physiology, ATP content, cell number, and cell morphology. In some embodiments, the at least one property measured is cell adhesion, cell number, cell growth, and/or cell morphology, and wherein the cell adhesion, cell number, cell growth, and/or cell morphology, is determined by measuring electrical impedance across a culture vessel surface in the culture.

In a first method thereof, target cells are cultured in a pre-treatment medium under at least one stress-inducing condition, followed by administering a conditioned medium or a secretome-, extracellular vesicle-, and/or sEV-containing composition, to the cell culture. The target cells are then cultured in the presence of the conditioned medium or the secretome-, extracellular vesicle-, and/or sEV-containing composition, and at least one property of the cultured cells is measured one or more times during the culturing. In some embodiments, the at least one property is measured multiple times during the culturing in the presence of the conditioned medium or the secretome-, extracellular vesicle-, and/or sEV-containing composition (such as, for example, 5 minutes to 10 hours apart from each other; 10 minutes to 4 hours apart from each other; or 30 minutes to 2 hours apart from each other).

In some embodiments of this first method, the culturing in the presence of the conditioned medium or the secretome-, extracellular vesicle-, and/or sEV-containing composition, occurs in the presence of the at least one stress-inducing condition. In other embodiments of this first method, the culturing in the presence of the conditioned medium or the secretome-, extracellular vesicle-, and/or sEV-containing composition, occurs in the absence of the at least one stress-inducing condition.

In some embodiments of this first method, the pre-treatment medium is removed from the cells before the culturing in the presence of the conditioned medium or the secretome-, extracellular vesicle-, and/or sEV-containing composition. Thus, in embodiments of the first method where the at least one stress-inducing condition is provided by the pre-treatment medium (e.g., by a stress-inducing agent present in the pre-treatment medium), the culturing in the presence of the conditioned medium or the secretome-, extracellular vesicle-, and/or sEV-containing composition, occurs in the absence of the at least one stress-inducing condition.

In other embodiments of this first method, the pre-treatment medium is not removed from the cells before the culturing in the presence of the conditioned medium or the secretome-, extracellular vesicle-, and/or sEV-containing composition. Thus, in embodiments of the first method where the at least one stress-inducing condition is provided by the pre-treatment medium (e.g., by a stress-inducing agent present in the pre-treatment medium), the culturing in the presence of the conditioned medium or the secretome-, extracellular vesicle-, and/or sEV-containing composition, occurs in the presence of the at least one stress-inducing condition.

In a second method, target cells are cultured in a pre-treatment medium, followed by administering a conditioned medium or a secretome-, extracellular vesicle-, and/or sEV-containing composition (and optionally thereafter, culturing the target cells in the presence of the conditioned medium or the secretome-, extracellular vesicle-, and/or sEV-containing composition). The target cells are then cultured under at least one stress-inducing condition, and at least one property of the cultured cells is measured one or more times during the culturing under the at least one stress-inducing condition (which also occurs in the presence of the conditioned medium or the secretome-, extracellular vesicle-, and/or sEV-containing composition). In some embodiments, the at least one property is measured multiple times during the culturing under the at least one stress-inducing condition (and in the presence of the conditioned medium or the secretome-, extracellular vesicle-, and/or sEV-containing composition), such as, for example, 5 minutes to 10 hours apart from each other; 10 minutes to 4 hours apart from each other; or 30 minutes to 2 hours apart from each other.

In some embodiments of this second method, the target cells are cultured in the presence of the conditioned medium or the secretome-, extracellular vesicle-, and/or sEV-containing composition, before being cultured under the at least one stress-inducing condition. In other embodiments of this second method, the target cells are not cultured in the presence of the conditioned medium or the secretome-, extracellular vesicle-, and/or sEV-containing composition, before being cultured under the at least one stress-inducing condition. In some embodiments of this second method, the conditioned medium or the secretome-, extracellular vesicle-, and/or sEV-containing composition, is removed from the target cells before the target cells are cultured in the presence of the at least one stress-inducing condition.

In some embodiments of the above first and second methods, the stress-inducing condition is culturing in the presence of a cellular stress agent. In some embodiments of the second method, the cellular stress agent is co-administered to the target cells with the conditioned medium or the secretome-, extracellular vesicle-, and/or sEV-containing composition.

In some embodiments of the above first and second methods, the cellular stress agent is one or more apoptosis-inducing agents.

The one or more apoptosis-inducing agents may be selected from, for example, doxorubicin, staurosporine, etoposide, camptothecin, paclitaxel, vinblastine, gambogic acid, daunorubicin, tyrphostins, thapsigargin, okadaic acid, mifepristone, colchicine, ionomycin, 24(S)-hydroxycholesterol, cytochalasin D, brefeldin A, raptinal, carboplatin, C2 ceramide, actinomycin D, rosiglitazone, kaempferol, berberine chloride, bioymifi, betulinic acid, tamoxifen, embelin, phytosphingosine, mitomycin C, birinapant, anisomycin, genistein, cycloheximide, and the like.

In some embodiments, the apoptosis-inducing agent is an indolocarbazole. In some embodiments, the apoptosis-inducing agent is an indolo (2,3-a) pyrrole (3,4-c) carbazole. In some embodiments, the apoptosis-inducing agent is staurosporine, or a derivative thereof. In other embodiments, the apoptosis-inducing agent is doxorubicin, or a derivative thereof.

In some embodiments of the above first and second methods, the stress-inducing condition is culturing in the presence of a chemotherapeutic agent; and the conditioned medium or the secretome-, extracellular vesicle-, and/or sEV-containing composition, is analyzed in a chemotherapy-induced cardiomyopathy assay. In some embodiments thereof, the chemotherapeutic agent is an anthracycline. In some embodiments thereof, the anthracycline is one more of aclarubicin, daunorubicin, doxorubicin, epirubicin, idarubicin, mitoxantrone, and valrubicin. In some embodiments thereof, the chemotherapeutic agent is or comprises doxorubicin.

In some embodiments thereof, the chemotherapy-induced cardiomyopathy assay comprises treating cells, such as cardiomyocytes, with a chemotherapeutic agent to induce cardiomyopathy, before culturing the treated cells in the presence of the conditioned medium or the secretome-, extracellular vesicle-, and/or sEV-containing composition. In some embodiments thereof, the induction of the chemotherapy-induced cardiomyopathy; and/or the treating or reducing of the chemotherapy-induced cardiomyopathy, is measured by analyzing ATP content. In some embodiments, the induction of the chemotherapy-induced cardiomyopathy; and/or the treating or reducing of the chemotherapy-induced cardiomyopathy, is measured by analyzing mitochondrial function, for example, using a Seahorse method (e.g., Seahorse Mito Stress Test (Seahorse XFp Cell Mito Stress Test Kit, Agilent)).

In some embodiments of the first and second methods, at least one property measured is viability of the cultured cells. The viability may be measured, for example, using a DNA-labeling dye or a nuclear-staining dye. In some embodiments thereof, the DNA-labeling dye or the nuclear-staining dye is a fluorescent dye, such as a far-red fluorescent dye.

In some embodiments, a conditioned media; or a secretome-, extracellular vesicle-, and/or sEV-containing composition, may be analyzed in a chemotherapy-induced cardiomyopathy animal model. In some embodiments thereof, the animal model is a rat model of chemotherapy-induced cardiomyopathy. In some embodiments thereof, the chemotherapeutic agent is an anthracycline. In some embodiments thereof, the anthracycline is one more of aclarubicin, daunorubicin, doxorubicin, epirubicin, idarubicin, mitoxantrone, and valrubicin. In some embodiments thereof, the chemotherapeutic agent is or comprises doxorubicin. In some embodiments, the induction, treatment, or reduction, of chemotherapy-induced cardiomyopathy may be measured by one or more of: echocardiography (to determine LVESV and LVEDV, for example); electrocardiography; blood pressure (systolic, diastolic, etc.) measurements; functional status assessed by an NYHA score; quality of life; measurements of LVEF and LV volumes; maximum oxygen consumption during exercise; immune response by detection of antibodies specific to donor cells after each infusion; and assay of pro- and anti-inflammatory cytokines. In some embodiments, a conditioned media; or a secretome-, extracellular vesicle-, and/or sEV-containing composition, may have the capacity to counter the energetic stress (a metabolic/energy-related pathology) induced by the chemotherapy (e.g., mitochondrial damage, insufficient energy production).

In some embodiments of the first and second methods, one or more of the culturing of the target cells with: (a) the pre-treatment medium; (b) the conditioned medium or a secretome-, extracellular vesicle-, and/or sEV-containing composition; and (c) at least one stress-inducing condition, may occur in the absence of serum. In some embodiments, the target cells may be deprived of serum before administering the conditioned media or the secretome-, extracellular vesicle-, and/or sEV-containing composition. In other embodiments, the target cells may be deprived of serum after administering the conditioned media or the secretome-, extracellular vesicle-, and/or sEV-containing composition. In some embodiments, the cells are deprived of serum before and after administering the conditioned media or the secretome-, extracellular vesicle-, and/or sEV-containing composition.

In embodiments of the first and second methods, the target cells can be cultured in the pre-treatment medium for differing lengths of time. For instance, the target cells can be cultured in the pre-treatment medium for 30 minutes to 10 hours, 1 hour to 5 hours, or more than, less than, or about, 1 hour, 2 hours, 3 hours, 4 hours, or 5 hours.

In embodiments of the first and second methods, the target cells are cultured with the conditioned medium or the secretome-, extracellular vesicle-, and/or sEV-containing composition, for at least 30 minutes, at least 1 hour, at least 2 hours, at least 4 hours, at least 6 hours, at least 8 hours, at least 12 hours, at least 18 hours, at least 24 hours, at least 36 hours, or at least 48 hours.

In some embodiments of the first and second methods, the target cells are cultured in vitro prior to culturing in the pre-treatment medium. For instance, the target cells may be cultured in vitro for between 1-21 days, between 3-17 days, between 5-14 days, or less than 20 days, less than 18 days, less than 16 days, less than 14 days, less than 12 days, less than 10 days, less than 8 days, less than 6 days, less than 4 days, or less than 2 days, prior to culturing in the pre-treatment medium. In certain embodiments in which the target cells are cultured in vitro prior to culturing in the pre-treatment medium, the target cells are supplied with fresh culture medium prior to culturing in the pre-treatment medium. For instance, the target cells may be supplied with fresh culture medium 6-72 hours, 8-60 hours, 10-48 hours, 12-36 hours, prior to culturing in the pre-treatment medium.

In embodiments of the first and second methods, the culturing of the target cells may be two-dimensional or three-dimensional cell culturing. For instance, in some embodiments, the culture vessel used for culturing may be a flask, flask for tissue culture, hyperflask, dish, petri dish, dish for tissue culture, multi dish, micro plate, micro-well plate, multi plate, multi-well plate, micro slide, chamber slide, tube, tray, CellSTACK® Chambers, culture bag, roller bottle, bioreactor, stirred culture vessel, spinner flask, microcarrier, or a vertical wheel bioreactor, for example.

In embodiments in which culturing comprises two-dimensional cell culture, such as on the surface of a culture vessel, the culture surface (to which the cells are intended to adhere) may be coated with one or more substances that promote cell adhesion. Such substances useful for enhancing attachment to a solid support include, for example, type I, type II, and type IV collagen, concanavalin A, chondroitin sulfate, fibronectin, fibronectin-like polymers, gelatin, laminin, poly-D and poly-L-lysine, Matrigel, thrombospondin, and/or vitronectin.

In embodiments of the first and second methods, the at least one property may also be analyzed with reference to one or more control samples.

For instance, the first and second methods may further comprise culturing positive control cells in parallel, wherein the positive control cells are not administered the conditioned medium or the secretome-, extracellular vesicle-, and/or sEV-containing composition, and are not cultured under the at least one stress-inducing condition. Thus, in embodiments in which the stress inducing condition is the presence of an apoptosis-inducing agent, the positive control cells are not administered the apoptosis-inducing agent.

The first and second methods may comprise culturing negative control cells in parallel, wherein the negative control cells are not administered the conditioned medium or the secretome-, extracellular vesicle-, and/or sEV-containing composition. In some embodiments, the negative control cells comprise negative control cells subjected to the same steps as the target cells, except that they are not administered the secretome.

In certain embodiments, the negative control cells comprise negative control cells cultured in the pre-treatment medium under the at least one stress-inducing condition. The at least one property measured in the target cells may also then be measured in the negative control cells, either during or after they are cultured in the pre-treatment medium under the at least one stress-inducing condition.

In some embodiments, the negative control cells comprise negative control cells to which a mock conditioned medium or a mock secretome-, extracellular vesicle-, and/or sEV-containing composition is added. In specific embodiments thereof, the mock conditioned medium or the mock secretome-, extracellular vesicle-, and/or sEV-containing composition is produced by omitting cells from the process of producing a conditioned medium or a secretome-, extracellular vesicle-, and/or sEV-containing composition, such as a process of the present disclosure.

The use of such a negative control(s) allows an activity, functionality and/or potency, of a conditioned medium or a secretome-, extracellular vesicle-, and/or sEV-containing composition, to be evaluated. For instance, where the at least one property measured is viability of the cultured cells, a conditioned medium or a secretome-, extracellular vesicle-, and/or sEV-containing composition, may be determined to have an activity, functionality, potency (and/or exhibit a therapeutic effect), when the viability of the target cells is higher than the viability of the negative control cells.

Alternatively, for instance, where the at least one property measured is cell adhesion, cell growth, and/or cell number, and wherein the cell adhesion, cell growth, and/or cell number is determined by measuring electrical impedance across a culture vessel surface in the culture, a conditioned medium or a secretome-, extracellular vesicle-, and/or sEV-containing composition may be determined to have an activity, functionality, potency (and/or exhibit a therapeutic effect), when the electrical impedance across a culture vessel surface in the culture is higher than the electrical impedance across a culture vessel surface in a culture of negative control cells.

Any one or more samples, and/or any one or more positive and/or negative controls, may be performed in replicate, such as, for example, in duplicate, in triplicate, etc. In some embodiments thereof in which cell viability is measured, and where replicate cultures are performed, the number of positive control cells in the replicate cultures may be averaged to produce an average maximum cell number (and the number of target cells in each replicate test culture may be normalized to the average maximum cell number, to calculate cell viability).

To more accurately compare an activity, functionality, and/or potency, between different conditioned media or secretome-, extracellular vesicle-, and/or sEV-containing compositions, it may be beneficial to determine the amount of the conditioned medium or the secretome-, extracellular vesicle-, and/or sEV-containing composition, added to target cells. This can be determined, for example, based on one or more of: the amount of secreting cells that produced the secretome; the protein content of said secretome; the RNA content of said secretome; the exosome amount of said secretome; and particle number.

### Methods of Treatment Using the Compositions of the Present Disclosure

The present disclosure further contemplates using the compositions of the present disclosure for the treatment or prevention of various diseases and conditions in a subject in need thereof. The methods of treatment or prevention contemplated herein include, for example, treatment or prevention of cardiovascular diseases and conditions, such as myocardial infarction, heart failure, myocarditis, cardiomyopathy, ischemic cardiomyopathy, dilated cardiomyopathy, post-chemotherapy induced heart failure, ventricular dysfunction, atrial dysfunction, or arrhythmia.

In some embodiments, the heart failure is acute heart failure, chronic heart failure, ischemic heart failure, non-ischemic heart failure, heart failure with ventricular dilation, heart failure without ventricular dilation, heart failure with reduced left ventricular ejection fraction, or heart failure with preserved left ventricular ejection fraction.

In some embodiments cardiomyopathy is chemotherapy-induced cardiomyopathy. In some embodiments the chemotherapy-induced cardiomyopathy is anthracycline-induced cardiomyopathy. In some embodiments, anthracycline is doxorubicin.

In some embodiments, the methods of the present disclosure improve cardiac performance, fitness, endurance or recovery of a subject.

In some embodiments, the methods of the present disclosure improve angiogenesis, improve cardiomyocyte viability, improve endothelial cell survival, health and function, reduce fibrosis in cardiac fibroblasts, improve survival of stressed cardiomyocytes, improve survival, viability and proliferation of stressed endothelial cells, improve cell migration and/or wound healing capabilities in a subject, for example, by improving migration and/or wound healing capabilities of stressed endothelial cells. In some embodiments the cardiac fibroblasts in which fibrosis is reduced by the methods of the present disclosure are stimulated cardiac fibroblasts, such as cardiac fibroblasts stimulated by TGF-β1. In some embodiments, the methods reduce the expression of the pro-fibrotic marker, POSTN, in TGF-β1-stimulated cardiac fibroblasts to level prior to stimulation with TGF-β1 or below.

In some embodiments, the methods of the present disclosure improve or maintain Left Ventricular End Systolic Volume (LVESV). In some embodiments, the methods maintain LVESV within 20%, within 15%, within 10%, within 5%, or within 2% of the pre-treatment LVESV.

In some embodiments, the methods of the present disclosure do not induce an allogeneic inflammatory response in a subject, do not induce an allogeneic peripheral blood mononuclear cell (PBMC) activation, do not induce a significant increase in the percentage of IFNg or IL-2 expressing PBMCs, do not induce allogeneic natural killer (NK) cell degranulation, or do not significantly increase the percentage of CD107 expressing NK cells.

In some embodiments, the compositions of the present disclosure are administered to a subject by intravenous infusion, direct cardiac injection, intra-arterially or by interventional cardiology methods, such as by a catheter-based administration.

The dose, route of administration, frequency of administration and duration of treatment with the compositions of the present invention may be determined by considering the disease or condition for which the treatment is administered, severity and duration of the disease or condition, medical history and overall health of the subject being treated, tolerability of the composition, adverse effects and other factors.

The compositions of the present disclosure may be administered, for example, at a dose containing secretome obtained from 0.1 to 10 million cells per kg weight of the subject being treated, from 0.5 to 5 million cells per kg weight of said subject, from 1 to 3 million cells per kg weight of said subject, from 1 to 2 million cells per kg weight of said subject, or 1 million cells per kg weight of said subject. The dose may be administered at onetime, e.g., per each intravenous infusion, cardiac injection or intra-arterial administration, or over several administrations. In some embodiments, the cells are cardiomyocyte progenitor cells, cardiac progenitor cells, cardiovascular progenitor cells, or mixtures thereof.

The compositions of the present disclosure may be administered, for example, at a dose containing from 1×10⁹ to 60×10⁹ particles per kg weight of the subject, from 10×10⁹ to 60×109 particles, per kg weight of the subject, or from 10×10⁹ to 40×10⁹ particles per kg weight of the subject, from 20×10⁹ to 40×10⁹ particles per kg weight of the subject, 20×10⁹ particles per kg weight of the subject, or 10×10⁹ particles per kg weight of the subject. The number of particles may be measured, for example, by Nanoparticle Tracking Analysis (NTA). The dose may be administered at one time, e.g., per each intravenous infusion, direct cardiac injection or intra-arterial administration or over several administrations.

The compositions of the present disclosure may be administered, for example, at a cumulative daily dose containing from 20×10⁹ to 200×10⁹ particles per kg weight of the subject, from 30×10⁹ to 100×10⁹ particles per kg weight of the subject, 60×10⁹ particles per kg weight of the subject, 50×10⁹ particles per kg weight of the subject, 40×10⁹ particles per kg weight of the subject, 30×10⁹ particles per kg weight of the subject, 20×10⁹ particles per kg weight of the subject, or 10×10⁹ particles per kg weight of the subject. The number of particles may be measured by NTA. The cumulative daily dose may be administered at one time, e.g., one intravenous infusion, direct cardiac injection or intra-arterial administration or over several administrations.

The compositions of the present disclosure may be administered, for example, 1 to 10 times per day, 3 to 6 times per day, 1 to 5 times per day, 3 times per day, 2 times per day, or once per day.

The duration of treatment may be, for example, 65 days or less, 5 to 50 days, 10 to 50 days, 20 to 45 days, 42 days, 21 days, 14 days, or 7 days.

During the course of the treatment, the compositions of the present disclosure may be administered, for example, every day, every other day, at a frequency of from every day to every 30 days, from every 7 days to every 21 days, every 21 days, every 14 days, every 7 days.

Testing products on non-human mammalian species is important for modeling complex diseases that affect the biology and/or physiology of multiple cells, tissues, organs, and systems. Using animal models enables testing the effect of products on physiology of tissues, organs, and organisms. A new animal model of heart failure is described herein. While the term ‘heart failure’ is typically used for human subjects, its use herein is extended to the animal models. Two different heart failure models are described. One is a post-ischemia chronic heart failure model in mice which is induced by surgical means (permanent occlusion of the left ventricular coronary artery), and the other is a non-ischemic, chemotherapy drug-induced cardiomyopathy with left ventricular dysfunction and other signs of heart failure. Both models involve left ventricular dysfunction.

In the chemotherapy-induced cardiomyopathy (CCM) model described herein, left ventricular dysfunction is induced in rats through the administration of doxorubicin, which belongs to the anthracycline class of anticancer therapies and is one of the most widely used antineoplastic drugs, thanks to its broad spectrum of activity. Anthracyclines are chemotherapy agents known to induce heart failure in some patients. The model described herein effectively recapitulates many physiological features of chemotherapy-induced cardiomyopathy (CCM) in humans, such as progressively larger Left Ventricular End Systolic Volume (LVESV) and Left Ventricular End Diastolic Volume (LVEDV), decreasing ejection fraction (LVEF), decreased systolic elastance, and a slower LV-depolarization (increased QTc at EKG). In human patients, these characteristics are features of degrading cardiac function and are associated with worse prognosis. The model described herein is useful for determining the effect of extracellular vesicle (EV)-containing compositions on heart physiology. Beneficial effects of the EV-containing compositions in this non-ischemic model are expected to be predictive of the beneficial effects of the EV-containing compositions on the function of hearts in human subjects in non-ischemic heart failure, including subjects with chemotherapy-induced heart failure.

The beneficial effects of the EV-containing compositions in the post-ischemic model of chronic heart failure are expected to be predictive of the beneficial effects of the EV-containing compositions on the function of hearts in human subjects in post-ischemic heart failure, such as patients who have had a myocardial infarction, for example.

Using the presented rat model of CCM, it is shown herein that CTC1-EV final formulation, has a favorable effect on the physiology of the failing heart. The present inventors have demonstrated that the animals treated with the EV-containing compositions have reduced progression of left ventricular dysfunction as compared to controls as shown by less heart volume enlargement over the study period. The controls, by contrast, continue to deteriorate over the study period, as shown by the increase in systolic and diastolic heart volumes.

In addition to testing efficacy of a product in animal models, it is useful to test products in an in vitro model of human disease using human cells. In vitro models are key to understanding the specific effects a product has on specific cell types by enabling direct and specific analysis of a given cell type. The most useful in vitro cell models are on human cells, as this most closely matches the intended use of a product designed for treating human subjects. If desired, in vitro models can be limited to a single cell type, so that the biological effects of the EV-containing compositions on that specific cell type can be interrogated. Additionally, co-culture models and mixed cell models are also useful for exploring the interplay of different cell types. In this specification, the effect of the EV-containing compositions on four human cell types in monoculture, and one mixed peripheral blood mononuclear cell (PBMC) model is described. All five of these cell types/mixtures are relevant to the pathology of heart failure, including ischemic and non-ischemic heart failure, including chemotherapy-induced heart failure. Inventors found positive biological effects of the EV-containing compositions on human cardiomyocytes, human endothelial cells, human cardiac fibroblasts, and a lack of allogeneic activation of human NK cells or human PBMCs.

Cardiomyocytes are stressed in heart failure and can be in programmed cell death (apoptosis). Preservation of cardiomyocyte health and survival will have a beneficial effect on hearts in failure or hearts in ventricular dysfunction. The present inventors have shown that the EV-containing compositions promote survival of human cardiomyocytes when under apoptosis-inducing stress. The EV-containing compositions are expected to promote cardiomyocyte health and survival in human subjects in heart failure. The EV-containing compositions are expected to improve cardiomyocyte-related functions of a heart in failure in a human subject.

Endothelial cells are integral components of blood vessels and lymphatic tissues of the heart. A lack of sufficient circulation and drainage into and out of the heart tissues contributes to deteriorating heart function in failing hearts. Promoting endothelial cell survival, proliferation and migration under stress will encourage beneficial remodeling of the heart tissue and or reduce negative remodeling of the heart tissues. Together this will improve or help preserve heart function. The present inventors have shown that the EV-containing compositions support in vitro human endothelial cell survival in two different forms of stress, in vitro human endothelial cell proliferation and in vitro human endothelial cell migration, when these cells are under stress. The EV-containing compositions are expected to promote endothelial cell survival, proliferation and migration in human subjects in heart failure, in which vessel health and wound healing capabilities are compromised. Increasing vascularization of the failing heart tissue would support maintaining the health of the heart tissue. The EV-containing compositions are expected to improve endothelial cell-related functions of a heart in failure in a human subject.

In heart failure, there is increased fibrosis. Fibrosis contributes to the negative remodeling of a failing heart. Decreasing fibrosis will support better heart function in a failing heart. The present inventors have shown that the EV-containing compositions reduce signs of fibrosis in human cardiac fibroblast cells that have been stressed into a state of increased fibrosis. The EV-containing compositions are expected to reduce fibrosis in human subjects in heart failure and to reduce the negative effects of fibrosis on the functioning of the heart.

Heart failure leads to a pro-inflammatory state in humans, which contributes to the deterioration of heart function. The present inventors have shown in vitro and in vivo, that the EV-containing compositions of the present invention do not stimulate allogeneic NK degranulation. The present inventors have also shown that the EV-containing compositions do not induce allogenic PBMC activation in vitro. The EV-containing compositions of the current invention does not promote NK degranulation in human subjects in heart failure, and to not induce PBMC activation in human subjects in heart failure. The EV-containing compositions of the present invention are immunologically neutral or anti-inflammatory when administered to human patients in heart failure.

In vivo pre-clinical safety testing was performed on the EV-containing compositions (refer to clinical trial NCT05774509, which is incorporated by reference hereby in its entirety). GLP mouse and GLP rat models were used to show pre-clinical safety aspects of the EV-containing compositions of the present invention. Immunocompetent animal models selected showed no signs of acute immune reaction after repeated administrations, even with this being a xenograft. Both the mouse and rat models showed no toxicity of the EV-containing compositions when administered at high doses. Furthermore, a GLP immunocompromised mouse model showed no tumorigenicity of the EV-containing compositions. It is important to prove in an animal model, which is more complex than an in vitro model, as it has a functional immune system, circulatory system, etc. The animal model test results demonstrated that the EV-containing compositions of the present invention have a good safety profile, which is necessary to establish prior to testing in human subjects.

The EV-containing compositions of the present invention are non-toxic and not tumorigenic when administered to human subjects.

Another aspect of the EV-containing compositions of the present invention is the combination of multiple, parallel, beneficial biological effects expected to affect the biology of multiple cell types in a beneficial way in a patient in need of treatment, together with a positive safety profile. The EV-containing compositions of the present invention are a complex mixture of biological molecules, enabling the simultaneous protective, therapeutic, or regenerative properties described above, supporting the health, survival and function of multiple cell types in concert, contributing to the physiological effects observed in the animal models of chemotherapy-induced cardiomyopathy (CCM) as shown here as the therapeutic effects when used to treat humans.

In addition to novel and unexpected biological and therapeutic effects of the EV-containing compositions disclosed of the present invention, the manufacturing process is designed, optimized, and tested at phase 1 clinical manufacturing scale, using GMP compatible methods, materials and reagents, for the manufacturing of a EV-containing compositions for use in human subjects. The use of multi-layer cell stacks, in-line clarification process, TFF (tangential flow filtration), ability to work from frozen cells if desired, ability to freeze TFF retentates for future pooling if needed, and lack of centrifugation steps, make the process defined herein are scalable to phase 2 and phase 3 manufacturing and to commercial manufacturing scales.

Additionally, the inventors have disclosed a novel, groundbreaking, inventive and comprehensive panel of in-process quality control tests, and release tests for Quality Control, which ensure reproducibility, stability, safety, and potency of the EV-enriched secretome or EVs and compositions comprising thereof as a therapeutic. Features of the panel described herein may be applied to the quality control of other EV-enriched therapeutics, ensuring process control, the reproducibility of EV-containing compositions, the safety of EV-containing compositions, the potency of EV-containing compositions, and the stability of EV-containing compositions, to enable their use in a human subject.

### EXAMPLES

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for. It should be understood that these Examples are given by way of illustration only and are not intended to limit the scope of what the inventor regards as various embodiments of the present invention. Not all of the following steps set forth in each Example are required nor must the order of the steps in each Example be as presented.

### Example 1

Generation of Cardiovascular Progenitor Cells from iPSCs

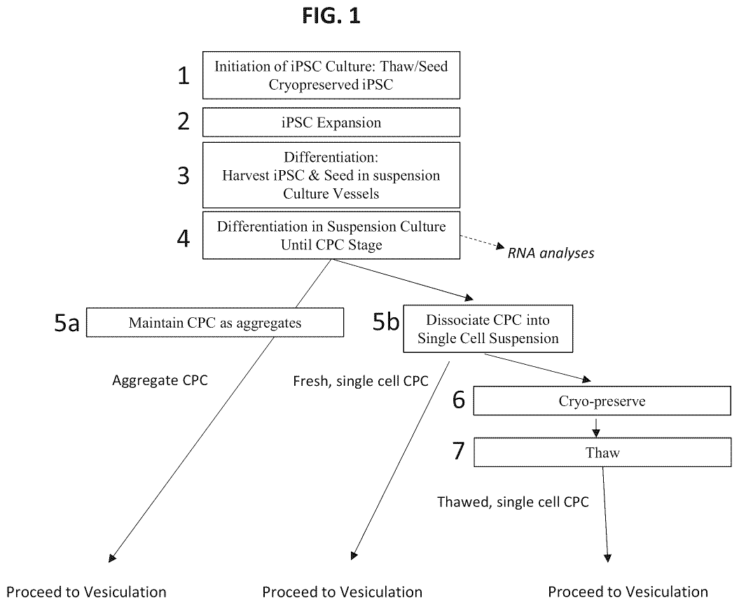

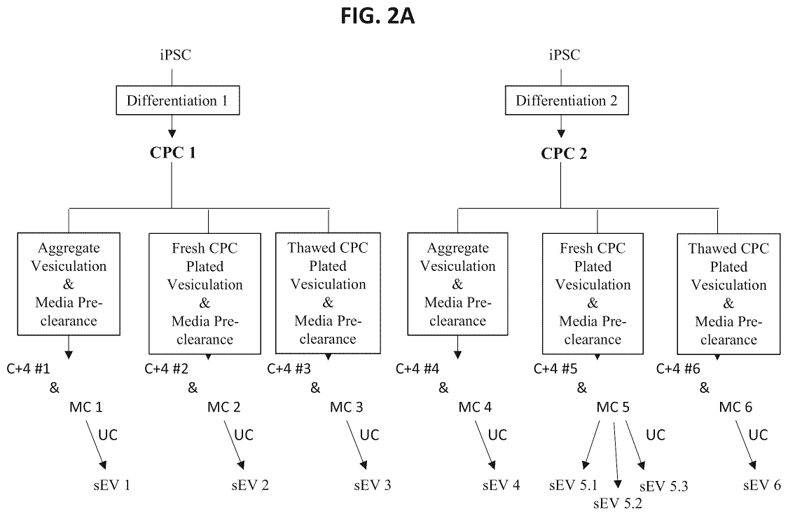

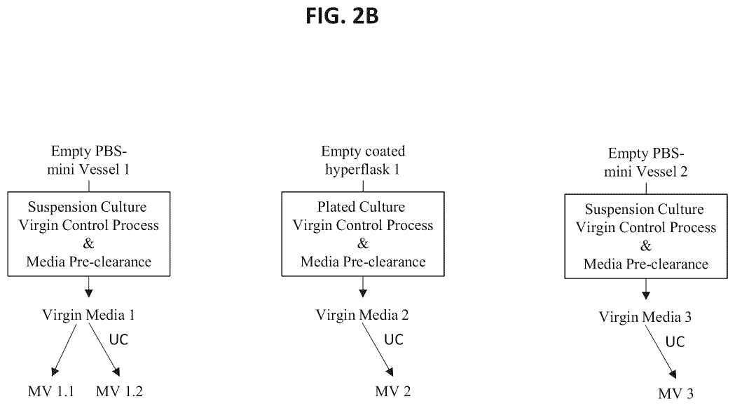

Human iPS cells (iPSCs) were expanded and differentiated into cardiovascular progenitor cells (CPCs) by suspension culture in PBS-mini vessels (PBS MINI 0.5 L Bioreactor Single Use Vessels; PBS Biotech ref: 1A-0.5-D-001), using the process depicted in FIG. 1. At the end of the CPC differentiation period, cells were counted as follows. A small sample (5-10 mL) of cell aggregates in suspension was removed from the suspension culture vessels, cell aggregates were gravity settled, supernatant removed and aggregates were resuspended in 3-5 mL of room temperature TrypLE Select (Invitrogen ref: 12563029), and incubated for 3-10 min at 37 °C. Digestions were stopped using double the volume of RPMI-B27 Quench media (RPMI 1640 Medium (Gibco ref: 118875-085) supplemented with B-27 XenoFree, CTS grade 50× (Gibco ref: A14867-01, fc=1×), filter sterilized using a 0.2 μm filter (ThermoScientific ref 567-0020)). Cell suspensions were then centrifuged at 300×g for 5 minutes, and the resulting supernatant was discarded. The remaining cell pellets were delicately loosened, and the cells were resuspended in 5-10 mL of MEM alpha media base (MEM alpha, GlutaMAX™, no nucleosides, Gibco ref 32561.37). Of these resuspended cells, one or two 500 μL samples were counted using a ViCell XR cell viability analyzer (Beckman Coulter), according to the manufacturer's directions. The viable cells per mL were noted. Two distinct differentiation runs were performed, as depicted in FIG. 2, and similar yields of CPC per input iPSC were obtained.

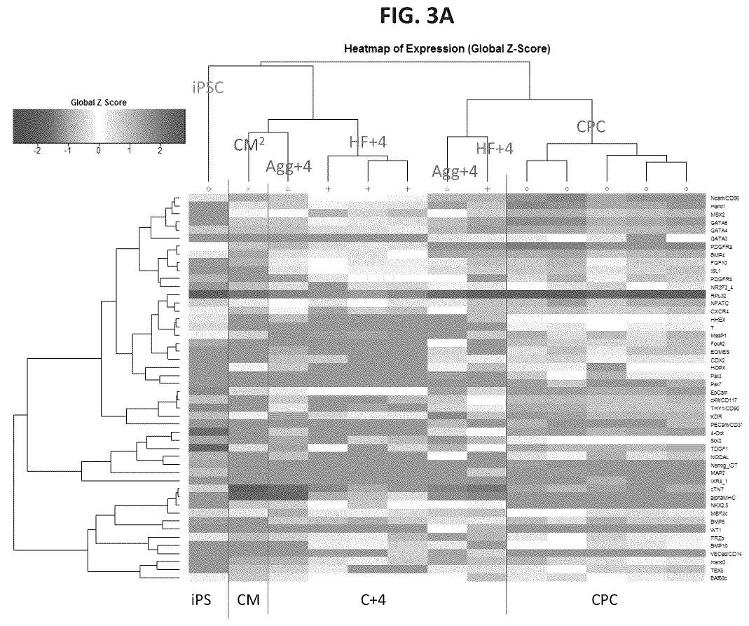

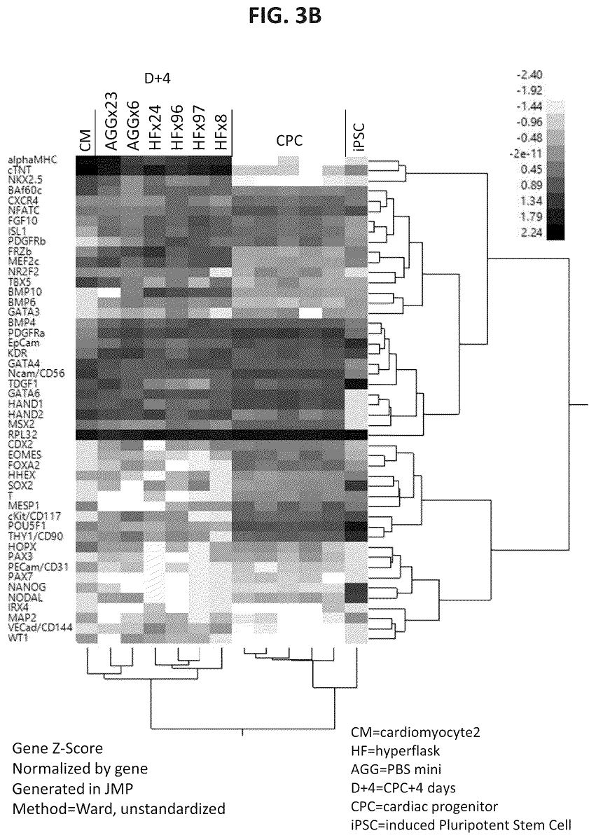

To confirm that the resulting cells were indeed CPCs, RNA expression by the resulting cells was analyzed. Specifically, between 1 and 2 million cells from the cell samples were removed and lysed in RLT plus buffer (Qiagen 1030963) for RNA extraction. RNA was extracted from the lysates using the Qiasymphony RNA kit (Qiagen, Ref: 931636), following the manufacturer's directions. mRNA levels for 48 custom selected genes were evaluated using the qPCR Fluidigm platform. Unsupervised hierarchical clustering was performed on raw data using the “SINGuLAR Analysis Toolset” package in R v3.1.1 (FIG. 3A). Unsupervised hierarchical clustering was also performed on gene z-scores using JMP software v17 (method=ward, unstandardized) (FIG. 3B). RNA expression by the resulting cells was compared to RNA expression by iPSC and cardiomyocyte control cells, confirming that gene expression by the resulting cells was consistent with them being CPCs (FIG. 3A, FIG. 3B and TABLE 1). TABLE 1 presents the Ct data needed to produce FIG. 3A and FIG. 3B.

To dissociate CPC aggregates to single cells, 300-800 mL of the aggregate suspension of CPCs were collected from the differentiation suspension cultures and allowed to settle for approximately 5 minutes in 500 mL conical tubes. Spent media was then removed, and cell aggregates were washed in DPBS−/−. The washed cell aggregates were then resuspended in room temperature TrypLE (in approximately 25 mL TrypLE for 100 mL original aggregate suspension volume) and were allowed to dissociate for 10 min at 37 °C. The cell aggregate dissociations were quenched with an equal volume of RPMI-B27 Quench media, and the dissociated cells were spun at 400×g for 5 minutes. The resulting cell pellets were resuspended in RPMI-B27 Quench media, and then strained (Falcon 100 μm Cell strainer, Corning ref: 352360) into conical tubes and counted using a ViCell XR cell viability analyzer (Beckman Coulter).

A subset of these cells were re-spun at 300×g for 5 minutes, resuspended for fresh CPC plated vesiculation culture in alpha-MEM complete media (MEM alpha media base (MEM alpha, GlutaMAX™, no nucleosides, Gibco ref 32561-37); Gentamicin (Gibco ref 15750060, final concentration (fc)=0.025 mg/mL); glucose supplement (Gibco ref A2494001, at a ratio of 1:200); Flexbumin (with 25% w/vol human serum albumin, Baxter ref: NDC0944-0493-02 code 2G0012, fc HSA=2 mg/mL); B27 (minus insulin) (50×, Gibco ref A1895601, fc=1×); Human FGF-2 Premium grade (Miltenyi Biotec ref: A12873-01, fc=1 μg/mL); filter sterilized using a 0.2 μm filter (ThermoScientific ref 567-0020); media were used the same day). The freshly harvested single cells were again counted using a ViCell XR cell viability analyzer (Beckman Coulter) and plated (see Example 2 below). The remainder of the single cell suspensions were spun at 400×g for 5 minutes, and the cells were resuspended in cryopreservation media (CryoStor CS-10, BioLife Solution ref: 210102) at 25 million cells/mL, frozen at −80 °C, and then stored in liquid nitrogen for later use in thawed CPC plated vesiculation culture.

Ct data that was used to produce FIG. 3A and FIG. 3B are shown in TABLE 1.

TABLE 1
Ct values for genes of interest as used to generate FIG. 3A and FIG. 3B heatmaps.

| Row | Cell | | | | | | |
|---|---|---|---|---|---|---|---|
| Labels | CM2 | ×17 | ×18 | ×19 | ×2 | x23 | x24 |
| Alias | CM2 | CPCx17 | CPCx18 | CPCx19 | CPCx2 | AGGx23 | HFx24 |
| alphaMHC | 11.26366 | 30 | 30 | 30 | 30 | 11.38343 | 17.92243 |
| BAf60c | 16.95455 | 18.60862 | 17.84539 | 19.01982 | 19.5239 | 18.63056 | 19.62956 |
| BMP10 | 30 | 20.38452 | 20.09079 | 21.09441 | 21.50712 | 30 | 17.34695 |
| BMP4 | 23.50502 | 15.68946 | 15.1075 | 16.12797 | 16.95911 | 18.0676 | 19.94063 |
| BMP6 | 30 | 22.08222 | 21.43901 | 22.13802 | 23.18637 | 22.1365 | 22.10986 |
| CDX2 | 30 | 18.51421 | 17.34912 | 18.35283 | 19.09789 | 22.45642 | 30 |
| cKit/ | 30 | 16.10706 | 16.27416 | 16.91359 | 16.77759 | 22.06352 | 22.75957 |
| CD117 | | | | | | | |
| cTNT | 10.44261 | 23.26787 | 22.89221 | 30 | 23.70497 | 11.7444 | 14.53216 |
| CXCR4 | 22.46703 | 16.71893 | 16.66023 | 17.31923 | 17.50155 | 20.52114 | 22.47112 |
| EOMES | 30 | 16.56751 | 16.45705 | 16.77061 | 18.76848 | 23.28122 | 26.6845 |
| EpCam | 21.60821 | 14.69916 | 14.92292 | 15.45622 | 15.49569 | 18.76239 | 19.78977 |
| FGF10 | 23.0229 | 17.58082 | 16.18582 | 17.27638 | 19.18301 | 18.83039 | 18.99908 |
| FOXA2 | 30 | 16.54783 | 17.28414 | 17.86014 | 17.55439 | 25.16689 | 24.62348 |
| FRZb | 22.7325 | 20.59801 | 20.23027 | 20.8853 | 21.92514 | 17.25015 | 16.21595 |
| GATA3 | 30 | 19.7223 | 19.55812 | 30 | 20.95908 | 25.11612 | 25.38188 |
| GATA4 | 16.58343 | 15.6833 | 15.27618 | 16.23516 | 16.65387 | 16.48396 | 18.84512 |
| GATA6 | 17.08989 | 14.58751 | 13.85639 | 15.26443 | 15.79381 | 16.88499 | 18.11083 |
| HAND1 | 18.20682 | 14.45485 | 14.11678 | 15.36694 | 15.38437 | 17.02864 | 21.25982 |
| HAND2 | 15.56473 | 18.57454 | 17.62014 | 19.27542 | 18.10014 | 15.94225 | 17.09179 |
| HHEX | 24.80875 | 19.19474 | 19.74566 | 20.11403 | 19.93811 | 22.5132 | 30 |
| IIOPX | 20.49305 | 19.61298 | 19.04043 | 19.71005 | 23.8345 | 22.27466 | 30 |
| ISLI | 25.42068 | 16.84052 | 16.44759 | 17.47883 | 18.39275 | 18.3001 | 19.06662 |
| IRX4 | 30 | 30 | 30 | 30 | 23.21517 | 30 | 26.07159 |
| KDR | 20.56798 | 15.99381 | 14.95174 | 16.01848 | 16.21918 | 15.23473 | 18.57565 |
| MAP2 | 24.86559 | 25.27786 | 23.80363 | 30 | 26.50288 | 30 | 25.48328 |
| MEF2c | 18.60827 | 20.9067 | 20.16558 | 21.50403 | 21.39207 | 15.76996 | 18.08313 |
| MESPI | 21.96158 | 16.13532 | 18.04827 | 18.17134 | 16.4107 | 30 | 30 |
| MSX2 | 20.33569 | 16.0783 | 15.53208 | 16.54816 | 17.19404 | 20.28166 | 22.67127 |
| NANOG | 25.48702 | 24.21377 | 24.44051 | 26.4385 | 25.45691 | 26.49297 | 30 |
| Ncam/ | 15.66073 | 14.24168 | 13.55462 | 14.38049 | 15.32116 | 16.94808 | 19.1866 |
| CD56 | | | | | | | |
| NFATC | 20.82348 | 15.46345 | 14.84845 | 15.80272 | 17.14907 | 19.70818 | 21.09258 |
| NKX2.5 | 16.90193 | 26.17298 | 30 | 30 | 26.43753 | 17.29332 | 20.12351 |
| NODAL | 30 | 22.13915 | 20.50368 | 21.28922 | 23.23781 | 22.57846 | 30 |
| NR2F2 | 22.67374 | 21.07157 | 18.14037 | 19.43376 | 21.95928 | 21.40558 | 21.8139 |
| PAX3 | 30 | 20.77993 | 19.95921 | 20.71007 | 23.10344 | 24.44189 | 30 |
| PAX7 | 30 | 23.20888 | 22.27914 | 24.32081 | 30 | 30 | 30 |
| PDGFRa | 22.31478 | 13.65739 | 13.07557 | 14.35491 | 14.38209 | 16.13815 | 18.2058 |
| PDGFRb | 30 | 17.23489 | 15.68661 | 16.6125 | 19.0393 | 23.70142 | 19.4209 |
| PECam/ | 24.33947 | 22.84019 | 25.82285 | 24.11197 | 24.06361 | 26.65284 | 30 |
| CD31 | | | | | | | |
| POUSF1 | 22.04512 | 14.84653 | 14.66078 | 15.40687 | 15.97971 | 22.17436 | 24.84687 |
| RPL32 | 12.55278 | 10.31978 | 9.803309 | 10.67006 | 11.52642 | 13.44857 | 13.7893 |
| SOX2 | 30 | 20.05358 | 19.39118 | 19.60016 | 19.89659 | 23.4711 | 22.73228 |
| T | 30 | 18.89876 | 18.47438 | 19.0065 | 19.87647 | 30 | 26.44332 |
| TBX5 | 16.63119 | 22.39453 | 19.80195 | 21.91069 | 21.92285 | 17.23512 | 24.06157 |
| TDGFI | 18.00629 | 15.34781 | 16.16808 | 16.33819 | 15.94133 | 15.07678 | 21.6196 |
| THYI/ | 24.4455 | 16.70915 | 15.7346 | 16.69246 | 16.94283 | 21.00244 | 23.99151 |
| CD90 | | | | | | | |
| VECad/ | 30 | 26.5752 | 25.61447 | 30 | 30 | 25.97629 | 21.85341 |
| CD 144 | | | | | | | |
| WTI | 24.00986 | 30 | 30 | 30 | 30 | 30 | 24.01042 |
| Row | | | | Line A IPS | | | |
| Labels | ×3 | x6 | x8 | Control | x96 | ×97 | |
| Alias | CPCx3 | AGGx6 | HFx8 | iPSC | HFx96 | HFx97 | |
| alphaMHC | 24.17089 | 13.97702 | 15.63403 | 30 | 13.36424 | 16.48421 | |
| BAf60c | 18.66913 | 19.66141 | 20.29825 | 20.62465 | 16.58441 | 19.80364 | |
| BMP10 | 19.71569 | 18.47407 | 18.89633 | 24.4618 | 14.63675 | 19.08601 | |
| BMP4 | 15.47248 | 15.28805 | 18.91018 | 20.90372 | 15.50876 | 18.30138 | |
| BMP6 | 22.74485 | 18.16037 | 22.52508 | 23.73495 | 18.47632 | 23.38024 | |
| CDX2 | 17.64946 | 18.11747 | 22.07651 | 26.53733 | 20.76707 | 24.77529 | |
| cKit/ | 16.24805 | 17.1142 | 30 | 16.6272 | 19.76444 | 30 | |
| CD117 | | | | | | | |
| cTNT | 23.58176 | 13.33099 | 13.63346 | 22.02662 | 12.50779 | 15.44804 | |
| CXCR4 | 17.1007 | 20.03498 | 23.55947 | 20.65495 | 16.50624 | 21.93014 | |
| EOMES | 18.23162 | 21.80055 | 25.56396 | 25.62369 | 25.35035 | 30 | |
| EpCam | 14.69381 | 15.09028 | 19.69977 | 13.91224 | 17.67198 | 19.9864 | |
| FGF10 | 17.11268 | 18.46002 | 19.52852 | 23.88509 | 16.56885 | 18.55132 | |
| FOXA2 | 16.49455 | 19.81392 | 30 | 23.40466 | 30 | 30 | |
| FRZb | 20.98541 | 14.84445 | 18.4701 | 21.07607 | 16.00962 | 19.0364 | |
| GATA3 | 18.67485 | 21.22586 | 30 | 24.09646 | 18.79167 | 25.35354 | |
| GATA4 | 15.33113 | 16.18984 | 17.99905 | 22.24036 | 14.84549 | 18.24952 | |
| GATA6 | 13.74961 | 16.53074 | 19.1077 | 30 | 15.15617 | 18.64279 | |
| HAND1 | 13.61611 | 15.09119 | 19.13238 | 30 | 16.73274 | 20.9627 | |
| HAND2 | 15.91002 | 15.28712 | 17.9429 | 30 | 16.55998 | 18.82618 | |
| HHEX | 19.29868 | 23.96854 | 30 | 21.02633 | 22.58836 | 24.00085 | |
| IIOPX | 20.91569 | 20.50756 | 25.10521 | 30 | 22.57909 | 30 | |
| ISLI | 17.04385 | 17.90523 | 19.48577 | 24.19049 | 17.20725 | 19.18548 | |
| IRX4 | 30 | 30 | 30 | 30 | 30 | 30 | |
| KDR | 14.60084 | 13.74249 | 17.52833 | 16.24971 | 16.78835 | 18.74422 | |
| MAP2 | 30 | 24.36605 | 26.38731 | 22.56005 | 22.17101 | 30 | |
| MEF2c | 19.75844 | 17.07608 | 18.49308 | 21.70791 | 16.5148 | 18.999 | |
| MESPI | 15.75588 | 30 | 23.81424 | 20.55074 | 22.21051 | 23.69722 | |
| MSX2 | 15.70464 | 19.83834 | 22.97709 | 30 | 17.30647 | 21.66805 | |
| NANOG | 23.75736 | 30 | 30 | 15.16575 | 30 | 30 | |
| Ncam/ | 14.17104 | 16.09588 | 19.48839 | 21.20879 | 15.96588 | 20.27415 | |
| CD56 | | | | | | | |
| NFATC | 16.54836 | 18.63015 | 21.07824 | 17.2185 | 16.53625 | 19.95729 | |
| NKX2.5 | 27.49359 | 19.64049 | 20.04222 | 30 | 19.18648 | 22.27032 | |
| NODAL | 21.36537 | 20.03662 | 30 | 15.66882 | 30 | 30 | |
| NR2F2 | 19.61881 | 19.11945 | 30 | 30 | 18.37241 | 21.48961 | |
| PAX3 | 22.17603 | 23.65339 | 25.02021 | 30 | 30 | 30 | |
| PAX7 | 23.7539 | 27.44128 | 30 | 30 | 30 | 30 | |
| PDGFRa | 12.77209 | 14.30515 | 18.00193 | 19.89885 | 13.9535 | 18.57049 | |
| PDGFRb | 17.82758 | 19.51133 | 21.09537 | 23.00603 | 14.58452 | 19.41088 | |
| PECam/ | 23.05761 | 22.78934 | 30 | 30 | 25.23108 | 30 | |
| CD31 | | | | | | | |
| POUSF1 | 15.07294 | 21.46787 | 25.38904 | 11.68464 | 20.02053 | 24.31997 | |
| RPL32 | 10.385 | 11.83094 | 14.22406 | 10.59714 | 10.62461 | 14.67264 | |
| SOX2 | 18.94332 | 30 | 30 | 14.95169 | 22.74563 | 21.95187 | |
| T | 19.11492 | 30 | 30 | 21.51207 | 30 | 30 | |
| TBX5 | 18.4364 | 18.53076 | 20.76727 | 24.86746 | 18.12694 | 25.82213 | |
| TDGFI | 15.26674 | 15.93489 | 19.92675 | 11.16106 | 19.55706 | 24.55766 | |
| THYI/ | 15.45055 | 17.51302 | 30 | 13.89524 | 18.90213 | 23.50249 | |
| CD90 | | | | | | | |
| VECad/ | 30 | 23.5593 | 23.76612 | 30 | 21.82464 | 26.35283 | |
| CD 144 | | | | | | | |
| WTI | 30 | 19.807 | 30 | 30 | 22.82724 | 25.25032 | |

### Example 2

### Vesiculation Culture of Cardiovascular Progenitor Cells

CPCs were cultured in the vesiculation process as fresh aggregates in suspension culture, as fresh single cells plated onto hyperflasks, or as thawed single cells plated onto hyperflasks after having been cryopreserved and maintained at −80 °C or less until time of use. Specifically, CPCs produced in Example 1 were used in suspension vesiculation culture and in adherent vesiculation culture in hyperflasks as described below.

For suspension vesiculation culture, the volumes of aggregates in PBS-mini vessels at the end of the CPC differentiation process were noted (300-400 mL per vessel; “day+0” volumes). The cell aggregates underwent a 100% media exchange according to the following steps: (1) cell aggregates were transferred from PBS-mini vessels to conical tubes and allowed to settle for approximately 15 min; (2) PBS-mini vessels were rinsed three times with MEM alpha media base (MEM alpha, GlutaMAX™, no nucleosides, Gibco ref 32561.37); (3) spent media was removed from settled cell aggregates; (4) cell aggregates were washed three times with an appropriate volume of MEM alpha media base; and (5) washed cell aggregates were re-seeded into their original (washed) PBS-mini vessels in alpha-MEM complete media (as described above) at their day+0 volumes to maintain cell density.

The seeded cell aggregates were then cultured in suspension (37 °C, 5% CO₂, at atmospheric oxygen) with agitation at 40 rpm for 2 days (until “day+2”). At day+2, cell aggregates underwent a 100% media exchange following three rinses in MEM alpha media base. For this day+2 media exchange, the cell aggregates were re-seeded into their original PBS-mini vessel in alpha-MEM poor media (MEM alpha media base (MEM alpha, GlutaMAX™, no nucleosides, Gibco ref 32561.37), supplemented with Gentamicin (Gibco ref 15750060, final concentration (fc)=0.025 mg/mL), and glucose supplement (Gibco ref A2494001, at a ratio of 1:200), filter sterilized using a 0.2 μm filter (ThermoScientific ref 567-0020)), at the same volumes as their day+0 volumes. The cell aggregates were then cultured (37 °C, 5% CO₂, at atmospheric oxygen) in suspension, with agitation at 40 rpm for another 2 days, until the end of the vesiculation period (“day+4”).

For Hyperflask adherent culture, fresh single cell CPCs were seeded at 100,000 cells/cm² onto vitronectin-coated hyperflasks in alpha-MEM complete media (“day+0”). In addition, cryopreserved CPCs were thawed at 37 °C for 3 min, transferred to an empty conical tube, then resuspended (dropwise) in alpha-MEM complete media. The thawed cell suspensions were centrifuged, and the cell pellets were resuspended in alpha-MEM complete media. The thawed CPCs were seeded at 100,000 cells/cm² onto vitronectin-coated hyperflasks in alpha-MEM complete media (“day+0”). The seeded cells for both fresh and thawed CPCs were then cultured (37 °C, 5% CO₂, at atmospheric oxygen) for 2 days (until “day+2”). At day+2, spent media was removed, and the flasks were rinsed three times with 50-100 mL of pre-warmed MEM alpha media base. The culture vessels were then filled with alpha-MEM poor media, according to the manufacturer's directions, and incubated for 2 more days (37 °C, 5% CO₂, at atmospheric oxygen) until the end of the vesiculation period (“day+4”).

At day+2 and day+4, cells in the suspension cultures were counted as described above in Example 1. At day+4, cells in the adherent cultures were harvested by 1/rinsing the cells with DPBS, 2/incubating cells with 100 mL of pre-warmed 0.05% Trypsin-EDTA (Gibco, 15400-054, diluted in DPBS) for 2-3 minutes at room temperature, 3/quenching the harvest with 100 mL aMEM+glutamax supplemented with B27 (minus insulin) (f.c. 1×), 4/collecting the bulk cell suspension into a 500 mL conical centrifuge tube, 5/rinsing harvested flasks with basal aMEM media to recover any remaining cells and adding this rinse to the bulk cell suspension. The concentration of cells in the suspensions were determined using the ViCell Automated Cell Counter, and the cells per cm² from the harvested vessels were back-calculated.

In addition to the CPC adherent and suspension vesiculation cultures, virgin media controls were also performed for adherent and suspension cultures.

For the suspension vesiculation culture virgin media controls, new 0.5 L PBS-mini vessels were filled with 400 mL alpha-MEM complete media (at “day+0”), and incubated for 2 days (37 °C, 5% C02, at atmospheric oxygen), with agitation at 40 rpm. After the two days (“day+2”), the spent culture media was removed, and vessels were rinsed thoroughly (three times each with 50-100 mL of pre-warmed MEM alpha media base). The PBS-mini vessels were then filled with 400 mL alpha-MEM poor media, and incubated for 2 more days (37 °C, 5% CO₂, at atmospheric oxygen), until “day+4.”

For the adherent vesiculation culture virgin media controls, vitronectin-coated hyperflasks were filled with alpha-MEM complete media and incubated for 2 days (37 °C, 5% CO₂, at atmospheric oxygen). After these two days (“day+2”), the spent culture media was removed, and the vessels were rinsed thoroughly (three times each with 50-100 mL of pre-warmed MEM alpha media base). The hyperflasks were then filled with alpha-MEM poor media, and incubated for 2 more days (37 °C, 5% CO₂, at atmospheric oxygen), until “day+4.”

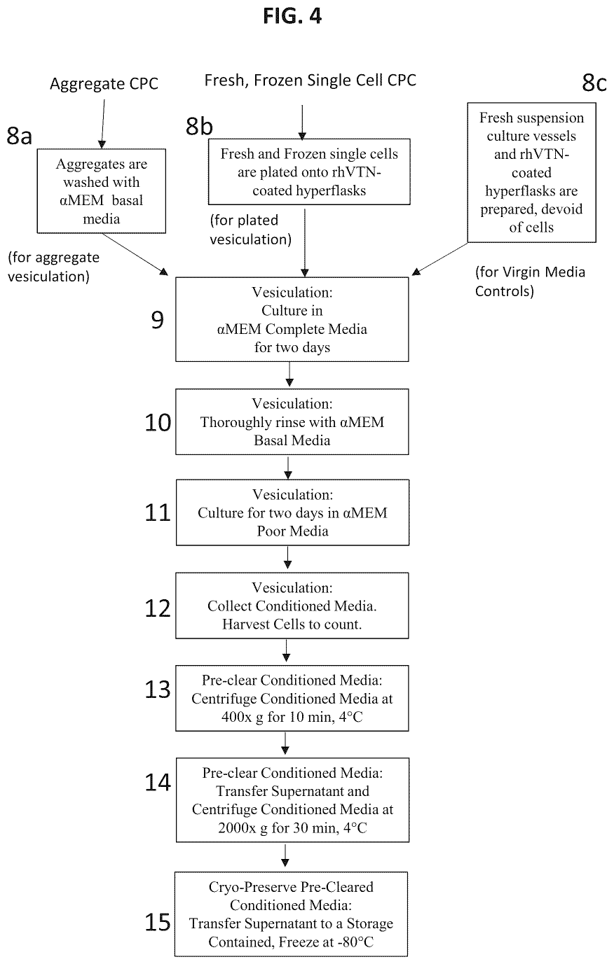

At day+4, media from the suspension and adherent cell cultures (conditioned media, MC), as well as day+4 media from the virgin control vessels (virgin media, MV), were collected, and pre-cleared by serial centrifugation (400×g for 10 minutes at 4 °C, then 2000×g for 30 minutes at 4 °C). The pre-cleared media was then aliquoted into conical tubes, and frozen at −80 °C. FIG. 4 depicts a process flow diagram for the generation of conditioned media and virgin media controls.

### Example 3

Preparation of Small Extracellular Vesicle-Enriched Fraction (sEV)

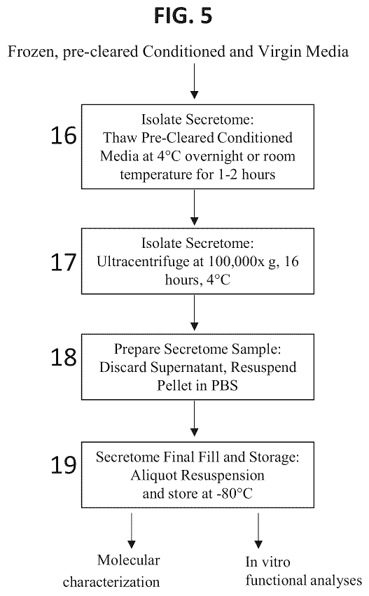

To validate the vesiculation process, samples of the conditioned and control media were subjected to ultracentrifugation, in order to generate sEV and MV preparations for molecular characterization and in vitro functional analyses. Two biological replicates of each sample type were prepared. FIG. 5 depicts a process flow diagram for the isolation of sEV or mock (virgin media) control samples.

MC and MV were thawed at room temperature for 1-4 hours, or overnight at 4 °C. After thawing, MC and MV were ultracentrifuged at 100,000×g for 16 hours at 4 °C (wX+Ultra Series Centrifuge, ThermoScientific; rotor: F50L-8×39; Acceleration: 9; Deceleration: 9), and the resulting supernatants were removed. The bottom of each tube was rinsed twice with 100 μL volumes of 0.1 μm filtered DPBS−/− (0.1 μm PES Filter Unit, ThermoFisher 565-0010) without disturbing the pellet, and then each pellet was resuspended in 0.1 μm filtered DPBS−/− by gentle agitation of the solvent with a sterilized glass stir bar. sEV preparations were collected, and tubes were rinsed with 0.1 μm filtered DPBS−/− for maximum product recovery (to a total resuspension plus rinse target volume as calculated based on the number of secreting cells giving rise to the conditioned media). 45 μL were targeted for every 1.4×10⁶ day+4 secreting cells as calculated by the following formula:

Target sEV Resuspension Volume=(Total Viable Cells at day+4÷Total Volume Conditioned Media at day+4)×Volume MC Centrifuged×(45 μL÷1.4×10⁶ Viable Cells).

Target resuspension volumes for MV controls were matched to the relevant MC target resuspension volumes. For MC and MV generated in PBS-mini vessels, sEV preparations were filtered at 0.65 μm (Ultrafree 0.65 μm DV Durapore, Millipore ref: UFC30DV05) to remove large particulates. sEV and MV control preparations were aliquoted and frozen at −80 °C.

sEV and MV control preparations were further analyzed, as described below.

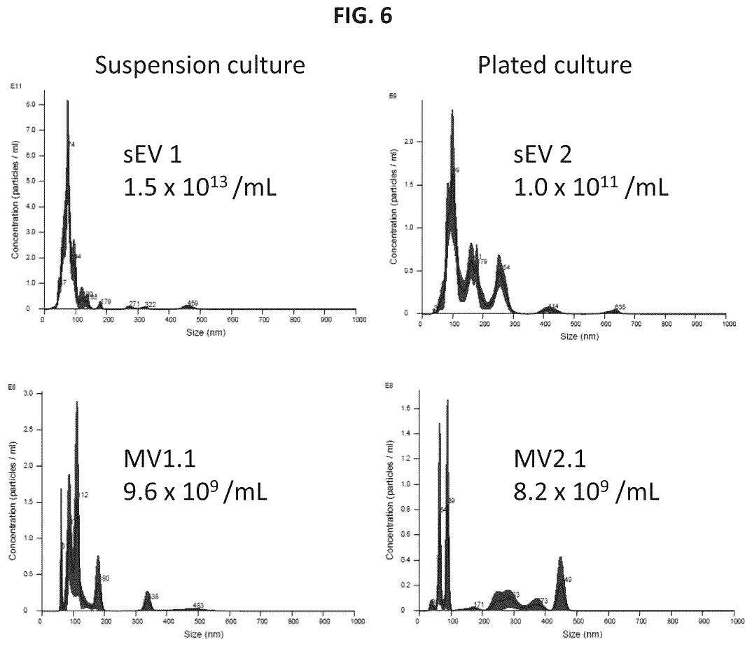

First, the particle concentration and size distribution in sEV and MV control preparations were determined by nanoparticle tracking analysis (NTA; NanoSight). The nanoparticle tracking analysis confirmed the presence of particles of the size of exosomes and microparticles in the sEV prepared from CPC conditioned media, but not in MV controls. FIG. 6 depicts representative size distribution curves from two sEVs and two control MV samples. Observable particle sizes ranged from approximately <30 nm to 300 nm or so, with a peak generally between 50-150 nm, corresponding to the size of exosomes or small microparticles.

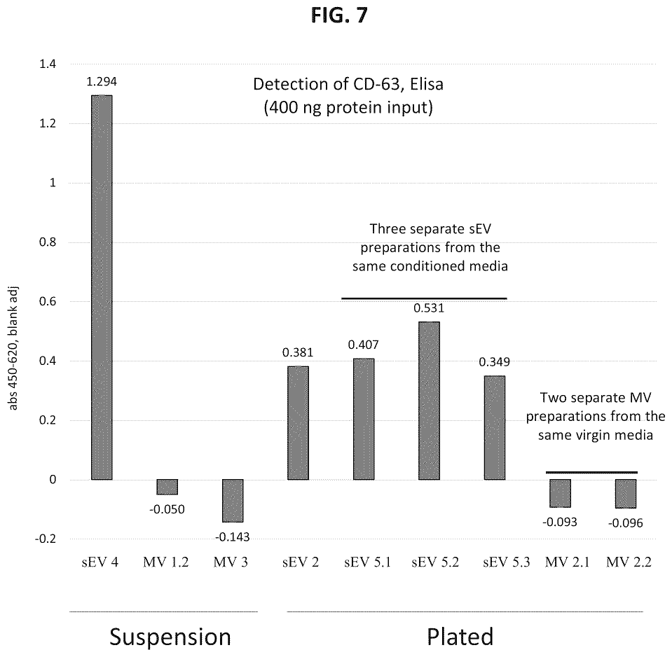

Second, the presence of the exosome-associated vesicle surface marker CD63 was also analyzed using the PS Capture Exosome ELISA Kit (Wako Chemicals, ref: 293-77601), with the primary antibody being an anti-CD63 antibody (Wako Chemicals, ref: 292-79251), and the secondary antibody being an HRP-conjugated Anti-mouse IgG antibody (Wako Chemicals, ref: 299-79261). Input volumes were set such that 400 ng protein from sEV and MV control preparations was added to each well. This anti-CD63 ELISA evaluation confirmed the presence of exosome-associated CD63 surface antigen in each of the sEV samples, but in none of the MV controls (FIG. 7). CD63 signal was higher in the aggregate sample than in the plated samples, although the CD63 signal was consistent between replicates of plated samples. The protein content of sEV and MV control preparations was determined by BCA analysis, using the Pierce Micro BCA kit (ThermoScientific ref: 23235).

### Example 4

### In Vitro Analysis of sEV Functionality

To analyze the functionality of the sEV preparations, three in vitro assays were used: a HUVEC scratch wound healing assay; a cardiomyocyte viability assay using serum-deprived H9c2 cells; and a cardiomyocyte viability assay using staurosporine-treated human cardiomyocytes.

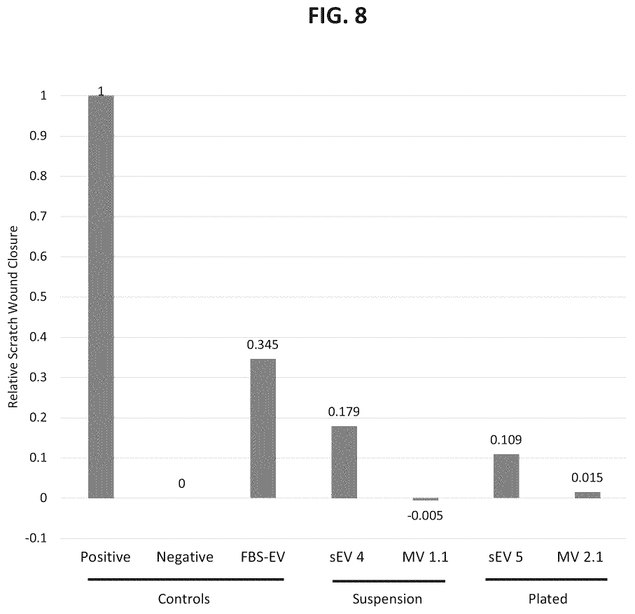

For the HUVEC scratch wound healing assay, a scratch wound healing assay (developed by Essen BioSciences, for the Incucyte) was employed, according to the manufacturer's directions. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete media for two days. Cultures were maintained at 37 °C (atmospheric oxygen, 5% CO₂) throughout maintenance and assay process. Wells were scratched using a Wound Maker (EssenBio, Ref: 4493) according to the manufacturer's directions, and cells were then rinsed with Endothelial Cell Basal Media and cultured overnight (either in HUVEC Complete Media alone, as a positive control; in Endothelial Cell Basal Media alone, as a negative control; or in Endothelial Cell Basal Media supplemented with sEV or MV preparations). Using an Incucyte with the Scratch Wound Healing Module, plates were imaged every three hours for a total of 18 hours. Wound closure was determined using the manufacturer's software, and values were baseline (negative control) subtracted, and normalized to the positive control. FIG. 8 depicts that the sEV preparations, but not the control MV preparation, promoted wound healing, indicating the functionality of the sEV preparation.

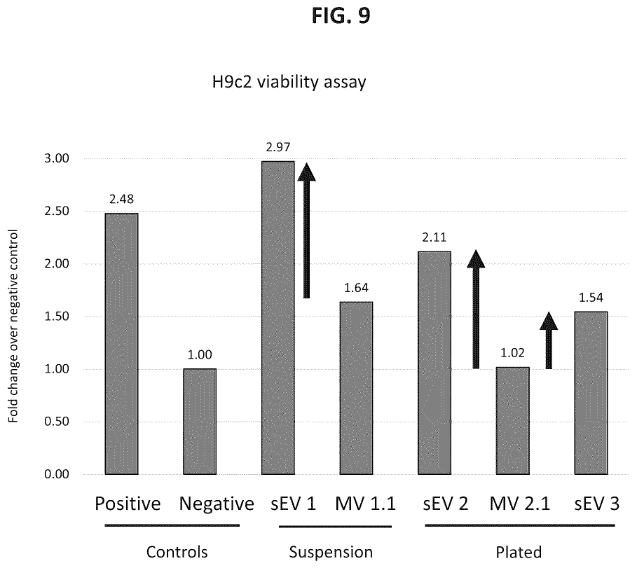

For the cardiomyocyte viability assay using serum-deprived H9c2 cells, the assay was performed essentially as described in El Harane et al. (Eur. Heart J, 2018; 39:1835-1847). In this assay, H9c2 cardiomyocytes are proliferative when culture media is rich in serum (e.g., cultured in H9c2 Complete Media), but cease to proliferate and lose viability when they are deprived of serum (e.g., cultured in H9c2 Poor Media). The capacity of sEV and MV preparations to promote H9c2 cardiomyocyte viability was determined by supplementing the H9c2 Poor Media with increasing concentrations of sEV and MV control preparations. FIG. 9 depicts that the sEV preparations, but not the control MV preparation, improved H9c2 cardiomyocyte viability in the absence of serum, indicating the functionality of the sEV preparation.

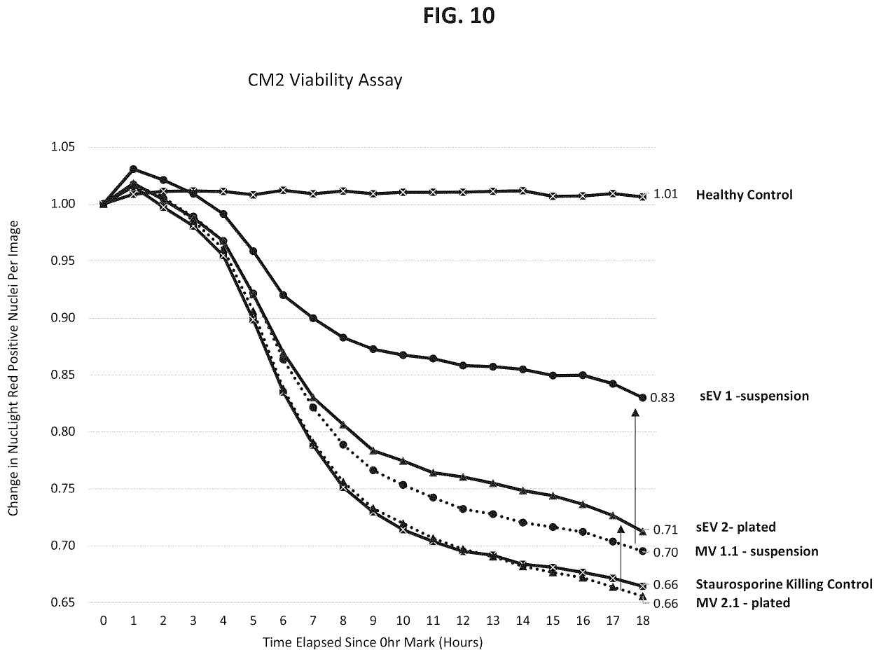

For the cardiomyocyte viability assay using staurosporine-treated human cardiomyocytes, iCell Cardiomyocytes² (Fujifilm Cellular Dynamics, Inc., ref: CMC-100-012-001) were plated at 50,000 cells/well of a fibronectin-coated 96-well plate in iCell Cardiomyocyte Plating Medium (Fujifilm Cellular Dynamics, Inc., ref: M1001), and cultured for 4 hours. The media was then exchanged for iCell Cardiomyocyte Maintenance Medium (iCMM, Fujifilm Cellular Dynamics, Inc., ref: M1003), and cells were cultured for up to 7 days, with full media exchanges every 2-3 days. After a minimum of 4 days, cells were exposed to iCMM with NucSpot Live 650 dye (Biotium, ref: 40082) (this served as a viable cell control); or to iCMM with NucSpot Live 650 dye, and staurosporine (Abcam, ref: ab146588) at a final in-well concentration of 2 M (this also served as an apoptotic cell control). Dye, PBS, and DMSO concentrations, and final well volumes, were equivalent in all wells. Cells were cultured in these pre-incubation media for four hours. After this incubation, the pre-incubation media was removed, and the wells were rinsed with iCMM. Cells were then fed with iCMM with NucSpot Live 650 dye and PBS, or iCMM with NucSpot Live 650 dye supplemented with increasing concentrations of sEV or MV control preparations while maintaining PBS final volumes. Wells were imaged in an Incucyte every hour for 24 hours, and nuclei counts were determined. FIG. 10 depicts that the sEV preparations, but not the control MV preparation, improved cardiomyocyte survival, indicating the functionality of the sEV preparation. The results depicted in FIG. 10 are detailed in TABLE 2.

TABLE 2
18-hour time point data points as depicted in FIG. 10
Change in NucLight Red Positive Nuclei per Image

| Additional Figure | | 18 Hour |
|---|---|---|
| Information | Figure Labels | Timepoint |
| | Healthy Control | 1.01 |
| | sEV 1 - Suspension | 0.83 |
| | sEV 2- Plated | 0.71 |
| Negative Control and | MV 1.1 - Suspension | 0.70 |
| Mock EV Controls with | Staurosporine Killing Control | 0.66 |
| no impact on CM² Viability | MV 2.1 - Plated | 0.66 |

### Example 5

Exemplary Good Manufacturing Practices (GMP)-Compatible Process for Producing Small Extracellular Vesicle-Enriched Fraction (sEV) Formulations of CTC1-EV

A first exemplary GMP-compatible process for producing sEV-containing formulations was developed. The production process included four main stages: vesiculation; conditioned media clarification; enrichment and concentration of small EV-enriched secretome; and production of the final sEV formulation. Flow diagrams outlining the GMP-compatible process that was performed are depicted in FIGS. 11A and 11B.

### Vesiculation

For the vesiculation step, cardiovascular progenitor cells (CPCs) that had been cryopreserved and stored under vapor-phase liquid nitrogen (or within a −150 °C freezer) were initially thawed for two minutes at 37 °C in a thawing medium (MEM alpha (MEM a, GlutaMAX™ Supplement, no nucleosides; Gibco/Life Technologies; ref: 32561-029); glucose (30%) supplement (Macopharma Ref: CARELIDE, to a final overall glucose concentration of 2 mg/mL; Ydralbum® (LFB), at a final concentration of 20 mg/mL; B-27™ Supplement (50×, Life Tech Ref: 17504001 at a final concentration of 1×); and Rock Inhibitor H1152 (Sigma Ref: 555550, at a final concentration of 0.392 g/mL), within an EVA bag (Corning). 18 mL of thawing medium was used per 1 mL of CPCs.

After thawing, CPCs were seeded onto vitronectin (Life Tech Ref: VTN-N; recombinant human protein, truncated (Ref: A31804); 5 g/mL, sterilized using a 0.22 μm filter (syringe filter 0.2 μm polyethersulfone (PES) membrane) coated culture flasks (8×10ST CellStack Culture Chambers, tissue culture (TC)-treated (Corning Ref: 3271); as well as 2×TC-treated, vitronectin-coated T75 flasks), at a seeding density of about 100,000 cells per cm², using 0.2 mL/cm² of complete medium (MEM a, GlutaMAX™ Supplement, no nucleosides; Gibco/Life Technologies; ref: 32561-029; glucose (30%) supplement (Macopharma Ref: CARELIDE, to a final overall glucose concentration of 2 mg/mL; Ydralbum (LFB; 200 g/L); B-27™ Supplement (50×, Life Tech Ref: 17504001 or 17504044, at a final concentration of 1×); Gentamicin (Panpharma, at a final concentration of 25 g/mL); and Human FGF-2 Premium grade (Miltenyi Biotec ref: A12873-01, at a final concentration of 1 g/mL)). Seeding was performed without prior centrifugation of the cell suspension. The seeded CPCs were then cultured in complete medium for three days at 37 °C, in the presence of 5% CO₂ and atmospheric oxygen.

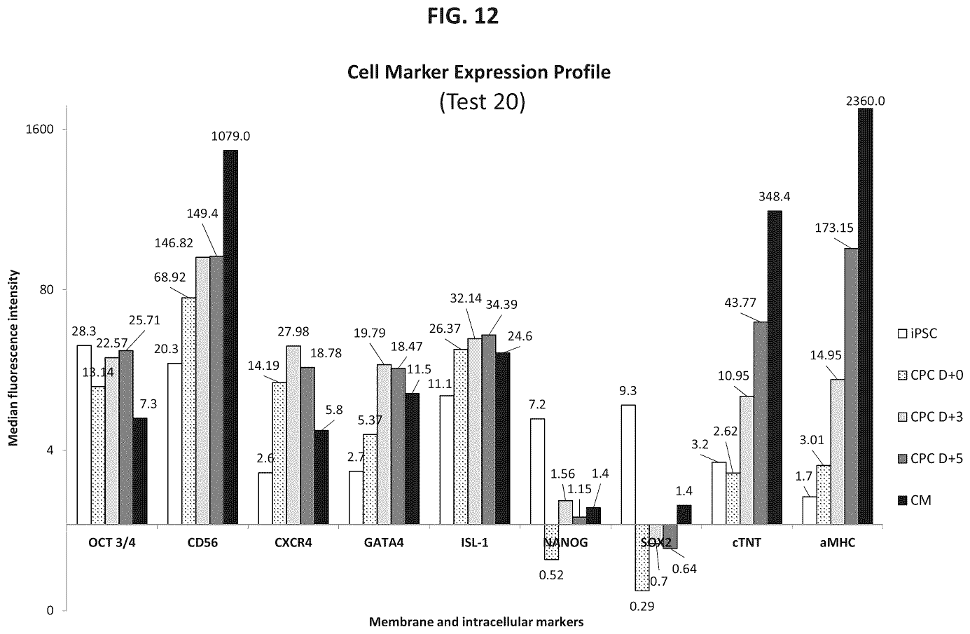

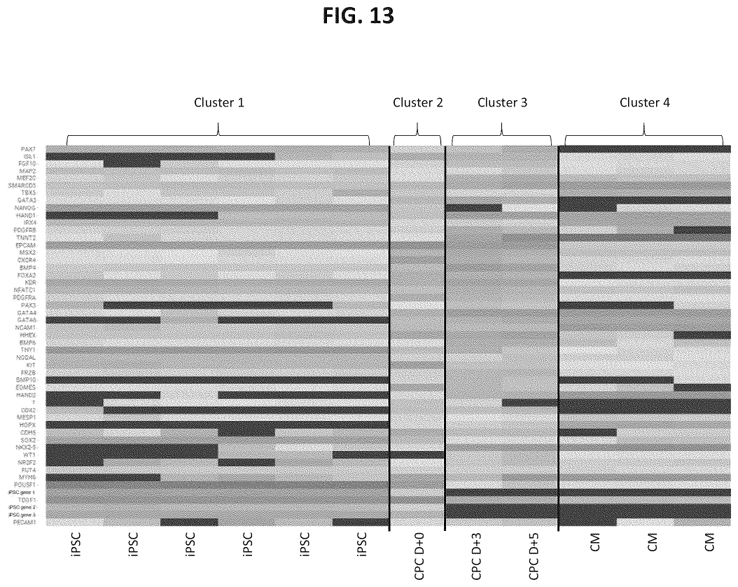

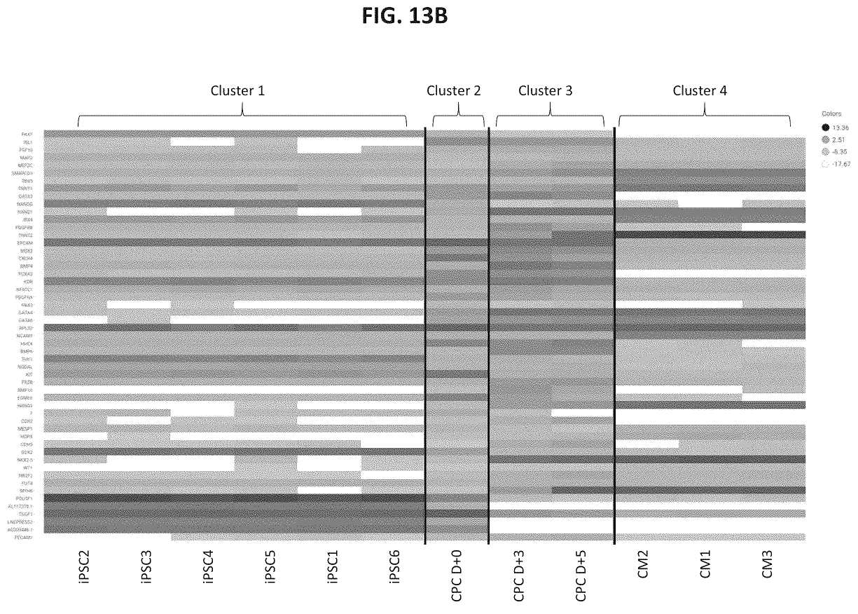

Immediately prior to seeding (“D+0”), cells were analyzed to determine the number and percentage of viable cells (see TABLE 5, column 2, *1 (Test 20)) using a NucleoCounter NC-200 (Chemometec) with DAPI/AO staining (Ph. Eur. 2.7.29); to determine their identity (see FIG. 12 and Example 7) by flow cytometry using a MACSQuant 10 Flow Cytometer; and to analyze their transcriptome (see FIG. 13 and Example 8).

After the 3-day culturing (“D+3”), the cells from one of the cultured T75 flasks were harvested. These harvested cells were analyzed to determine the number and percentage of viable cells (see TABLE 5, column 3, *2 (Test 20)) using a NucleoCounter NC-200 (Chemometec) with DAPI/AO staining (Ph. Eur. 2.7.29); to determine their identity (see FIG. 12 and Example 7) by flow cytometry using a MACSQuant 10 Flow Cytometer; and to analyze their transcriptome (see FIG. 13 and Example 8). Spent media from the 10ST CellStack Culture Chambers was also tested for sterility, and for the presence of mycoplasma and endotoxin.

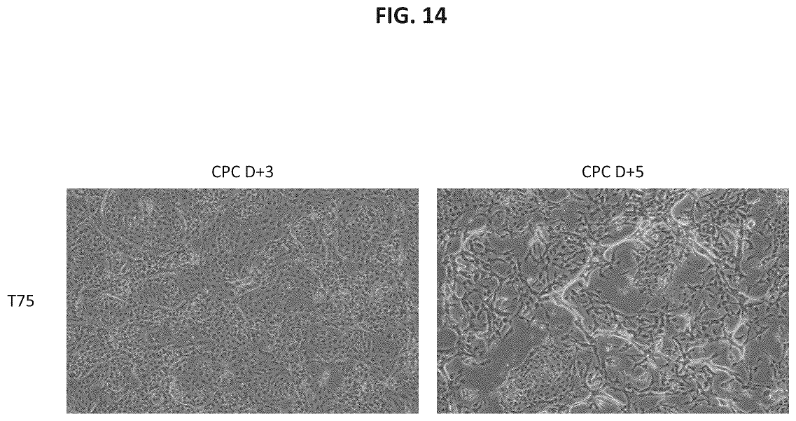

For the remaining flasks (8×10ST CellStack Culture Chambers; and 1×T75), the cells were visualized by microscopy to determine their morphology (see FIG. 14), and washed twice with a wash medium (MEM alpha (Macopharma Ref: BC0110021); glucose (30%) supplement (Macopharma Ref: CARELIDE, to a final overall glucose concentration of 2 mg/mL), before being cultured for 2 days at 37 °C, in the presence of 5% CO₂, in a starvation media (poor media) (MEM alpha (1000 mL of Macopharma Ref: BC0110021); glucose (30%) supplement (Macopharma Ref: CARELIDE, to a final overall glucose concentration of 2 mg/mL). After this 2-day incubation (“D+5”), the culture media (conditioned media) was collected, and the cells from the 10ST CellStack Culture Chambers and the remaining T75 flask were harvested.

As with the cells at D+3, the cells at D+5 were again visualized by microscopy to determine their morphology (see FIG. 14); and the cells harvested at D+5 were further analyzed to determine the number and percentage of viable cells (see TABLE 5, column 4, *3 (Test 20)); to determine their identity (see FIG. 12 and Example 7) by flow cytometry using a MACSQuant 10 Flow Cytometer; and to analyze their transcriptome (see FIG. 13 and Example 8). The collected conditioned media was tested for sterility, and for the presence of mycoplasma and endotoxin, before further processing.

### Conditioned Media Clarification

Clarification of the conditioned media was conducted via a series of four filtration steps. First, filtration was performed using a 200 μm drip chamber filter (Gravity Blood Set, BD careFusion Ref: VH-22-EGA). The resulting filtrate was then filtered with an infuser, using a 15 μm filter (DIDACTIC, Ref: PER1FL25). The resulting filtrate was then filtered using Sartoguard PES XLG MidiCaps (Pore sizes (prefilter+filter): 1.2 μm+0.2 μm, size 7 (0.065 m²); Sartorius Ref: 5475307F7-OO-A). Next, the resulting filtrate was further filtered using a Vacuum Filter/Storage Bottle System (0.22 μm, Pore 33.2 cm², PES Membrane; Corning Ref: 431097).

### Enrichment and Concentration

Following clarification of the conditioned media, the conditioned media was subjected to enrichment and concentration of the small EV secretome.

First, the clarified conditioned media was subjected to Tangential Flow Filtration (TFF), using a TFF Allegro™ CM150 (PALL/Sartorius). For the TFF manifold, a sterile single-use Flow Path Manual Valve P&F (PALL/Sartorius, reference: 744-69N) was used, together with a 5 L Retentate Assembly (sterile, single use; PALL/Sartorius Ref: 744-69L). For the TFF cassette, sterile single-use regenerated cellulose filters (30 kDa cut-off; 0.14 m²; Sartorius Ref: Opta filter assembly+3D51445901MFFSG) were used. For recovery of the retentate (i.e., what is retained in the TFF), a Bench Top TFF 1 L Bag was used (PALL/Sartorius, reference: 7442-0303P).

Initially, the TFF device was washed with 10 L of H₂O, and 1 L of 1×PBS (filter sterilized using a 0.2 μm filter) before operation. Next, after administration of the clarified conditioned media to the TFF device, the retentate was concentrated (to 500 mL; not exceeding 3 bars of pressure). After this initial concentration step, the retentate was subjected to diafiltration (6 diafiltration volumes; using 1×DPBS, filter sterilized using a 0.2 μM filter). After diafiltration, the retentate was further concentrated, to produce a total volume of at least 100 mL. The parameters of the TFF process were as follows: feed manifold pressure (PTO1)—0.86-2.1 bars; retentate manifold pressure (PTO2)— 0.11-0.14 bars; retentate manifold flow rate (FTO1)—0.03-0.32 L/min; transmembrane pressure (TMPO1)—0.4-1.1 bars; and quattroflow pump (P01)—18-23%.

### Example 6

### Formulation/Composition

After enrichment and concentration by TFF, retentate was processed as depicted in FIG. 11B. Briefly, retentate alone, retentate including 25 mM trehalose, and retentate including 5 g/L L-histidine, were each stored in glass vials (2 mL, bromobutyl cap; Adelphi Ref: VCDIN2RDLS1) and stored at −80 °C. Quality control testing was performed on these samples (the different stages at which quality control testing was undertaken are indicated with a “*,” e.g., *6, *7, etc.). Additionally, final sEV formulations were also prepared by filter sterilizing retentate (with or without 25 mM trehalose) using a 0.22 μm filter (Sterivex™-GP Pressure Filter Unit, 0.22 μm, Millipore, Ref: SVGPL10RC). After the sterilization step, the final formulations (with or without the addition of 25 mM trehalose) were bottled into glass vials (2 mL, bromobutyl cap; Adelphi Ref: VCDIN2RDLS1). In addition, any pharmaceutically suitable carrier may be utilized. Final formulations were stored at −80 °C for future use or testing.

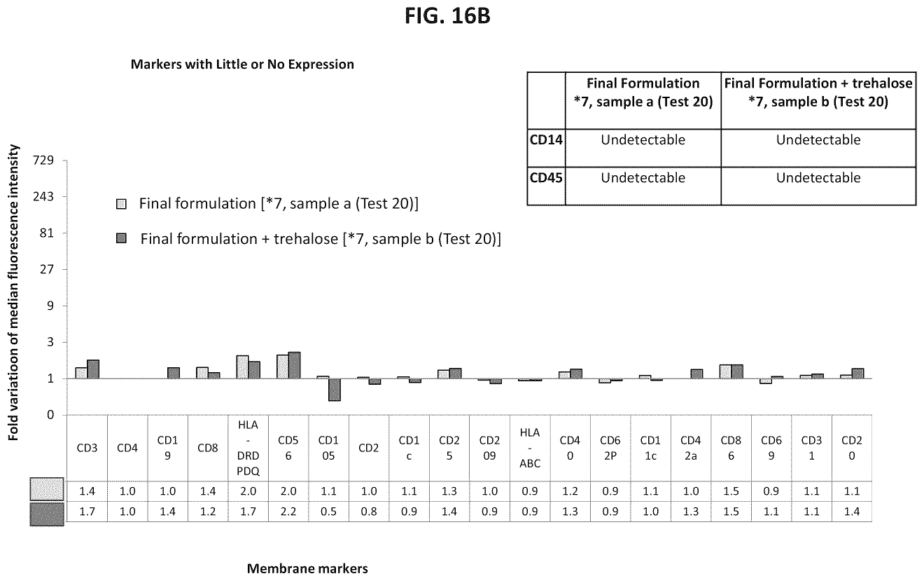

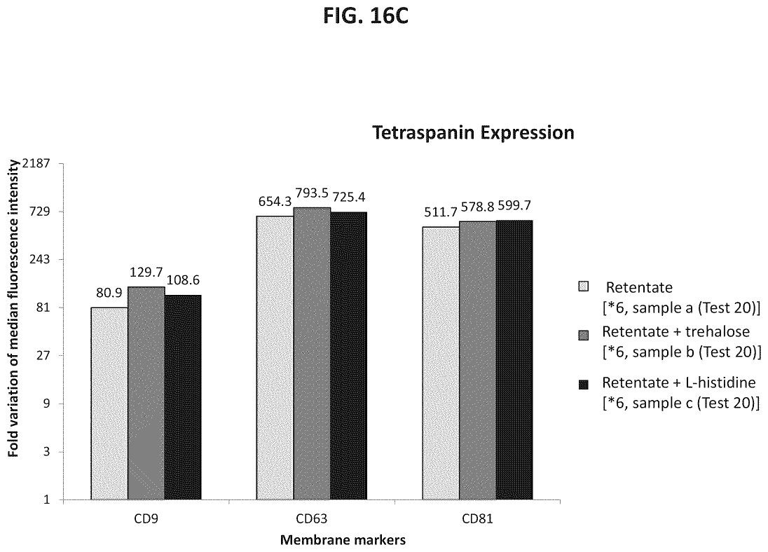

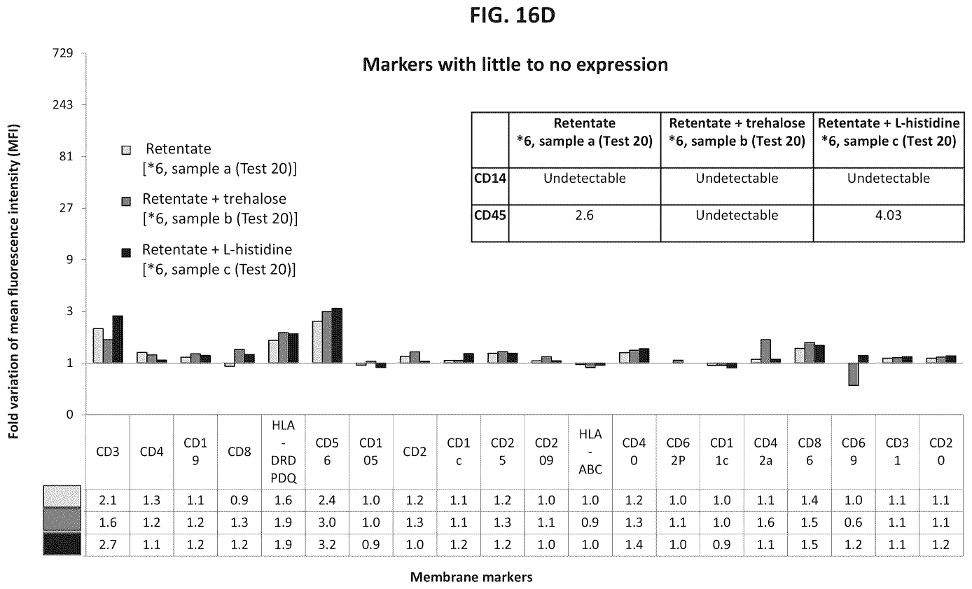

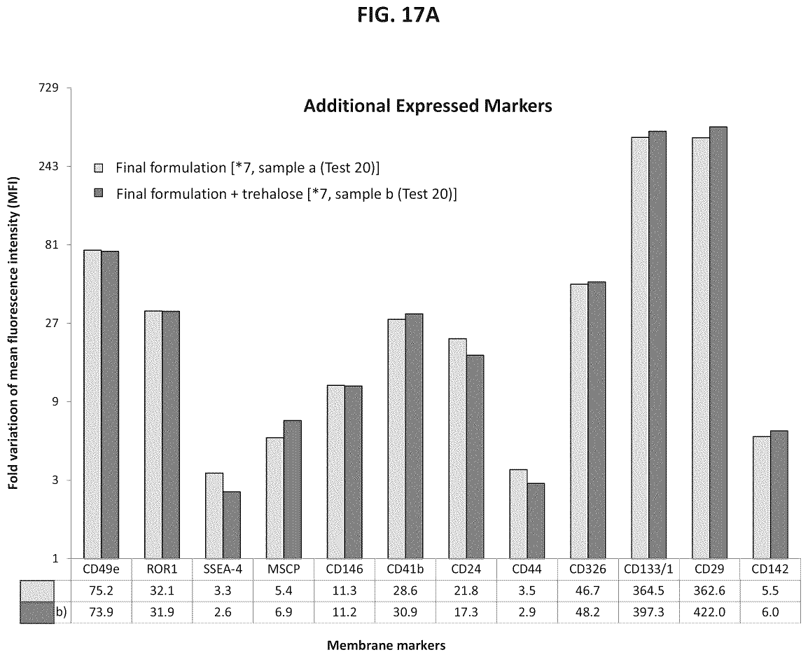

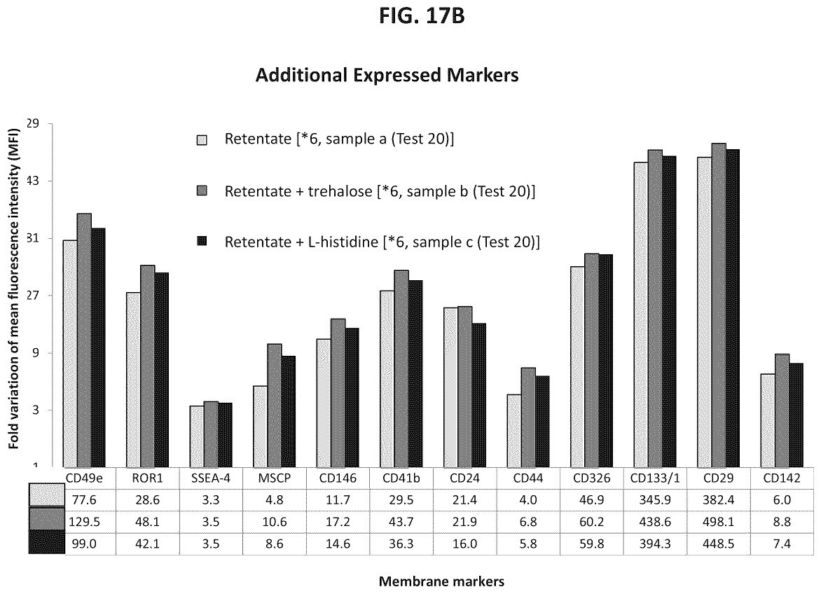

The final formulations, therefore, were in PBS (with or without trehalose), and were positive for CD9, CD63 and CD81 (canonical EV markers), as well as positive for the cardiac-related markers CD49e, ROR1, SSEA-4, MSCP, CD146, CD41b, CD24, CD44, CD236, CD133/1, CD29 and CD142, as detected by MACSPlex (as shown in FIGS. 16A, 16C, 17A and 17B).

### Example 7

### Characterization of the Identity of CPCs During Vesiculation in the GMP-Compatible Process

To assess the identity of the cells during the vesiculation process in Example 5, the D+0 CPCs, as well as the harvested cells at D+3 and D+5, were analyzed by flow cytometry. iPSCs and cardiomyocyte (CM) cells were included as controls. As shown in FIG. 12, flow cytometry analysis, performed using a MACSQuant 10 Flow Cytometer with iPSC-, CPC- and cardiac-markers, demonstrated that the CPCs became more mature over the five-day vesiculation period. Specifically, the CPCs maintained little to no NANOG or SOX2 protein expression, and exhibited a continued increase in CD56, cTNT, and aMHC, protein expression (however, they did not reach expression levels of CD56, cTNT, and aMHC similar to cardiomyocytes, indicating that they remained progenitors throughout the process). iPSC and CM control cells were analyzed separately, and the average values are presented in FIG. 12 for comparative purposes.

### Example 8

### Transcriptome Analysis of CPCs During Vesiculation in the GMP-Compatible Process

To assess the transcriptome of the cells during the vesiculation process in Example 5, RNA was extracted from the CPCs at D+0, and from the harvested cells at D+3 and D+5 of the vesiculation process. RNA was also extracted from iPSCs (pluripotent cell controls), and from iPSC-derived cardiomyocytes (differentiated cardiomyocyte controls). Total RNA was sequenced on the Illumina NovaSeq 6000 platform, and differential gene expression was determined on normalized data.

The heatmaps depicted in FIG. 13 and FIG. 13B were generated based on hierarchical clustering analysis using the UPGMA clustering method, with correlation distance metric in TIBCO Spotfire software v11.2.0. The genes included in the panel are expressed at different stages of differentiation (from iPSC through to beating cardiomyocytes), as well as related off-target cells. The gene expression analysis results depicted in FIG. 13 and FIG. 13B thus confirmed that the cells retained the characteristics of cardiovascular progenitors throughout the vesiculation process. The data used to generate the heatmaps for FIG. 13 and FIG. 13B are presented in TABLE 3.

TABLE 3
Gene names and log FPKM values corresponding to the data presented in the heatmap depicted in FIG, 13 and FIG. 13B.

| Gene | CM2.012 | CM2.390 | CM2.990 | CTC1+0. | CTC1+3. | CTC1+5. |
|---|---|---|---|---|---|---|
| Name | D13A | D13A | D13A | x331 | x506 | x507 |
| Alias | CM1 | CM2 | CM3 | CPC D+0 | CPC D+3 | CPC D+5 |
| EOMES | −4.93069 | −4.12089 | 17.6702 | 5.508136 | 3.64079 | 2.40622 |
| MSX2 | 1.95635 | 2.079638 | 2.131557 | 3.712693 | 5.159327 | 4.612603 |
| RPL32 | 7.803537 | 7.341461 | 7.600563 | 7.89441 | 6.806746 | 6.614746 |
| POUSF1 | −4.59453 | −2.56237 | −2.41242 | 5.470052 | −2.10233 | −5.47401 |
| NANOG | −17.6702 | −6.26626 | −4.11672 | 0.867215 | −7.39055 | −4.7862 |
| SOX2 | −4.3899 | −2.58011 | −2.69319 | 3.283494 | −0.96809 | −0.84339 |
| TDGF1 | 2.798607 | 2.477319 | 2.639293 | 8.106968 | 3.988754 | 3.480323 |
| MESP1 | 1.070132 | 0.504985 | 0.496239 | −0.85743 | −1.88294 | −5.18429 |
| T | −17.6702 | −17.6702 | −17.6702 | 2.47037 | −3.35506 | −17.6702 |
| EPCAM | 0.278948 | 0.475306 | 0.068866 | 7.711402 | 6.618857 | 7.026096 |
| CXCR4 | −1.04385 | −0.66692 | −1.51696 | 6.423817 | 4.415654 | 3.2946 |
| CDX2 | −17.6702 | −17.6702 | −17.6702 | −3.93324 | 1.793699 | 2.955975 |
| FGF10 | −2.48091 | −1.96649 | −2.10604 | −0.27379 | 1.992171 | 2.494637 |
| ISL1 | −2.58766 | −1.36276 | −1.62784 | 4.87507 | 4.069774 | 4.059169 |
| KDR | −0.61909 | −1.48162 | −0.80811 | 4.919707 | 6.272936 | 5.988755 |
| KIT | −2.36653 | −1.78798 | −2.25469 | 6.486542 | 2.0949 | 2.455783 |
| PDGFRA | −3.64284 | −2.11841 | −2.38349 | 3.779217 | 3.128421 | 2.367591 |
| PDGFRB | −6.89513 | −5.08582 | −17.6702 | −0.0682 | 4.613454 | 3.136563 |
| NCAM1 | 5.395119 | 5.633374 | 5.55992 | 1.872388 | 1.736935 | 2.053235 |
| HAND1 | 6.083721 | 6.048107 | 6.24644 | 2.634496 | 6.862401 | 6.87863 |
| HAND2 | 6.855635 | 6.630765 | 6.875506 | −2.53191 | 3.694809 | 3.947744 |
| TBX5 | 4.403699 | 4.828509 | 4.803637 | −3.80878 | −1.62649 | −1.02117 |
| HOPX | 2.837924 | 2.668974 | 2.546846 | −2.99056 | −2.48907 | −1.16708 |
| GATA4 | 6.45317 | 6.684524 | 6.620094 | 3.724098 | 6.572704 | 6.510904 |
| GATA6 | 5.248785 | 5.313554 | 5.406355 | 3.036861 | 4.196987 | 4.637657 |
| NKX2-5 | 8.531764 | 8.111706 | 8.290329 | −2.95621 | 6.74337 | 7.339022 |
| SMARC | 5.934004 | 5.753566 | 5.872929 | 1.689813 | 3.720093 | 4.637796 |
| D3 | | | | | | |
| MEF2C | 0.919444 | 1.223806 | 1.094438 | −3.89762 | 1.961938 | 2.967551 |
| WTI | −2.1939 | −1.87587 | −2.26648 | −6.62995 | −4.67871 | −5.3952 |
| FOXA2 | −17.6702 | −17.6702 | −17.6702 | 4.70754 | 4.689872 | 4.333305 |
| BMP4 | −2.68445 | −2.45953 | −2.30959 | 2.334512 | 6.456659 | 5.738226 |
| BMP6 | −2.9507 | −4.31065 | −5.16059 | −0.85606 | 5.024752 | 5.061907 |
| BMP10 | −17.6702 | −17.6702 | −3.34925 | −3.25395 | 4.142337 | 2.801309 |
| FRZB | −0.47353 | −0.0371 | −1.17665 | 0.69709 | 3.767637 | 4.147095 |
| MAP2 | −2.26962 | −2.50863 | −2.51622 | −2.06608 | −1.96798 | 0.197686 |
| CDH5 | −4.31678 | −17.6702 | −3.94193 | −7.16766 | 2.509654 | 3.05233 |
| PECAM | −7.01561 | 6.79081 | −6.64093 | −1.7134 | 0.454202 | 1.756716 |
| HHEX | −3.81186 | −5.17174 | −17.6702 | 5.218886 | 4.910112 | 5.61524 |
| THY I | −4.30538 | −3.23253 | −3.66752 | 0.256374 | 2.281531 | −0.57633 |
| PAX3 | −17.6702 | −17.6702 | −5.89729 | −1.69384 | −5.58727 | −3.69269 |
| PAX7 | −17.6702 | −17.6702 | −17.6702 | 0.059575 | −5.61861 | −6.59792 |
| NR2F2 | 0.093554 | 0.057954 | 0.417358 | −2.58127 | 1.397921 | 2.80826 |
| GATA3 | −17.6702 | −17.6702 | −17.6702 | 3.732925 | 5.37344 | 4.756712 |
| NODAL | −4.82002 | −4.59512 | −1.44532 | 0.157774 | 0.08716 | 1.64001 |
| NFATCI | 0.159469 | 0.341069 | 0.29334 | 0.605986 | 3.542907 | 2.788099 |
| TNNT2 | 10.16398 | 9.818392 | 10.03421 | −2.8652 | 3.659895 | 6.878018 |
| TNNTI | 6.663359 | 6.249284 | 6.378282 | 3.869569 | 3.115778 | 3.833164 |
| IRX4 | 5.723521 | 5.474485 | 5.582664 | −0.52898 | 0.512035 | −0.07584 |
| MYH6 | 7.906485 | 7.950674 | 8.108717 | −5.48167 | 3.748825 | 8.079681 |
| LNCPRE | −17.6702 | −17.6702 | −17.6702 | 2.715885 | −17.6702 | −17.6702 |
| SS2 | | | | | | |
| AL11737 | −17.6702 | −17.6702 | −17.6702 | 0.776447 | −17.6702 | −17.6702 |
| 8.1 | | | | | | |
| AC00944 | −17.6702 | −17.6702 | −17.6702 | 4.766971 | −17.6702 | −17.6702 |
| 6.1 | | | | | | |
| FUT4 | −0.98727 | −0.30549 | −1.33062 | 0.127895 | 0.247661 | 0.48857 |
| Gene | iPSC. | iPSC. | iPSC. | iPSC. | iPSC. | iPSC. |
| Name | x198 | x225 | x226 | x227 | x228 | x247 |
| Alias | iPSC1 | iPSC2 | iPSC3 | iPSC4 | iPSC5 | iPSC6 |
| EOMES | −1.20903 | −1.10168 | −1.27814 | −1.28834 | −1.7705 | −0.75971 |
| MSX2 | −2.62097 | −2.41409 | −2.86355 | −2.65964 | −2.28455 | −3.91335 |
| RPL32 | 5.704122 | 6.983356 | 7.156383 | 6.298661 | 7.137875 | 7.196639 |
| POUSF1 | 8.933974 | 9.712896 | 9.793549 | 9.16117 | 9.775374 | 9.716417 |
| NANOG | 6.384948 | 5.751742 | 5.814302 | 6.169852 | 5.816635 | 5.873866 |
| SOX2 | 6.426325 | 6.755212 | 6.784912 | 6.368943 | 6.815202 | 6.878672 |
| TDGF1 | 7.281038 | 7.373385 | 7.345499 | 7.464435 | 7.372176 | 7.430424 |
| MESP1 | −2.90494 | −1.4053 | −0.80969 | −2.98425 | −1.20865 | −2.23804 |
| T | −2.83278 | −2.04096 | −1.20093 | −17.6702 | −2.23334 | −2.38827 |
| EPCAM | 6.068035 | 6.468864 | 6.447723 | 6.368826 | 6.491555 | 6.566957 |
| CXCR4 | −1.49192 | −1.7065 | −2.24907 | −1.89315 | −1.25503 | −1.07655 |
| CDX2 | −17.6702 | −5.59637 | −17.6702 | −5.10506 | −6.46655 | −17.6702 |
| FGF10 | −17.6702 | −4.55848 | −3.27108 | −3.06717 | −2.55459 | −3.09855 |
| ISL1 | −17.6702 | −5.0802 | −5.79256 | −17.6702 | −4.36578 | −17.6702 |
| KDR | 5.674211 | 5.366761 | 5.308578 | 5.74616 | 5.252178 | 5.135717 |
| KIT | 4.311199 | 3.772485 | 3.806128 | 4.177291 | 3.682461 | 3.859465 |
| PDGFRA | −2.17083 | −2.55065 | −2.64167 | −2.39045 | −2.66212 | −1.95458 |
| PDGFRB | −0.28657 | −0.26973 | −0.23081 | −0.53971 | −0.12985 | −0.9031 |
| NCAM1 | 0.610855 | −0.2643 | −0.02992 | −0.06047 | −0.16995 | −0.0858 |
| HAND1 | −17.6702 | −5.35051 | −17.6702 | −17.6702 | −5.22099 | −5.11291 |
| HAND2 | −17.6702 | −17.6702 | −17.6702 | −17.6702 | −6.27168 | −17.6702 |
| TBX5 | −2.78598 | −5.67833 | −3.80613 | −4.18714 | −4.54898 | −5.44074 |
| HOPX | −17.6702 | −17.6702 | −7.21959 | −17.6702 | −17.6702 | −17.6702 |
| GATA4 | −3.22722 | −3.38289 | −5.41711 | −3.62844 | 5.57502 | 4.14521 |
| GATA6 | −17.6702 | −17.6702 | −5.24563 | −17.6702 | −17.6702 | −17.6702 |
| NKX2-5 | −17.6702 | −3.61954 | −17.6702 | −17.6702 | −1.90509 | −5.3817 |
| SMARC | 0.751414 | 1.079913 | 1.45421 | 0.604492 | 1.258924 | 1.237059 |
| D3 | | | | | | |
| MEF2C | −1.46101 | −2.64909 | −2.77663 | −1.97327 | −2.58666 | −2.03801 |
| WTI | −17.6702 | 17.6702 | −17.6702 | −17.6702 | −6.4634 | 6.35535 |
| FOXA2 | −2.45589 | −2.47407 | −4.06097 | −3.85706 | −3.48199 | −1.44795 |
| BMP4 | 0.715427 | 0.564244 | 1.041006 | 0.04211 | 0.8103 | 0.435453 |
| BMP6 | −1.47281 | −1.12154 | −0.69655 | −0.32757 | −0.94469 | −0.98344 |
| BMP10 | −17.6702 | −17.6702 | −17.6702 | −17.6702 | −17.6702 | 17.6702 |
| FRZB | 0.678456 | 0.785806 | 0.117695 | 0.36468 | 0.307667 | 0.764909 |
| MAP2 | 1.157076 | 1.107991 | 1.148634 | 1.467803 | 0.993608 | 1.42246 |
| CDH5 | −3.82379 | −5.54629 | −5.84369 | −5.63982 | 5.00181 | −17.6702 |
| PECAM | −3.74202 | −17.6702 | −17.6702 | −7.01693 | −5.3794 | −7.27053 |
| HHEX | 1.52657 | 1.649517 | 1.495883 | 1.601768 | 2.097881 | 1.765847 |
| THY I | 4.996074 | 5.565449 | 5.588593 | 5.315215 | 5.564195 | 5.47913 |
| PAX3 | −17.6702 | −4.76512 | −17.6702 | −6.27336 | −17.6702 | −17.6702 |
| PAX7 | 3.906573 | 3.934883 | 4.019054 | 4.117352 | 3.933968 | 3.723787 |
| NR2F2 | −6.93614 | −5.65936 | −5.95675 | −5.75289 | −7.11417 | −17.6702 |
| GATA3 | −1.94742 | 2.34821 | −2.26717 | −2.44176 | −2.80361 | −2.31702 |
| NODAL | 3.164671 | 2.91106 | 2.881441 | 2.785799 | 3.026054 | 2.990784 |
| NFATCI | 2.869312 | 2.334634 | 2.536342 | 2.737406 | 2.405227 | 2.425333 |
| TNNT2 | −4.10532 | −2.32563 | −2.31825 | −2.52936 | −2.37666 | −2.3681 |
| TNNTI | 2.635968 | 3.911363 | 3.942202 | 3.067618 | 3.761603 | 3.54891 |
| IRX4 | 3.20956 | 3.797426 | 3.82596 | 3.416459 | 3.937915 | 4.009723 |
| MYH6 | −17.6702 | −4.0822 | −4.96454 | −6.76009 | −5.12246 | −7.01372 |
| LNCPRE | 5.877491 | 5.653432 | 5.657131 | 5.522744 | 5.606096 | 5.538092 |
| SS2 | | | | | | |
| AL11737 | 6.376442 | 5.982486 | 5.938273 | 6.240889 | 5.974234 | 6.047174 |
| 8.1 | | | | | | |
| AC00944 | 6.394576 | 6.423589 | 6.166463 | 6.306408 | 6.274441 | 6.457644 |
| 6.1 | | | | | | |
| FUT4 | 0.994454 | 0.987114 | 1.00542 | 1.03246 | 0.96579 | 1.050983 |

### Example 9

### Analysis of EV Particle Concentration and EV Particle Size Distribution in the GMP-Compatible Process

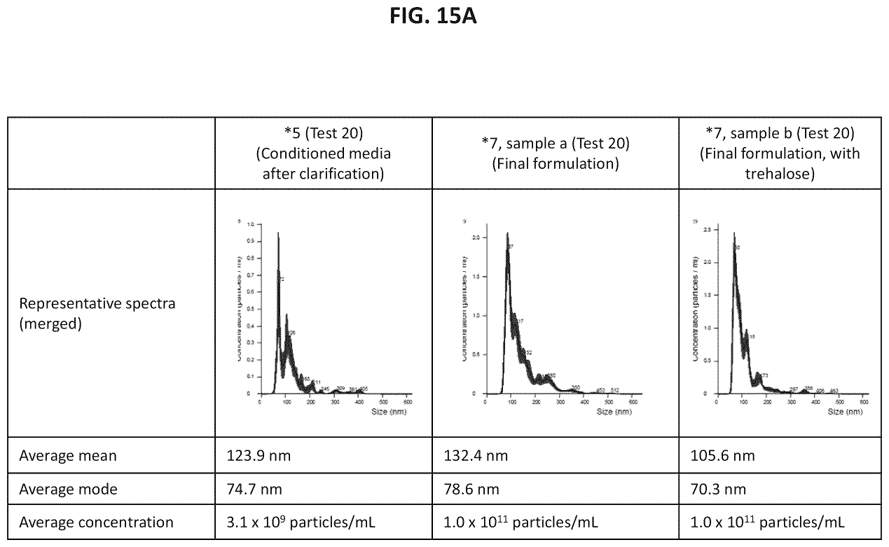

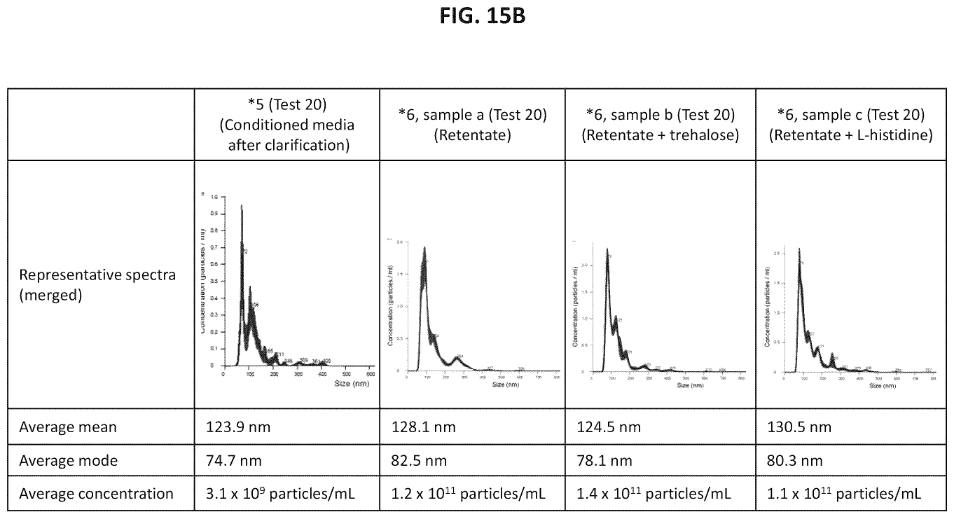

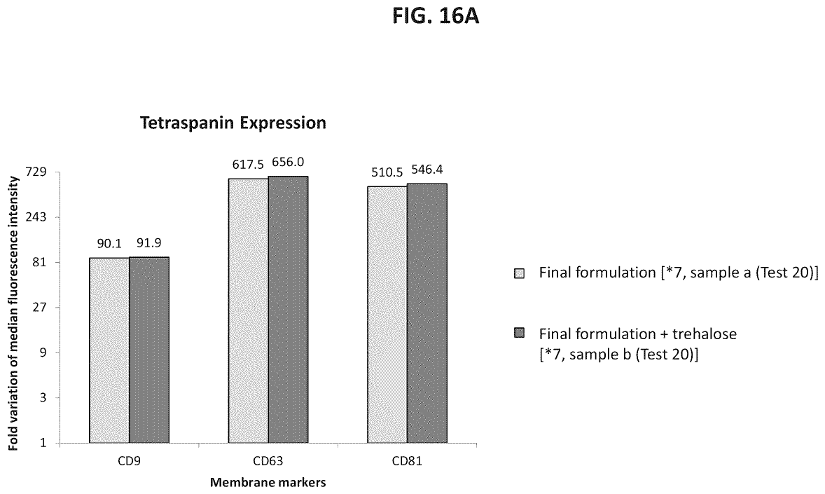

To assess the particle concentration and size distribution of EVs produced in Example 5, the clarified conditioned media (before TFF), and the final formulations (with and without trehalose), were analyzed by nanoparticle tracking analysis (NTA; NanoSight). FIG. 15A depicts representative size distribution curves for each sample. The overall size distributions, means and modes, were similar between samples. A peak was observed generally between about 50-150 nm, corresponding to the size of exosomes or small microparticles. The TFF step resulted in an approximately 32-fold concentration of particles. Similar experiments were also conducted on the stored retentate samples depicted in FIG. 11B (with and without trehalose or histidine) which were not filter sterilized (“*6,” samples a-c). The results of these experiments are shown in FIG. 15B.

### Example 10

### Analysis of EV Markers in CTC1-EV Produced by the GMP-Compatible Process

To assess the presence of EV markers in the clarified conditioned media (before TFF) and the final formulations (with and without trehalose) in Example 5, a MACSPlex Exosome Kit human (Miltenyi Ref: 130-108-813) was used to identify and quantify the presence of EV markers. As shown in FIG. 16A, the analysis confirmed the presence of extracellular vesicle tetraspanins (CD9, CD81 and CD63) in both the conditioned media (before TFF), and in the final formulation (with and without trehalose). Further still, as shown in FIG. 16B, the MACSPlex analysis also revealed a variety of markers that were found to be present either in low amounts (e.g., CD3, CD4, CD8, HLA-DRDPDQ, CD56, CD105, CD2, CD1c, CD25, CD40, CD11c, CD86, CD31 and CD20); or were substantially absent (CD19, CD209, HLA-ABC, CD62P, CD42a and CD69), in the conditioned media (before TFF), and/or in the final formulation (with and without trehalose). Similar experiments were also conducted on the stored retentate samples depicted in FIG. 11B (with and without trehalose or histidine) which were not filter sterilized (“*6,” samples a-c, Test 20). The results of these experiments are shown in FIGS. 16C and 16D.

Additionally, as shown by FIG. 17A, additional cardiac-related markers were also observed in the conditioned media (before TFF), and in the final formulation (with and without trehalose). Similar experiments were also conducted to confirm the presence of these additional cardiac-related markers in the stored retentate samples depicted in FIG. 11B (with and without trehalose or histidine) which were not filter sterilized (“*6,” samples a-c, Test 20). The results of these experiments are shown in FIG. 17B.

### Example 11

### In Vitro Analysis of the Potency of CTC1-EV Produced by the GMP-Compatible Process

To analyze the functionality and potency of the final formulations produced by the GMP-compatible process in Example 5, two in vitro assays were used: a HUVEC scratch wound healing assay; and a cardiomyocyte viability assay using staurosporine-treated human cardiomyocytes.

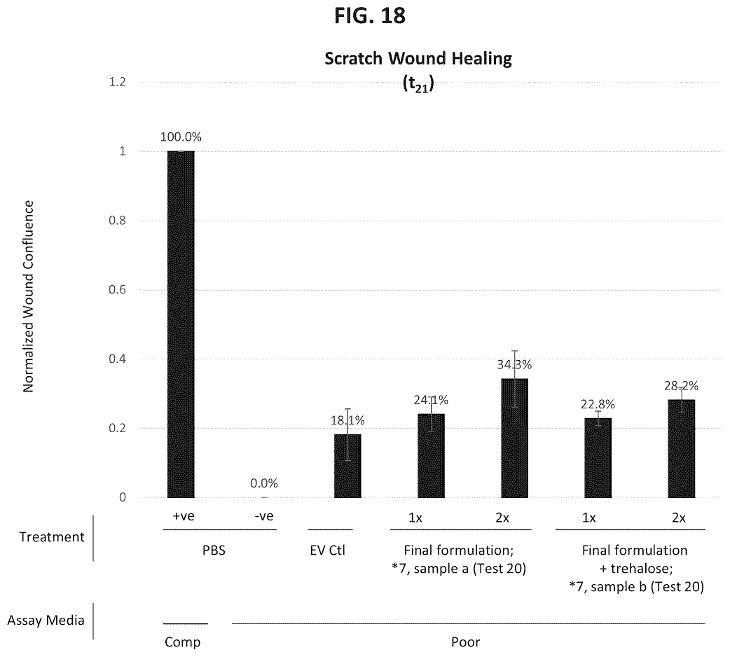

For the HUVEC scratch wound healing assay, a scratch wound healing assay (developed by Essen BioSciences, for the Incucyte) was employed, according to the manufacturer's directions. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete media for two days. Cultures were maintained at 37 °C (atmospheric oxygen, 5% CO₂) throughout the maintenance and assay process. Wells were scratched using a Wound Maker (EssenBio, Ref: 4493) according to the manufacturer's directions, and cells were then rinsed with Endothelial Cell Basal Media and cultured overnight (either in HUVEC Complete Media and PBS, as a positive control; in Endothelial Cell Basal Media and PBS, as a negative control; or in Endothelial Cell Basal Media supplemented with sEV preparations in PBS). Using an Incucyte with the Scratch Wound Healing Module, plates were imaged at 21 hours after treatment. Wound closure was determined using the manufacturer's software, and values were baseline (negative control) subtracted, and normalized to the positive control. FIG. 18 depicts that the final formulations with and without trehalose (sample b and a, respectively) promoted wound healing.

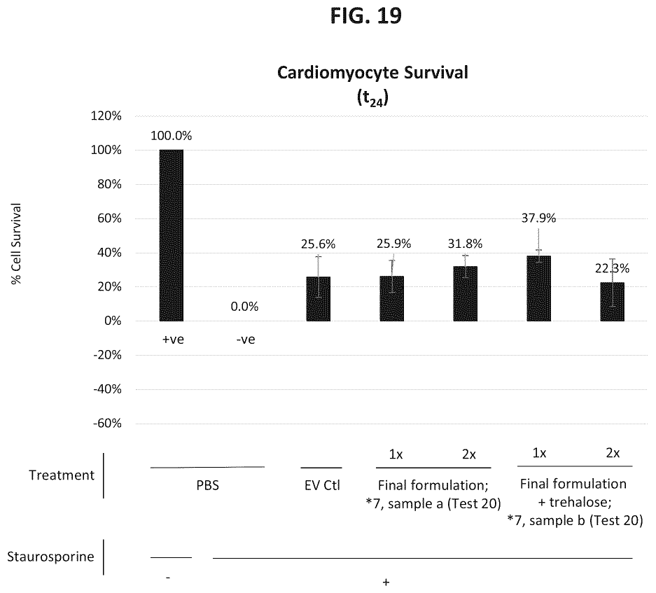

For the cardiomyocyte viability assay using staurosporine-treated human cardiomyocytes, iCell Cardiomyocytes² (Fujifilm Cellular Dynamics, Inc., ref: CMC-100-012-001) were plated at 50,000 cells/well of a fibronectin-coated 96-well plate in iCell Cardiomyocyte Plating Medium (Fujifilm Cellular Dynamics, Inc., ref: M1001), and cultured for 4 hours. The media was then exchanged for iCell Cardiomyocyte Maintenance Medium (iCMM, Fujifilm Cellular Dynamics, Inc., ref: M1003), and cells were cultured for up to 7 days, with full media exchanges every 2-3 days. After a minimum of 4 days, cells were exposed to iCMM with NucSpot Live 650 dye (Biotium, ref: 40082) (this served as a viable cell control); or to iCMM with NucSpot Live 650 dye, and staurosporine (Abcam, ref: ab146588) at a final in-well concentration of 2 M (this also served as an apoptotic cell control). Dye, PBS, and DMSO concentrations, and final well volumes, were equivalent in all wells. Cells were cultured in these pre-incubation media for four hours. After this incubation, the pre-incubation media was removed, and the wells were rinsed with iCMM. Cells were then fed with iCMM with NucSpot Live 650 dye and PBS, or iCMM with NucSpot Live 650 dye supplemented with increasing concentrations of sEV preparations (sample a and b) while maintaining PBS final volumes. Wells were imaged in an Incucyte at 24 hours, and nuclei counts were determined. FIG. 19 depicts that the final formulations with and without trehalose promoted cardiomyocyte survival.

The testing panel used with respect to the processes/products of Example 5, and as embodied, e.g., in Examples 6-11, is shown in TABLE 4. The results therefore are shown in TABLE 5. Additionally, TABLE 6 depicts the degree of enrichment, as compared to conditioned media after clarification, for the retentates and final formulations produced in Example 6.

TABLE 4
The secretome/extracellular vesicle process/product testing panel relating to Examples 5-17.

| | | Test Panel (Test material) | | | | | |
|---|---|---|---|---|---|---|---|
| | | *1 (Test 20) | | | *4 (Test 20) | *5 (Test 20) | |
| | | (D + 0 cells, | *2 | | (D + 5 | (Conditioned | *7 |
| | | final pooled cell | (Test 20) | | Conditioned | media | (Test 20) |
| | | suspension prior | (D + 3 | *3 (Test 20) | media before | after | (Final for- |
| Test | Method | to seeding) | material) | (D + 5 cells) | clarification) | clarification) | mulation) |
| Cells-Number of | NucleoCounter | Y | Y | Y | | | |
| Viable Cells | NC-200 | | (harvested | (harvested | | | |
| | (DAPI/AO staining) | | from T75) | from CS10s | | | |
| | (PhE 2.7.29) | | | and T75) | | | |
| Cells-% Viability | NucleoCounter | Y | Y | Y | | | |
| | NC-200 | | (harvested | (harvested | | | |
| | (DAPI/AO staining) | | from T75) | from CS10s | | | |
| | (PhE 2.7.29) | | | and T75) | | | |
| Cells-Morphology | Microscopy | | Y | Y | | | |
| | | | (as viewed | (as viewed | | | |
| | | | on T75 and | on T75 and | | | |
| | | | CS10) | CS10) | | | |
| Cells-Identity | Flow Cytometry | Y | Y | Y | | | |
| | (PhE 2.7.24) | | (harvested | (harvested | | | |
| | | | from T75) | from CS10s) | | | |
| Cells-Karyotype | Conventional | | | Y | | | |
| | Cytogenetics | | | (harvested | | | |
| | | | | from flasks) | | | |
| Cells- | RNA sequencing | | Y | Y | | | |
| Transcriptome | | | (harvested | (harvested | | | |
| | | | from T75) | from CS10s) | | | |
| Secretome-Particle | Nanoparticle Tracking | | | | Y | Y | Y |
| concentration, | Analysis (NanoSight) | | | | | | |
| particle size | | | | | | | |
| distribution | | | | | | | |
| Secretome-Protein | BCA assay | | | | Y | Y | Y |
| concentration | (PhE 2.5.33) | | | | | | |
| Secretome-Protein | Mass Spectrometry | | | | | | Y |
| profile content | | | | | | | |
| Secretome-RNA | RNA sequencing | | | | | | Y |
| profile | | | | | | | |
| Secretome-Potency | Scratch Wound | | | | | | Y |
| | Healing Assay | | | | | | |
| | CM Survival Assay | | | | | | |
| Secretome-EV | Flow Cytometry: CD9/ | | | | | Y | Y |
| marker identity | CD63/CD81 and other | | | | | | |
| | EV surface markers by | | | | | | |
| | Macsplex ® Miltenyi | | | | | | |
| | (PhE 2.7.24) | | | | | | |
| Secretome-Host | GRP94 and HLA | | | | | Y | Y |
| cell protein | Class I ELISA assays | | | | | | |
| assessment | (PhE 2.6.34) | | | | | | |
| Secretome- | Immunoassay | | Y | | Y | | |
| Antibiotics | | | | | | | |
| Secretome- | Qubit, | | | | | | Y |
| Residual DNA, | spectrophotometry, | | | | | | |
| quantification | qPCR (PhE 2.6.35) | | | | | | |
| Secretome- | Exome sequencing | | | | | | Y |
| Residual DNA, | | | | | | | |
| characterisation | | | | | | | |
| Adventitious agent | Bovine, porcine virus | | | Y | Y | | |
| testing | detection; retrovirus | | | | | | |
| | testing | | | | | | |
| Sterility | Aerobic + anaerobic | Y | Y | | Y | | Y |
| | bacterial cultures | | (spent | | | | |
| | (PhE 2.6.1/2.6.27) | | media, | | | | |
| | | | CS10) | | | | |
| Mycoplasma | qPCR | | Y | | Y | | Y |
| | (PhE 2.6.7) | | (spent | | | | |
| | | | media, | | | | |
| | | | CS10) | | | | |
| Endotoxin | Kinetic | | Y | | Y | | Y |
| | chromogenic LAL | | (spent | | | | |
| | (PhE 2.6.14) | | media, | | | | |
| | | | CS10) | | | | |
| Secretome- | Clarity and degree of | | | | | | Y |
| Appearance | opalescence of liquids | | | | | | |
| | (PhE 2.2.1) | | | | | | |
| | Degree of coloration | | | | | | |
| | of liquides | | | | | | |
| | (PhE 2.2.2) | | | | | | |

TABLE 5
The results for certain criteria shown in the testing panel in TABLE 49, with respect to Examples 5-11.

| | Test Panel, Samples Tested | | | | | |
|---|---|---|---|---|---|---|
| | *1 (Test 20) | | | *4 (Test 20) | | |
| | (D + 0 cells, | | | (D + 5 | *5 (Test 20) | *7 (Test 20) |
| | final pooled | *2 (Test 20) | | Conditioned | (Conditioned | (Final |
| | cell suspension | (D + 3 | *3 (Test 20) | media before | media after | formu- |
| Test | prior to seeding) | material) | (D + 5 cells) | clarification) | clarification) | lation) |
| Cells-Number of | 5,608E+06 viable cells | 9.5E+06 | 485E+06 +/− | | | |
| Viable Cells | recovered (160.2E+06 | viable cells | 81E+06 cells | | | |
| | cells per vial thawed) | | per CS10 | | | |
| | | | (average of 3 | | | |
| | | | counted CS10) | | | |
| | | | [T75 counts not | | | |
| | | | available this | | | |
| | | | example] | | | |
| Cells-% Viability | 88% | 87% | 92% | | | |
| Secretome-Protein | | | | | 12,400 mg | 171 mg |
| concentration | | | | | | |
| Secretome-Host | | | | | 76,000 ng for | 26,400 ng for |
| cell protein | | | | | GRP94 | GRP94 |
| assessment | | | | | and < detectable | and < dectable |
| | | | | | minimum for | minimum for |
| | | | | | HLA-I | HLA-I |
| Secretome- | | | | | 3,800 μg (Qubit) | 1,300 μg (Qubit) |
| Residual DNA, | | | | | | |
| quantification | | | | | | |
| Sterility | Negative | Negative | | Negative | | Negative |
| Mycoplasma | | Negative | | Negative | | Negative |
| | | (<10 CFU/ | | (<10 CFU/mL) | | (<10 CFU/mL) |
| | | mL) | | | | |
| Endotoxin | | Negative | | Negative | | Negative |
| | | (<2 | | (<2 EU/mL) | | (< 2 EU/mL) |
| | | EU/mL) | | | | |
| Secretome- | | | | | | Colorless |
| Appearance | | | | | | |

TABLE 6
The degree of enrichment (as calculated by the increase of particles
per unit protein), as compared to conditioned media after clarification,
for the retentates and final formulations produced in Example 6.

| | | Protein | | Particles/ |
|---|---|---|---|---|
| | | Concentration | Particles/ | mg |
| | Panel | (mg/mL) | mL | protein |
| Conditioned media | *5 (Test | 1.3 | 3.10E+09 | 2.38E+09 |
| after clarification | 20) | | | |
| Retentate | *6 - | 1.3 | 1.00E+11 | 7.69E+10 |
| | sample a | | | |
| | (Test 20) | | | |
| Retentate + | *6 - | 1.3 | 1.40E+11 | 1.08E+11 |
| trehalose | sample b | | | |
| | (Test 20) | | | |
| Retentate + | *6 - | 0.9 | 1.10E+11 | 1.22E+11 |
| L-histidine | sample c | | | |
| | (Test 20) | | | |
| Final formulation | *7 - | 1.2 | 1.00E+11 | 8.33E+10 |
| | sample a | | | |
| | (Test 20) | | | |
| Final formulation + | *7 - | 1.5 | 1.00E+11 | 6.67E+10 |
| trehalose | sample b | | | |
| | (Test 20) | | | |

### Example 12

Second Exemplary Good Manufacturing Practices (GMP)-Compatible Process for Producing Small Extracellular Vesicle-Enriched Fraction (sEV) Formulations of CTC1-EV

A second exemplary GMP-compatible process for producing sEV-containing formulations was developed. The production process included four main stages: vesiculation; conditioned media clarification; enrichment and concentration of small EV-enriched secretome; and production of the final sEV formulation. Flow diagrams outlining the GMP-compatible process that was performed are depicted in FIGS. 24A and 24B.

### Vesiculation

For the vesiculation step, cardiovascular progenitor cells (CPCs) that had been cryopreserved and stored under vapor-phase liquid nitrogen (or within a −150 °C freezer) were initially thawed for 2.5 minutes at 37 °C in a thawing medium (MEM alpha (1000 mL of Macopharma Ref: BC0110021); glucose (30%) supplement (Macopharma Ref: CARELIDE, to a final overall glucose concentration of 2 mg/mL; Ydralbum® (LFB), at a final concentration of 20 mg/mL; B-27™ Supplement (50×, Life Tech Ref: 17504001 at a final concentration of 1×); and Rock Inhibitor H1152 (Sigma Ref: 555550, at a final concentration of 0.392 μg/mL, sterilized using a 0.2 μm cellulose acetate (CA) membrane syringe filter), within an EVA bag (Corning). 18 mL of thawing medium was used per 1 mL of CPCs.

After thawing, CPCs were seeded onto vitronectin (Life Tech Ref: VTN-N; recombinant human protein, truncated (Ref: A31804); 5 μg/mL, sterilized using a 0.2 μm cellulose acetate (CA) membrane syringe filter) coated culture flasks (12×10ST CellStack Culture Chambers, tissue culture (TC)-treated (Corning Ref: 3271); as well as 2×TC-treated, vitronectin-coated T75 flasks), at a seeding density of about 100,000 cells per cm², using 0.2 mL/cm² of complete medium (MEM alpha (1000 mL of Macopharma Ref: BC0110021); glucose (30%) supplement (Macopharma Ref: CARELIDE, to a final overall glucose concentration of 2 mg/mL; Ydralbum® (LFB; 200 g/L); B-27™ Supplement (50×, Life Tech Ref: 17504001 or 17504044, at a final concentration of 1×); Gentamicin (Panpharma, at a final concentration of 25 μg/mL); and Human FGF-2 Premium grade (Miltenyi Biotec ref: A12873-01, at a final concentration of 1 μg/mL, sterilized using a 0.2 μm cellulose acetate (CA) membrane syringe filter)). Seeding was performed without prior centrifugation of the cell suspension. The seeded CPCs were then cultured in complete medium for three days at 37 °C, in the presence of 5% CO₂ and atmospheric oxygen.

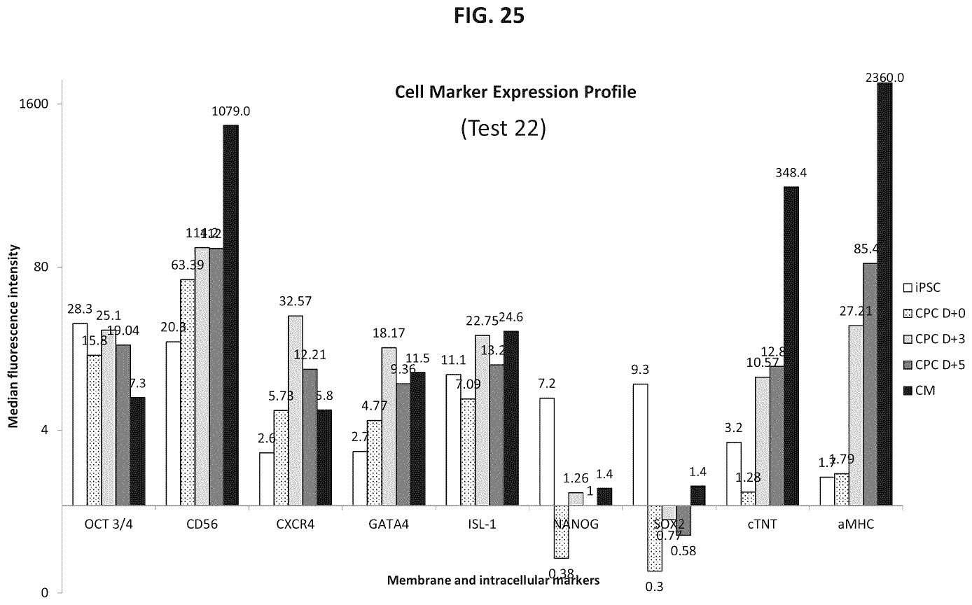

Immediately prior to seeding (“D+0”), cells were analyzed to determine the number and percentage of viable cells (see TABLE 7, column 1 (“D+0 cells”) using a NucleoCounter NC-200 (Chemometec) with DAPI/AO staining (Ph. Eur. 2.7.29); to determine their identity (see FIG. 25 and Example 14) by flow cytometry using a MACSQuant 10 Flow Cytometer.

After the 3-day culturing (“D+3”), the cells from one of the cultured T75 flasks were harvested. These harvested cells were analyzed to determine the number and percentage of viable cells (see TABLE 7, column 2 (“D+3 material”) using a NucleoCounter NC-200 (Chemometec) with DAPI/AO staining (Ph. Eur. 2.7.29); and to determine their identity (see FIG. 25 and Example 14) by flow cytometry using a MACSQuant 10 Flow Cytometer. Spent media from the 10ST CellStack Culture Chambers was also tested for sterility, and for the presence of mycoplasma and endotoxin.

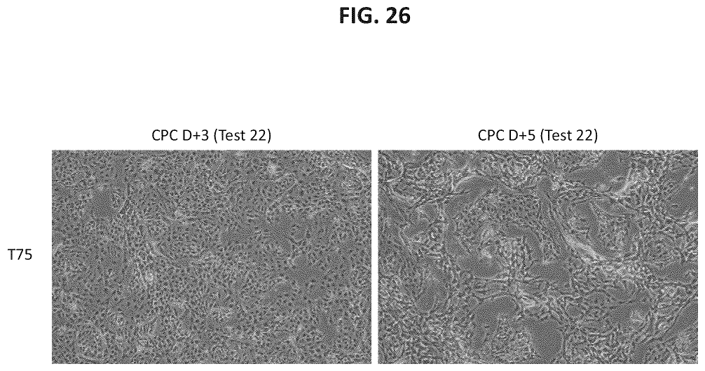

For the remaining flasks (12×10ST CellStack Culture Chambers; and 1×T75), the cells were visualized by microscopy to determine their morphology (see FIG. 26), and washed twice with a wash medium (MEM alpha (1000 mL of Macopharma Ref: BC0110021); glucose (30%) supplement (Macopharma Ref: CARELIDE, to a final overall glucose concentration of 2 mg/mL), before being cultured for 2 days at 37 °C, in the presence of 5% CO₂ and atmospheric oxygen, in a starvation media (poor media) (MEM alpha (1000 mL of Macopharma Ref: BCO110021); glucose (30%) supplement (Macopharma Ref: CARELIDE, to a final overall glucose concentration of 2 mg/mL). After this 2-day incubation (“D+5”), the culture media (conditioned media) was collected, and the cells from the 10ST CellStack Culture Chambers and the remaining T75 flask were harvested.

As with the cells at D+3, the cells at D+5 were again visualized by microscopy to determine their morphology (see FIG. 26); and the cells harvested at D+5 were further analyzed to determine the number and percentage of viable cells (see TABLE 7, column 3 (“D+5 cells”); and to determine their identity (see FIG. 25 and Example 14) by flow cytometry using a MACSQuant 10 Flow Cytometer. The collected conditioned media was tested for sterility, and for the presence of mycoplasma and endotoxin, before further processing.

TABLE 7
The results for certain criteria shown in the testing panel in TABLE 49, with respect to Examples 12-17.

| | Test Panel, Material Tested | | | | | |
|---|---|---|---|---|---|---|
| | *1 (Test 22) | | | *4 (Test 22) | | |
| | (D + 0 cells, final | | | (D + 5 | *5 (Test 22) | |
| | pooled cell | | | Conditioned | (Conditioned | *7 (Test 22) |
| | suspension prior | *2 (Test 22) | *3 (Test 22) | media before | media after | (Final |
| Test | to seeding) | (D + 3 material) | (D + 5 cells) | clarification) | clarification) | formulation) |
| Cells-Number of | 7.837E+06 viable | 7.8E+06 viable | 542E+06 +/− | | | |
| Viable Cells | cells recovered | cells | 6E+06 cells per | | | |
| | (150.7E+06 cells | | CS10 and 9E+06 | | | |
| | per vial thawed) | | viable cells per | | | |
| | | | T75 | | | |
| | | | (average of 2 | | | |
| | | | counted CS10) | | | |
| | | | [T75 counts not | | | |
| | | | available this | | | |
| | | | example] | | | |
| Cells-% Viability | 87% | 84% | 93% | | | |
| Secretome-Protein | | | | | 18,000 mg | 80 mg |
| concentration | | | | | | |
| Secretome-Host cell | | | | | | |
| protein assessment | | | | | | |
| Secretome-Residual | | | | | | |
| DNA, quantification | | | | | | |
| Sterility | Negative | Negative | | Negative | | Negative |
| Mycoplasma | | Negative | | Negative | | Negative |
| | | (<10 CFU/mL) | | (<10 CFU/mL) | | (<10 CFU/mL) |
| Endotoxin | | Negative | | Negative | | Negative |
| | | (<2 EU/mL) | | (<2 EU/mL) | | (<2 EU/mL) |
| Secretome- | | | | | | Colorless |
| Appearance | | | | | | |

### Conditioned Media Clarification

Clarification of the conditioned media was conducted via a series of three filtration steps. First, filtration was performed using a Sartopure PP3 MidiCaps 5 μm PES filter (Sartorius, Ref: 5055342P9-OO-A (Sartorius)). The resulting filtrate was then filtered using a Sartoguard PES MidiCaps filter (Pore sizes (prefilter+filter): 1.2 μm+0.2 μm; Sartorius Ref: 5475307F9-OO-A). The resulting filtrate was then filtered using a Sartopure 2 MidiCaps filter (Pore sizes (prefilter+filter): 0.45 μm+0.2 μm; Sartorius Ref: 5445307H8-OO-A).

### Enrichment and Concentration

Following clarification of the conditioned media, the conditioned media was subjected to enrichment and concentration of the small EV secretome.

First, the clarified conditioned media was subjected to Tangential Flow Filtration (TFF), using a TFF Allegro™ CM150 (PALL/Sartorius). For the TFF manifold, a sterile single-use Flow Path Manual Valve P&F (PALL/Sartorius, reference: 744-69N) was used, together with a 10 L Retentate Assembly (sterile, single use; PALL/Sartorius Ref: 744-69M). For the TFF cassette, sterile single-use regenerated cellulose filters (30 kDa cut-off; 0.14 m²; Sartorius Ref: Opta filter assembly+3D51445901MFFSG) were used. For recovery of the retentate (i.e., what is retained in the TFF), a Bench Top TFF 1 L Bag was used (PALL/Sartorius, reference: 7442-0303P).

Initially, the TFF device was washed with 10 L of H₂O, and 2 L of 1×PBS before operation. Next, after administration of the clarified conditioned media to the TFF device, the retentate was concentrated (to 500 mL; not exceeding 3 bars of pressure). After this initial concentration step, the retentate was subjected to diafiltration (6 diafiltration volumes; using 1×DPBS). After diafiltration, the retentate was further concentrated, to produce a total volume of at least 100 mL. The parameters of the TFF process were as follows: feed manifold pressure (PTO1)—0.94-2.1 bars; retentate manifold pressure (PTO2)—0.12-0.13 bars; retentate manifold flow rate (FTO1)—0.012-0.58 L/min; transmembrane pressure (TMPO1)—0.53-1.11 bars; and quattroflow pump (P01)—14-20%.

### Example 13

### Formulation/Composition

After enrichment and concentration by TFF, the final sEV formulation was then prepared by filter sterilizing the resulting retentate using a 0.22 μm filter (Sterivex™-GP Pressure Filter Unit, 0.22 μm, Millipore, Ref: SVGPL10RC). In some experiments, 25 mM trehalose was added before this sterilization step to avoid aggregation. After the sterilization step, the final formulation (with or without the addition of 25 mM trehalose) was bottled into glass vials (2 mL, bromobutyl cap; Adelphi Ref: VCDIN2RDLS1). Final product formulation was then stored at −80 °C for future use or testing. Additionally, final formulations were also tested in which the retentate was first frozen and stored at −80 °C before sterilizing filtration using either a 0.22 μm filter (Sterivex™-GP Pressure Filter Unit, 0.22 μm, Millipore, Ref: SVGPL10RC), or a Sartopure 2 filter (Pore sizes (prefilter+filter): 0.45 μm+0.2 μm; Sartorius Ref: 5441307H4-OO-B) to produce final formulations thereof, as shown in FIG. 24B.

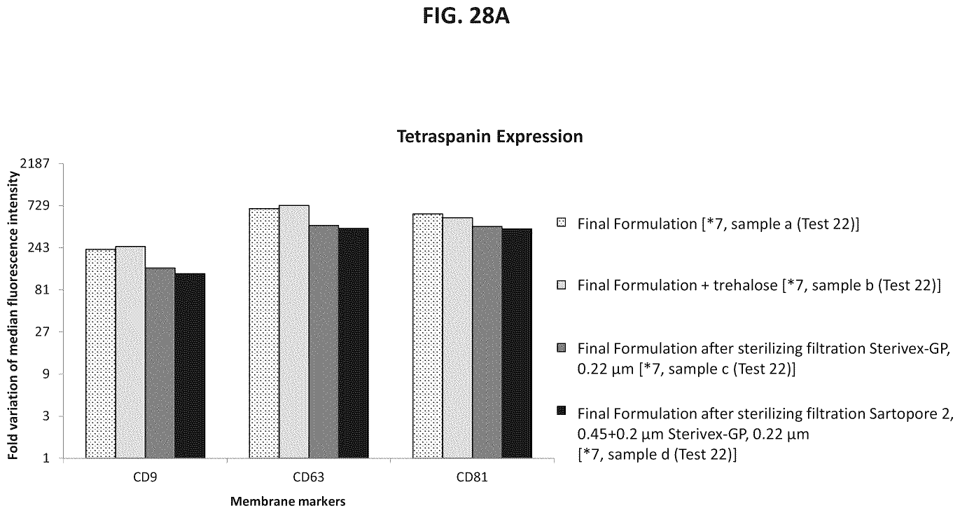

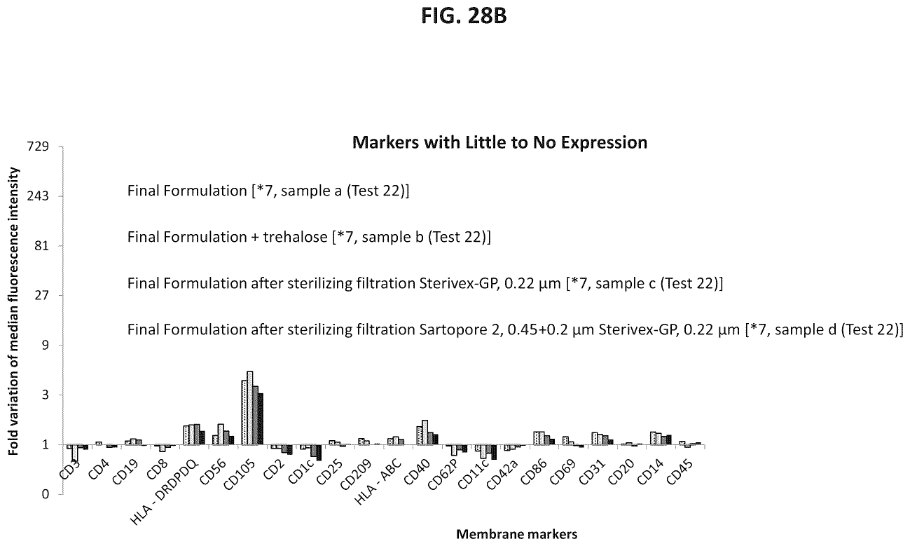

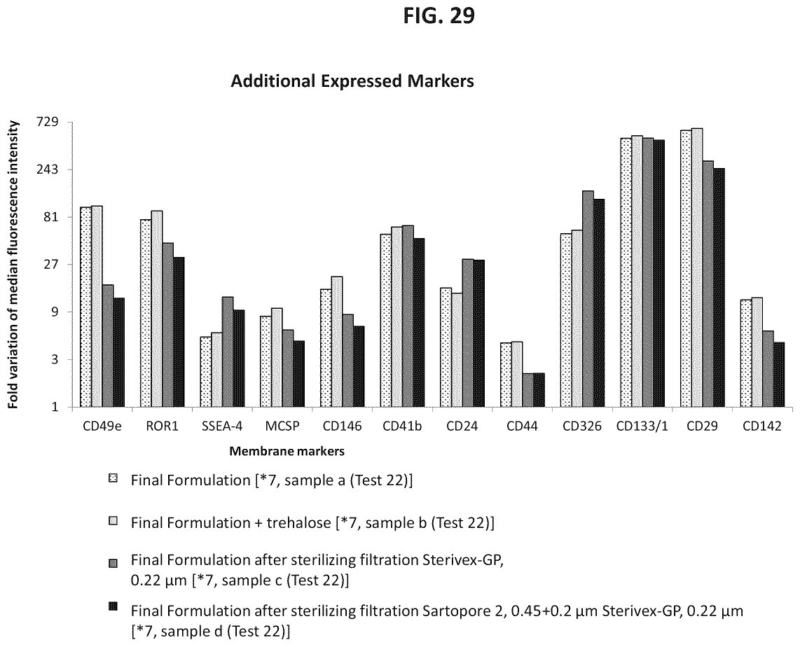

The final formulations, therefore, were in PBS (with or without trehalose), and were positive for CD9, CD63 and CD81 (canonical EV markers), as well as positive for the cardiac-related markers CD49e, ROR1, SSEA-4, MSCP, CD146, CD41b, CD24, CD44, CD236, CD133/1, CD29 and CD142, as detected by MACSPlex (as shown in FIGS. 28A and 29).

### Example 14

### Characterization of the Identity of CPCs During Vesiculation in the GMP-Compatible Process

To assess the identity of the cells during the vesiculation process in Example 12, the D+0 CPCs, as well as the harvested cells at D+3 and D+5, were analyzed by flow cytometry. iPSCs and cardiomyocyte (CM) cells were included as controls. As shown in FIG. 25, flow cytometry analysis, performed using a MACSQuant 10 Flow Cytometer with iPSC-, CPC- and cardiac-markers, demonstrated that the CPCs became more mature over the five-day vesiculation period. Specifically, the CPCs maintained little to no Nanog or SOX2 protein expression, and exhibited a continued increase in CD56, cTNT, and aMHC, protein expression (however, they did not reach expression levels of CD56, cTNT, and aMHC similar to cardiomyocytes, indicating that they remained progenitors throughout the process). iPSC and CM control cells were analyzed separately, and the average values are presented in FIG. 25 for comparative purposes.

### Example 15

### Analysis of EV Particle Concentration and EV Particle Size Distribution in CTC1-EV in the GMP-Compatible Process

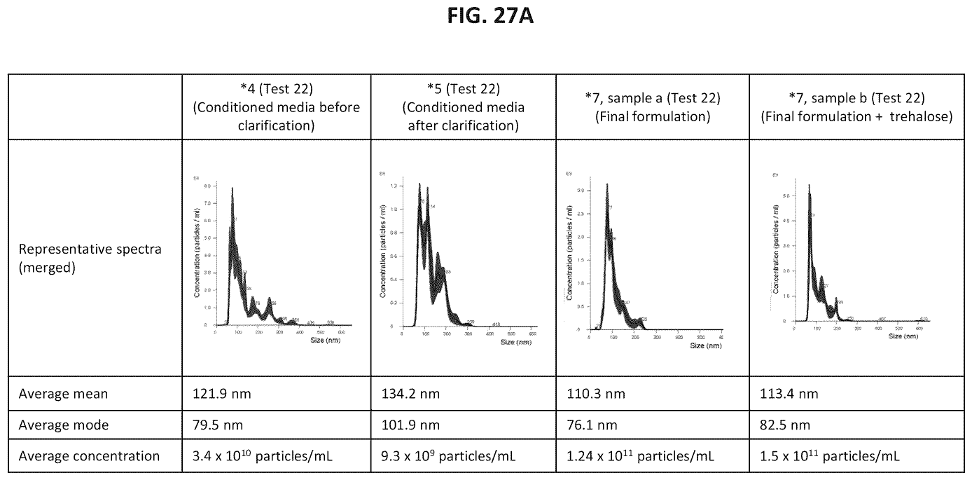

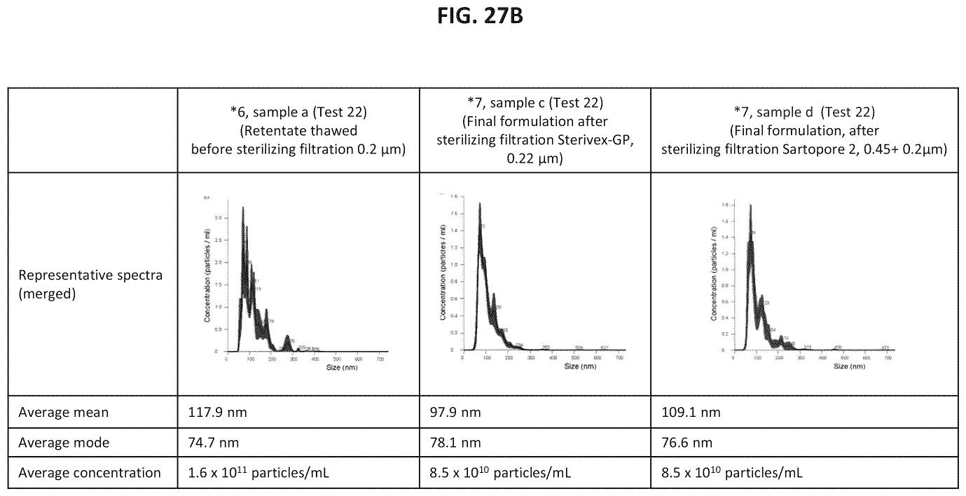

To assess the particle concentration and size distribution of EVs produced in Example 12 and Example 13, conditioned media prior to clarification (*4 (Test 22)) and after clarification (*5 (Test 22)), and the final formulations (with and without trehalose, samples b and a, respectively), were analyzed by nanoparticle tracking analysis (NTA; NanoSight). FIG. 27A depicts representative size distribution curves for each sample. The overall size distributions, means and modes, were similar between samples. A peak was observed generally between 50-150 nm, corresponding to the size of exosomes or small microparticles. The TFF step resulted in an approximately 32-fold concentration of particles. Similar experiments were also conducted on the previously-frozen retentate and final formulation samples (filtered with STerivex-GP or Sartopore 2) depicted in FIG. 24B (*6, sample a (Test 22); *7, sample c (Test 22); and *7, sample d (Test 22)). The results of these experiments are shown in FIG. 27B. The TFF step resulted in an approximately 20-fold concentration of particles, even though particles were lost during final sterilizing filtration (especially for the final formulations produced from thawed retentate).

### Example 16

### Analysis of EV Markers in CTC1-EV Final Formulation Produced by the GMP-Compatible Process

To assess the presence of EV markers in the clarified conditioned media (before TFF) and the final formulations (with and without trehalose) in Example 12, a MACSPlex Exosome Kit human (Miltenyi Ref: 130-108-813) was used to identify and quantify the presence of EV markers. As shown in FIG. 28A, the analysis confirmed the presence of extracellular vesicle tetraspanins (CD9, CD81 and CD63) in both the conditioned media (before TFF), and in the final formulation (with and without trehalose). Further still, as shown in FIG. 28B, the MACSPlex analysis also revealed a variety of markers that were found to be present either in low amounts (e.g., CD3, CD4, CD8, HLA-DRDPDQ, CD56, CD105, CD2, CD1c, CD25, CD40, CD11c, CD86, CD31 and CD20); or were substantially absent (CD19, CD209, HLA-ABC, CD62P, CD42a and CD69), in the conditioned media (before TFF), and/or in the final formulation (with and without trehalose).

Additionally, as shown by FIG. 29, additional cardiac-related markers were also observed in the conditioned media (before TFF), and in the final formulation (with and without trehalose).

### Example 17

### In Vitro Analysis of the Potency of CTC1-EV Final Formulation Produced by the GMP-Compatible Process

To analyze the functionality and potency of the final formulations produced by the GMP-compatible process in Example 12, two in vitro assays were used: a HUVEC scratch wound healing assay; and a cardiomyocyte viability assay using staurosporine-treated human cardiomyocytes.

For the HUVEC scratch wound healing assay, a scratch wound healing assay (developed by Essen BioSciences, for the Incucyte) was employed, according to the manufacturer's directions. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete media for two days. Cultures were maintained at 37 °C (atmospheric oxygen, 5% CO₂) throughout the maintenance and assay process. Wells were scratched using a Wound Maker (EssenBio, Ref: 4493) according to the manufacturer's directions, and cells were then rinsed with Endothelial Cell Basal Media and cultured overnight (either in HUVEC Complete Media with PBS, as a positive control; in Endothelial Cell Basal Media with PBS, as a negative control; or in Endothelial Cell Basal Media supplemented with sEV preparations in PBS). Using an Incucyte with the Scratch Wound Healing Module, plates were imaged at 18 hours after treatment. Wound closure was determined using the manufacturer's software, and values were baseline (negative control) subtracted, and normalized to the positive control. FIG. 30A depicts that the final formulations with and without trehalose (*7, samples b and a, (Test 22) respectively) promoted wound healing. FIG. 30B depicts that the previously-frozen final formulations without trehalose (*7, samples c and d, (Test 22)) promoted wound healing.

For the cardiomyocyte viability assay using staurosporine-treated human cardiomyocytes, iCell Cardiomyocytes² (Fujifilm Cellular Dynamics, Inc., ref: CMC-100-012-001) were plated at 50,000 cells/well of a fibronectin-coated 96-well plate in iCell Cardiomyocyte Plating Medium (Fujifilm Cellular Dynamics, Inc., ref: M1001), and cultured for 4 hours. The media was then exchanged for iCell Cardiomyocyte Maintenance Medium (iCMM, Fujifilm Cellular Dynamics, Inc., ref: M1003), and cells were cultured for up to 7 days, with full media exchanges every 2-3 days. After a minimum of 4 days, cells were exposed to iCMM with NucSpot Live 650 dye (Biotium, ref: 40082) (this served as a viable cell control); or to iCMM with NucSpot Live 650 dye, and staurosporine (Abcam, ref: ab146588) at a final in-well concentration of 2 μM (this also served as an apoptotic cell control). Dye, PBS, and DMSO concentrations, and final well volumes, were equivalent in all wells. Cells were cultured in these pre-incubation media for four hours. After this incubation, the pre-incubation media was removed, and the wells were rinsed with iCMM. Cells were then fed with iCMM with NucSpot Live 650 dye and PBS, or iCMM with NucSpot Live 650 dye supplemented with increasing concentrations of sEV preparations while maintaining PBS final volumes. Wells were imaged in an Incucyte at 24 hours, and nuclei counts were determined. FIG. 31A depicts that the final formulations with and without trehalose (*7, samples b and a, respectively, (Test 22)) promoted cardiomyocyte survival. FIG. 31B depicts that the previously-frozen final formulations without trehalose (*7, samples c and d, (Test 22)) promoted cardiomyocyte survival.

The testing panel used with respect to the processes/products of Example 12, and as embodied, e.g., in Examples 13-17, is shown in TABLE 4. The results therefore are shown in TABLE 7. Additionally, TABLE 8 depicts the degree of enrichment (as calculated by the increase of particles per unit protein), as compared to conditioned media after clarification, for the retentates and final formulations produced in Example 12.

TABLE 8
The degree of enrichment (as calculated by the increase in particles per unit
protein), as compared to conditioned media after clarification, for the retentates,
and final formulations produced in Example 12 and Example 13.

| | | Protein | | Particles/ | |
|---|---|---|---|---|---|
| | | Concentration | Particles/ | mg | Enrichment |
| Description | Sample | (mg/mL) | mL | protein | Factor |
| Conditioned | *4 (Test 22) | 1.3 | 3.40 × 10¹⁰ | 2.62 × 10¹⁰ | 3 |
| media before | | | | | |
| clarification | | | | | |
| Conditioned | *5 (Test 22) | 1.2 | 9.30 × 10⁹ | 7.75 × 10⁹ | 1 |
| media after | | | | | |
| clarification | | | | | |
| Retentate | *6, sample a | 0.5 | 2.00 × 10¹¹ | 4.00 × 10¹¹ | 52 |
| | (Test 22) | | | | |
| Final | *7, sample a | 0.6 | 1.20 × 10¹¹ | 2.00 × 10¹¹ | 26 |
| formulation | (Test 22) | | | | |
| Final | *7, sample b | 0.7 | 1.50 × 10¹¹ | 2.14 × 10¹¹ | 28 |
| formulation | (Test 22) | | | | |

### Example 18

### Analysis of the Effect of Cardiovascular Progenitor Cell (CPC) EVs on Cardiac Function in a Mouse Heart Failure Model

To analyze the in vivo functionality and potency of sEV preparations produced in accordance with methods described in the present disclosure, a mouse model was used to determine the effect of sEV preparations on cardiac function (in mice in which heart failure had been induced).

Heart failure was induced in C57BL/6 mice essentially as described in Kervadec et al. (J. Heart Lung Transplant, 2016, 35(6): 795-807; incorporated by reference herein in its entirety). Briefly, surgical occlusion of the left coronary artery was performed in 42 mice in total, to induce chronic heart failure (CHF). At three weeks post-occlusion, 22 of the mice were treated with either PBS vehicle control (60 μL, n=11) or sEV (60 μL, n=11), delivered by percutaneous injections under echocardiographic guidance into the peri-infarct myocardium (as described in Kervadec et al.). The administered sEV was produced in accordance with the “sEV 5.3” scheme depicted in FIG. 2 (whereby the sEV was prepared by ultracentrifugation from clarified “MC5”), and the resulting EV were resuspended in half the typical PBS volume (to generate a 2-fold concentrated sEV preparation, containing the secretome from 6.22E+04 cells per L of sEV preparation).

At four weeks post—μocclusion, cardiac function was assessed by echocardiography. The results thereof are shown in FIG. 34. Amongst the CHF mice, significantly fewer sEV-treated mice (as compared to the PBS-treated mice) had severely progressive LVESV hypertrophy (defined here as an equal to or greater than 9.1 uL increase in Left Ventricular End Systolic Volume, LVESV; p<0.05). Amongst the CHF mice, significantly fewer sEV-treated mice (as compared to the PBS-treated mice) had severely progressive LVEDV hypertrophy (defined here as equal to or greater than a 4 uL increase in Left Ventricular End Diastolic Volume, LVEDV; p<0.05). And although not significant, there was a strong trend that fewer sEV-treated CHF mice (as compared to the PBS-treated mice) had severely progressive loss of Ejection Fraction, EF, (as defined here as a loss of 5.5% or more in EF; p<0.056). Further, although not statistically significant, the Average Ejection Fraction of the PBS group deteriorated 2.5-fold more than the sEV-treated group (−4% vs −1.6%, respectively; ns). The results confirmed the ability of the sEV preparation to improve cardiac function in vivo.

### Example 19

Production of Small Extracellular Vesicle-Enriched Fraction (sEV) Clinical Candidate (CTC1-EV) Formulations; CTC1-EV Final Formulation

Three CPC sEV clinical candidate formulations (Test Examples 25, 26 and 27 herein) were generated for further analysis. Test Examples 25 and 26 were generated from CPCs essentially as described in Example 12 herein, however the TFF used a total volume of 15 L; a TFF cassette having a 0.28 m² size filter (30 KDa cut-off, 0.28 m2; Sartorius ref: Opta filter assembly+SFM-OP-1445921) was used; and a TFF feed pressure of 3.5 bars was employed.

FIG. 90 illustrates the process used to generate Test Example 25. In this example, FCDI CTC1 cardiovascular progenitor cells were thawed and plated onto vitronectin coated flasks. This lot of cells is referred to as “Clin001”. After thawing and prior to plating, a sample of cells was taken (“*1 (Test 25)”). Twelve 10-layer cell stacks (CS10) and two t-75 flasks were seeded with FCDI CTC1 cells at a density of 100 thousand cells per square centimeter in complete media. They were cultured in a humidified incubator for three days at 37 degrees Celsius, at 5% CO2. After three days of expansion, cells from one of the T-75 flasks were harvested for in-process characterization. This sample is called “*2 (Test 25)”. After three days of expansion, the spent media was removed from all of the remaining vessels, and “Poor Media” was added back to the vessel (“change to poor media”) on the third day, post plating (“D+3”). The cells were maintained in culture in this poor media for two more days, at 37 degrees Celsius, in a humidified incubator with 5% C02. This is what is referred to as the vesiculation media. Five days after plating the cells, the spent media was collected from the twelve CS10 flasks. This media is referred to as “*4 (Test 25)”. An aliquot of this material was used for in-process testing. Five days after plating the cells, cells from one of the T-75 flasks were harvested for-in process testing. Cells from three of the twelve 10-layer cell stacks were harvested for in-process testing. Cells harvested at this stage of the process (five days after plating, Day+5, “D+5”) are collectively referred to as “*3 (Test 25)”. The *4 (Test 25) material then underwent clarification.

To clarify the spent media, the media was filtered three times using first the Sartopure®PP3 filter with a filter size of 5 μm; next with a Sartoguard PES filter with 0.2 um nominal filter size, and lastly with a Sartopore®2 filter with a filter size of “(0.45+0.2 um)”. The resulting filtrate after these three filtrations is the “Conditioned media after clarification” or “Conditioned Media” for short. Fifteen liters of Conditioned Media were produced in this Test Example 25. This Conditioned Media is referred to as “*5 (Test 25)”. An aliquot of this material was used for in-process testing. An aliquot of 30 mL of this material was submitted to ultracentrifugation to produce an EV-enriched secretome for additional testing. This material is referred to as “*5a.uc (Test 25)”.

The Conditioned media after clarification was then processed by tangential flow filtration (“TFF”) using regenerated cellulose filters with a 30 kilodalton cut-off. The filter used for Test Example 25 was 0.28 square meters in surface area. The TFF process concentrated the retentate first and then six volumes of DPBS was used to perform a diafiltration of the retentate. The diafiltered retentate was then again concentrated. Aliquots of the resulting retentate (referred to as “*6 (Test 25)”) was analyzed by in-process controls. For Test Example 25, the final retentate was concentrated 50×. The remaining Retentate was stored in a 1 L bag over night at four degrees Celsius, and then placed in a minus 80 degree freezer until needed for Test Example 27.

FIG. 96 illustrates the process used to generate Test Example 26. In this example, FCDI CTC1 cardiovascular progenitor cells were thawed and plated onto vitronectin coated flasks. This lot of cells is referred to as “Clin002”. After thawing and prior to plating, a sample of cells was taken (“*1(Test 26)”). Twelve 10-layer cell stacks (CS10) and two t-75 flasks were seeded with FCDI CTC1 cells at a density of 100 thousand cells per square centimeter in complete media. They were cultured in a humidified incubator for three days at 37 degrees Celsius, at 5% C02. After three days of expansion, cells from one of the T-75 flasks were harvested for in-process characterization. This sample is called “*2(Test 26)”. After three days of expansion the spent media was removed from all of the remaining vessels, and “Poor Media” was added back to the vessel (“change to poor media) on the third day, post plating (“D+3”). The cells were maintained in culture in this poor media for two more days, at 37 degrees Celsius, in a humidified incubator with 5% CO2. This is what is referred to as the vesiculation media. Five days after plating the cells, the spent media was collected from the twelve CS10 flasks. This media is referred to as “*4 (Test 26)”. An aliquot of this material was used for in-process testing. Five days after plating the cells, cells from one of the T-75 flasks were harvested for in-process testing. Cells from three of the twelve 10-layer cell stacks were harvested for in-process testing. Cells harvested at this stage of the process (five days after plating, Day+5, “D+5”) are collectively referred to as “*3(Test 26)”. The *4 (Test 26) material then underwent clarification.

To clarify the spent media, the media was filtered three times using first the Sartopure®PP3 filter with a filter size of 5 um; next with a Sartoguard PES filter with 0.2 um nominal filter size, and lastly with a Sartopore®2 filter with a filter size of “(0.45+0.2 um)”. The resulting filtrate after these three filtrations is the “Conditioned media after clarification” or “Conditioned Media” for short. Fifteen liters of Conditioned Media were produced in this Test Example 26. This Conditioned Media is referred to as “*5 (Test 26).” An aliquot of this material was used for in-process testing. An aliquot of 30 mL of this material was submitted to ultracentrifugation to produce an EV-enriched secretome for additional testing. This material is referred to as “*5b.uc (Test 26)”.

The Conditioned media after clarification was then processed by tangential flow filtration (“TFF”) suing regenerated cellulose filters with a 30 kilodalton cut-off. The filter used for Test Example 26 was 0.28 square meters in surface area. The TFF process concentrated the retentate first and then six volumes of DPBS was used to perform a diafiltration of the retentate. The diafiltered retentate was then again concentrated. Aliquots of the resulting retentate (referred to as “*6 (Test 26)”) was analyzed by in-process controls.

For Test Example 26, the final retentate was concentrated 46×;

The remaining Retentate was stored in a 1 L bag over night at four degrees Celsius until needed for Test Example 27.

FIG. 97 illustrates the process used to generate Test Example 27. To produce Test Example 27, The frozen retentate obtained at the end of Test Example 25 was thawed overnight at four degrees Celsius. This material is referred to as “Thawed Retentate Test 25.” A sample of the Thawed Retentate Test Example 25 was collected for in-process testing. This material is referred to as “*7 (Test 25).” The retentate obtained at the end of the Test Example 26 was retrieved from the four-degree equipment where it had been stored overnight. This material is referred to as “Retentate fresh Test 26”. A sample of the Retentate fresh Test 26 was taken for in-process testing. This material is referred to as “*7 (Test 26).” The Thawed Retentate Test 25 and the Retentate fresh Test 26 were pooled (combined) together (referred to as “Pool”). A sample of the pool was taken for in-process testing. This material is referred to as “*8 (Test 27)”. The Pool was sterile filtered using a Sartopore® 2, Sterile Capsule (Pore size (prefilter+filter): 0.45 μm+0.2 μm; Sartorius Ref: 5441307H4-OO-B) to produce the final formulation (without trehalose). The sterilized material was vialed into glass cryopreservation tubes and stored at −80 degrees Celsius under further use. The material in the glass cryopreservation tubes is referred to as the “CTC1-EV Final Formulation”. It is also referred to as “*9 (Test 27).

### Example 20

### Transcriptomic Analysis of CTC1-EV Final Formulation

To assess the RNA transcriptome of the CTC1-EV Final Formulation, RNA was extracted from the CPCs (during the vesiculation phase) that were used to produce Test Examples 25 and 26 at D+3 (samples *2 (Test 25), *2 (Test 26)) and D+5 (samples *3 (Test 25) and *3 (Test 26)); and from the CTC1-EV composition in the *9 (Test 27) whose preparation is describes in detail in Example 19.

For the RNA extraction from CPCs (Test Examples 25 and 26), cell lysates (from 1 million cells per 450 μL RLT buffer (Qiagen, USA)) were obtained at D+3 and D+5, from the CPCs used to produce Test Examples 25 and 26. Small RNA was enriched from the CPCs using the mirVana RNA Isolation kit (Thermofisher, Ref: AM1561), according to the manufacturer's protocol, and the resulting RNA was eluted with 100 μL of Nuclease-Free Water (Teknova, Ref: W3330). 2 μL of this RNA preparation was then used for assessing RNA concentration, using the Lunatic (Unchained Labs). The results of the Lunatic analysis for the cellular RNA extracted from Test Examples 25 and 26 (D+5), showing the RNA concentration, are depicted in FIG. 35. Additionally, an aliquot of each of the D+3 and D+5 RNA samples was sent to the University of Wisconsin Gene Expression Center for quality control (QC) testing and sequencing. For the QC testing, High Sensitivity RNA ScreenTape (Agilent Technologies) was used. The results of the QC testing for Test Example 25 (D+5) are depicted in FIG. 36, and the results of the QC testing for Test Example 26 (D+5) are depicted in FIG. 37, confirming the quality of the extracted cellular RNA.

RNA extraction was performed on 200 μL of the *9 (Test 27) using the Wako microRNA Extractor SP kit (Wako, Ref: 295-71701), according to the manufacturer's protocol. The extracted RNA was eluted with 50 μL of Nuclease-Free Water (Teknova, Ref: W3330). 2 μL of this RNA preparation was then used for assessing RNA concentration, using the Lunatic (Unchained Labs). The results of the Lunatic analysis for the EV RNA extracted from *9 (Test 27), showing the RNA concentration, are depicted in FIG. 38. Additionally, an aliquot was sent to the University of Wisconsin Gene Expression Center for QC testing and sequencing. For the QC testing, a 2100 Bioanalyzer was used with the Eukaryote Total RNA Pico Assay (Agilent Technologies). The results of the QC testing for *9 (Test 27) are depicted in FIG. 39, confirming the quality of the extracted EV RNA.

Additionally, small RNA libraries were prepared. The small RNA libraries were prepared using a QIAseq miRNA Library kit (Qiagen, USA), using 5 μL of input RNA for each sample (namely, the extracted RNA from the cells from Test Examples 25 and 26; and from *9 (Test 27), as discussed above). To each sample, 3′ and 5′ adaptors (at a 1:5 dilution) and reverse transcriptase initiator were added, and the adapter-ligated RNA was then reverse transcribed. The resulting cDNA was purified using QMN beads (Qiagen, USA), and the cDNA libraries were then amplified for 16 cycles, and purified twice. The amplified libraries were resuspended in 19.5 μL of nuclease-free water, and 17 μL was recovered. The libraries were quantified with Qubit in singlet, using a 1:100 dilution, and QC tested using an Agilent Bioanalyzer HS DN chip (Agilent Technologies, USA). The results of the QC testing for Test Examples 25 (D+5), 26 (D+5) and from EVs (*9 (Test 27)) are depicted in FIG. 40, confirming the quality of the cDNA libraries. Sequencing was performed using NovaSeq6000 on the Illumina NGS Systems.

For bioinformatic analysis of the sequencing data, FastQC v0.11.9 was used to determine the quality of the raw read fastq files. Trimmomatic v0.39 was used to trim the adaptors from the raw reads. The trimmed reads were used for read mapping, and quantification was performed using miRge3.0 pipeline implemented in python with default settings (miRge 3.0 uses Bowtie v1.3.0 and SAMtools v1.7 for read mapping and quantification). FIG. 41 depicts the results of the analysis of the sequencing read lengths for *9 (Test 27), showing a peak at a read-length of 22 nucleotides. Different read lengths correspond to different RNA biotypes (with reads of 22 nucleotides typically mapping to a microRNA biotype). FIG. 42 depicts the prevalence (read distributions) of different RNA biotypes in *9 (Test 27), as determined by the sequence mapping.

Additionally, miRbase22 was used for microRNA annotations. The top 100 highest-expressing microRNAs were used for over-representation analysis to determine the top categories of tissue-specific expression (Tissue Atlas), localization (RNALocate), and GO term biological processes (miRPathDB), using a web-based interface program (miEAA 2.0 at an FDR 5.0%, adjusting the p-values for each category independently). FIG. 43 depicts the results of the analysis of read distributions for isomirs of the top 20 microRNAs identified in *9 (Test 27) RNA (isomiRs are microRNA sequences that have variations with respect to the reference sequence). As can be seen from FIG. 43, most reads mapped to canonical isomiRs. FIG. 44 depicts the top 40 most abundant microRNAs identified in *9 (Test 27) RNA, displayed as a honeycomb representation, showing that hsa-miR-302a-5p, hsa-miR-16-5p, hsa-miR-93-5p, hsa-miR-126-3p are the most highly-expressed miRs. TABLE 9 lists the results used to generate FIG. 44. Additionally, FIG. 45 shows a wordcloud indicating the top localization terms (with respect to *9 (Test 27)) RNA sequences), as identified by RNALocate.

TABLE 9
The top 40 most abundant miRNA identified in the RNA
extracted from *9 (Test 27). The RNA extract from *9
(Test 27) is named “45RNA”. The results given
in this table are also depicted in graphical form in FIG. 44.

| | 45RNA Read per Million |
|---|---|
| miRNA | (RPM) values |
| hsa-miR-302a-5p | 358085.3379 |
| hsa-miR-16-5p | 140971.6146 |
| hsa-miR-93-5p | 102923.8994 |
| hsa-miR-126-3p | 64271.8446 |
| hsa-miR-148a-3p | 17817.1072 |
| hsa-miR-21-5p | 15617.0486 |
| hsa-miR-20a-5p | 14655.5942 |
| hsa-miR-143-3p | 11864.2571 |
| hsa-miR-335-5p | 11794.1017 |
| hsa-miR-218-5p | 10874.3107 |
| hsa-miR-101-3p | 10601.0187 |
| hsa-miR-302d-3p | 9450.095 |
| hsa-miR-25-3p | 8847.4638 |
| hsa-miR-126-5p | 7976.3903 |
| hsa-miR-423-5p | 7840.5985 |
| hsa-miR-532-5p | 7282.0555 |
| hsa-miR-1246 | 7184.345 |
| hsa-miR-302a-3p | 6636.1627 |
| hsa-miR-20b-5p | 6338.7324 |
| hsa-miR-148b-3p | 6278.2213 |
| hsa-miR-34a-5p | 6263.7273 |
| hsa-miR-1-3p | 6077.3442 |
| hsa-miR-191-5p | 6051.3322 |
| hsa-miR-26b-5p | 5723.2605 |
| hsa-miR-151a-3p | 5624.7233 |
| hsa-miR-103a-3p/107 | 5624.4477 |
| hsa-miR-660-5p | 5553.135 |
| hsa-miR-320a-3p/320b/320c/320d/320e | 4930.5538 |
| hsa-miR-130a-3p | 4543.4591 |
| hsa-miR-19b-3p | 4452.527 |
| hsa-miR-27a-3p/27b-3p | 4262.672 |
| hsa-miR-186-5p | 3933.4431 |
| hsa-miR-26a-5p | 3464.6748 |
| hsa-miR-125b-5p | 3344.9752 |
| hsa-miR-7-5p | 3137.2645 |
| hsa-miR-24-3p | 2938.4265 |
| hsa-miR-483-5p | 2526.8627 |
| hsa-miR-99b-5p | 2368.8063 |
| hsa-miR-205-5p | 2303.3353 |
| hsa-miR-302b-3p | 2134.7529 |

A library of 54 EV-enriched secretomes was generated from over 18 different human cell types (human iPSC and human iPSC-derived cells) from apparently health normal donors (AHN). RNA was extracted from these EV-enriched secretomes essentially as described above and evaluated by small RNA sequencing as described above. Similarly, RNA was extracted from CTC1-EV samples *7, sample b (Test 20); *7, sample b (Test 22); *9 (Test 27). RNA was extracted from *7, sample b (Test 20) three times, using different RNA extraction protocols: Wako RNA extraction kit described above (Wako, Ref: 295-71701), as well as the mirVana RNA extraction kit (Thermofisher, Ref: AM1561), using the small RNA method and the total RNA method. Small RNA sequencing was performed as described above. The gene-wise 10^{th} percentile of log 2 FPKM values of CTC1-EV sample replicates and 90^{th} percentile of all the other samples in the study was determined and plotted. The data are tabulated in TABLE 80. A scatter plot was generated to compare the CTC1-EV miR with the miR in the rest of the study and is depicted in FIG. 45.1.

FIG. 45.1 shows a scatterplot identifying a miRNAs signature in CTC1-EV as compared to extracellular vesicles from other cell types included in this study (astrocyte, cardiac fibroblast, cardiomyocyte, neurons (GABAergic, Glutamatergic, Dopaminergic, motor, and induced by forward reprogramming), endothelial, hematopoietic progenitor cells, hepatocyte, induced pluripotent stem cell, microglia, macrophage, mesenchymal stem cells, pericytes, and retinal pigment epithelial). The CTC1 EV miR signature was extracted by calculating the gene-wise 10^{th} percential of log 2FPKM values of CTC1-EV sample replicates and 90^{th} percentile of all the other samples in the study.

The miR signature identified contains 18 miR as depicted in TABLE 10.

TABLE 10
miR signature identified in CTC1-EV.
miR Signature

hsa-miR-1-5p
hsa-miR-11401
hsa-miR-1263-3p
hsa-miR-3085-3p
hsa-miR-3161-5p
hsa-miR-3678-3p
hsa-miR-3942-5p
hsa-miR-4652-5p
hsa-miR-4758-5p
hsa-miR-4760-5p
hsa-miR-4779-3p
hsa-miR-508-5p
hsa-miR-548ad-3p
hsa-miR-5580-5p
hsa-miR-559-5p
hsa-miR-6791-5p
hsa-miR-6889-5p
hsa-miR-96-3p

Importantly, all CTC1-EV replicates contain all 18 of these markers. No other sample in the data set contains more than any 6 of thes miR, where ‘contains’ is defined as a log 2FPKM value of −1.1 or greater. This emphasizes the comparability of the CTC1-EV from these different production lots.

Interestingly, and surprisingly, and for the first time, miR-1-5p was found as a component of a secretome. miR-1-5p is expressed in all CTC1-EV samples (5 of 5) and in certain cardiac EV samples such as cardiomyocyte-EV samples (3 of 3). Interestingly and surprising, it is not consistently expressed in any other cell type. This is in stark contrast to miR-1-3p, which is ubiquitously and highly expressed in all samples, the gene-wise 10^{th} percential of log 2FPKM values of CTC1-EV sample replicates and 90^{th} percentile of all the other samples in the study were 11.51 and 10.73, respectively. This is a unique aspect of CTC1-EV, the clinical product.

### Example 21

### Proteomic Analysis of CTC1-EV Final Formulation

Analysis performed as follows. The protein mixture was reduced, alkylated and digested before LC-MS/MS analysis, and protein molar and mass percentages were estimated by using Top 3, as described in Silva et al., (“Absolute quantification of proteins by LCMSE: a virtue of parallel MS acquisition”. Mol. Cell. Proteomics 2006, 5, 144-156) as the Protein Quantification Index and the direct proportionality model as described in Ahrné et al., (Critical assessment of proteome-wide label free absolute abundance estimation strategies”. Proteomics, 13:2567-2578).

The top results of the nano-LC-MS/MS analysis are shown in TABLE 11. The table depicts the most abundant proteins in the EV sample (namely, constituting 0.1% o mass or more of the total protein content of the EV sample), ranked in descending order according to % mass.

A full table of the 2303 proteins identifies in the sample, along with the name of the coding gene, mass percentage (0) in each of the five technical replicates analysed, and a description of the protein are provided in the extended TABLE 81.

TABLE 11
*9 (Test 27) whose preparation is described in detail in Example 19 protein profile.

| | Average | | | |
|---|---|---|---|---|
| | percent | | | |
| Gene & Synonyms | mass (%) | SD | MW (kDa) | Description |
| ALB | 22.011 | 3.55 | 69.3 | SWISS-PROT: P02769 |
| | | | | (Bos taurus) Bovine |
| | | | | serum albumin |
| | | | | precursor |
| FN1, FN | 19.847 | 0.92 | 272.3 | Fibronectin |
| ALB, GIG20, GIG42, PRO0903, | 18.078 | 1.54 | 69.4 | RecName: Full = Serum |
| PRO1708, PRO2044, PRO2619, | | | | albumin; Flags: |
| PRO2675, UNQ696/PRO1341 | | | | Precursor |
| HSPG2 | 2.565 | 0.22 | 468.8 | Basement membrane- |
| | | | | specific heparan sulfate |
| | | | | proteoglycan core |
| | | | | protein |
| SPARC, ON | 1.229 | 0.14 | 34.6 | SPARC |
| CDH2, CDHN, NCAD | 0.876 | 0.08 | 99.8 | Cadherin-2 |
| MASP1, CRARF, CRARF1, | 0.566 | 0.40 | 79.2 | Mannan-binding lectin |
| PRSS5 | | | | serine protease 1 |
| FLNA, FLN, FLN1 | 0.566 | 0.19 | 280.7 | Filamin-A |
| VCAN, CSPG2 | 0.533 | 0.04 | 372.8 | Versican core protein |
| AGRN, AGRIN | 0.516 | 0.05 | 217.3 | Agrin |
| FBLN2 | 0.500 | 0.04 | 126.6 | Fibulin-2 |
| FSTL1, FRP | 0.390 | 0.05 | 35.0 | Follistatin-related |
| | | | | protein 1 |
| PROS1, PROS | 0.370 | 0.17 | 75.1 | Vitamin K-dependent |
| | | | | protein S |
| COL2A1 | 0.369 | 0.04 | 141.8 | Collagen alpha-1(II) |
| | | | | chain |
| HAPLN1, CRTL1 | 0.335 | 0.01 | 40.2 | Hyaluronan and |
| | | | | proteoglycan link |
| | | | | protein 1 |
| LAMA5, KIAA0533, KIAA1907 | 0.328 | 0.11 | 399.7 | Laminin subunit alpha-5 |
| A2M, CPAMD5, FWP007 | 0.290 | 0.01 | 163.3 | Alpha-2-macroglobulin |
| LAMA1, LAMA | 0.290 | 0.05 | 337.1 | Laminin subunit alpha-1 |
| VIM | 0.285 | 0.01 | 53.7 | Vimentin |
| | 0.265 | 0.03 | 24.4 | SWISS-PROT: |
| | | | | P00761\|TRYP_PIG |
| | | | | Trypsin - Sus scrofa |
| | | | | (Pig). |
| ACTN4 | 0.257 | 0.09 | 104.9 | Alpha-actinin-4 |
| DAG1 | 0.246 | 0.03 | 97.4 | Dystroglycan |
| FBN2 | 0.238 | 0.02 | 314.8 | Fibrillin-2 |
| MYH9 | 0.236 | 0.18 | 226.5 | Myosin-9 |
| LAMB1 | 0.232 | 0.02 | 198.0 | Laminin subunit beta-1 |
| ENO1, ENO1L1, MBPB1, | 0.228 | 0.01 | 47.2 | Alpha-enolase |
| MPB1 | | | | |
| SPTAN1, NEAS, SPTA2 | 0.222 | 0.01 | 284.5 | Spectrin alpha chain, |
| | | | | non-erythrocytic 1 |
| FLNB, FLN1L, FLN3, TABP, | 0.208 | 0.07 | 278.2 | Filamin-B |
| TAP | | | | |
| IGFBP7, MAC25, PSF | 0.195 | 0.02 | 29.1 | Insulin-like growth |
| | | | | factor-binding protein 7 |
| COL5A1 | 0.192 | 0.00 | 183.6 | Collagen alpha-1(V) |
| | | | | chain |
| FLNC, ABPL, FLN2 | 0.187 | 0.01 | 291.0 | Filamin-C |
| C5, CPAMD4 | 0.183 | 0.02 | 188.3 | Complement C5 |
| FSCN1, FAN1, HSN, SNL | 0.177 | 0.01 | 54.5 | Fascin |
| ALDOA, ALDA | 0.160 | 0.01 | 39.4 | Fructose-bisphosphate |
| | | | | aldolase A |
| COL6A1 | 0.158 | 0.01 | 108.5 | Collagen alpha-1(VI) |
| | | | | chain |
| SERPING1, C1IN, C1NH | 0.148 | 0.01 | 55.2 | Plasma protease C1 |
| | | | | inhibitor |
| CLSTN1, CS1, KIAA0911 | 0.146 | 0.02 | 109.8 | Calsyntenin-1 |
| COL3A1 | 0.146 | 0.01 | 138.6 | Collagen alpha-1(III) |
| | | | | chain |
| COL1A2 | 0.135 | 0.01 | 129.3 | Collagen alpha-2(I) |
| | | | | chain |
| IGFBP2, BP2, IBP2 | 0.134 | 0.01 | 34.8 | Insulin-like growth |
| | | | | factor-binding protein 2 |
| FASN, FAS | 0.128 | 0.01 | 273.4 | Fatty acid synthase |
| PTPRK, PTPK | 0.127 | 0.01 | 162.1 | Receptor-type tyrosine- |
| | | | | protein phosphatase |
| | | | | kappa |
| DSC2, CDHF2, DSC3 | 0.127 | 0.01 | 100.0 | Desmocollin-2 |
| NUCB1, NUC | 0.127 | 0.01 | 53.9 | Nucleobindin-1 |
| TF, PRO1400 | 0.125 | 0.01 | 77.1 | Serotransferrin |
| TTN | 0.125 | 0.10 | 3816.0 | Titin |
| THBS1, TSP, TSP1 | 0.123 | 0.01 | 129.4 | Thrombospondin-1 |
| ACTN1 | 0.122 | 0.01 | 103.1 | Alpha-actinin-1 |
| TIMP1, CLGI, TIMP | 0.120 | 0.01 | 23.2 | Metalloproteinase |
| | | | | inhibitor 1 |
| TNC, HXB | 0.120 | 0.02 | 240.9 | Tenascin |
| COL1A1 | 0.117 | 0.01 | 138.9 | Collagen alpha-1(I) |
| | | | | chain |
| LRP2 | 0.114 | 0.00 | 522.0 | Low-density |
| | | | | lipoprotein receptor- |
| | | | | related protein 2 |
| IGF2R, MPRI | 0.108 | 0.00 | 274.4 | Cation-independent |
| | | | | mannose-6-phosphate |
| | | | | receptor |
| PGK1, PGKA, MIG10, OK/SW- | 0.104 | 0.01 | 44.6 | Phosphoglycerate |
| cl.110 | | | | kinase 1 |
| PRTG | 0.102 | 0.01 | 127.1 | Protogenin |
| COL11A1, COLL6 | 0.101 | 0.01 | 181.1 | Collagen alpha-1(XI) |
| | | | | chain |
| LAMC1, LAMB2 | 0.100 | 0.01 | 177.6 | Laminin subunit |
| | | | | gamma-1 |

TABLE 81 depicts all of the 2303 proteins identified in the sample. The %~mass is given for each of 5 technical replicates.

### Example 22

### Electron Microscopy of CTC1-EV Final Formulation

CTC1-EV (*9 (lest 27) in this example) were further analyzed by electron microscopy (EM), as follows. First, Quantifoil R 1.2/1.3 Cu 200 mesh grids were flow discharged for 60 seconds (at 20 mA) with GloQube. Multiple applications (2 μL per drop) of *9 (lest 27) were applied to each grid. Samples were then plunge frozen using a Thermo Fisher Mark IV Vitrobot (Vitrobot conditions were: 4 °C; 9500 humidity; 0.5 second drain time; 30 second wait time, for all grids). Grids were loaded onto the microscope for screening, and high-resolution images were collected on the Talos Arctica at 200 kV, using a Gatan K3 direct electron detector in counting mode (with an energy filter at a 20 eV slit width). Magnification for high-resolution imaging used the following parameters: 79kx, spot size 4, C2 aperture 70, C2 lens power 40.939%, Objective aperture 100, pixel size 1.1 Å, dose 48.0 e-/Å2, exposure time 4.2 sec, defocus −2 μm. FIG. 46 and FIG. 47 depict cryo-electron micrographs of EV identified in *9 (Test 27) (scale bar=100 nm). In FIG. 46 and FIG. 47, the EVs are visible as a round structure, approximately 100 nm in diameter, with a clearly visible bilipid membrane. Luminal, transmembrane, and surface material are visible. In FIG. 46, the dark material on the left side of the image is the metal grid upon which the sample was prepared.

Additionally, a multivesicular body was also detected during the EM analysis. FIG. 48 depicts the multivesicular body, which appears to consist of a large bilipid membrane vesicle of approximately 200 nm in diameter, which contains therein a second bilipid membrane vesicle of a similar diameter as well as a third, smaller (approximately 50 nm in diameter) bilipid membrane. Material is clearly visible within the lumen of each of the three structures, as well as across the membranes and on the surface of the largest vesicle.

### Example 23

### Analysis of CTC1-EV in a HUVEC Plating Assay

CTC1-EV were analyzed in a HUVEC plating assay, using *5b.uc (Test 26). Briefly, 30 mL clarified media from Test Example 26 (after thawing) was ultracentrifuged at 100,000×g for 16 hours, and the resulting pellets were resuspended in 0.1 μm filtered dPBS, to produce EV preparations. These EV preparations were then aliquoted, and frozen at −80 °C. As negative controls, mock EV formulations were prepared from virgin media (using the same culture media used in Test Example 26). As positive controls, EV were produced from fetal bovine serum (“FBS-EV”), by ultracentrifuging commercially available FBS at 100,000×g for 16 hours.

To prepare HUVEC cells for the plating assay, HUVEC cells (Promega, USA) were and passaged in complete media (Promega, USA) four times. After the fourth passage, cells were harvested by trypsinization, and cryopreserved in CS10 (Cryostore, ref: 210102) by controlled rate freezing. Cells were stored under liquid nitrogen, vapor phase, and thawed before use.

For HUVEC seeding, 115 μL of positive control media (HUVEC Complete Media: Endothelial Cell Basal Media (PromoCell, Ref: C-22210), supplemented with the Endothelial Cell Growth Medium Supplement Pack (PromoCell, Ref: C-39210)) was added to the green wells (of the 96-well plate) depicted in the platemap in FIG. 49. Further, 115 μL of negative control mastermix (prepared by combining, per well, 100 μL of basal media with 15 μL of dPBS) was added to the yellow wells depicted in the platemap in FIG. 49. Further still, test condition mastermixes (prepared by combining, per well: 100 μL of basal media; up to 15 μL of EV (“EV 481” and “EV 457”) or mock-EV control (“FBS-EV”); and dPBS to a final volume of 115 μL) were added to the 96-well plate as shown in the platemap in FIG. 49. Duplicate plates were prepared to permit a comparison between two different quantification methods. dPBS was added to the remaining wells on the 96-well plates to reduce the evaporation of the experimental wells, and plates were incubated in a humidified incubator (37 °C, 5% CO₂) for 30 minutes to adjust the pH of the culture media prior to plating. Following this incubation, the thawed HUVEC cells (in complete media to a final volume of 91.5 L) were added to the experimental wells (15,000 viable cells per well; viable cell concentration was determined using an automated cell counter). Seeded plates were then incubated for 48 hours in a humidified incubator (37 °C, 5% CO₂). The cultured cells were then analyzed according to two different assays.

In the first assay, the cultured cells were analyzed by microscopy to determine the number of viable cells at the end of the 48-hour incubation. Briefly, following the 48-hour incubation, the assay plate was removed from the incubator, spent culture media was removed, and 150 μL of CyQuant dye master mix was added to each experimental well (the CyQuant dye master mix was prepared by mixing 0.1 μm filtered dPBS with Nucleic Acid Stain and Background Suppressor Dye). The assay plate was then incubated for 1 hour at room temperature.

Following this 1-hour incubation, the CyQuant dye was removed from the assay plate, the cells were rinsed with 100 μL of HBSS+/+, and then 100 μL of fresh HBSS+/+ was added to each experimental well. Wells were then imaged on the Incucyte (Essen BioSciences), and live cells were quantified using the Incucyte software. The Incucyte data was double normalized as follows: (1) the average number of live cells for the three negative control wells (see FIG. 49) was determined and subtracted from all other values; and (2) the average number of live cells for the positive control wells (see FIG. 49) was calculated, and all values were expressed as a percentage of that average. Where multiple experimental wells were tested (i.e., in replicate), an average+/−SD is shown. The results of the Incucyte analysis are shown in FIG. 50. As FIG. 50 shows, the CTC1-EV (*5b.uc (Test 26)) were found to dose-dependently increase the number of viable HUVEC cells present in the wells (after having been plated and cultured for 48 hours in poor media). Although the mock-EV treatment demonstrated some effect, the effect of the CTC1-EV treatment was significantly greater, consistent with the view that CTC1 cells secrete factors into the media which support endothelial cell survival and/or proliferation under stressful conditions. This effect (improved cell seeding, survival, and/or proliferation) of the CTC1-EV treatment was also readily detectable upon visual inspection of the wells of the plate, as shown in FIG. 51 (the nuclei of living cells are labeled in green).

In the second assay, the cell cultures in another 96-well plate were analyzed for ATP content. Briefly, at the end of the 48-hour incubation described above, the 96-well plate was removed from the incubator, and 50 μL of spent culture medium was removed from each experimental well (reducing the total well volume to 150 L). An equivalent volume (150 μL) of CellTiter-Glo® Reagent was added to each well, and the plate contents were then mixed for 2 minutes on an orbital shaker (to induce cell lysis). The ATP content therein was then quantified using the CellTiter-Glo® Luminescent Cell Viability Assay (Promega) according to the manufacturer's directions. The resulting signal was analyzed using a Tecan for Life Science® plate reader. The results are depicted in FIG. 52. The Tecan data was double normalized as follows: (1) the average number of live cells for the three negative control wells (see FIG. 49) was determined and subtracted from all other values; and (2) the average number of live cells for the positive control wells (see FIG. 49) was calculated, and all values were expressed as a percentage of that average. Where multiple experimental wells were tested (i.e., in replicate), an average+/−SD is shown. As FIG. 52.1 shows, the results obtained from the first- (cell quantification using the Incucyte, FIG. 52.1) and the second (ATP quantification using the CellTiter-Glo® Luminescent Cell Viability Assay, FIG. 52) were similar.

### Example 24

### Analysis of CTC1-EV Final Formulation in a HUVEC Stress Assay

The functionality and potency of the CTC1-EVs was further analyzed using a HUVEC stress assay. Specifically, *9 (Test 27), as well as MSC-EV and iCell-CPC-EV, were evaluated for their ability to improve HUVEC survival in the HUVEC stress assay.

For this assay, HUVECs were first passaged in complete media (Endothelial Cell Growth Medium; Promocell, Heidelberg, Germany) at 37 °C, in a humidified atmosphere containing 9% air and 5% CO₂, for between 2-6 passages. The passaged HUVECs were then plated onto 0.1% gelatin-coated 96-well plates and cultured under the same conditions until the cells reached confluency.

After reaching confluency, the complete media was replaced with a serum-deprived medium (DM) containing 0.01 μM of staurosporine (Cell Signaling Technology, Danvers, MA, USA), and the HUVECs were incubated for 1 hour at 37 °C. After this incubation, the HUVECs were rinsed twice with DM, and then incubated with 100 μL of DM containing either: 5×10⁹ MSC-derived EV (“MSC-EV”); 5×10⁹ iCell CPC-derived EV; 5×10⁹ CTC1-EV (*9 (Test 27)); or dPBS (as a positive (vehicle) control). As additional controls, wells containing either complete medium (“Complete”) or DM (“Poor”) without the addition of staurosporine were incubated in parallel as negative controls. After 20 hours incubation, Cell Counting Kit-8 (Sigma-Aldrich, Saint Louis, MO, USA) reagents were added to each well in accordance with the manufacturer's directions (at a 1/10 dilution), followed by a further incubation for 3 hours at 37 °C. After this incubation, cell viability was assessed by measuring absorbance at 450 nm. Results were normalized to the positive control (cells stressed with staurosporine in DM; “vehicle control”). As shown in FIG. 53, all the tested materials (*9 (Test 27)), as well as MSC-EV and iCell-CPC-EV) improved HUVEC survival by at least 40%.

### Example 25

### Analysis of EV-CPC and CTC1-EV Final Formulation in an In-Vitro Chemotherapy-Induced Cardiomyopathy Assay

EV-CPC were also tested in an in-vitro chemotherapy-induced cardiomyopathy assay, to determine their functionality in treating or reducing chemotherapy-induced cardiomyopathy.

Briefly, to produce EV-CPC, cardiovascular progenitor cells (CPCs) differentiated from human induced pluripotent stem cells (iPSC; iCell® Cardiac Progenitor Cells, FCDI, Madison, WI) were cultured in enriched medium (William's E Medium supplemented by Cocktail B from Hepatocyte Maintenance Supplement Pack, human bFGF and Gentamicin) for 4 days on a fibronectin pre-coated culture plate (CellBIND® Surface HYPERFlask®), according to the manufacturer's directions. On day 2 of the culture, the enriched medium was removed and replaced with a serum-free medium, and culturing was continued for a further 2 days. The serum-free medium from the last 2 days of culture (“conditioned media”) was then collected and pre-cleared by a series of centrifugations. EV-CPC were then isolated by vertical flow ultrafiltration (Amicon Ultra-15, PLTK, membrane Ultracel-PL, 30 kD) and cryo-preserved at −80 °C.

To set up the in-vitro chemotherapy-induced cardiomyopathy assay, frozen human iPSC-differentiated cardiomyocytes (CM; iCell Cardiomyocytes Kit, 01434, FCDI) were thawed and then phenotypically characterized (by FACS and immunofluorescence labeling). Specifically, to confirm their differentiation state, late differentiation-specific cardiac marker expression (Actin and Troponin) was confirmed (cardiac Troponin I was also confirmed by immunofluorescence); Islet1 and Gata4 expression was assessed (markers for an earlier progenitor stage); while pluripotency markers (Nanog and Sox2) were found to no longer be detectable.

The cardiomyocytes were then seeded onto a culture surface at a density of 63,000 viable cells/cm² to form a monolayer, and after 4 days of culture, the cardiomyocytes formed electrically-connected syncytial layers that beat in synchrony. At this time, the cardiomyocyte cultures were subjected to three sequential 48-hour exposures to doxorubicin (0.2 M Doxorubicin, Hydrochloride, Merck Millipore) at days 0, 2 and 4. The cardiomyocytes were then treated with EV-CPC (“EV-CPC”; 1.7×10⁹ particles per 50,000 cardiomyocytes) once (at day 6) or twice (at days 6 and 8) in maintenance medium, and culturing was continued. Control cells received only the maintenance medium (doxorubicin+Placebo), or the maintenance medium supplemented by the ultra-filtered CPC virgin culture medium (“VM-CPC”). The same volumes of VM-CPC and EV-CPC were used. Outcomes were assessed at day 10 by measuring intracellular Adenosine Triphosphate (ATP) levels, assessed using an ATP Luminescence Assay (ATPlite Luminescence Assay System, PerkinElmer).

As shown in FIG. 54A, following the addition of doxorubicin, ATP content fell by 50% (as compared to the non-stressed (placebo) cardiomyocytes at day 6). This shortage of energy was maintained beyond the termination of doxorubicin exposure and was still present on the last day of measurements (day 10).

Although a single exposure to EV-CPC (at day 6) did not improve the energy production of doxorubicin-stressed cardiomyocytes by more than 10% on Day 8 (as compared to the non-stressed cardiomyocytes on Day 8) (FIG. 54B), administering the EV-CPC twice (at days 6 and 8) increased intracellular ATP levels on Day 10 by about 30% in doxorubicin-stressed cardiomyocytes (as compared to the non-stressed (placebo) cardiomyocytes at day 10) (FIG. 54C). Indeed, while the cardiomyocytes exposed to placebo, or exposed to the CPC virgin medium, incurred statistically significant declines in ATP levels (as compared to the non-stressed (placebo) cardiomyocytes at day 10; p=0.0154), this was not the case for the cardiomyocytes exposed to the EV-CPC (effect size Hedges' g index of =0.9).

As can be seen from FIG. 54B and FIG. 54C, the CPC-EV containing secretome has an effect on the ATP content. This effect is independent of any materials from the culture media that may have been co-isolated with the secretome, if any. This is demonstrated by the fact that the same result on ATP was not observed when doxorubicin-stressed cardiomyocytes were treated with the CPC virgin medium control (“DOX+VM-CPC”).

The CTC1-EV Final Formulation was also assayed using the in-vitro Chemotherapy-Induced Cardiomyopathy Assay method described above, with the following differences. CTC1-EV Final Formulation assayed here was produced as described in detail in Example 5 and Example 6. CM (iCell Cardiomyocytes Kit, 01434, Fujifilm Cellular Dynamics) were seeded at 63,000 viable cells/cm². After 4 days of culture (day 0 of the assay), when CM formed electrically connected syncytial layers that beat in synchrony, they were subjected to three sequential 48-hour exposures to doxorubicin (0.01 uM—Doxorubicin ACCORD) at day 0, 2 and 4 of the assay. CM were then exposed to CTC1-EV (sample*7, sample a (Test 20)) at a dose of 1.7×10⁹ particles for 50,000 CM per treatment twice; cells were treated once at day 6 and once at day 8 of the assay in maintenance medium. Results for the CTC1-EV treated condition are shown in FIG. 54D as the grey bar on the far right labeled “DOX+CTC1-EV (prod 20)”. Control cells received no doxorubicin stress or CTC1-EV treatment (“MC”; positive control; black bar on the far left); doxorubicin stress and only the maintenance medium for treatment (‘DOX’; negative control; middle grey bar). Outcomes were assessed on day 10 of the assay for intracellular Adenosine Triphosphate (ATP) levels as assessed by ATP Luminescence Assay (ATPlite Luminescence Assay System, PerkinElmer). Results were calculated as the ATP signal per cell for each condition, and then normalized to the negative control. Results are therefore expressed as a fold change of ATP/cell over the negative control as measured on D10.

As can be seen from FIG. 54D, CTC1-EV Final Formulation improved (increased) the amount of intracellular ATP per cell in the doxorubicin stressed cardiomyocytes by 40% over the stressed control. This result shows that CTC1-EV was able to promote cardiomyocyte metabolic health in each surviving cell.

### Example 26

### Analysis of CTC1-EV Final Formulation and MSC-EV in an Anti-Fibrosis Assay

Primary human cardiac fibroblasts (HCF) were obtained from Promocell (Heidelberg, Germany), and used in an anti-fibrosis assay at between passage number three to five. HCF cells were plated into 24-well plates at a density of 80,000 cells per well and cultured in fibroblast growth medium 3 (Promocell), according to the manufacturer's directions. HCF were then stimulated with complete medium containing TGF-β1 (Peprotech, Rocky Hill, NJ, USA) at a final concentration of 2 ng/mL. Either 15 hours before the TGF-β1 stimulation (“pre-conditioned”), or at the same time as the TGF-β1 stimulation (“co-treated”), the HCF were treated with either 5×10⁹ EV (“1×”) or 1×10¹⁰ EV (“2×”). Two different types of EV were used: *9 (Test 27), or MSC-EV.

At 48 hours after EV addition, the cells were lysed and total RNA was extracted using an RNeasy Micro kit protocol (QIAGEN, Les Ulis, France). cDNA was then synthesized from the extracted RNA using a QuantiTech Reverse Transcription kit (QIAGEN), and the synthesized cDNA was analyzed by quantitative reverse transcription polymerase chain reaction (RT-qPCR) to determine expression of the following genes (the primer sequences used to detect the expression of each gene are provided in parentheses): MMP2 (forward primer sequence: GCGAGTGGATGCCGCCTTTAACTG; reverse primer sequence: GTCCACGACGGCATCCAGGTTATC); Periostin (forward primer sequence: GGAGAAACGGTGCGATTCACATAT; reverse primer sequence: AGAGCATTTTTGTCCCGTATCAGA); GAPDH (forward primer sequence: ATGGGGAAGGTGAAGGTCGGAG; reverse primer sequence: TCGCCCCACTTGATTTTGCAGG). The RT-qPCR was conducted using a SensiFAST SYBR Hi-ROX kit (meridian, Tennessee, USA), on a StepOnePlus Real Time PCR system (ThermoFisher Scientific). Results were normalized to the positive control (i.e., cells stimulated with complete medium containing TGF-01 at a final concentration of 2 ng/mL; “+ve”). Unstimulated cells were used as negative controls (“−ve”). The results for the MMP2 expression analysis are shown in FIG. 55. The results for the Periostin (“POSTN”) expression analysis are shown in FIG. 56. As shown in FIGS. 55 and 56, both the *9 (Test 27) and MSC-EVs exhibited anti-fibrotic activity, as measured by expression of fibrotic markers.

### Example 26.1

### Establishing a Rat Model of CCM

Previous development work had established a mouse model of chemotherapy-induced cardiomyopathy (CCM) in immunocompetent BALB/c mice. For this model, animals received 3 weekly Intra-Peritoneal (IP) injections of doxorubicin (DOX; 4 mg/kg each; cumulative dose: 12 mg/kg). They were then intravenously (IV) injected three times with EV (total dose: 30 billion particles as measured by NTA) over 2 weeks and finally assessed 9 to 11 weeks later by cardiac magnetic resonance imaging (FLASH cine sequences on a 4.7T preclinical Bruker scanner) as described in Desgres et al. (PMID: 37485274). Data show induction of left ventricular dysfunction in this model. Of note, it was determined that female mice would be excluded from the model since cyclical hormone levels appeared to protect against pathology resulting in variable decreases in cardiac function in anthracycline-treated female mice.

When a new rat model was needed to test the therapeutic potential of CTC1-EV in a rat model of chemotherapy induced cardiomyopathy, a first experiment was undertaken in which 6 Wistar male rats received 5 injections of doxorubicin (“DOXO” in the figure) as depicted in FIG. 58.1. At the end of the study period, an insufficient number of rats survived to be evaluated at their final echo (“echo #3” in the figure). Specifically, only 30% of rats survived the whole study period. A second experiment was undertaken which included both male and female rats (bottom row in FIG. 58.1). The survival rate of female rats was much greater than male rats in this experiment (91% vs 40%, respectively) and thus female rats were selected as the best subjects for the rat CCM model moving forward.

### Example 27

### Analysis of CTC1-EV in a Rat Chemotherapy-Induced Cardiomyopathy Model

CTC1-EV were tested in a rat model of chemotherapy-induced cardiomyopathy.

The CTC1-EV were manufactured as follows. Human iPSC-derived CPC were produced at the Innovation Facility for Advanced Cell Therapy (iFACT, FUJIFILM Cellular Dynamics, Inc, Madison, USA). CPC generation was performed in a GMP suite using a novel differentiation process with GMP-compatible methods, materials and reagents at a Phase 1 clinical manufacturing scale. These CPC were then cryo-preserved and shipped to the MEARY Cell and Gene Therapy Center, AP-HP Paris, France, where they were thawed and processed for vesiculation as described in detail in Example 5. Following collection of the conditioned medium, EV were isolated using tangential flow filtration according to the GMP-compatible procedures described in detail in Example 5.

To test CTC1-EV in a rat chemotherapy-induced cardiomyopathy (CCM) model, female Wistar rats (twenty-seven 8-week-old rats (RjHan: W), obtained from Janvier Labs) initially received five injections (intraperitoneally, IP) of doxorubicin (“DOX” or “Dox”) (3 mg/kg each, with a total cumulative dose of 15 mg/kg). These were the DOX rats. Specifically, the five doxorubicin administrations were given on days 0, 2, 4, 7 and 9. A healthy control group consisted of rats that received no DOX injection (“Sham”).

All rats received echocardiographic assessments (“echo”) on day 0 (“echo #1”) and day 10 (“echo #2”). At day 10, DOX animals were equitably allocated to CTC1-EV treatment and NaCl injection (Placebo) groups. Allocations were made on the basis of weight loss from day 0 through to day 10, which was taken as a surrogate of failing health. This was done to avoid any random bias in the average health of animals between the groups.

Starting on day 11, and again on day 14 and 16, DOX rats received either NaCl injection (by caudal vein) (Placebo group) or CTC1-EV (100×10⁹ particles, as counted on the NTA, cumulative dose) (where CTC1-EV for this experiment was *7, sample a (Test 20) whose preparation is described in Example 5). A summary of the DOX administration, CTC1-EV /Placebo treatment, and echo schedules is given in FIG. 56.1. The three CTC1-EV administrations were made in series, starting two days after the last administration of DOX. Specifically, the five doxorubicin administrations were given on days 0, 2, 4, 7 and 9 and the three CTC1-EV administrations were given on days 11, 14 and 16. Healthy control rats were not given any doxorubicin. These are referred to as the sham-operated animals, or Sham. Negative control rats were treated with doxorubicin on days 0, 2, 4, 7, and 9 as above but received no CTC1-EV. Rather, they were injected on days 11, 14 and 16 with NaCl. This is the Placebo group. In order to avoid selection bias, prior to CTC1-EV or placebo treatments, the doxorubicin-treated rats were equitably allocated to the CTC1-EV treatment group and Placebo group. This was done prior to any CTC1-EV treatment. This was done to balance the starting clinical status of the two groups. This was done to ensure that the endpoints could be reasonably compared between groups. The clinical status assessment was primarily based on the change in weight from baseline. Rats were sacrificed at day twenty after the first CTC1-EV injection.

Before sacrifice (end-study, or end-of-study), the rats' cardiac function was functionally assessed by echocardiography. Echocardiography was performed (on anesthetized rats, using 1.5-2% isoflurane anesthesia) using a two dimensional-echocardiography (VisualSonics® 2100 Ultrasound System (FUJIFILM, Toronto, Canada) equipped with a 20-MHz transducer probe. The echocardiography data were acquired: (1) at baseline, before doxorubicin treatment; (2) after the doxorubicin treatment, but before the CTC1-EV treatment (day 10); and (3) 28 or 29 days after the first doxorubicin injection (at end-study). Body temperature, respiratory and heart rates were controlled during echocardiography measurements.

Echocardiographic values of LV End Diastolic Volume (LVESV, or LV-ESV) and LV End Systolic Volume (LVEDV, or LV-EDV) were calculated from parasternal long axis views in B-mode (VEVO Lab). Cardiac function is related to heart volumes. Two types of heart volumes are examined here: the left ventricular end systolic volume (LVESV, or LV-ESV) and the left ventricular end diastolic volume (LVEDV, or LV-EDV). During heart failure, these two volumes increase. The more they increase, the worse the heart failure has progressed. The two volumes are measured by echocardiography (echo). The two volumes for each animal are measured once before doxorubicin injection (or before sham injections for “Sham” animals) (baseline echo, echo #1), then on the tenth day after the first doxorubicin administration/sham injection (which is before any CTC1-EV treatment or placebo administration; echo #2), and finally at the end of the study period on or around 28 or 29 days after the first doxorubicin injection/sham injection (echo #3). The CTC1-EV test products (this group of animals is referred to as “Dox+GMP-EV” in FIG. 57 (A, B)) or vehicle control (which is isotonic buffer, NaCl 0.9%, “Placebo”; this group of animals is referred to as “DOX+Placebo” in FIG. 57 (A, B)) are administered 11, 14 and 16 days after the first doxorubicin injection. The more the volumes increase between the pre-treatment echo (echo #2) and the post-treatment echo (echo #3), the more the heart failure has progressed in that animal during that time period. The “Sham” animals are not in heart failure. The Sham animals were not administered any doxorubicin.

In the experiment depicted in FIG. 57 (A, B), the results are expressed as a percent change (Median+/−IQR) from day 10 post-DOX administration (echo #2) to the end of the study period (echo #3). There were 6 Sham animals, 11 Placebo injected animals and 12 animals injected with CTC1-EV. (Sham: n=6; DOX+Placebo: n=11, DOX+GMP-EV: n=12).

For the LV-ESV results (depicted in FIG. 57A), the Sham group, on average, had a −3.0% change in volume; the DOX+Placebo group, on average, increased LVESV by 28.1%; the Dox+GMP-EV increased LVESV by 12.9%, which means that their heart failure progressed less than one half as much as the placebo group as determined by change in LV-ESV, which is a 2.2-times improvement in outcome.

For the LV-EDV results (depicted in FIG. 57B), the Sham group, on average, had a −0.1% change in volume; the DOX+Placebo group, on average, increased LVEDV by 19.2%; the Dox+GMP-EV increased LVEDV by 0.7%, which means that their heart failure progressed less than 4-tenths (0.7/19.2) as much as the placebo group as determined by change in LV-ESV, which is a 27-times improvement in outcome.

While LVESV volumes were significantly increased in placebo-injected hearts (p=0.033) compared with Sham, they were preserved by GMP-EV injections (effect size Hedges' g index of 0.4). Likewise, the percentages of responder rats which did not increase their LVEDV volumes by more than 5% from their post-DOX pre-treatment values were 58% (7 out of 12) vs. 28% (3 out of 11) in the Dox+GMP-EV and DOX+Placebo rat hearts, respectively (effect size Hedges' g index of 0.5, OR=3.7). See FIG. 57 (A, B). Outlines were traced on echocardiographic images by the same operator blinded to the treatment group: (1) at baseline, before doxorubicin treatment; (2) after the doxorubicin treatment, but before the CTC1-EV treatment (day 10); and (3) 29 days after the first doxorubicin injection (at end-study). Measurements were double-checked by a senior cardiologist. At end-of-study, the rats were lightly anesthetized (1.5% isoflurane). Electrocardiograms were performed with the EKG Analysis Module for LabChart® & PowerLab® as previously described, and blood pressure measurements were performed with a noninvasive Volume Pressure Recording (VPR) technology (CODA® High Throughput System, Kent Corporation), in which measurements of the physiological characteristics of the returning blood flow after an inflated occlusion tail cuff were registered.

Of the 27 rats that began the study, 4 died prior to the end-study timepoint. Thus, 6 sham-operated rats, 11 placebo-treated rats, and 12 CTC1-EV-injected rats, remained for the final analysis at end-study.

At the end-study timepoint, the DOX administration protocol was shown to have successfully induced LV dysfunction in the DOX+Placebo group. This was evidenced by a decrease in LVEF (FIG. 58A), an impaired ventricular compliance (calculated as the diastolic blood pressure/LVEDV ratio) (FIG. 58B) without a noticeable elevation of diastolic pressure (FIG. 58C), and a slower LV-depolarization (i.e., longer QTc, FIG. 58D). QT interval was corrected for heart rate. (Median+/−IQR) **p<0.001; (Mann Whitney test). Additionally, the End Systolic Elastance was impaired (decreased), which is calculated by the ratio of systolic blood pressure (SBP) to LV-ESV, which is taken as a surrogate marker for ventricular contractility. Furthermore, from the last DOX injection until end-study, every DOX-injected rat developed abdominal ascites supportive of the occurrence of heart failure symptoms. Taken together, the physiological changes in the DOX+Placebo rats described in this example validate the validity of the novel chemotherapy-induced cardiomyopathy model presented here.

To adjust for the variability in the response of rats to chemotherapy, end-study data for LVEF are presented as percent changes from those recorded at day 10 (echo #2).

While LVESV volumes were significantly increased in placebo-injected hearts compared with Sham (p=0.033), they were preserved by CTC1-EV injections (labeled “GMP-EV” in the related figures) (effect size Hedges' g index of 0.4). Likewise, the percentages of responder rats which did not increase their LVEDV volumes by more than 5% from their post-DOX pre-treatment values were 58% (7 out of 12) vs. 28% (3 out of 11) in GMP-EV- and placebo-injected hearts, respectively (effect size Cohen's d index of 0.5).

In the rat model, IV injections of GMP-EV also preserved left ventricular end-systolic and end-diastolic volumes compared with untreated controls.

For data interpretation, all data were submitted to GraphPad Prism Outlier tool (ROUT; Q=10%) before creating graphs and calculating statistics. Normality of each variable distribution was tested by D'Agostino & Pearson and Shapiro-Wilk tests. A Mann Whitney U test was used to compare 2 groups of non-parametric distribution. A Kruskal-Wallis test by ranks was used to compare 3 or more independent experimental groups of non-parametric distribution. Comparisons between groups were then performed by using Dunn's multiple comparisons test. Reciprocally, when variables presented a normal distribution, an unpaired t-test was used to compare 2 groups, and a one-way ANOVA was used to compare 3 or more independent experimental groups corrected with Tuckey method for pairwise group comparisons.

To estimate the effect size of EV treatment, Cohen's d index for unequal variance was calculated as follows (SD=Standard deviation, S=average variance):

$$Cohen ’ s d = \frac{\vert {Mean}_{E V} - {Mean}_{D o x} \vert}{S}$$

$$where S = \sqrt{\frac{( {SD}_{EV}^{2} + {SD}_{DOX}^{2} )}{2}}$$

Interpretation of this index was based on a commonly accepted stratification where small, medium, large and very large effect sizes are considered for values from 0.2, 0.5, 0.8 and 1.3, respectively. In one control rat for which the end-of-study recording could not be done, imputation of the missing data was done according to Last Observation Carried Forward (LOCF) method.

All statistical analyses were double-checked by an independent statistician.

In one control rat for which the end-study recording could not be done, imputation of the missing data was done according to Last Observation Carried Forward (LOCF) method.

Intravenously-injected extracellular vesicles derived from CPC have cardio-protective effects which may make them an attractive user-friendly option for the treatment of CCM. CTC1-EV could act on specific chemo-triggered abnormalities which primarily include DNA damage, oxidative and energetic stress leading to inflammation, extracellular matrix remodeling and defects in heart contractility, all of which can contribute to Left Ventricular (LV) dysfunction.

### Example 28

### Optimization of Post-Thaw Cell Viability

Cryopreserved CTC1 cells are fragile at thaw and typically display poor plating efficiency and expansion, unlike freshly plated, never-frozen CTC1 cells. Experiments were performed to optimize post-thaw viability and platability of CTC1 cells.

First, post-thaw cell viability was measured using a control media (Complete Media A), and this was compared to post-thaw cell viability using medias containing high protein concentration (Complete Media B with High Flex), containing a ROC Inhibitor (Complete Media B with H1152), or both (Complete Media C). Additionally, the effect of centrifugation of post-thaw cells was analyzed.

Four Complete Media types were prepared as shown in TABLE 12 through to TABLE 15 (4 tables).

TABLE 12
Components of “Complete Media A”.

| | | Actual Volume | | |
|---|---|---|---|---|
| | | Used/Container | | Final |
| Material | Reference | Size | Unit | Concentration |
| MEMa GlutaMAX | Gibco, Ref # 32561-037 | 96.6 | mL | 100% |
| B27 minus insulin | Gibco, Ref # A18956-01 | 2.00 | mL | 1X |
| Supplement (50X) | | | | (1:50) |
| Glucose | Gibco, Ref # A2494001 | 0.500 | mL | 1 mg/mL |
| (200 mg/mL) | | | | (1:200) |
| Flexbumin (25% w/vol | Baxter, Ref # | 0.800 | mL | 0.2% or |
| HSA), Baxter (250 mg/mL) | NDC0944-0493-02 | | | 2 mg/mL |
| | Code 2G0012 | | | (1:125) |
| Human FGF-2, Premium | Miltenyi Biotech, Ref # | 0.1000 | mL | 1 μg/mL |
| grade (1000 μg/mL) | 130093843 | | | (1:1000) |

TABLE 13
Components of “Complete Media B, High Flex”.

| | | Actual Volume | | |
|---|---|---|---|---|
| | | Used/Container | | Final |
| Material | Reference | Size | Unit | Concentration |
| MEMa GlutaMAX | Gibco, Ref # 32561-037 | 89.4 | mL | 100% |
| B27 minus insulin | Gibco, Ref # A18956-01 | 2.00 | mL | 1X |
| Supplement (50X) | | | | (1:50) |
| Glucose (200 mg/mL) | Gibco, Ref # A2494001 | 0.50 | mL | 1 mg/mL |
| | | | | (1:200) |
| Flexbumin (25% w/vol | Baxter, Ref # | 8.00 | mL | 2% or 20 |
| HSA), Baxter (250 mg/mL) | NDC0944-0493-02 | | | mg/mL |
| | Code 2G0012 | | | (1:125) |
| Human FGF-2, Premium | Miltenyi Biotech, Ref # | 0.100 | mL | 1 μg/mL |
| grade (1000 μg/mL) | 130093843 | | | (1:1000) |

TABLE 14
Components of “Complete Media B, H1152”.

| | | Actual Volume | | |
|---|---|---|---|---|
| | Target Volume/ | Used/Container | | Final |
| Material | Container Size | Size | Unit | Concentration |
| MEMa GlutaMAX | Gibco, Ref # 32561-037 | 95.6 | mL | 100% |
| B27 minus insulin | Gibco, Ref # A18956-01 | 2.00 | mL | 1X |
| Supplement (50X) | | | | (1:50) |
| Glucose | Gibco, Ref # A2494001 | 0.50 | mL | 1 mg/mL |
| (200 mg/mL) | | | | (1:200) |
| Flexbumin (25% w/vol | Baxter, Ref # | 0.800 | mL | 0.2% or |
| HSA), Baxter (250 mg/mL) | NDC0944-0493-02 | | | 2.0 mg/mL |
| | Code 2G0012 | | | (1:125) |
| Human FGF-2, Premium | Miltenyi Biotech, Ref # | 0.100 | mL | 1 μg/mL |
| grade (1000 μg/mL) | 130093843 | | | (1:1000) |
| H1152, 100 μM | FCDI, Ref # Q072 | 1.00 | mL | 1 μM |
| | | | | (1:100) |

TABLE 15
Components of “Complete Media C”.

| | | Actual Volume | | |
|---|---|---|---|---|
| | | Used/Container | | Final |
| Material | Reference | Size | Unit | Concentration |
| MEMa GlutaMAX | Gibco, Ref # 32561-037 | 88.40 | mL | 100% |
| B27 minus insulin | Gibco, Ref # A18956-01 | 2.00 | mL | 1X |
| Supplement (50X) | | | | (1:50) |
| Glucose (200 mg/mL) | Gibco, Ref # A2494001 | 0.500 | mL | 1 mg/mL |
| | | | | (1:200) |
| Flexbumin (25% w/vol | Baxter, Ref # | 8.0 | mL | 2% or |
| HSA), Baxter (250 mg/mL) | NDC0944-0493-02 | | | 20 mg/mL |
| | Code 2G0012 | | | (1:125) |
| Human FGF-2, Premium | Miltenyi Biotech, Ref # | 0.1000 | mL | 1 μg/mL |
| grade (1000 μg/mL) | 130093843 | | | (1:1000) |
| H1152, 100 μM | FCDI, Ref # Q072 | 1.000 | mL | 1 μM |
| | | | | (1:100) |

Briefly, cryopreserved CTC1 cells from a single lot were thawed in the different complete medias listed above. Cell viability was determined directly in the post-thaw cell suspension (without centrifugation; ‘No Cent’); or cell suspensions were first centrifuged at 400 g for 3 minutes, then pellets were gently resuspended in their respective thaw media, and then cell viabilities were determined (with centrifugation-; ‘Cent’). As shown in FIG. 59, the post-thaw viability of CTC1 cells is increased when a high HSA concentration (High Flexbumin) or both high HSA and a ROC inhibitor are included in the thaw medium. Cell viability is also increased when thawed cells are not centrifuged prior to plating. Further, as shown in FIG. 60, the post-thaw viability of CTC1 cells is increased when H1152 is included in the thaw media and cells are not centrifuged (Media C out-performed Media B, H1152).

Next, the platability of cells thawed in C media, and plated in either A or C media, was analyzed. To test this, CTC1 cells were thawed in A media, centrifuged or not, and plated in A media. The same lot of cells were thawed in C media, centrifuged or not and plated in C media. The same lot of cells were thawed in C media, centrifuged or not and plated in A media. Cells were cultured in their plating medias for 48 hours in a 37 °C, 5% CO₂ humidified incubator. Cells were harvested on Day+2, and viable cells were counted using an automated cell counter. An illustration of the experimental design is shown in FIG. 61. As shown in FIG. 62, on Day+2, more cells were recovered in conditions where cells were thawed in media C. The most cells were recovered when cells were thawed in C media, cells were not centrifuged, and they were plated and cultured in Complete Media A.

Additionally, for clinical (large) scale processes, it was observed that centrifuging the cells after thaw led to a decrease in post-thaw cell viability, as shown in TABLE 16.

TABLE 16
Comparison of viable cell number in a large scale process
with and with out a centrifugation step after thaw.

| | | Cells - Number | Cells - |
|---|---|---|---|
| | | of viable Cells | % |
| | | (10⁶) | viability |
| Thaw 1 vial of CTC1 | Without | 132 | 70 |
| (lot# EVDEM004) | centrifuging | | |
| | Centrifuging | 94 | 53 |

### Example 29

### Optimization of Cell Culture Vessel for Cell Culturing

For clinical (large) scale processes, the effect of different cell culture vessels for cell culturing was determined. Briefly, T75, HYPERFlasks, CellStack 2-layer, and CellStack 10-layer vessels were used for cell culturing, coated with vitronectin. Each vessel was plated with 100,000 viable cells (CTC1) per cm² and cultured for 2 (D+2) and/or 3 (D+3) days. The results are shown in FIG. 63. CellStack 2-layer and CellStack 10-layer vessels were superior to HYPERFlasks for the large scale culture of CTC1 cells. Likely due to reduced gas exchange in the HYPERFlasks, cells appeared to grow less well, resulting in large holes in the monolayers, which are partially visible in FIG. 63. Furthermore, HYPERFlasks are less compatible for clinical manufacturing applications, since the daily observation of cells is difficult through the many layers of HYPERFlasks. It is apparent in FIG. 63, that images of these flasks are less sharp that images taken from the CellStack 2-layer and CellStack 10-layer flasks. In addition, adequate single cell recovery of the cells, which is necessary for in-process quality control testing, was not achieved from HYPERFlasks. Passaging attemps resulted in large clumps, loss of cells and an inability to isolate single cells for accurate cell counting. Single cell recovery from CellStack 2-layer and 10-layer flasks was easily achieved, and cells could be recovered for counting, flow cytometry, RNA analysis and any other in-process control tests needed.

### Example 30

### Optimization of Insulin Concentration in Culture Media

To maximise cell yield throughout the vesiculation process, the effect of insulin concentration on cell yield was analyzed. During the analysis, it was observed that freshly differentiated and harvested cells plated and expanded well in media without insulin. However, cells that had been cryo-preserved and then thawed did not expand as well. Accordingly, the effect of insulin addition to Complete Media on improving cell yield at the end of vesiculation was assessed. For this experiment, four solutions were prepared as whoin in TABLE 17 through to TABLE 21 below (5 tables).

TABLE 17
Components of “Poor Media”.

| Item | Vendor | Part No | Volume | |
|---|---|---|---|---|
| MEMa GlutaMAX | Gibco | 32561-037 | 99.5 | mL |
| Glucose (200 mg/mL) | Gibco | A2494001 | 0.5 | mL |

TABLE 18
Components of “Complete Media A”.

| Item | Vendor | Part No | Volume | |
|---|---|---|---|---|
| MEMa GlutaMAX | Gibco | 32561-037 | 57.96 | mL |
| Glucose (200 mg/mL) | Gibco | A2494001 | 1.2 | mL |
| B27 Minus Insulin | Gibco | A18956-01 | 0.3 | mL |
| Supplement (50X) | | | | |
| Flexbumin (25% w/vol | Baxter | NDC0944-0493-02 | 0.48 | mL |
| HSA), Baxter (250 mg/mL) | | Code 2G0012 | | |
| Human FGF-2, Premium | Miltenyi | 130093843 | 0.06 | mL |

| | |
|---|---|
| grade (1000 μg/mL) | Biotec |

TABLE 19
Components of “Complete Media A with Insulin”.

| Item | Vendor | Part No | Volume | |
|---|---|---|---|---|
| MEMa GlutaMAX | Gibco | 32561-037 | 57.96 | mL |
| Glucose (200 mg/mL) | Gibco | A2494001 | 1.2 | mL |
| B27 Supplement (50X) | Gibco | 17504-044 | 0.3 | mL |
| Flexbumin (25% w/vol | Baxter | NDC0944-0493-02 | 0.48 | mL |
| HSA), Baxter (250 mg/mL) | | Code 2G0012 | | |
| Human FGF-2, Premium | Miltenyi | 130093843 | 0.06 | mL |

| | |
|---|---|
| grade (1000 μg/mL) | Biotec |

TABLE 20
Components of “Complete Media C”.

| Item | Vendor | Part No | Volume |
|---|---|---|---|
| MEMa GlutaMAX | Gibco | 32561-037 | 44.2 mL |
| Glucose (200 mg/mL) | Gibco | A2494001 | 1.0 |
| B27 Minus Insulin | Gibco | A18956-01 | 0.250 |
| Supplement (50X) | | | |
| Flexbumin (25% w/vol | Baxter | NDC0944-0493-02 | 4.0 |
| HSA), Baxter (250 mg/mL) | | Code 2G0012 | |
| Human FGF-2, Premium | Miltenyi | 130093843 | 0.05 |
| grade (1000 μg/mL) | Biotec | | |
| H1152, 100 μM | FCDI | Q072 | 0.5 |

TABLE 21
Components of “Quench” media.

| Item | Vendor | Part No | Volume | |
|---|---|---|---|---|
| MEMa GlutaMAX | Gibco | 32561-037 | 98 | mL |
| B27 Minus Insulin Supplement (50X) | Gibco | A18956-01 | 2 | mL |

Three separate lots of CTC1 cells were thawed in Complete Media C, incubated at room temperature for 10 min, divided into two conical tubes each, and centrifuged at 400 g for 3 min. The pellets were gently resuspended in Complete Media A or Complete Media A with insulin and plated in their respective medias at approximately 100,000 cells/cm². The plates used were CellBind 6-well plates (Corning, part #3335), which had been coated with recombinant vitronectin (ThermoFisher, A14700).

The cells were cultured at 37 °C, 5% CO₂ in a humidified incubator for 48 hours. Spent media was removed and each well was rinsed three times with MEMa Glutamax basal media, and then Poor Media was added to each well. Cells were cultured at 37 °C, 5% CO₂ in a humidified incubator for a further 48 hours. Images were taken and cells were harvested (with 0.05% Trypsin-EDTA), quenched with Quench, centrifuged and resuspended in Quench. Viable cells were counted using an automated cell counter (ViCell). The number of cells per square centimeter from the culture vessels were calculated. An illustration of the experimental design is shown in FIG. 64.

For all three lots, there were significantly more cells at the end of the four-day process when Complete Media A with Insulin was used for the first two days of culture, as shown in FIG. 65. Accordingly, it was found that insulin in the Complete Media is important in increasing cell yield of previously cryopreserved CTC1 cells.

### Example 31

### Optimization of Growth Factor Concentration in Culture Media

To optimize the amount of growth factor in culture media, FGF concentration was adjusted and the effect on vesiculation was analyzed. Specifically, three different FGF concentrations (in Complete Media formulations) were tested: High (1 μg/mL), Medium (500 ng/mL), and Low (100 ng/mL). Poor media was also used a control, which contained no FGF. Following vesiculation, EV/secretomes from CTC1 were collected from each condition and were evaluated for bioactivity in cardiomyocyte survival assays and HUVEC scratch wound healing assays. An illustration of the experimental design is shown in FIG. 66. The results are shown in FIGS. 67-69.

As shown in FIG. 67, which depicts a line graph of cell counts of the three culture conditions, FGF concentration did not appear to significantly affect cell number. However, as shown in FIG. 68, which depicts the results of the scratch wound healing experiments, the effectiveness of the CTC1-EV (in the scratch wound healing assay) is clearly affected by the amount of FGF present in the expansion phase of the vesiculation protocol (Complete Media). TABLE 22 calls out the 18 hour data points as depicted in FIG. 68. Note that the last two days of culture for all conditions were without FGF (Poor Media). Further, as shown in FIG. 69, which depicts the results of the cardiomyocyte survival assay, dose responses were evident for both the High and Medium FGF concentration protocols, with the 2× dose of EV from the High FGF protocol performing the best. TABLE 23 calls out the 19 hour data points as depicted in FIG. 69. Of the tested concentrations, it was determined that the High (1p g/mL) FGF concentration was optimal.

TABLE 22
Comparison of FGF concentration on effect
in HUVEC scratch wound assay.

| | |
|---|---|
| Additional Information | % Wound Confluence |

| for FIG. 68 | Figure Labels | 18 Hour Timepoint |
|---|---|---|
| | Positive Control | 39.68 |
| Standard Protocol | High FGF, MC | 23.47 |
| | Mid FGF, MC | 9.87 |
| | Low FGF, MC | 6.10 |
| Negative Control and | Low FGF, MV | 6.94 |
| Mock EV Controls | Mid FGF, MV | 5.66 |
| with no impact on | High FGF, MV | 4.72 |
| Wound Confluence | Negative Control | 1.31 |

TABLE 23
Comparison of FGF concentration on effect
in cardiomyocyte viability assay.

| | % Positive NucLight Red | |
|---|---|---|
| | | 19 Hour |
| Additional Informationfor FIG. 69 | Figure labels | Timepoint |
| | Complete Media | 1.00 |
| | High FGF 2x | 0.916 |
| | Mid FGF 2x | 0.910 |
| | FBS | 0.894 |
| | High FGF 1x | 0.892 |
| | Low FGF 1x | 0.876 |
| | Mid FGF 1x | 0.876 |
| | Low FGF 2x | 0.872 |
| Negative Control and Mock EV | Staurosporine | 0.889 |
| controls with no impact on CM² | Mid FGF 1x MV | 0.868 |
| Viability | Low FGF 1x MV | 0.865 |
| | Mid FGF 2x MV | 0.864 |
| | Low FGF 2x MV | 0.861 |
| | High FGF 2x MV | 0.858 |
| | High FGF 1x MV | 0.854 |

### Example 32

### Optimization of Filter Cut-Off Size for Purifying EV/Secretomes

To optimize filter cut-off size for purifying EV/secretomes, EV from CTC1 from fresh or frozen/thawed spent media were prepared by ultracentrifugation (UC) and by ultrafiltration (UF) at various pore-size cut-offs. Preparations were evaluated for in vitro function using a HUVEC scratch wound healing assay and a cardiomyocyte survival assay (staurosporine assay). In vitro assays showed some differences between samples that were prepared from fresh vs frozen/thawed spent medias. In all cases, significant functionality was observed in <100 kDa samples, and some functionality of frozen/thawed media was also found in the <50 kDa for frozen/thawed media in the cardiomyocyte survival assay. Accordingly, based on these data, a cutoff size of less than 50 kDa would capture most of the functionality observed in these assays.

Further, human iPCS were expanded, differentiated, and cryo-preserved to CTC1 cells at development scale. The cryopreserved CTC1 cells were thawed, plated, and vesiculated using a 4-day process, using a complete media formulation without insulin. The spent media were collected, clarified by differential centrifugation (400 g for 10 min, 2000 g for 30 min), and split into two. One portion was utilized immediately (fresh media), and the other portion was frozen at −80 °C and processed later as “frozen media.” Virgin media controls were prepared and processed in parallel.

A sample of clarified media was ultracentrifuged at 100,000 g for 16 hours. Pellets were resuspended in 0.1 um filtered dPBS, aliquoted and stored at −80 °C until further use.

Additionally, a 15 mL sample of clarified media was added to the top of a 0.2 μm ultrafiltration (UF) unit (VivaSpin20 PES ultrafiltration unit; Sartorius VS201s1/1208M08). All UF units were sterilised with 70% ethanol and rinsed with 1.0 μm filtered dPBS prior to use. As per the manufacturer's directions, the media and UF unit were centrifuged at 3,000 g for up to 15 min, or until the maximum of the liquid had passed through the filter. The retentate contained primarily material>0.2 μm in size.

The flow-through was collected and added to the next smallest filter-pore-size (100 kDa size cut-off) and the process was repeated. The retentate was then enriched for material ranging from 100 kDa to 0.2 μm in size. This process was repeated using, in sequence, UF filters having 50 kDa, 30 kDa, 10 kDa, and 5 kDa cut-offs. Thus, additional retentates were generated which were enriched for material of size ranges of 50-100 kDa, 30-50 kDa, 10-30 kDa and 5-10 kDa, respectively. Subsequently, the first retentate was washed three times with dPBS, and the flow throughs were added to the next smallest UF in order that the next retentate be rinsed three times. The flow-through was again passed to the next smallest filter's retentate and so on, such that all retentates were rinsed three times with the wash solution. Each washed retentate was collected into an Eppendorf tube, volumes were increased to approximately 1 mL with 0.1 μm filtered dPBS and stored at −80 °C until further use.

For the HUVEC scratch wound healing assay, secretome samples were dosed into the assay at a 3× doses (where 1× doses were the secretome/EV/secretome fraction produced by 150,000 mother cells). MV controls were volume matched. As shown in FIG. 70A and FIG. 70B, which depict the results of a time course monitoring scratch wound healing, for fresh media samples, 1× UC has the strongest effect. Fresh UF fractions 5-10K, 50-100K and 100k-0.2 μm (3× dose) also have a positive effect on endothelial cell migration into the scratch. Datapoints from FIG. 70A and FIG. 70B are given in TABLE 24, and TABLE 25, respectively.

TABLE 24
Fresh vs. frozen UC and fresh UF samples in a HUVEC
scratch assay, 18 hour timepoint as seen in FIG. 70A.

| | | % Wound Confluence | |
|---|---|---|---|
| Additional Labels | | | 18 Hour |
| for FIG. 70A | Figure Labels | Series Name | Timepoint |
| | Complete Media Control | Complete Media Control | 33.23 |
| | Fresh UC generated EV | Fresh UC generated EV | 15.09 |
| | FBS Control | FBS Control | 13.13 |
| UF Retentates | 50 kDa-100 kDa | 50 kDa-100 kDa | 12.61 |
| | | (50k Fresh UF) | |
| | 100 kDa-0.2 μm | (100k Fresh UF) | 8.85 |
| | | (100 kDa-0.2 μm) | |
| | 30 kDa-50 kDa | (30k Fresh UF) | 6.34 |
| | | (30 kDa-50 kDa) | |
| | 10 kDa-30 kDa | (10k Fresh UF) | 5.81 |
| | | (10 kDa-30 kDa) | |
| | 5 kDa-10 kDa | (5k Fresh UF) | 5.42 |
| | | (5 kDa-10 kDa) | |
| Mock-EV controls | | (5k Fresh UF MV) | 4.59 |
| and Poor Media | | (5 kDa-10 kDa MV) | |
| Control (4.59 | | 0.2 μm Fresh UF MV | 4.53 |
| and below) | Poor Media Control | Poor Media Control | 4.28 |
| | | 30k Fresh UF MV | 3.64 |
| | | (30 kDa-50 kDa MV) | |
| | | 50k Fresh UF MV | 3.37 |
| | | (50 kDa-100 kDa MV) | |
| | | 10k Fresh UF MV | 3.02 |
| | | (10 kDa-30 kDa MV) | |
| | | 0.2 μm Fresh UF | 2.77 |
| | | 100k Fresh UF MV | 2.18 |
| | | (100 kDa-0.2 μm) | |
| | | Fresh UC MV | 1.67 |

TABLE 25
Fresh vs. frozen UC and fresh UF samples in a HUVEC
scratch assay, 18 hour timepoint as seen in FIG. 70B.

| | | % Wound Confluence | |
|---|---|---|---|
| Additional Labels | | | 18 Hour |
| for FIG. 70B | Figure Labels | Sample Name | Timepoint |
| | Complete media | Complete Media | 33.23 |
| | control | Control | |
| | UC generated | Fresh UC | 15.09 |
| | EV | generated EV | |
| | FBS Control | FBS Control | 13.13 |
| UF Retentates | 50 kDa-100 kDa | 50k Fresh UF | 12.61 |
| | | (50 kDa-100 kDa) | |
| | 100 kDa-0.2 μm | 100k Fresh UF | 8.85 |
| | | (100 kDa-0.2 μm) | |
| | 30 kDa-50 kDa | 30k Fresh UF | 6.34 |
| | | (30 kDa-50 kDa) | |
| | 10 kDa-30 kDa | 10k Fresh UF | 5.81 |
| | | (10 kDa-30 kDa) | |
| | 5 kDa-10 kDa | 5k Fresh UF | 5.42 |
| | | (5 kDa-10 kDa) | |
| Poor media and | | 5k Fresh UF MV | 4.59 |
| mock EV controls | | (5 kDa-10 kDa MV) | |
| | | 0.2 μm Fresh UF MV | 4.53 |
| | | Poor Media Control | 4.28 |
| | | 30k Fresh UF MV | 3.64 |
| | | (30 kDa-50 kDa MV) | |
| | | 50k Fresh UF MV | 3.37 |
| | | (50 kDa-100 kDa MV) | |
| | | 10k Fresh UF MV | 3.02 |
| | | (10 kDa-30 kDa MV) | |
| | | 0.2 μm Fresh UF | 2.77 |
| | | 100k Fresh UF MV | 2.18 |
| | | (100 kDa-0.2 μm) | |
| | | Fresh UC MV | 1.67 |

FIG. 70.1 is an alternative depiction of the data presented in FIG. 70A. In addition, it depicts the results of a CTC1-EV secretome composition prepared by ultracentrifugation of previously frozen CTC1 conditioned medium (labeled “EV 181 Frozen UC” in the figure) and its mock-EV control (labeled “EV 189 Frozen UC MV” in the figure). The 18-hour time point results depicted in FIG. 70.1 are given in TABLE 26.

TABLE 26
18-hour time point data for results depicted in FIG. 70.1.

| | | % Wound Confluence | |
|---|---|---|---|
| Additional Labels | | | 18 Hour |
| for FIG. 70.1 | Figure Labels | Sample Name | Timepoint |
| | Complete | Complete | 33.23 |
| | EV 163 Fresh UC | EV 163 Fresh UC | 15.09 |
| | EV 181 Frozen UC | EV 181 Frozen UC | 14.75 |
| | FBS | FBS | 13.13 |
| | EV 158 Fresh 50K UF | EV 158 Fresh 50k UF | 12.61 |
| | | (50 kDa-100 kDa) | |
| | EV 157 Fresh 100K UF | EV 157 Fresh 100k UF | 8.85 |
| | | (100 kDa-0.2 μm) | |
| Mock EV controls, Poor | EV 159 Fresh 30K UF | EV 159 Fresh 30k UF | 6.34 |
| media and Conditioned | | (30 kDa-50 kDa) | |
| media fractions with | | EV 189 Frozen UC MV | 6.19 |
| minimal impact on Wound | | EV 160 10k Fresh UF | 5.81 |
| Confluence | | (10 kDa-30 kDa) | |
| | | EV 161 5k Fresh UF | 5.42 |
| | | (5 kDa-10 kDa) | |
| | | EV 169 5k Fresh UF MV | 4.59 |
| | | (5 kDa-10 kDa MV) | |
| | | EV 164 0.2 μm Fresh | 4.53 |
| | | UF MV | |
| | | Poor Media Control | 4.28 |
| | | EV 167 30k Fresh UF | 3.64 |
| | | MV | |
| | | (30 kDa-50 kDa MV) | |
| | | EV 166 50k Fresh UF | 3.37 |
| | | MV | |
| | | (50 kDa-100 kDa MV) | |
| | | EV 168 10k Fresh UF | 3.02 |
| | | MV | |
| | | (10 kDa-30 kDa MV) | |
| | | EV 156 0.2 μm Fresh UF | 2.77 |
| | | EV 165 100k Fresh UF | 2.18 |
| | | MV | |
| | | (100 kDa-0.2 μm) | |
| | | EV 171 Fresh UC MV | 1.67 |

Further, as shown in FIG. 71, which depicts the results of a time course monitoring scratch wound healing, for frozen/thawed media samples, 1×UC has the strongest effect. Frozen UF fraction 50k-100 k (3× dose) also had a positive effect on endothelial cell migration into the scratch. Datapoints from FIG. 71 are given in TABLE 27.

TABLE 27
Fresh vs. frozen UC and frozen UF samples in a HUVEC
scratch assay, 18 hour timepoint as seen in FIG. 71.

| | | % Wound Confluence | |
|---|---|---|---|
| Additional Information | Figure | | 18 Hour |
| for FIG. 71 | Labels | Series Name | Timepoint |
| | Complete | Complete | 40.14 |
| | Frozen | Frozen UC | 30.69 |
| | | (EV 181 CTC-1 d + 4 | |
| | | Frozen UC) | |
| | Fresh UC | Fresh UC | 25.01 |
| | | (EV 163 CTC-1 d + 4 Fresh UC) | |
| | Frozen 50K | Frozen 50K UF | 21.49 |
| | UF | (EV 176 Frozen 50K UF) | |
| | FBS | FBS | 18.56 |
| Mock EV controls, Poor | | (EV 186 Frozen 10K UF-MV) | 13.13 |
| media and Conditioned | | (EV 189 CTC-1 d + 4 Frozen | 12.17 |
| media fractions with | | UC MV) | |
| no impact on Wound | | (EV 187 Frozen 5K UF-MV) | 11.60 |
| Confluence | | (EV 184 Frozen 50K UF-MV) | 11.42 |
| | | (EV 179 Frozen 5K UF) | 11.03 |
| | | (EV 175 Frozen 100K UF) | 10.59 |
| | | (EV 183 Frozen 100K UF-MV) | 10.55 |
| | Poor | Poor | 9.73 |
| | | (EV 177 Frozen 30K UF) | 9.58 |
| | | (EV 182 Frozen 0.2 μm UF- | 9.54 |
| | | MV) | |
| | | EV 185 Frozen 30K UF-MV | 9.12 |
| | | EV 178 Frozen 10K UF | 8.95 |
| | | EV 171 CTC-1 d + 4 Fresh UC | 7.29 |
| | | MV | |
| | | EV 174 Frozen 0.2 μm UF | 6.97 |

Samples were also tested in a second assay (a cardiomyocyte viability assay). In the cardiomyocyte viability assay, 1× doses of UC and UF samples were tested. As the samples were dilute, it was not possible to test a 3× dose in this assay. CM viability was robustly increased by UC (fresh and frozen) samples, and somewhat increased by 1× doses of fresh UF in the 50-100k fraction and the frozen UF 50 kDa-100 kDa+30 kDa-50 kDa+5 kDa-10 kDa. As shown in FIG. 72, which depicts a histogram of double normalized data from cardiomyocyte survival assays, fresh and frozen UC samples exhibited a strong effect on cardiomyocyte viability. As shown in FIG. 73, which depicts a histogram of double normalized data from cardiomyocyte survival assays, fresh UF samples exhibited an effect on cardiomyocyte viability. As shown in FIG. 74, which depicts a histogram of double normalized data from cardiomyocyte survival assays, frozen UF samples exhibited an effect on cardiomyocyte viability. These data suggested that a filter cut-off size of 30 kDa would be appropriate to capture the majority of the secretome function on CM viability and endothelial cell activation coming from the CTC1 secretome. These data also show that the additive effect of compounds in different size fractions is important for strong functionality.

### Example 33

### Optimization of Sterilizing Filtration Prior to Vialing

To optimize filter sterilization prior to vialing of EV/secretomes, different sterilizing filter formats were tested for clogging, and for their suitability for use in a closed system. In these experiments, filtration was assessed by analyzing particle number (by nanoparticle tracking analysis; NIA) before and after filtration to determine yield, as well as assessing protein concentration. TABLE 28 depicts the results. Additionally, different sterilizing filter formats were tested, the results of which are shown in TABLE 29.

TABLE 28
The results of filter sterilization experiments on particle number and protein
concentration, using different filters as described in Example 33.

| | Particles/ml | Total particles | | Protein | Total |
|---|---|---|---|---|---|
| | (NTA; | (NTA; | Particles/ | (BCA; | protein |
| Sample Description | particles/mL) | particles) | cell | mg/mL) | (BCA; mg) |
| Retentate | 5.6 × 10¹⁰ | 6.2 × 10¹² | 20 000 | 0.2 | 20 |
| before sterilizing | | | | | |
| filtration 0.2 μm | | | | | |
| Final formulation | 4.6 × 10¹⁰ | 4.2 × 10¹¹ | 17 000 | 0.2 | 20 |
| after filtration PES | | | | | |
| PALL filter (PALL) | | | | | |
| Final formulation | 5.8 × 10¹⁰ | 2.3 × 10¹¹ | 21 000 | 0.2 | 20 |
| after filtration PES/ | | | | | |
| PVDF PALL (PALL) | | | | | |
| Final formulation | 5.8 × 10¹⁰ | 14 × 10¹¹ | 21 000 | 0.2 | 20 |
| after filtration | | | | | |
| Steriflip PES | | | | | |
| (Millipore) | | | | | |
| Final formulation | 3.3 × 10¹⁰ | 7.9 × 10¹¹ | 12 000 | 0.3 | 30 |
| after filtration | | | | | |
| Steriflip PVDF | | | | | |
| (Millipore) | | | | | |

TABLE 29
The results of filter sterilization experiments on particle number and protein
concentration, using different filters as described in Example 33.

| | | Total | | | Total |
|---|---|---|---|---|---|
| | Particles/mL | particles | Particles/ | Protein | protein |
| Samples | (NTA) | (NTA) | cell | (BCA) | (BCA) |
| Retentate before | 1.6 × 10¹¹ | 2.1 10¹³ | 5 200 | 0.5 mg/mL | 67 mg |
| sterilizing | | | | | |
| filtration 0.2 μm | | | | | |
| Final formulation | 8.5 × 10¹⁰ | 1.1 × 10¹³ | 2 700 | 0.7 mg/mL | 93 mg |
| after filtration | | | | | |
| Sterivex ™; | | | | | |
| GP 0.2 μm filter unit | | | | | |
| Final formulation | 8.5 × 10¹⁰ | 1.1 × 10¹³ | 2 700 | 0.7 mg/mL | 93 mg |
| after filtration | | | | | |
| Sartopore 2 | | | | | |

### Example 34

### Quantification and Characterization of Residual DNA

Exemplary techniques useable to analyze the different components of a secretome composition are shown in TABLE 30.

TABLE 30
Exemplary techniques useable to analyze
components of a secretom composition.

| Composition | Characterization techniques |
|---|---|
| Extracellular | Size distribution by Nanoparticle |
| vesicles, or particles | tracking analysis (NTA) |
| | Particle quantification by MACSPlex |
| | exosome kit (Miltenyi) using flow |
| | cytometry |
| | Quantification of particles by NTA |
| | Surface protein (phenotyping by MACSPlex |
| | exosome kit Miltenyi by flow cytometry) |
| | Micro RNA content of particles by |
| | transcriptomics |
| DNA (may be contained | Quantification of residual DNA by |
| within the vesicles | quantitative PCR |
| or on the surface) | Analysis of fragment size by |
| | quantitative PCR |
| | Residual DNA sequencing |
| Proteins contained in | Protein concentration by BCA |
| the secretome including | Proteomic analysis by mass spectrometry |
| those in the extracellular | |
| vesicles | |

For biological products, such as vaccines, antibodies, gene therapies, and lentiviral-based products, there is an expectation from regulatory bodies that the final product will have limited impurities of host-DNA (see European Pharmacopeia (EP), section 2.6.35: Quantification and characterisation of residual DNA). Therefore, during the development of the EV product, the amount of DNA was measured using a Nanodrop and Qubit, which measure the concentration of double stranded DNA molecules. A level of DNA was found which was significantly higher than the recommended 10 ng/dose described in the World Health Organization, Requirements for the use of animal cells as in vitro substrates for the production of biologicals. World Health Organization (WHO) Technical Report. Series 878, 1998, Annex 1.

The simplest solution would have been to digest the DNA using a product such as Benzonase. However, Yokoi et al, Science Advances, 20 Nov. 2019, “Mechanisms of nuclear content loading to exosomes”; Rufino-Ramos et al, Journal of Controlled Release, 28 Sept 2017, “Extracellular vesicles: Novel promising delivery systems for therapy of brain diseases”; Fischer et al, PLoS One, 29 Sept. 2016, “Indication of Horizontal DNA gene transfer by Extracellular vesicles”; and Cai et al. Experimental Cell Research, 15 Nov. 2016, “Functional transferred DNA within extracellular vesicles” teach that EV can contain DNA as part of their functional cargo. Therefore, it may be unreasonable to expect complete absence of DNA in the final product. Furthermore, destruction of the DNA in the final product may damage the vesicles or destroy part of their therapeutic cargo. Therefore, an alternative method to evaluate the risk of the DNA content in the final product was sought.

The “Method A—Real time Quantitative PCR” section of the EP 2.6.35, teaches that it may be possible to more precisely characterise DNA in a product using a qPCR method. This method suggests that “For the quantification of residual host-cell DNA, qPCR targeting either a stable sequence within a highly conserved host-cell region or targeting repetitive elements to enhance the sensitivity of the test can be used.” The FDA teaches in “Guidance for Industry: Characterization and Qualification of Cell Substrates and other biological materials used in the production of viral vaccines for infectious disease indications” that to reduce the risk of oncogenic and/or infectivity potential, the residual DNA fragments should be less than 200 base pairs in length.

Therefore, an assay was developed to interrogate the oncogenic risk of the residual DNA using a two-step approach: 1/the DNA would be quantified and its length characterized using a qPCR approach, to determine if fragments were >200 bp, and 2/assess the oncogene risk of any residual DNA through exosome sequencing and risk assessment using the oncogene panel defined as the list of genes in OncoKB Cancer Gene List and the Integragen CAncerGEnes (for a total of 1142 genes). The analytical methods developed to characterize residual DNA are summarized in TABLE 31 and described below.

TABLE 31
Analytical methods developed to characterize residual DNA.

| | | | Acceptance |
|---|---|---|---|
| Assay | Method | Reference | criteria |
| Quantification of | Quantitative | EP 2.6.35 | For information |
| residual DNA | PCR | | only (FIO) |
| Analysis of fragment | Bioanalyzer | NA | For information |
| sizes of residual DNA | | | only (FIO) |
| Residual DNA | Sequencing | NA | For information |
| sequencing | | | only (FIO) |

### Quantification of Residual DNA by Quantitative PCR

Since the reporting of fragment sizes of residual DNA may become necessary as a regulatory requirement, experiments were performed to develop an assay for determining the size of residual DNA. Briefly, two methods were developed to detect DNA fragments of −200 bp:
detection of ALU sequences of 80 and 221 bp (Alu sequences were Universal real-time PCR assay for quantification and size evaluation for residual cell DNA in human viral vaccines;
detection of human 18S rRNA genes of 123 and 254 bp.

The ALU sequence detection assay:

Method used: Impurities: Residual DNA (EP 2.6.35): equipment QuantStudio 5 (QS5) 96well, 0.2 mL (Applied Biosystems).

The ALU sequence assay is a quantitative real-time PCR (qPCR) method using specific fluorescent Taqman® probes, as permitted by EP 2.6.35 (quantification and characterization of residual host cell DNA). The search for residual DNA by qPCR focuses on a sequence of interest recognized by the probes. This target sequence corresponds to a sequence ubiquitously present in the producer cells (conserved repetitive sequences). The method used here searches for two target sequences of 80 and 221 base pairs contained in an ALU consensus sequence, a repetitive and ubiquitously present sequence in human cells. Targeting two fragment lengths made it possible to qualitatively assess the distribution of DNA fragments present in the medium tested, according to their size.

An internal positive control was added to the test material (spike-in) and then DNA was extracted with the DNeasy Blood and Tissue Extraction Kit (Qiagen, ref #69504). The spike-in was a control DNA of known concentration which made it possible to establish the extraction yield.

In order to obtain a quantitative result, a standard curve was prepared using commercially available human genomic DNA (Human genomic DNA, Promega, ref #G304A). The DNA concentration of the samples was quantified relative to this range.

The reagents used to carry out the ALU sequence detection by qPCR are given in TABLE 32.

TABLE 32
The reagents used to carry out the ALU sequence detection by qPCR.

| Reagent | Reference | Manufacturer | Sequence |
|---|---|---|---|
| Human Genomic DNA | G304A | Promega | NA |
| TaqPath ™ ProAmpTM | A30866 | Applied | NA |
| Master Mix | | biosystems | |
| TaqMan ™ Fast | 4444557 | Applied | NA |
| Advanced Master Mix | | biosystems | |
| TaqMan ™ Universal | A39175 | Applied | NA |
| DNA Spike-In Control | | biosystems | |
| TaqMan ™ Gene | 4448490 | Applied | NA |
| Expression Assay, VIC | | biosystems | |
| Sequence Detection | 4304971 | Applied | TACAAAAATTAGCCGGGCGTGG |
| Primer ALU_80_for | | biosystems | |
| Sequence Detection | 4304971 | Applied | GTTCAAGCGATTCTCCTGCCTC |
| Primer ALU_80_rev | | biosystems | |
| Custom TaqMan ™ | 4482779 | Applied | CGCGCCTGTAATCCCAGCTACT |
| Probe ALU_80_FAM | | biosystems | CGG |
| Sequence Detection | 4304971 | Applied | CTCACGCCTGTAATCCCAGCAC |
| Primer ALU_221_for | | biosystems | |
| Sequence Detection | 4304971 | Applied | ATCTCGGCTCACTGCAACCTCC |
| Primer ALU_221_rev | | biosystems | |
| Custom TaqMan ™ | 4304971 | Applied | CGAGACCAGCCCGGCCAACACG |
| Probe ALU_221_ABY | | biosystems | GTGA |

The qPCR reaction and the analysis of the results were performed on a Quant Studio 5 (QSS; 96-well, 0.2 mL; Applied Biosystems®).

The results of the ALU sequence detection assay on two samples (*5 (Test 25), *5 (Test 26)) whose preparations are described in Example 19 are shown in TABLE 33.

TABLE 33
ALU sequence detection assay results for *5 (Test 25); *5 (Test 26).

| | | Clarified | Clarified |
|---|---|---|---|
| | | conditioned media | conditioned media |
| | | (after clarification | (after clarification |
| | | and before TFF) - | and before TFF) - |
| | | Clinical production | Clinical production |
| Analytical technique | Specification | 1 (*5 (Test 25)) | 2 (*5 (Test 26)) |
| Residual DNA impurities: | FIO | 194 | 143 |
| fragments >80 bp (in ng/mL) | | | |
| Residual DNA impurities: | FIO | 2,910,000 | 2,145,000 |
| fragments >80 bp (total ng) | | | |
| Residual DNA impurities: | FIO | 40.6 | 59 |
| fragments >221 bp (in | | | |
| ng/mL) | | | |
| Residual DNA impurities: | FIO | 609,000 | 885,000 |
| fragments >221 bp (total ng) | | | |

The results of the ALU sequence detection assay *9 (Test 27) are shown in TABLE 34.

TABLE 34
The ALU sequence detection assay results
for CTC1-EV sample *9 (Test 27).

| | | Results from *9 |
|---|---|---|
| Analytical Technique | Specification | (Test 27) |
| quantification and characterization of | FIO | 2.2 μg/mL |
| residual DNA from the mother cell by | | |
| qPCR: fragments >80 pb | | |
| quantification and characterization of | FIO | 1.3 μg/mL |
| residual DNA from the mother cell by | | |
| qPCR: fragments >200 pb | | |
| quantification and characterization of | FIO | 0.9 μg/mL |
| residual DNA from the mother cell by | | |
| qPCR: fragments <200 pb | | |

The 18S rRNA sequence detection assay is described below:

Method used: Impurities: Residual DNA (EP 2.6.35): equipment QuantStudio 5 (QS5) 96well, 0.2 mL (Applied Biosystems).

The 18S rRNA sequence assay is a quantitative real-time PCR (qPCR) method using specific fluorescent Taqman® probes, as permitted by EP 2.6.35 (quantification and characterization of residual host cell DNA). The search for residual DNA by qPCR focuses on a sequence of interest recognized by the probes. This target sequence corresponds to a sequence ubiquitously present in the producer cells.

An internal positive control was added to the test material (spike-in) and then DNA was extracted with the DNeasy Blood and Tissue Extraction Kit (Qiagen, ref #69504). The spike-in was a human control DNA of known concentration which made it possible to establish the extraction yield.

In order to obtain a quantitative result, a standard curve was prepared using commercially available human genomic DNA (Human genomic DNA, Promega, ref 9 G304A). The DNA concentration of the samples was quantified relative to this range.

Reagents used are listed in TABLE 35.

TABLE 35
The reagents used to carry out the qPCR.

| Reagent | Reference | Manufacturer | Sequence |
|---|---|---|---|
| Human Genomic DNA | G304A | Promega | NA |
| TaqPath ™ ProAmpTM Master Mix | A30866 | Applied | NA |
| | | biosystems | |
| TaqMan ™ Fast Advanced Master | 4444557 | Applied | NA |
| Mix | | biosystems | |
| TaqMan ™ Universal DNA Spike-In | A39175 | Applied | NA |
| Control | | biosystems | |
| TaqMan ™ Gene Expression Assay, | 4448490 | Applied | NA |
| VIC | | biosystems | |
| TaqMan probe | | Applied | AAGTCCCTGCC |
| | | biosystems | CTTTGTACACAC |
| | | | CG |
| Primer 123 pb_forward | | Applied | GCAATTATTCCC |
| | | biosystems | CATGAACG |
| Primer 254 pb_forward | | Applied | AACAGGTCTGT |
| | | biosystems | GATGCCCTT |
| Primer_reverse | | Applicd | GGCCTCACTAA |
| | | biosystems | ACCATCCAA |

The qPCR reaction and the analysis of the results were performed on a Quant Studio 5 (QS5, 96-well, 0.2 mL; Applied Biosystems®).

### Analysis of Fragments Sizes of Residual DNA

Library preparation, exome capture, and sequencing were performed by IntegraGen SA (Evry, France). Upon receipt of DNA extracts, IntegraGen SA evaluated DNA fragment size using a Bioanalyser, following standard protocols.

The results obtained for *9 (Test27) are shown in FIG. 84.1. The majority peak present in *9 (Test 27) has a size of 179 base pairs.

### Residual DNA Sequencing

Part two of the method was to sequence the residual DNA and look for any oncogenic mutations. To achieve this step, DNA was extracted from CTC1-EV, which is *9 (Test 27) in this experiment, using standard methods. Library preparation, exosome capture, sequencing and data analysis were completed by IntegraGen SA (Evry, France). The extracted DNA is then high-throughput sequenced using a human exome analysis protocol for circulating DNA. This pair-end sequencing is performed on the Illumina® Novaseq® 6000 platform in 2×100 bases using the Twist Bioscience® Human Core exome capture kit (Consensus CDS). Sequences obtained were compared to oncogene panels to ensure no concerning mutations were present.

### Example 35

### Characterization of Test Example 25, Test Example 26 and Test Example 27 Samples

In-process samples of material generated during Test Example 25, Test Example 26 and Test Example 27 as described in Example 19 were analyzed essentially as described in previous examples.

During Test Example 25, three in-process cell samples were taken. For Test Example 25, these samples are referred to as *1 (Test 25), *2 (Test 25), and *3 (Test 25). The *1 (Test 25) sample contains cells immediately after thawing the vials of FCDI CTC1 (day zero of the process; “CPC D+0”). The *2 (Test 25) sample contains cells collected after the first three days of culture in Complete Media (day three of the process; “CPC D+3”). The *3 (Test 25) sample contains cells on the last day of post-thaw culture, which is the day the spent media are collected (day five of the process; “CPC D+5”).

During Test Example 26, three in-process cells samples were taken. For Test Example 26, these samples are referred to as *1 (Test 26), *2 (Test 26), and *3 (Test 26). The *1 (Test 26) sample contains cells immediately after thawing the vials of FCDI CTC1 (day zero of the process; “CPC D+0”). The *2 (Test 26) sample contains cells collected after the first three days of culture in Complete Media (day three of the process; “CPC D+3”). The *3 (Test 26) sample contains cells on the last day of post-thaw culture, which is the day the spent media are collected (day five of the process; “CPC D+5”).

Samples of iPSC cells and cardiomyocytes (CM) were obtained and analyzed by flow cytometry. The five cell types described above (CPC D+O, CPC D+3, CPC D+5, iPSC and CM) were analyzed by flow cytometry for nine proteins of interest. CPC flow cytometry profile was evaluated as described in Example 7. The results are depicted in FIG. 77. During the period which produced the spent media (that is from the time the Poor media were added to the cells in culture on D+3 until the time the spent media were harvested on D+5), the CPC expressed more cardiovascular lineage marker protein (higher MFI) for CD56, CXCR4, GATA4, cTNT and aMHC than the iPSC controls, and less cTNT and aMHC (less than 1 tenth the MFI) than the CM controls. The CPC D+5 expressed less OCT 3/4, NANOG and SOX2 than the iPSC controls. The CPC D+5 expressed similar levels of ISL-1 than the iPSC controls.

Transcriptomic data for cells at day+3 and day+5 were analyzed as described in Example 8. Results are depicted in FIG. 78. The data (log₂FPKM) used to generate the heatmap depicted in FIG. 78 are presented in TABLE 36.

Cell morphology on day+3 and day+5 was evaluated as described in Example 5. Results are depicted in FIG. 79.

Particle concentration, mean and mode for samples *5, *6, *7 for both Test Example 25 and Test Example 26 and samples *8 and *9 for Test Example 27 were evaluated as described in Example 9. Results are depicted in FIGS. 80 and 81.

CTC1-EV surface marker expression was evaluated as described in Example 10 on *4 (Test 25), *5 (Test 25), *4 (Test 26), *5 (Test 26), *8 (Test 27), *9 (Test 27). Results are for 13 μl of sample tested are depicted in FIGS. 82-84.

TABLE 36
The data (log2FPKM) used to generate the heatmap depicted in FIG. 78 are presented.

| Gene | CM2. | CM2. | CM2. | iPSC. | iPSC. | iPSC. |
|---|---|---|---|---|---|---|
| Name | 012D13A | 390D13A | 990D13A | x225 | x226 | x227 |
| Alias | CM1 | CM2 | CM3 | iPSC2 | iPSC3 | iPSC4 |
| EOMES | −4.93069 | −4.12089 | −17.6702 | −1.10168 | −1.27814 | 1.28834 |
| MSX2 | 1.95635 | 2.079638 | 2.131557 | −2.41409 | −2.86355 | −2.65964 |
| RPL32 | 7.803537 | 7.341461 | 7.600563 | 6.983356 | 7.156383 | 6.298661 |
| POU5F1 | −4.59453 | −2.56237 | −2.41242 | 9.712896 | 9.793549 | 9.16117 |
| NANOG | −17.6702 | −6.26626 | −4.11672 | 5.751742 | 5.814302 | 6.169852 |
| SOX2 | −4.3899 | −2.58011 | −2.69319 | 6.755212 | 6.784912 | 16.368943 |
| TDGF1 | 2.798607 | 2.477319 | 2.639293 | 7.373385 | 7.345499 | 7.464435 |
| MESPI | 1.070132 | 0.504985 | 0.496239 | −1.4053 | −0.80969 | −2.98425 |
| T | −17.6702 | −17.6702 | −17.6702 | −2.040961 | −1.20093 | −17.6702 |
| EPCAM | 0.278948 | 0.475306 | 0.068866 | 6.468864 | 6.447723 | 6.368826 |
| CXCR4 | −1.04385 | −0.66692 | −1.51696 | −1.7065 | −2.24907 | −1.89315 |
| CDX2 | −17.6702 | −17.6702 | −17.6702 | −5.596371 | −17.6702 | −5.10506 |
| FGF10 | −2.48091 | −1.96649 | −2.10604 | −4.55848 | −3.27108 | −3.06717 |
| ISLI | −2.58766 | −1.36276 | −1.62784 | −5.0802 | −5.79256 | −17.6702 |
| KDR | −0.61909 | −1.48162 | −0.80811 | 5.366761 | 5.308578 | 5.74616 |
| KIT | −2.36653 | −1.78798 | −2.25469 | 3.772485 | 3.806128 | 4.177291 |
| PDGFRA | −3.64284 | −2.11841 | −2.38349 | −2.550651 | −2.64167 | −2.39045 |
| PDGFRB | 6.89513 | −5.08582 | −17.6702 | −0.26973 | −0.23081 | −0.53971 |
| NCAMI | 5.395119 | 5.633374 | 5.55992 | −0.2643 | −0.02992 | −0.06047 |
| HANDI | 6.083721 | 6.048107 | 6.24644 | −5.35051 | −17.6702 | −17.6702 |
| HAND2 | 6.855635 | 6.630765 | 6.875506 | −17.6702 | −17.6702 | −17.6702 |
| TBX5 | 4.403699 | 4.828509 | 4.803637 | −5.67833 | −3.80613 | −4.18714 |
| HOPX | 2.837924 | 2.668974 | 2.546846 | −17.6702 | −7.21959 | −17.6702 |
| GATA4 | 6.45317 | 6.684524 | 6.620094 | −3.382891 | −5.41711 | −3.62844 |
| GATA6 | 5.248785 | 5.313554 | 5.406355 | −17.6702 | −5.24563 | −17.6702 |
| NKX2−5 | 8.531764 | 8.111706 | 8.290329 | −3.619541 | −17.6702 | −17.6702 |
| SMARCD3 | 5.934004 | 5.753566 | 5.872929 | 1.079913 | 1.45421 | 0.604492 |
| MEF2C | 0.919444 | 1.223806 | 1.094438 | −2.64909 | −2.77663 | −1.97327 |
| WTI | −2.1939 | −1.87587 | −2.26648 | −17.6702 | −17.6702 | −17.6702 |
| FOXA2 | −17.6702 | −17.6702 | −17.6702 | −2.47407 | −4.06097 | −3.85706 |
| BMP4 | −2.68445 | −2.45953 | −2.30959 | 0.564244 | 1.041006 | 0.04211 |
| BMP6 | −2.9507 | −4.31065 | −5.16059 | −1.121541 | −0.69655 | −0.32757 |
| BMP10 | −17.6702 | −17.6702 | −3.34925 | −17.6702 | −17.6702 | −17.6702 |
| FRZB | −0.47353 | −0.0371 | −1.17665 | 0.785806 | 0.117695 | 0.36468 |
| MAP2 | −2.26962 | −2.50863 | −2.51622 | 1.107991 | 1.148634 | 1.467803 |
| CDH5 | −4.31678 | −17.6702 | −3.94193 | −5.54629 | −5.84369 | −5.63982 |
| PECAMI | −7.01561 | −6.79081 | −6.64093 | −17.6702 | −17.6702 | −7.01693 |
| HHEX | −3.81186 | −5.17174 | −17.6702 | 1.649517 | 1.495883 | 1.601768 |
| THYI | −4.30538 | −3.23253 | −3.66752 | 5.565449 | 5.588593 | 5.315215 |
| PAX3 | −17.6702 | −17.6702 | −5.89729 | −4.76512 | −17.6702 | −6.27336 |
| PAX7 | −17.6702 | −17.6702 | −17.6702 | 3.934883 | 4.019054 | 4.117352 |
| NR2F2 | 0.093554 | 0.057954 | 0.417358 | −5.65936 | −5.95675 | −5.75289 |
| GATA3 | −17.6702 | −17.6702 | −17.6702 | −2.3482 | −2.26717 | −2.44176 |
| NODAL | −4.82002 | −4.59512 | −1.44532 | 2.91106 | 2.881441 | 2.785799 |
| NFATCI | 0.159469 | 0.341069 | 0.29334 | 2.334634 | 2.536342 | 2.737406 |
| TNNT2 | 10.16398 | 9.818392 | 10.03421 | −2.32563 | −2.31825 | −2.52936 |
| TNNTI | 6.663359 | 6.249284 | 6.378282 | 3.911363 | 3.942202 | 3.067618 |
| IRX4 | 5.723521 | 5.474485 | 5.582664 | 3.797426 | 3.82596 | 13.416459 |
| MYH6 | 7.906485 | 7.950674 | 8.108717 | −4.0822 | −4.96454 | −6.76009 |
| LNCPRES | −17.6702 | −17.6702 | −17.6702 | 5.653432 | 5.657131 | 5.522744 |
| S2 | | | | | | |
| AL117378. | −17.6702 | −17.6702 | −17.6702 | 5.982486 | 5.9382731 | 6.240889 |
| 1 | | | | | | |
| AC009446. | −17.6702 | −17.6702 | −17.6702 | 6.423589 | 6.166463 | 6.306408 |
| 1 | | | | | | |
| FUT4 | −0.98727 | −0.30549 | −1.33062 | 0.987114 | 1.00542 | 1.03246 |
| Gene | PSC. | CTC1+5. | CTC1+3. | CTC1+5. | CTC1+3. | |
| Name | x228 | x538 | x537 | x536 | x535 | |
| Alias | iPSC5 | Test 26 D+3 | Test 26 D+3 | Test 25 D+5 | Test 25 D+3 | |
| EOMES | −1.7705 | 3.476573 | 3.702294 | 2.724655 | 2.563229 | |
| MSX2 | −2.28455 | 5.113977 | 4.874779 | 4.796524 | 4.766457 | |
| RPL32 | 7.137875 | 6.186857 | 6.451049 | 6.096785 | 6.315066 | |
| POU5F1 | 9.775374 | −4.07585 | −1.1891 | −4.19797 | −1.76622 | |
| NANOG | 5.816635 | −7.29383 | −5.50087 | −5.35788 | −17.6702 | |
| SOX2 | 6.815202 | −2.02329 | −0.775 | −1.15213 | −1.31004 | |
| TDGF1 | 7.372176 | 4.208319 | 4.136477 | 3.862727 | 3.134172 | |
| MESPI | −1.20865 | −6.10763 | −2.31434 | −17.6702 | −3.86867 | |
| T | −2.23334 | −17.6702 | 3.72755 | −4.05844 | −4.4339 | |
| EPCAM | 6.491555 | 7.210451 | 6.748917 | 7.118262 | 6.688535 | |
| CXCR4 | −1.25503 | 4.294759 | 4.533862 | 3.686631 | 4.463137 | |
| CDX2 | −6.46655 | 3.048474 | 1.202469 | 2.043046 | 0.954213 | |
| FGF10 | −2.55459 | 2.494293 | 2.090093 | 2.511696 | 2.164639 | |
| ISLI | −4.36578 | 4.112923 | 4.481432 | 3.67514 | 3.811328 | |
| KDR | 5.252178 | 5.797009 | 6.351969 | 5.449501 | 6.372186 | |
| KIT | 3.682461 | 3.011244 | 2.280593 | 2.696149 | 2.444605 | |
| PDGFRA | −2.66212 | 1.673612 | 2.793527 | 1.84439 | 3.01312 | |
| PDGFRB | −0.12985 | 2.468269 | 4.110004 | 2.185978 | 4.071079 | |
| NCAMI | −0.16995 | 2.252302 | 1.474893 | 2.582878 | 1.513515 | |
| HANDI | −5.22099 | 6.075564 | 5.661909 | 6.284767 | 6.76171 | |
| HAND2 | −6.27168 | 2.37258 | 3.234639 | 2.04471 | 3.100753 | |
| TBX5 | −4.54898 | 0.23426 | −0.47659 | −0.07969 | −0.77253 | |
| HOPX | −17.6702 | −1.75247 | −2.52231 | −2.68015 | −2.15296 | |
| GATA4 | −5.57502 | 6.020635 | 6.308941 | 6.122291 | 6.431288 | |
| GATA6 | −17.6702 | 4.23229 | 3.875683 | 4.385223 | 4.062589 | |
| NKX2−5 | −1.90509 | 7.071842 | 6.590431 | 6.77832 | 6.549436 | |
| SMARCD3 | 1.258924 | 4.26947 | 3.1934 | 4.623933 | 3.808118 | |
| MEF2C | −2.58666 | 3.105997 | 2.947089 | 3.259529 | 2.841975 | |
| WTI | −6.4634 | −3.90392 | −3.37325 | −4.48168 | −4.07961 | |
| FOXA2 | −3.48199 | 4.457299 | 4.746128 | 4.282216 | 4.576528 | |
| BMP4 | 0.8103 | 5.840976 | 6.062596 | 5.340844 | 6.084232 | |
| BMP6 | 0.94469 | 5.103508 | 4.90672 | 4.814588 | 5.204769 | |
| BMP10 | −17.6702 | 3.011755 | 4.817081 | 3.169576 | 4.187664 | |
| FRZB | 0.307667 | 2.909959 | 3.112467 | 2.771401 | 3.372877 | |
| MAP2 | 0.993608 | 1.573892 | 0.099408 | 1.779433 | −0.696 | |
| CDH5 | 5.00181 | 2.135384 | 2.777958 | 2.110895 | 3.63255 | |
| PECAMI | −5.3794 | 0.593929 | 0.939823 | 0.306886 | 1.528158 | |
| HHEX | 2.097881 | 5.136391 | 4.864308 | 5.236888 | 4.983931 | |
| THYI | 5.564195 | −1.34317 | 1.006951 | −2.00843 | 1.192394 | |
| PAX3 | −17.6702 | −4.49066 | −4.54491 | −3.37341 | −5.25122 | |
| PAX7 | 3.933968 | −5.93677 | 3.85385 | −17.6702 | −4.11278 | |
| NR2F2 | −7.11417 | 3.159961 | 1.83851 | 3.388924 | 2.110458 | |
| GATA3 | −2.80361 | 4.990903 | 5.665541 | 4.560487 | 5.443569 | |
| NODAL | 3.026054 | 0.420985 | −2.82963 | 0.798776 | −0.92464 | |
| NFATCI | 2.405227 | 2.762739 | 3.496196 | 2.349935 | 3.095585 | |
| TNNT2 | −2.37666 | 6.453037 | 4.337703 | 6.93186 | 4.645659 | |
| TNNTI | 3.761603 | 3.825163 | 3.254491 | 3.892383 | 2.712471 | |
| IRX4 | 3.937915 | 0.597233 | 0.538314 | 0.709079 | −0.69136 | |
| MYH6 | −5.12246 | 8.759474 | 6.00471 | 8.844688 | 5.758267 | |
| LNCPRES | 5.606096 | −17.6702 | −17.6702 | −17.6702 | −17.6702 | |
| S2 | | | | | | |
| AL117378. | 5.974234 | −17.6702 | −17.6702 | −17.6702 | −17.6702 | |
| 1 | | | | | | |
| AC009446. | 6.274441 | −17.6702 | −4.5045 | −17.6702 | −17.6702 | |
| 1 | | | | | | |
| FUT4 | 0.96579 | 0.244453 | 0.213476 | 0.478353 | 0.413052 | |

### Example 36

### In Vitro Functionality of CTC1-EV in a HUVEC Proliferation Assay

Validation of the bioactivity of CTC1-EV, which is *9 (Test) 27 in this example, by proliferation of Human Umbilical cord Vein Endothelial Cells (HUVEC) measured by BrdU incorporation was performed. (Specification criterion: >20% more BrdU incorporation than the control, as described in TABLE 37).

TABLE 37
Example Criterion for the HUVEC proliferation assay

| | | Applicable | |
|---|---|---|---|
| Criteria | Method | regulation | Example Criterion |
| In vitro | HUVEC | Not applicable | Proliferation Increased |
| functionality | proliferation | | by >20% over controls |

### Principle of the Test:

To develop the EV-containing product, a simple, quantitative, reproducible in vitro assay to measure its biological function was needed. Enough difference between the positive and negative controls to observe the effect of the product was required. However, the negative (or stress) control needed to be not so harsh as to be un-treatable.

To be usable as a release assay and stability assay, the assay needed to be quantitative and reproducible, and would ideally be simple, and straightforward. Previously, it was observed in numerous HUVEC scratch wound healing assays that the confluence of HUVEC cells in the un-scratched sections of the wells appeared to be higher when treated with CTC1-EV compositions than in control wells. It was concluded that the EV had an effect on HUVEC proliferation, adherence, or survival. A preliminary experiment was performed where HUVEC were plated in poor media with and without CTC1-EV. It was observed that CTC1-EV treated wells contained cells, while controls were largely devoid of cells.

Next, a method to quantify HUVEC proliferation was developed. WST-1 staining and BrdU incorporation was tested. WST-1 staining gave variable results in this assay. BrdU incorporation gave more consistent results and was selected as the method for this purpose.

The first iterations of the assay using BrdU were still too variable. Therefore, four parameters which enabled consistent results with a measurable effect of the CTC1-EV compositions were optimized. These four key parameters were: HUVEC seeding density, BrdU incorporation time, BrdU detection time, and the type of plate used for the assay.

### Selection of the Parameters for WST-1:

### Choice of Number of Cells:

Protocol: Increasing the number of HUVEC in round bottom 96-wells plate in complete medium for 24 hours. Starvation for 24 hours. Addition of WST-1 for 4 hours and spectrophotometer reading at 420 nm.

In order to select the number of cells to seed in each well, various seeding densities were tested. The preferred seeding density will have a good separation in OD between the result in “Complete medium” and in “Medium without protein”, and not be too sparcely seeded. If too sparse, there will be a tendency for a large variation in seeding densities between wells, which will add a source of technical variation to the assay. For the “20,000 HUVEC” condition, the well-to-well reproducibility is the smallest among the conditions tested. This condition also has a good separation on OD between the “Complete medium” and the “Medium without protein” conditions. Results for this experiment are given in TABLE 38.

TABLE 38
OD results at various HUVEC seeding density conditions.

| | 3 000 HUVEC | | 6 000 HUVEC | 10,000 HUVEC | | 20,000 HUVEC | |
|---|---|---|---|---|---|---|---|
| OD 420 nm | well n°1 | well n°2 | well n°1 | well n°1 | well n°2 | well n°1 | well n°2 |
| Complete medium | 0.6617 | 0.6221 | 1.4194 | 1.3511 | 1.1799 | 1.6893 | 1.6542 |
| Medium without protein | 0.31 | 0.4128 | 0.5357 | 0.7578 | 0.7073 | 1.1356 | 1.0409 |

In order to choose the number of cells to be seeded per well, the difference in OD between the “complete medium” condition and the “medium without protein” condition was evaluated. The quantity of cells per well during seeding is important to limit variability between the wells. A low number of seeded cells will result in greater variability. For the “20,000 HUVEC” condition, the reproducibility between wells 1 and 2 is the least important.

The 20,000 HUVEC per well was selected as the preferred seeding density.

### Choice of Plate Type and Repetability

Protocol: 20,000 HUVEC in round bottom 96-wells plate in complete medium for 24 hours. Starvation for 24 hours. Addition of WST-1 for 4 hours and spectrophotometer reading at 420 nm. Results are given in TABLE 39 and TABLE 40.

TABLE 39
OD results to track HUVEC proliferation in various conditions, experiments 1 and 2.

| | Experiment 1 | | | | | | | Experiment 2 | | | | | | | |
|---|---|---|---|---|---|---|---|---|---|---|---|---|---|---|---|
| | well | well | well | well | | | CV | well | well | well | well | well | | | CV |
| OD 420 nm | n°1 | n°2 | n°3 | n°4 | Mean | SD | (in %) | n°1 | n°2 | n°3 | n°4 | n°5 | Mean | SD | (in %) |
| Complete | 2.6582 | 2.5209 | 2.0257 | 3.7888 | 2.75 | 0.74 | 27 | 2.1051 | 1.4998 | 1.2561 | 3.8998 | 2.085 | 2.17 | 1.04 | 48 |
| medium | | | | | | | | | | | | | | | |
| Medium | 1.859 | 1.8998 | 1.8187 | 1.8752 | 1.86 | 0.03 | 2 | 1.2761 | 1.3981 | 1.2055 | 0.8949 | 0.8557 | 1.13 | 0.24 | 21 |
| without | | | | | | | | | | | | | | | |
| protein | | | | | | | | | | | | | | | |
| Medium | 2.1556 | 2.4674 | 1.9327 | 1.8007 | 2.09 | 0.29 | 14 | 1.0316 | 0.9386 | 0.8407 | 2.6294 | 1.6592 | 1.42 | 0.75 | 53 |
| without | | | | | | | | | | | | | | | |
| protein + 50 | | | | | | | | | | | | | | | |
| μL CTC1- | | | | | | | | | | | | | | | |
| EV | | | | | | | | | | | | | | | |
| Medium | 1.6848 | 1.596 | 1.5156 | 1.4415 | 1.56 | 10.10 | 7 | 1.2192 | 0.8916 | 0.8174 | 1.4305 | 1.0428 | 1.08 | 0.25 | 23 |
| without | | | | | | | | | | | | | | | |
| protein + 50 | | | | | | | | | | | | | | | |
| μL PBS 1X | | | | | | | | | | | | | | | |
| Medium | 0.0856 | 0.0865 | 0.0874 | 0.0831 | 0.09 | 0.00 | 2 | | | | | | | | |
| without | | | | | | | | | | | | | | | |
| protein + no | | | | | | | | | | | | | | | |
| WST-1 | | | | | | | | | | | | | | | |

TABLE 40
OD results to track HUVEC proliferation in various conditions, experiment 3.

| | Experiment 3 | | | | | | | |
|---|---|---|---|---|---|---|---|---|
| | | | | | | | | CV |
| OD 420 nm | well n°1 | well n°2 | well n°3 | well n°4 | well n°5 | Mean | SD | (in %) |
| Complete medium | 1.2177 | 0.9113 | 1.3305 | 1.0238 | 1.008 | 1.10 | 0.17 | 16 |
| Medium without protein | 0.8106 | 0.8262 | 0.5996 | 1.0537 | 0.8352 | 0.83 | 0.16 | 19 |
| Medium without protein + | 0.7872 | 0.7668 | 1.1123 | 1.0203 | 0.7975 | 0.90 | 0.16 | 18 |
| 50 μL CTC1-EV | | | | | | | | |
| Medium without protein + | 0.8246 | 0.7552 | 2.0614 | 1.4715 | 1.3889 | 1.30 | 0.53 | 41 |
| 50 μL PBS 1X | | | | | | | | |
| Medium without | | | | | | | | |
| protein + no WST-1 | | | | | | | | |

Round bottom plates have the advantage of improving the physical contact between the HUVEC cells and the EV test material. The contact surface is higher in the round bottom plates than for flat bottom plates.

Selection of plate type: round-bottomed 96-well plates.

Protocol: 20,000 HUVEC in round bottom 96-wells plate in complete medium for 24 hours. Starvation for 24 hours. Addition of WST-1 for 4 hours and spectrophotometer reading at 420 nm. Results are given in TABLE 41.

TABLE 41
OD results to track HUVEC proliferation in various conditions by WST-1.

| | Experiment 1 | | | | | | | Experiment 2 | | | | | | | |
|---|---|---|---|---|---|---|---|---|---|---|---|---|---|---|---|
| OD | well | well | well | well | | | CV | well | well | well | well | well | | | CV |
| 420 nm | n°1 | n°2 | n°3 | n°4 | Mean | SD | (in %) | n°1 | n°2 | n°3 | n°4 | n°5 | Mean | SD | (in %) |
| Complete | 1.3105 | 1.0511 | 1.787 | 1.6295 | 1.44 | 0.33 | 23 | 1.6147 | 1.2457 | 1.1822 | 1.0063 | 0.9158 | 1.19 | 0.27 | 23 |
| medium | | | | | | | | | | | | | | | |
| Medium | 0.3076 | 0.6875 | 0.4585 | 0.672 | 0.53 | 0.18 | 34 | 0.2653 | 0.5109 | 0.6272 | 0.6261 | 1.1306 | 0.63 | 0.32 | 50 |
| without | | | | | | | | | | | | | | | |
| protein | | | | | | | | | | | | | | | |
| Medium | 0.427 | 0.522 | 0.3992 | 0.3994 | 0.44 | 0.06 | 13 | 0.542 | 0.4689 | 0.9299 | 0.6848 | 0.4177 | 0.61 | 0.21 | 34 |
| without | | | | | | | | | | | | | | | |
| protein + | | | | | | | | | | | | | | | |
| 50 μL | | | | | | | | | | | | | | | |
| CTC1- | | | | | | | | | | | | | | | |
| EV | | | | | | | | | | | | | | | |
| Medium | 0.5508 | 0.296 | 0.4511 | 0.2641 | 0.39 | 0.13 | 34 | 0.2724 | 0.2324 | 0.2215 | 0.3379 | 0.2045 | 0.25 | 0.05 | 21 |
| without | | | | | | | | | | | | | | | |
| protein + | | | | | | | | | | | | | | | |
| 50 μL | | | | | | | | | | | | | | | |
| PBS | | | | | | | | | | | | | | | |
| 1X | | | | | | | | | | | | | | | |
| Medium | 0.0752 | 0.0767 | 0.0783 | 0.0775 | 0.08 | 0.00 | 2 | | | | | | | | |
| without | | | | | | | | | | | | | | | |
| protein + | | | | | | | | | | | | | | | |
| no | | | | | | | | | | | | | | | |
| WST-1 | | | | | | | | | | | | | | | |

For BrdU:

### Choice of Number of Cells

Protocol: increasing number of HUVEC in round bottom 96-wells plate in complete medium for 24 hours. Starvation for 24 hours. Addition of BrdU for 24 hours, revelation and spectrophotometer reading at 370 nm during 1800 sec. Results are described in TABLE 42.

TABLE 42
OD results to track HUVEC proliferation in various conditions by BrdU.

| | 3 000 HUVEC | | 10,000 HUVEC | | 20,000 HUVEC | | 30,000 HUVEC | |
|---|---|---|---|---|---|---|---|---|
| OD 370 nm | well n°1 | well n°2 | Well n°1 | well n°2 | well n°1 | well n°2 | well n°1 | well n°2 |
| Complete medium | 0.1004 | 0.1102 | 0.1342 | 0.1816 | 0.3999 | 0.4525 | 0.3753 | 0.3342 |
| Medium without protein | 0.0922 | 0.0948 | 0.1132 | 0.1304 | 0.1714 | 0.1857 | 0.1539 | 0.1352 |

In order to select the number of cells to seed in each well, various seeding densities were tested. The preferred seeding density will have a good separation in OD between the result in “Complete medium” and in “Medium without protein”, and not be too sparcely seeded. If too sparse, there will be a tendency for a large variation in seeding densities between wells, which will add a source of technical variation to the assay. For WST-1 assay, seeding 30,000 HUVEC cells per well was determined to be too high. This led to higher confluence at the end of the three days of culture at the moment of analysis for the “Complete medium” condition, which meant an unreliable difference in OD between the “Complete medium” condition and the “Medium without protein” condition.

The 20,000 HUVEC per well was selected as the preferred seeding density.

### Choice of Timing of Reading

Protocol: 20,000 HUVEC in round bottom 96-wells plate in complete medium for 24 hours. Starvation for 24 hours. Addition of BrDu for 24 hours, revelation and spectrophotometer reading at 370 nm at 0 sec, 5 min, 10 min and 15 min. Results are given in TABLE 43.

TABLE 43
OD results for different timings of readings.

| | | | | | | | | CV |
|---|---|---|---|---|---|---|---|---|
| | OD 370 nm | well n°1 | well n°2 | well n°3 | well n°4 | Mean | SD | (in %) |
| 0 sec | Complete medium | 0.1719 | 0.3999 | 0.4525 | 0.2319 | 0.31 | 0.13 | 43 |
| | Medium without protein | 0.1714 | 0.1857 | 0.1960 | 0.1973 | 0.19 | 0.01 | 6 |
| 5 min | Complete medium | 0.9679 | 1.2317 | 1.6015 | 1.1208 | 1.23 | 0.27 | 22 |
| | Medium without protein | 0.4893 | 0.6128 | 0.4982 | 0.7598 | 0.59 | 0.13 | 21 |
| 10 min | Complete medium | 2.8840 | 2.3232 | 3.0284 | 2.6359 | 2.72 | 0.31 | 11 |
| | Medium without protein | 1.0722 | 1.2132 | 0.7786 | 1.3433 | 1.10 | 0.24 | 22 |
| 15 min | Complete medium | 3.2273 | 3.3538 | 3.4432 | 3.3040 | 3.33 | 0.09 | 3 |
| | Medium without protein | 1.2847 | 1.7920 | 1.0146 | 1.9449 | 1.51 | 0.43 | 29 |

At 0 min there was insufficient signal in the complete medium. At the 10 and 15 min times point the complete medium signal were beginning to saturate. The 5 min time was sufficient to see good signal development and to avoid signal saturation.

### Repetability

Protocol: 20,000 HUVEC in round bottom 96-wells plate in complete medium for 24 hours. Starvation for 24 hours. Addition of BrDu for 24 hours, revelation and spectrophotometer reading at 370 nm at 5 min. Results are given in TABLE 44

TABLE 44
OD readings for three experiments at the selected conditions to evaluate reproducibility.

| | Experiment 1 | | | | | | | Experiment 2 | | | | | | | |
|---|---|---|---|---|---|---|---|---|---|---|---|---|---|---|---|
| OD | well | well | well | well | | | CV | well | well | well | well | well | | | CV |
| 370 nm | n°1 | n°2 | n°3 | n°4 | Mean | SD | (in %) | n°1 | n°2 | n°3 | n°4 | n°5 | Mean | SD | (in %) |
| Complete | 0.9340 | 2.6606 | 2.8862 | 2.3351 | 2.20 | 0.88 | 40 | 2.9739 | 3.1739 | 3.2782 | 3.1472 | 2.8666 | 3.09 | 0.17 | 5 |
| medium | | | | | | | | | | | | | | | |
| Medium | 0.8275 | 1.3030 | 1.5939 | 1.3594 | 1.27 | 0.32 | 25 | 0.6601 | 1.2687 | 1.5245 | 1.6169 | 1.2255 | 1.26 | 0.37 | 30 |
| without | | | | | | | | | | | | | | | |
| protein | | | | | | | | | | | | | | | |
| Medium | 1.0473 | 1.2300 | 1.3787 | 0.8546 | 1.13 | 0.23 | 20 | 1.069 | 1.4028 | 1.3941 | 1.5583 | 1.6206 | 1.41 | 0.21 | 15 |
| without | | | | | | | | | | | | | | | |
| protein + | | | | | | | | | | | | | | | |
| 50 μL | | | | | | | | | | | | | | | |
| CTC1-EV | | | | | | | | | | | | | | | |
| Medium | 0.8497 | 1.3094 | 1.1583 | 1.4400 | 1.19 | 0.25 | 21 | 0.5184 | 0.6219 | 0.8002 | 0.8771 | 0.7242 | 0.71 | 0.14 | 20 |
| without | | | | | | | | | | | | | | | |
| protein + | | | | | | | | | | | | | | | |
| 50 μL PBS | | | | | | | | | | | | | | | |
| 1X | | | | | | | | | | | | | | | |

OD readings for three experiments at the selected conditions to evaluate reproducibility.

| | Experiment 3 | | | | | | | |
|---|---|---|---|---|---|---|---|---|
| OD | well | well | well | well | well | | | CV |
| 370 nm | n°1 | n°2 | n°3 | n°4 | n°5 | Mean | SD | (in %) |
| Complete | 1.9560 | 2.5771 | 2.6038 | 2.4923 | 2.4720 | 2.42 | 0.27 | 11 |
| medium | | | | | | | | |
| Medium | 0.2900 | 0.5933 | 0.6121 | 0.6181 | 0.6463 | 0.55 | 0.15 | 27 |
| without | | | | | | | | |
| protein | | | | | | | | |
| Medium | 0.8362 | 1.1584 | 1.0065 | 1.1080 | | 1.03 | 10.14 | 14 |
| without | | | | | | | | |
| protein + | | | | | | | | |
| 50 μL | | | | | | | | |
| CTC1-EV | | | | | | | | |
| Medium | 0.3792 | 0.5836 | 0.5754 | 0.5662 | 0.7142 | 0.56 | 0.12 | 21 |
| without | | | | | | | | |
| protein + | | | | | | | | |
| 50 μL PBS | | | | | | | | |
| 1X | | | | | | | | |

From the two assays presented, the BrdU assay was selected. The effects of the CTC1-EV were greater in this assay than in the WST-1-based assay. For the WST-1, the test material is in contact with the HUVEC for 4 hours, whereas, in the BrdU-based assay, the contact time is extended to 24 hours. This may explain the stronger results. A stronger result is preferred for this assay.

HUVEC are mature human endothelial cells. They are utilized throughout the scientific literature as a tool to study, among other things, cellular proliferation, which is an indirect marker of cell health. HUVECs are thus a justifiable model to use for the development and release testing of therapies, especially for regenerative medicine, and especially for indications (like heart failure) where a loss of blood vessels is part of the pathology.

The proliferation of HUVEC can be determined by measuring the level of incorporation of BrdU into DNA, which occurs only during DNA replication (and thus, during proliferation). BrdU can be detected and quantified using commercially available BrdU ELISA kits (example: Cell proliferation ELISA, BrdU (colorimetric), ref 11 647 229 001 (Roche®)).

Method description: HUVEC (ref C2519A, Lonza®) were seeded into round-bottomed 96-well plates (20,000 cells/well) into EBM-2 media (ref CC-3156, Lonza®) with supplement EGM-2MV (ref CC-4147, Lonza®) (“Complete Media”) and incubated at 37 °C, 5% C02 in a humidified incubator. After 24 hours of culture, the media was exchanged with Complete Media (Positive Control), EBM-2 media without supplement (“Poor Media” Negative Control), Poor Media with EV-containing test material (in this example, 50 μL of *9 (Test 27) was used); or Poor Media with matched Vehicle Control (in this example, 50 L PBS was used). Cells are incubated for 24 hours at 37 °C 5% C02 in a humidified incubator. After 24 hours of incubation, BrdU was added to each well according to the manufacturer's directions. The HUVEC were returned to the 37 °C incubator. After 24 hours, HUVEC proliferation rate was determined through the detection of BrdU using the anti-BrdU detection kit according to the manufacturer's directions. The colorometric signal was detected at 370 nm by Multiskan™ Sky Spectrophotometer (Thermo Scientific).

This assay was used to determine the potency of the secretome product at the end of various development runs, as well as to determine the stability of CTC1-EV over time, which is *9 (Test 27) in this experiment.

Results: 5 different vials of *9 (Test 27) were tested for in vitro potency using the HUVEC Proliferation test. These CTC1-EV increased HUVEC proliferation rate>20% over the Vehicle Control as reported in TABLE 45.

TABLE 45
Results of HUVEC proliferation assay on 5 different vials of *9 (Test 27).

| | Vial #1 | Vial #2 | Vial #3 | Vial #4 | Vial #5 | |
|---|---|---|---|---|---|---|
| HUVEC | 36 +/− | 51 +/− | 51 +/− | 51 +/− | 32 +/− | Mean: 44 % |
| Proliferation | 7% | 8% | 13% | 9% | 15% | SD: 9%; |
| (BrdU) | | | | | | CV: 21% |

### Example 37

### In Vitro Immunogenicity Safety Testing of CTC1-EV

CTC1-EV, which was *9 (Test 27) in this experiment, was tested for the activation of allogeneic Peripheral Blood Mononuclear Cells (PBMC), as measured by the secretion levels of IL-2 and IFNγ (specification: no increased secretion compared to the negative control).

This CTC1-EV was tested for allogeneic Natural Killer (NK) cell degranulation, as measured by the expression of CD107a (specification criterion: no increased NK CD107a expression as compared with negative control). Criteria and their justification are given in TABLE 46.

TABLE 46
In vitro immunogenicity testing methds criteria and justification.

| | | Applicable | Specification | Justification of |
|---|---|---|---|---|
| Criteria | Method | regulations | criterion | specifications |
| In vitro | Activation of | ICH S8 | Absence of | Defined in ICH S8 |
| immunogenicity | allogeneic | | secretion of | Immunotoxicity |
| testing | PBMC | | IL-2 and IFNγ | studies for human |
| | Degranulation | | Absence of | pharmaceuticals - |
| | of allogeneic | | CD107a | Scientific guideline; |
| | NK | | degranulation | European |
| | | | | Medicines Agency |

1. Secretion of IL-2 and IFN-Gamma by PBMC Upon Contact with Allogeneic EV/Secretome Product.

Principle of the test: The goal of this test is to demonstrate the absence of pro-inflammatory cytokine secretion when the test material is put into contact with allogenic PBMC. In the presence of allogeneic activation, the PBMC will secrete pro-inflammatory cytokines interferon gamma (IFN-g) and interleukine-2 (IL-2). Cytokines can be detected either in the PBMC cell culture supernatant or directly in the cytoplasm of PBMC if brefeldin A is added to the culture medium. Indeed, with brefeldin A, cellular secretion is blocked, and IFN-g and IL-2 will accumulate in the cytoplasm of activated PBMC. The accumulation of these proteins can easily be detected by flow cytometry for intra-cellular proteins.

### Method Description:

Cytokine detection by intra-cellular staining: PBMC were suspended in culture medium (RPMI-1640 Medium with L-glutamine and sodium bicarbonate; ref R8758, Sigma/Merck) at 2E6 cells/mL. Test material (50 μL of *9 (Test 27) or vehicle control (50 μL of PBS 1× in this example) was incubated with 100 L of PBMC suspension and cultured in a 37 °C, 5% C02 incubator. After 1 hour of incubation, Brefeldin was added (final concentration 1×; eBioscience™ Brefeldine A 1000×, ref 00-4506-51, ThermoFisher) and incubated for 3 more hours in a 37 °C 5% C02 incubator. After 3 hours, the cells were collected, stained (according to the manufacturer's instructions) with BD FastImmune™ APC Mouse Anti-Human IL-2, ref: 341116, BD Biosciences, and with IFN-γ Secretion Assay—Detection Kit (PE), human; ref 130-054-202 Miltenyi Biotec) and analyzed by flow cytometry using a MacsQuant10 (MQ10, Miltenyi®) flow cytometer. As a positive control, PBMCs were activated using either PMA/ionomycin (eBioscience™ Cell Stimulation Cocktail (500×); ref: 00-4970-93) added of 1× brefeldin A, or Duractive 1 (ref C 11101, Beckman Coulter) (according to the manufacturer's directions). Results show that these CTC1-EV did not induce PBMC activation. Results from one experiment are given in TABLE 47.

TABLE 47
Results of PBMC activation assay from one experiment.

| Conditions | % IFNg⁺ cells | % IL-2⁺ cells |
|---|---|---|
| 50 μl PBS 1X | 0.14% | 0.19% |
| 50 μl CTC1-EV (*9(Test 27)) | 0.16% | 0.12% |
| Positive control (PMA/ | 5.21% | 6.46% |
| ionomycin/brefeldin A) | | |

Cytokine detection in cell culture supernatant: PBMC were suspended in culture medium (RPMI-1640 Medium with L-glutamine and sodium bicarbonate; ref R8758, Sigma/merck). Test material (50 μl of *9 (Test 27)) or vehicule control (50 μl of PBS 1× in this example) was incubated with 100 L of a 2×10⁶ cells/mL PBMC suspension and cultured in a 37 °C incubator for 4 hours. After 4 hours, supernatants were collected after a 2000 rpm 7 minutes centrifugation and analysed by flow cytometry using a MacsQuant10 (MQ10, Miltenyi®) flow cytometer with MACSPlex Cytotoxic T/NK Cell Kit (Miltenyi Biotech, ref: 130-125-800). As a positive control, PBMCs were activated either with 1×PMA/ionomycin (eBioscience™ Cell Stimulation Cocktail (500×); ref: 00-4970-93), or with Duractive 2 (ref C11102, Beckman Coulter) (according to the manufacturer's directions). Results show that these CTC1-EV did not induce PBMC activation. Results from one representative experiment are given in the TABLE 48.

TABLE 48
Results of one representative experiment
to measure PBMC activation.

| | IFNg | | IL-2 | |
|---|---|---|---|---|
| Conditions | (R1-A Median) | | (R1-A Median) | |
| PBS 1X | <3.2 | pg/mL | <0.6 | pg/mL |
| CTC1-EV (*9 (Test27)) | <3.2 | pg/mL | <0.6 | pg/mL |
| T+ (PMA/ionomycin) | 82.6 | pg/mL | 50.2 | pg/mL |

2. NK Degranulation Upon Contact with Allogeneic EV/Secretome Product.

Principle of the test: Natural Killer (NK) cells are lymphocytes in the innate immune system. These cells play a cytotoxic role. The NK cells detect non-self (in the case of MHC class I in-compatibility). It will also detect over-expression of class I MHC and participates in antibody-dependent cellular cytotoxicity (ADCC). Cytotoxicity of NK cells is mediated by perforin and granzyme degranulation, and by the secretion of interferon gamma (IFNg).

CD107a (also called LAMP-1) is a surface marker which is overexpressed on NK cells after class I MHC activation. The expression of CD107a is correlated with NK-cell-dependent cell lysis activity perform/granzyme degranulation and/or IFN-g secretion (Alter et al., 2004). Therefore, a cytotoxicity assay can be designed where the measure of the NK surface expression of CD107a can be taken as a surrogate for activation of the cytotoxic activity of NK cells.

### Method Description:

NK cells were isolated from PBMC from healthy donor blood by negative selection using immune-magnetic cell sorting (Miltenyi Biotech, #130-092-657) according to the manufacturer's instructions. Experiments were conducted with NK cells primed overnight with recombinant human interleukine-15 (IL-15) (50 ng/mL) (Sigma) in RPMI-1640 complete medium supplemented with 10% FBS to ensure their proper expression of NK cell activating receptors and functionality. Cytokine-activated NK cells were cultured alone or in the presence of K562 cells (ATCC, #CCL-243; positive control), 50 μL PBS 1× (as negative control), 50 μL of *9(Test27), or PMA/ionomycin (eBioscience™ Cell Stimulation Cocktail (500×); ref: 00-4970-93, or Duractive 2 (ref C11102, Beckman Coulter)) and labelled for 4 hours with an anti-CD107a-APC antibody (Miltenyi Biotech, #130-111-847) and brefeldin A in a 37 °C 5% CO2 incubator. After 4 h, cells were harvested, washed, and stained with CD16-PE, CD56-PE, CD8-APC Vio770, CD4 Viogreen specific antibodies and 7-AAD (Miltenyi Biotech), and the expression of CD107a was analysed on CD16+CD56+NK cells by flow cytometry on MACSQuant® 10 Flow Cytometer (MQ10, Miltenyi Biotec).

Results are given in TABLE 50.

TABLE 50
Percent NK cells expressing CD107.

| | % NK cells expressing CD107a | | | |
|---|---|---|---|---|
| Conditions | Experiment 1 | Experiment 2 | Experiment 3 | Experiment 4 |
| 50 μl CTC1-EV | 0.5 | 1.3 | 13.9 | 0.0 |
| (*9 (Test 27)) | | | | |
| Positive control: K562 | 6.4 | 30.0 | 65.4 | 26.7 |
| Positive control: | 21.8 | 72.4 | 77.0 | 61.9 |
| PMA/ionomycin | | | | |
| Negative control: NK | 11 | 3.7 | 8.2 | 3.9 |
| cells alone with | | | | |
| PBS 1X | | | | |

### Example 37.1

### In Vitro Analysis of the Potency of CTC1-EV in a Scratch Wound Healing Assay

To analyze the functionality and potency of CTC1-EV (which is *9 (Test 27) in this experiment), a HUVEC scratch wound healing assay was used as described in Example 3. Briefly, two days prior to assay, HUVEC aliquots were thawed, and plated onto ImageLock 96-well plates (EssenBio, Ref: 4379) at 10,000 cells/well, and grown in HUVEC Complete media for two days. Cultures were then maintained at 37 °C (atmospheric oxygen, 5% C02) throughout the maintenance and assay process. Wells were scratched using a Wound Maker (EssenBio, Ref: 4493) according to the manufacturer's directions, and cells were then rinsed with Endothelial Cell Basal Media and cultured overnight (either in HUVEC Complete Media alone, as a positive control; in Endothelial Cell Basal Media alone, as a negative control; in Endothelial Cell Basal Media supplemented with the pelleted material resulting from FBS ultracentrifugation; in Endothelial Cell Basal Media supplemented with *9 (Test 27) at doses of 0.25×, 0.5×, 0.75×, 1×, 2×, 2.8×, where 1× is 5.79 μL of *9 (Test 27), which is the volume of Final Formulation derived from 150,000 mother cells. Using an Incucyte with the Scratch Wound Healing Module, plates were imaged every three hours for a total of 24 hours. Percent wound closure was determined using the manufacturer's software and displayed as a time course. The values obtained at the 18 hour timepoint were then double normalized to control values at the 18-hour timepoint (baseline subtracted and normalized to the positive control; baseline is the negative control) and graphed as a histogram.

The results depicted in FIG. 85 show that all tested doses of *9 (Test 27) showed an improvement in scratch wound healing over the negative control, where the highest doses had the greatest scratch wound healing, as measured by % wound confluence. The highest dose tested, 2.8×, attained 51.70% wound confluence after 24 hours, which is 3.9-fold higher than the negative control. TABLE 51shows the 24 hour data points for FIG. 85.

TABLE 51
*9 (Test 27) effects in the HUVEC wound healing
assay, 24 hour timepoint as depicted in FIG. 85.

| | |
|---|---|
| Additional Figure | % Wound Confluence |

| Label Information | Figure Labels | 24 Hour Timepoint |
|---|---|---|
| Positive Control | Complete | 89.33 |
| | *9 (Test 27) 2.8x | 51.70 |
| | *9 (Test 27) 2x | 41.48 |
| | Control EV | 41.29 |
| | *9 (Test 27) 1x | 39.35 |
| | *9 (Test 27) 0.75x | 27.61 |
| | *9 (Test 27) 0.25x | 21.67 |
| | *9 (Test 27) 0.5x | 21.66 |
| Negative Control | Poor | 13.22 |

FIG. 86 depicts the results for the 18-hour time timepoint. The 18-hour timepoint value for the negative control was used as a baseline. The depicted values were obtained by subtracting the negative control value at the 18-hour time point (baseline subtraction) and then normalizing the remainder to the baseline-subtracted result for the positive control at the 18-hour time point. This method of baseline subtraction and normalization to the positive control is referred to as ‘double normalization’ and the resulting values are ‘double normalized’. The results depicted in FIG. 86 show that all tested doses showed an improvement in scratch wound healing over the negative control, where the highest dose resulted in the greatest scratch wound healing, as measured by normalized wound confluence. The highest dose tested, 2.8×, attained 42% relative wound confluence after 18 hours.

### Example 37.2

### In Vitro Analysis of the Potency of CTC1-EV in a Cardiomyocyte Survival Assay

An in vitro cardiomyocyte survival assay was performed as described in Example 11. Briefly, iCell Cardiomyocytes2 were exposed to iCMM with NucSpot Live 650 dye (Biotium, ref: 40082) (this served as a viable cell control, “Complete Media Control”; positive control); or to iCMM with NucSpot Live 650 dye, and staurosporine (Abcam, ref: ab146588) at a final in-well concentration of 2 μM (this also served as an apoptotic cell control; “Stress” control; negative control). Dye, PBS, and DMSO concentrations, and final well volumes, were equivalent in all wells. Cells were cultured in these pre-incubation media for four hours. After this incubation, the pre-incubation media was removed, and the wells were rinsed with iCMM. Cells were then fed with iCMM with NucSpot Live 650 dye and PBS, or iCMM with NucSpot Live 650 dye supplemented with increasing concentrations of *9 (Test 27), while maintaining PBS final volumes, where 1× was 5.79 uL, which was the *9 (Test 27) sample volume derived from 150,000 mother cells (secreting cells).

Cells were detected in the assay using Incucyte image analysis, which counts the number of live cells, identified by NucLight Red staining. Dead cells were gated out of the analysis. Dead cells were identified by very bright red staining and a shrunken appearance. Viable cells were detected by the software through a masking process which identifies live cells based on intensity of red staining and cell size and morphology. The greater the % Positive NucLight Red cells, the greater the survival of cells in this assay. To account for any well-to-well variation in cell seeding, each well was normalized to its time 0 cell count, which is the first image taken after adding the NucLight Red dye. This dye is compatible with cell culture. Images were taken during the assay phase to generated the time-course shown in FIG. 87.

In FIG. 87, the top most data (dotted line) are for the Complete Media, no stress, positive control (“Complete Media (positive control)”). The results show that the cell viability is maintained throughout the assay period at or around 100%. At the 24-hour timepoint, the % Positive Nuclight Red is at 98% (98% survival). The dashed line is for the stress control, the negative control, the “Staurosporine (negative control)”. The result at the 24-hour timepoint is 78.5% survival. The Control EV, which is produced by ultracentrifugation of an MSC-conditioned media, is the dotted-dashed line. The result at 24-hours is a survival of 86% as shown in FIG. 87. The *9 (Test 27) sample results are shown in the solid lines. The dose used for each condition is given in the labels to the right of the graph. The 1× dose resulted in about the same survival as the negative control (0.4 percentage points lower than the negative control). Both the 2× and 2.6× doses resulted in improved cardiomyocyte survival, with the 2.6× dose having the highest survival at 24-hours, which was 81.9% survival, which is a 5% improvement in cell survival as compared to the negative control at this timepoint. TABLE 52 shows the 24 hour data points for FIG. 87.

TABLE 52
*9 (Test 27) effects in the cardiomyocyte survival
assay, 24 hour timepoint as depicted in FIG. 87.

| | |
|---|---|
| Additional Figure | % Positive NucLight Red; Normalized to T0 |

| Label Information | Figure Labels | 24 Hour Timepoint |
|---|---|---|
| Positive Control | Complete Media | 0.978 |
| | Control EV | 0.860 |
| | *9 (Test 27) 2.6x | 0.819 |
| | *9 (Test 27) 2x | 0.800 |
| Negative Control | Staurosporine | 0.785 |
| | *9 (Test 27) 1x | 0.781 |

Thus, the results demonstrate that the *9 (Test 27) sample improved the survival of stressed cardiomyocytes throughout the 24-hour time course.

FIG. 88 depicts the results at the 24-hour timepoint. The results from FIG. 87 were baseline (negative control) subtracted and normalized to the positive control. Both the 2× and 2.6× doses of *9 (Test 27) showed a beneficial effect on the survival of stressed cardiomyocytes. At the 2.6× dose, *9 (Test 27) improved relative cardiomyocyte survival by 18 percentage points over the negative control. Increasing doses of *9 (Test 27) increased the positive effect on cardiomyocyte survival. This result predicts that CTC1-EV (*9 (Test 27)) Test Example 27 CTC1-EV Final Formulation (sample *9) will promote the survival of cardiomyocytes in failing hearts, such as in human subjects in heart failure.

Thus, the results demonstrate that the *9 (Test 27) sample improved the survival of stressed cardiomyocytes at the 24-hour timepoint.

### Example 38

### In Vitro Analysis of the Potency of CTC1-EV in a Scratch Wound Healing Assay

To analyze the functionality and potency of CTC1-EV (*9 (Test 27) in this experiment), a second version of a HUVEC scratch wound healing assay was used. This assay was performed essentially as described in El Harane et al. (PMID: 29420830), where the controls were 1/HUVEC cultured in Complete Medium (Endothelial Cell Basal Media (PromoCell; catalog ref #: C-22210), supplemented with the Endothelial Cell Growth Medium Supplement Pack (PromoCell, ref: C-39210)); 2/HUVEC cultured in Poor medium (Endothelial Cell Basal Media (PromoCell; ref: C-22210); and 3/HUVEC cultured in Poor Medium supplemented with 5×10⁹ particles of FBS-EV. Four separate vials of *9 (Test 27) were tested at concentrations ranging from 1×10⁹ to 5×10⁹ particles per well, as measured by NTA. The results are depicted in FIG. 89.

Results show a strong and dose-dependent improvement in wound confluence compared to the Poor medium control for all CTC1-EV Final Formulation conditions tested. Specifically, when normalized to the Complete medium control (i.e., the wound confluence of the Complete medium control is set to 100%), the Poor medium control showed only 12.7% wound confluence. The FBS-EV control showed 81.4% wound confluence. Three vials were tested at 1×10⁹ particles per well dose. For all three, wound confluence was greater than 20.0%. Four vials were tested at 2×10⁹ particles per well dose. For all four, wound confluence was greater than 30.0%. Four vials were tested at 5×10⁹ particles per well dose. For all four, wound confluence was greater than 50.0%.

### Example 39

### CTC1-EV GLP Safety Testing

CTC1-EV (*9 (Test 27) in this experiment) was tested for toxicity in mice, for toxicity in rats, and for tumorigenicity in nude mice, using standard techniques and in accordance with regulatory requirements. Groups and conditions are given in Table 53.

TABLE 53
Summary of GLP safety studies on *9 (Test27).

| | | Number | | | | | |
|---|---|---|---|---|---|---|---|
| | | of animals | | | Route of | Maximum | |
| Study | Animal | exposed | Groups | Dose | administration | follow up | Examination |
| Toxicity | Rats | 5 males | 7-Exposed | 4 × 100 | IV | 14 days | Mortality, morbidity, |
| study | (Sprague | 5 females | (5 + 5) | particles/kg | | | clinical examination, |
| in rats | Dawley) | | 2-Control | (or 2.05 mL | | | functional and |
| | | | (DPBS; | DPBS/kg for | | | neurobehavioral tests, |
| | | | 5 + 5) | vehicle | | | weight, food |
| | | | | control) | | | consumption, ophthalmology, |
| | | | | On days 0, 2 | | | hematology and |
| | | | | and 4 | | | coagulation parameters, |
| | | | | | | | blood chemistry, urinalysis, |
| | | | | | | | necropsy examination, |
| | | | | | | | histopathology |
| Toxicity | Mice | 5 males | 1-Exposed | 4 × 1011 | IV | 14 days | Mortality, morbidity, |
| study | (BAL,B/c) | 5 females | (5 + 5) | particles/kg | | | clinical examination, |
| in mice | | | 2-Control | (or 2.05 mL | | | functional and |
| | | | (DPBS; | DPBS/kg for | | | neurobehavioral tests, |
| | | | 5 + 5) | vehicle | | | weight, food |
| | | | | control) | | | consumption, ophthalmology, |
| | | | | On days 0, 2 | | | hematology and |
| | | | | and 4 | | | coagulation parameters, |
| | | | | | | | blood chemistry, urinalysis, |
| | | | | | | | necropsy examination, |
| | | | | | | | histopathology |
| Tumorigenicity | Mice | 5 males | 1-Exposed | 4 × 101 | Subcutaneous | 91 days | Mortality, morbidity, |
| study in mice | (BALB/ | 5 females | (5 + 5) | particles/kg | | | clinical examination, |
| | cNude J) | | 2-Negative | (or 2.05 ml | | | tumor examination, |
| | | | control | DPBS/kg for | | | weight, food |
| | | | (DPBS; | vehicle | | | consumption, hematology, |
| | | | 5 + 5) | control | | | blood chemistry, |
| | | | 3-Positive | or 107 HeLa | | | necropsy examination, |
| | | | control | cells for | | | histopathology |
| | | | (HeLa cells; | positive control | | | |
| | | | 5 + 5) | On day 1 | | | |

For CTC1-EV, *9 (Test 27), the study concluded the following:
Rats: Under the experimental conditions adopted, intravenous (bolus) administration of *9 (Test 27) on Days 0, 2 and 4 at 4×10¹¹ particles/kg to Sprague-Dawley rats, was well tolerated and did not induce any signs of toxicity.
Mice: Under the experimental conditions adopted, intravenous (bolus) administration of *9 (Test 27) on Days 0, 2 and 4 at 4×10¹¹ particles/kg to BALB/c mice, was well tolerated and did not induce any signs of toxicity throughout the treatment period.
Mice: Under the experimental conditions adopted, the *9 (Test 27) administered once by the subcutaneous route to male and female nude BALB/c mice induced no tumors during the 91-days observation period.

The data that led to the conclusions described in TABLE 53 are given in TABLE 54 through to TABLE 68.

TABLE 54
14-Day Intravenous Toxicity Study in Mice;
Body Weight by Animal Over Time.

| Body Weight (g) | Animal nº | D0 | D3 | D6 | D10 | D13 |
|---|---|---|---|---|---|---|
| Male- | 1 | 24.3 | 25.3 | 23.4 | 24.9 | 24.8 |
| group 1 (control) | 2 | 26.6 | 27.6 | 27.1 | 28.0 | 28.6 |
| | 3 | 24.4 | 25.5 | 25.2 | 25.9 | 26.2 |
| | 4 | 23.4 | 23.7 | 23.7 | 24.1 | 24.4 |
| | 5 | 26.1 | 26.6 | 26.6 | 26.9 | 27.3 |
| Male- | 1 | 24.9 | 25.0 | 25.3 | 26.5 | 26.4 |
| group 2 (EV) | 2 | 27.1 | 28.3 | 28.2 | 29.1 | 29.2 |
| | 3 | 27.8 | 27.3 | 27.8 | 28.6 | 29.1 |
| | 4 | 24.9 | 25.2 | 25.7 | 26.6 | 25.6 |
| | 5 | 24.4 | 24.5 | 25.6 | 26.1 | 26.1 |
| Female- | 1 | 19.3 | 19.0 | 19.8 | 19.3 | 18.9 |
| group 1 (control) | 2 | 19.5 | 19.3 | 20.6 | 20.4 | 20.3 |
| | 3 | 20.7 | 21.5 | 20.5 | 21.0 | 21.3 |
| | 4 | 18.4 | 19.2 | 19.1 | 19.7 | 19.5 |
| | 5 | 18.3 | 18.5 | 18.4 | 19.0 | 19.9 |
| Female- | 1 | 18.2 | 18.6 | 19.0 | 18.8 | 19.0 |
| group 2 (EV) | 2 | 18.2 | 18.5 | 18.6 | 18.9 | 19.4 |
| | 3 | 19.0 | 19.3 | 19.8 | 19.6 | 20.1 |
| | 4 | 18.8 | 18.7 | 18.9 | 19.2 | 20.5 |
| | 5 | 18.8 | 19.3 | 19.1 | 20.3 | 20.6 |

Animal nº = Animal identification number.

TABLE 55
14-Day Intravenous Toxicity Study in Mice; Hematology and Coagulation by Animal.

| Hematology | | | | | | | | | |
|---|---|---|---|---|---|---|---|---|---|
| and | Animal | | | | | | | | |
| Coagulation | nº | WBC | RBC | HGB | HCT | THR | NEUT | EOSI | BASO |
| Male- | 1 | 6.0 | 10.1 | 16 | 51 | 953 | 1.22 | 0.14 | 0.01 |
| group 1 | 2 | 3.7 | 10.4 | 16.3 | 52 | 245 | 0.87 | 0.12 | 0.01 |
| (control) | 3 | 7.4 | 10.7 | 16.8 | 53 | 1036 | 1.41 | 0.2 | 0.02 |
| | 4 | 3.9 | 11.0 | 17.3 | 54 | 861 | 0.99 | 0.17 | 0 |
| | 5 | 7.3 | 11.1 | 17.5 | 54 | 1047 | 1.17 | 0.13 | 0.01 |
| Male- | 1 | 7.8 | 10.4 | 16.5 | 52 | 106 | 1.16 | 0.18 | 0.02 |
| group 2 | 2 | 6.8 | 10.3 | 15.9 | 51 | 1017 | 1.31 | 0.13 | 0.01 |
| (EV) | 3 | 5.6 | 10.5 | 16.3 | 53 | 1081 | 0.97 | 0.15 | 0.01 |
| | 4 | 6.8 | 10.9 | 17.2 | 54 | 186 | 1.37 | 0.14 | 0.01 |
| | 5 | 5.7 | 11.0 | 17 | 54 | 1076 | 1.03 | 0.2 | 0.01 |
| Female- | 1 | 7.9 | 10.2 | 16.4 | 49 | 058 | 1.08 | 0.2 | 0.03 |
| group 1 | 2 | 5.8 | 10.2 | 16.3 | 50 | 906 | 68 | 0.2 | 0.01 |
| (control) | 3 | 3.5 | 10.3 | 16 | 49 | 963 | 0.77 | 0.12 | 0 |
| | 4 | 2.5 | 10.61 | 16.5 | 51 | 1211 | 0.42 | 0.06 | 0.01 |
| | 5 | | | | | | | | |
| Female- | 1 | 6.3 | 10.6 | 16.8 | 52 | 1123 | 0.71 | 0.11 | 0.02 |
| group 2 | 2 | 7.7 | 10.5 | 16.2 | 51 | 146 | 1.42 | 0.21 | 0.01 |
| (EV) | 3 | 5.2 | 10.5 | 16.6 | 50 | 110 | 0.7 | 0.16 | 0.02 |
| | 4 | 4.2 | 10.2 | 16.3 | 49 | 178 | 0.85 | 0.17 | 0.01 |
| | 5 | 2.7 | 10.6 | 16.6 | 51 | 1111 | 0.54 | 0.1 | 0 |
| Hematology | | | | | | | | | |
| and | | | | | | | | | |
| Coagulation | LYMP | MONO | LUC | RET | RDW | MCV | MCH | MCHC | MPV |
| Male- | 4.5 | 0.08 | 0.04 | 289 | 13 | 50 | 15.7 | 31 | 5 |
| group 1 | 2.7 | 0.05 | 0.01 | 290 | 13.8 | 50 | 15.7 | 32 | 5.4 |
| (control) | 5.5 | 0.17 | 0.06 | 307 | 13.1 | 50 | 15.7 | 32 | 4.9 |
| | 2.7 | 0.03 | 0.02 | 324 | 13.5 | 49 | 15.8 | 32 | 5.1 |
| | 5.8 | 0.14 | 0.07 | 280 | 12.8 | 49 | 15.8 | 32 | 4.7 |
| Male- | 6.3 | 0.15 | 0.07 | 331 | 13.6 | 51 | 15.9 | 32 | 5.4 |
| group 2 | 5.1 | 0.16 | 0.04 | 383 | 13.2 | 49 | 15.5 | 32 | 5 |
| (EV) | 4.3 | 0.1 | 0.03 | 317 | 13.2 | 50 | 15.4 | 31 | 5 |
| | 5.2 | 0.08 | 0.05 | 244 | 12.8 | 49 | 15.8 | 32 | 5.2 |
| | 4.3 | 0.09 | 0.03 | 299 | 12.6 | 49 | 15.5 | 32 | 4.8 |
| Female- | 6.5 | 0.07 | 0.03 | 397 | 12.9 | 48 | 16.1 | 34 | 4.9 |
| group 1 | 4.7 | 0.1 | 0.06 | 348 | 12.8 | 49 | 16.1 | 33 | 4.9 |
| (control) | 2.5 | 0.04 | 0.0 | 260 | 12.8 | 48 | 15.6 | 32 | 4.8 |
| | 2 | 0.01 | 0.01 | 407 | 12.8 | 48 | 15.6 | 33 | 5.5 |
| Female- | 5.4 | 0.04 | 0.05 | 337 | 13.2 | 49 | 15.8 | 32 | 4.7 |
| group 2 | 5.9 | 0.06 | 0.05 | 461 | 13.3 | 49 | 15.5 | 32 | 4.9 |
| (EV) | 4.3 | 0.05 | 0.03 | 373 | 13 | 48 | 15.9 | 33 | 5.1 |
| | 3.4 | 0.03 | 0.01 | 309 | 12.9 | 48 | 15.9 | 33 | 5.3 |
| | 2.1 | 0.01 | 0.01 | 410 | 12.8 | 48 | 15.7 | 33 | 5.1 |

White blood cell count = WBC; Giga/L; Red blood cell count = RBC; Tera/L; Haemoglobin = HGB; g/100 mL; Haematocrit = HCT; %; Thrombocyte count = THR; Giga/L; Neutrophil count = NEUT; Giga/L; Eosinophil count = EOSI; Giga/L; Basophil count = BASO; Giga/L; Lymphocyte count = LYMP; Giga/L; Monocyte count = MONO; Giga/L; Large unstained cell count = LUC; Giga/L; Reticulocytes = RET; Giga/L; Red distribution width = RDW; %; Mean Corpuscular Volume = MCV; fL; Mean corpuscular haemoglobin = MCH; pg; Mean corpuscular haemoglobin concentration = MCHC; g/100 mL; Mean platelet volume = MPV; fL

TABLE 56
14-Day Intravenous Toxicity Study in Mice;
Clinical Chemistry by Animal.

| Clinical | Animal | | | | | |
|---|---|---|---|---|---|---|
| Chemistry | nº | ALT | AST | TBIL | CREA | UREA |
| Male- | 1 | 43 | 57 | 1.11 | 18 | 6.3 |
| group 1 | 2 | 94 | 118 | 1 | 20 | 8.2 |
| (control) | 3 | 32 | 59 | 1.88 | 17 | 5.8 |
| | 4 | 56 | 142.0 | 1.39 | 26 | 6.9 |
| | 5 | 50 | 119 | | 22 | 7.2 |
| Male- | 1 | 39 | 94 | 1 | 26 | 5.7 |
| group 2 | 2 | 178 | 124 | | 25 | 8.8 |
| (EV) | 3 | 45 | 76 | 1.16 | 23 | 12.1 |
| | 4 | 38 | 76 | 1.75 | 19 | 8.6 |
| | 5 | | | | | |
| Female- | 1 | | | | 19 | 6.2 |
| group 1 | 2 | | | | | 11.2 |
| (control) | 3 | 154 | 165 | | 23 | 8.4 |
| | 4 | 41 | 107 | 1.24 | 27 | 11.3 |
| | 5 | | | | | 8.2 |
| Female- | 1 | 32 | 53 | | 17 | 5.6 |
| group 2 | 2 | 30 | 97 | LLD | 21 | 7.3 |
| (EV) | 3 | 53 | | | 25 | 10.8 |
| | 4 | | | | | |
| | 5 | 41 | 75 | 2.36 | 21 | 10.1 |

Alanine aminotransferase = ALT; IU/L; Aspartate aminotransferase = AST; IU/L; Total Bilirubin = TBIL; μmol/L; Creatinine = CREA; μmol/L; Urea nitrogen = UREA; mmol/L; Lower Limit of Detection = LLD

TABLE 57
14-Day Intravenous Toxicity Study in Mice;
Food Consumption by Animal Over Time.

| Food Consumption | Ani- | | |
|---|---|---|---|
| (g)/Animal/Day | mals no | Week 1 | Week 2 |
| Male - group 1 (control) | 1 | 3.4 | 3.8 |
| | 2 | 4.3 | 5.1 |
| | 3 | 4.7 | 4.1 |
| | 4 | 3.9 | 3.5 |
| | 5 | 4.6 | 4.3 |
| Male - group 2 (EV) | 1 | 4.2 | 4.3 |
| | 2 | 4.4 | 4.8 |
| | 3 | 5.7 | 5.1 |
| | 4 | 4.0 | 3.6 |
| | 5 | 4.5 | 4.1 |
| Female - group 1 (control) | 1 | 2.9 | 2.8 |
| | 2 | | |
| | 3 | | |
| | 4 | | |
| | 5 | | |
| Female - group 2 (EV) | 1 | 3.0 | 3.0 |
| | 2 | | |
| | 3 | | |
| | 4 | | |
| | 5 | | |

TABLE 58
14-Day Intravenous Toxicity Study in Mice; Organ Weight by Animal.

| Organ | Animal | | | | | | | | | | | |
|---|---|---|---|---|---|---|---|---|---|---|---|---|
| Weight (g) | nº | Spleen | Liver | Kidneys | Adrenals | Testes | Epididymides | Thymus | Heart | Brain | Ovaries | Uterus |
| Male- | 1 | 0.0829 | 1.17 | 0.373 | 0.0068 | 0.206 | 0.0673 | 0.0368 | 0.194 | 0.388 | NA | NA |
| group 1 | 2 | 0.0899 | 1.5 | 0.431 | 0.01 | 0.188 | 0.0652 | 0.0559 | 0.174 | 0.405 | NA | NA |
| (control) | 3 | 0.0944 | 1.22 | 0.443 | 0.0072 | 0.05 | 0.0456 | 0.0394 | 0.21 | 0.395 | NA | NA |
| | 4 | 0.068 | 1.12 | 0.544 | 0.00525 | 0.192 | 0.0685 | 0.0317 | 0.178 | 0.385 | NA | NA |
| | 5 | 0.0789 | 1.35 | 0.479 | 0.0028 | 0.219 | 0.0728 | 0.0366 | 0.164 | 0.395 | NA | NA |
| Male- | 1 | 0.0826 | 1.33 | 0.429 | 0.0115 | 0.185 | 0.0718 | 0.034 | 0.16 | 0.373 | NA | NA |
| group 2 | 2 | 0.0906 | 1.45 | 0.485 | 0.0046 | 0.2 | 0.0748 | 0.0473 | 0.169 | 0.403 | NA | NA |
| (EV) | 3 | 0.0955 | 1.57 | 0.464 | 0.0034 | 0.212 | 0.0651 | 0.0513 | 0.204 | 0.418 | NA | NA |
| | 4 | 0.0624 | 1.2 | 0.415 | 0.0048 | 0.19 | 0.0584 | 0.0279 | 0.145 | 0.339 | NA | NA |
| | 5 | 0.0822 | 1.19 | 0.432 | 0.0053 | 0.191 | 0.068 | 0.045 | 0.139 | 0.392 | NA | NA |
| Female- | 1 | 0.083 | 0.846 | 0.241 | 0.0052 | NA | NA | 0.0564 | 0.12 | 0.379 | 0.0098 | 0.0895 |
| group 1 | 2 | 0.0748 | 0.889 | 0.255 | 0.0065 | NA | NA | 0.0592 | 0.117 | 0.385 | 0.0124 | 0.0865 |
| (control) | 3 | 0.0762 | 0.996 | 0.285 | 0.0075 | NA | NA | 0.0576 | 0.116 | 0.373 | 0.0125 | 0.1506 |
| | 4 | 0.0713 | 0.862 | 0.262 | 0.0062 | NA | NA | 0.0574 | 0.103 | 0.381 | 0.0102 | 0.1181 |
| | 5 | 0.0811 | 0.905 | 0.253 | 0.0075 | NA | NA | 0.0488 | 0.112 | 0.382 | 0.0117 | 0.1522 |
| Female- | 1 | 0.0819 | 0.931 | 0.238 | 0.0106 | NA | NA | 0.0522 | 0.146 | 0.381 | 0.0164 | 0.05757 |
| group 2 | 2 | 0.11 | 0.909 | 0.253 | 0.0078 | NA | NA | 0.0833 | 0.11 | 0.386 | 0.0214 | 0.1179 |
| (EV) | 3 | 0.0785 | 0.862 | 0.25 | 0.01 | NA | NA | 0.0611 | 0.129 | 0.379 | 0.013 | 0.0781 |
| | 4 | 0.0919 | 0.949 | 0.238 | 0.01 | NA | NA | 0.0727 | 0.113 | 0.37 | 0.015 | 0.096 |
| | 5 | 0.068 | 0.897 | 0.263 | 0.0084 | NA | NA | 0.0472 | 0.124 | 0.375 | 0.0162 | 0.0749 |

TABLE 59
14-day Intravenous Toxicity Study in Rat;
Body Weight by Animal Over Time.

| Body | Animal | | | | | |
|---|---|---|---|---|---|---|
| Weight (g) | nº | D0 | D3 | D6 | D10 | D13 |
| Male- | 1 | 214.3 | 242.6 | 266.5 | 287.0 | 316.9 |
| group 1 | 2 | 230.4 | 270.7 | 294.4 | 323.2 | 337.6 |
| (control) | 3 | 211.0 | 236.2 | 254.3 | 271.7 | 287.0 |
| | 4 | 207.3 | 230.4 | 240.9 | 259.8 | 279.7 |
| | 5 | 197.7 | 231.3 | 253.1 | 283.1 | 307.6 |
| Male- | 1 | 210.6 | 242.1 | 267.6 | 290.2 | 304.7 |
| group 2 | 2 | 213.9 | 245.0 | 273.2 | 294.5 | 312.9 |
| (EV) | 3 | 240.2 | 272.8 | 312.5 | 348.2 | 367.9 |
| | 4 | 227.1 | 259.7 | 279.3 | 309.6 | 338.1 |
| | 5 | 201.6 | 227.3 | 249.0 | 269.6 | 287.6 |
| Female- | 1 | 187.1 | 199.0 | 201.0 | 212.1 | 219.2 |
| group 1 | 2 | 181.0 | 189.2 | 193.1 | 204.0 | 208.7 |
| (control) | 3 | 194.7 | 206.3 | 211.1 | 217.1 | 222.7 |
| | 4 | 212.2 | 228.6 | 233.8 | 238.3 | 237.2 |
| | 5 | 183.4 | 194.4 | 207.2 | 219.6 | 219.9 |
| Female- | 1 | 180.5 | 188.3 | 191.4 | 201.1 | 205.9 |
| group 2 | 2 | 190.8 | 206.7 | 219.2 | 227.8 | 239.8 |
| (EV) | 3 | 197.7 | 208.2 | NA | NA | NA |
| | 4 | 183.7 | 200.7 | 205.5 | 213.6 | 225.8 |
| | 5 | 202.5 | 213.8 | 220.7 | 224.9 | 234.9 |

TABLE 60
14-day Intravenous Toxicity Study in Rat;
Hematology and Coagulation by Animal.

| Hematology | | | | | | | | | | |
|---|---|---|---|---|---|---|---|---|---|---|
| and | Animal | | | | | | | | | |
| Coagulation | nº | WBC | RBC | HGB | HCT | THR | NEUT | EOSI | BASO | LYMP |
| Male- | 1 | 7.9 | 7.5 | 15.7 | 47 | 1354 | 1.02 | 0.05 | 0.02 | 6.6 |
| group 1 | 2 | 6.6 | 7.5 | 16.0 | 48 | 1273 | 0.91 | 0.06 | 0.01 | 5.4 |
| (control) | 3 | 8.6 | 8.3 | 16.4 | 49 | 1079 | 1.24 | 0.19 | 0.02 | 7 |
| | 4 | 6.1 | 7.8 | 15.8 | 47 | 1138 | 0.96 | 0.03 | 0.01 | 4.8 |
| | 5 | 7.8 | 7.2 | 15.1 | 45 | 1199 | 1.11 | 0.07 | 0.02 | 6.3 |
| Male- | 1 | 7.2 | 7.4 | 15.2 | 46 | 1302 | 0.87 | 0.05 | 0.02 | 6 |
| group 2 | 2 | 7.6 | 7.8 | 15.9 | 48 | 1263 | 0.92 | 0.05 | 0.02 | 6.3 |
| (EV) | 3 | 6.8 | 7.1 | 15.3 | 47 | 1061 | 0.89 | 0.03 | 0.02 | 5.6 |
| | 4 | 5.5 | 7.5 | 15.7 | 47 | 1297 | 0.81 | 0.04 | 0.01 | 4.3 |
| | 5 | 7.6 | 7.9 | 16.3 | 49 | 1278 | 1.38 | 0.04 | 0.03 | 6 |
| Female- | 1 | 6.1 | 8.0 | 16.5 | 48 | 866 | 0.77 | 0.13 | 0.01 | 4.9 |
| group 1 | 2 | 6.1 | 7.8 | 15.5 | 46 | 1307 | 0.97 | 0.09 | 0.02 | 4.7 |
| (control) | 3 | 5.2 | 7.8 | 15.7 | 45 | 124 | 0.73 | 0.06 | 0.01 | 4.2 |
| | 4 | 9.0 | 7.7 | 16.4 | 48 | 164 | 0.62 | 0.06 | 0.01 | 2.5 |
| | 5 | 3.4 | 8.0 | 15.7 | 46 | 1115 | 0.69 | 0.1 | 0.03 | 7.9 |
| Female- | 1 | 5.3 | 7.8 | 15.4 | 45 | 1213 | 0.86 | 0.1 | 0 | 4.2 |
| group 2 | 2 | 5.2 | 8.3 | 15.9 | 47 | 1272 | 0.57 | 0.05 | 0.01 | 4.4 |
| (EV) | 3 | 5.6 | 6.1 | 12.6 | 41 | 877 | 1.04 | 0.08 | 0.01 | 4.3 |
| | 4 | 4.5 | 8.0 | 16.0 | 47 | 1258 | 0.38 | 0.03 | 0.01 | 4 |
| | 5 | 6.4 | 8.1 | 16.6 | 48 | 1066 | 0.56 | 0.06 | 0.02 | 5.5 |
| Hematology | | | | | | | | | | |
| and | | | | | | | | | | |
| Coagulation | MONO | LUC | RET | RDW | MCV | MCH | MCHC | MPV | APTT | PT |
| Male- | 0.16 | 0.05 | 272 | 1.2 | 63 | 21 | 33 | 6.9 | 20.2 | 118.3 |
| group 1 | 0.18 | 0.05 | 226 | 11.3 | 64 | 21.2 | 33 | 6.5 | 19.5 | 19.4 |
| (control) | 0.19 | 0.03 | 216 | 10.7 | 60 | 19.9 | 33 | 6.4 | 19 | 18.9 |
| | 0.25 | 0.05 | 217 | 11.7 | 61 | 20.2 | 33 | 6.7 | 21.7 | 17.5 |
| | 0.24 | 0.04 | 297 | 11.4 | 63 | 20.9 | 33 | 6.4 | 21.3 | 18.6 |
| Male- | 0.2 | 0.09 | 214 | 11.3 | 63 | 20.5 | 33 | 6.3 | 20 | 17.9 |
| group 2 | 0.23 | 0.05 | 284 | 11.1 | 61 | 20.2 | 33 | 6.5 | 16.6 | 18.4 |
| (EV) | 0.18 | 0.05 | 293 | 11.6 | 65 | 21.4 | 33 | 6.6 | 18.1 | 16.7 |
| | 0.27 | 0.04 | 266 | 11 | 63 | 21 | 33 | 6.4 | 17.3 | 17.7 |
| | 0.18 | 0.04 | 234 | 11 | 62 | 20.6 | 34 | 6.3 | 19.8 | 18.3 |
| Female- | 0.19 | 0.02 | 142 | 11 | 60 | 20.8 | 35 | 6.8 | 21.7 | 18 |
| group 1 | 0.23 | 0.04 | 137 | 10.1 | 59 | 19.8 | 34 | 6.4 | 20.5 | 18.5 |
| (control) | 0.17 | 0.05 | 124 | 10.1 | 58 | 20.1 | 35 | 6.6 | 19 | 17.5 |
| | 0.22 | 0.02 | 95 | 10 | 57 | 19.8 | 35 | 6.7 | 16.9 | 18.1 |
| | 0.17 | 0.07 | 143 | 10.5 | 62 | 21.5 | 35 | 6.8 | 17.1 | 18.1 |
| Female- | 0.11 | 0.03 | 110 | 10.4 | 57 | 19.7 | 34 | 6.4 | 19 | 19.1 |
| group 2 | 0.14 | 0.04 | 175 | 10.6 | 57 | 19.2 | 34 | 6.2 | 14.8 | 17.9 |
| (EV) | 0.14 | 0.05 | 250 | 1.6 | 67 | 20.7 | 31 | 7.7 | | |
| | 0.08 | 0.04 | 189 | 10.4 | 59 | 20.1 | 34 | 6.5 | 16.4 | 18 |
| | 0.24 | 0.04 | 183 | 9.8 | 60 | 20.6 | 35 | 6.5 | 18.8 | 19.4 |

White blood cell count = WBC; Giga/L; Red blood cell count = RBC; Tera/L; Haemoglobin = HGB; g/100 mL; Haematocrit = HCT; %; Thrombocyte count = THR; Giga/L; Neutrophil count = NEUT; Giga/L; Eosinophil count = EOSI; Giga/L; Basophil count = BASO; Giga/L; Lymphocyte count = LYMP; Giga/L; Monocyte count = MONO; Giga/L; Large unstained cell count = LUC; Giga/L; Reticulocytes = RET; Giga/L; Red distribution width = RDW; %; Mean Corpuscular Volume = MCV; IL; Mean corpuscular haemoglobin = MCH; pg; Mean corpuscular haemoglobin concentration = MCHC; g/100 mL; Mean platelet volume = MPV; fL; Activated partial thromboplastin time = APTT; sec; Prothrombin time = PT; sec

TABLE 61
4-day Intravenous Toxicity Study in Rat; Blood Clinical Chemistry by Animal.

| Blood | | | | | | | | | | |
|---|---|---|---|---|---|---|---|---|---|---|
| Clinical | Animal | | | | | | | | | |
| Chemistry | n° | ALT | AST | ALP | TBIL | PROT | CREA | UREA | GLU | CHOL |
| Male- | 1 | 28 | 87 | 183 | 1.22 | 56 | 24 | 2.8 | 7.3 | 1.32 |
| group 1 | 2 | 32 | 89 | 257 | 1.66 | 56 | 38 | 4.0 | 11.1 | 1.32 |
| (control) | 3 | 30 | 93 | 283 | 1.30 | 56 | 31 | 3.0 | 4.9 | 1.36 |
| | 4 | 33 | 89 | 242 | 1.48 | 57 | 32 | 3.5 | 4.5 | 1.44 |
| | 5 | 35 | 83 | 259 | 1.24 | 58 | 31 | 3.1 | 4.5 | 1.43 |
| Male- | 1 | 36 | 87 | 162 | 0.79 | 58 | 23 | 2,2 | 4.7 | 1.43 |
| group 2 (EV) | 2 | 29 | 83 | 214 | 1.69 | 56 | 36 | 3.3 | 4.0 | 1.81 |
| | 3 | 27 | 89 | 234 | 1.38 | 59 | 31 | 3.3 | 5.9 | 1.51 |
| | 4 | 34 | 94 | 209 | 1.07 | 61 | 34 | 3.0 | 5.2 | 1.87 |
| | 5 | 26 | 90 | 183 | 1.15 | 56 | 32 | 3.4 | 4.9 | 1.70 |
| Female- | 1 | 23 | 86 | 107 | 1.76 | 57 | 41 | 3.9 | 5.3 | 1.90 |
| group 1 | 2 | 24 | 74 | 95 | 1.54 | 64 | 46 | 3.8 | 6.0 | 1.60 |
| (control) | 3 | 2.2 | 84 | 127 | 2.17 | 66 | 42 | 3.9 | 5.6 | 1.53 |
| | 4 | 23 | 75 | 153 | 1.45 | 59 | 43 | 4.1 | 5.9 | 1.88 |
| | 5 | 25 | 91 | 108 | 1.29 | 64 | 39 | 3.0 | 6.2 | 1.22 |
| Female- | 1 | 24 | 77 | 171 | 1.51 | 60 | 39 | 4.1 | 5.7 | 1.57 |
| group 2 (EV) | 2 | 28 | 89 | 142 | 1.58 | 59 | 42 | 3.3 | 6.8 | 1.76 |
| | 3 | 40 | 142 | 244 | 2.13 | 44 | 46 | 3.5 | 16.26 | 1.33 |
| | 4 | 21 | 83 | 186 | 1.75 | 60 | 40 | 3.3 | 7.5 | 1.98 |
| | 5 | 26 | 79 | 171 | 1.80 | 62 | 42 | 5.0 | 5.9 | 1.31 |
| Blood | | | | | | | | | | |
| Clinical | | | | | | | | | | |
| Chemistry | CI | K | Na | ALB | TRIG | Ca | P | GLOB | A/G | |
| Male- | 98 | 3.6 | 146 | 30 | 0.52 | 2.7 | 2.8 | 26 | 1.15 | |
| group 1 | 99 | 3.9 | 142 | 31 | 0.36 | 2.76 | 2.5 | 25 | 1.24 | |
| (control) | 102 | 4.0 | 143 | 30 | 0.38 | 2.67 | 3.12 | 26 | 1.15 | |
| | 101 | 3.8 | 145 | 30 | 0.88 | 2.79 | 3.01 | 27 | 1.11 | |
| | 101 | 3.6 | 145 | 32 | 0.58 | 2.74 | 2.9 | 26 | 1.23 | |
| Male- | 101 | 3.7 | 143 | 31 | 0.55 | 2.71 | 2.72 | 27 | 1.15 | |
| group 2 (EV) | 102 | 3.7 | 144 | 30 | 0.42 | 2.68 | 2.94 | 26 | 1.15 | |
| | 100 | 4.0 | 143 | 32 | 0.79 | 2.86 | 3.03 | 27 | 1.19 | |
| | 100 | 3.8 | 144 | 31 | 0.46 | 2.85 | 2.96 | 30 | 1.03 | |
| | 101 | 3.8 | 145 | 29 | 0.39 | 2.8 | 12.96 | 27 | 1.07 | |
| Female- | 100 | 3.7 | 143 | 32 | 0.33 | 2.8 | 12.54 | 25 | 1.28 | |
| group 1 | 101 | 3.7 | 145 | 35 | 0.43 | 2.85 | 2.34 | 29 | 1.21 | |
| (control) | 102 | 3.7 | 145 | 35 | 0.35 | 2.79 | 2.11 | 31 | 1.13 | |
| | 101 | 4.0 | 144 | 35 | 0.54 | 2.72 | 2.43 | 29 | 1.21 | |
| | 101 | 4.4 | 144 | 33 | 0.32 | 2.82 | 2.61 | 26 | 1.27 | |
| Female- | 101 | 3.7 | 143 | 34 | 0.23 | 2.77 | 2.31 | 26 | 1.31 | |
| group 2 (EV) | 101 | 3.9 | 144 | 34 | 0.37 | 2.75 | 2.26 | 25 | 1.36 | |
| | 96.9 | 3.24 | 141 | 25 | 0.41 | 2.28 | 1.96 | 19 | 1.32 | |
| | 102 | 3.7 | 145 | 34 | 0.46 | 2.74 | 2.16 | 26 | 1.31 | |
| | 101 | 3.7 | 146 | 33 | 0.29 | 2.77 | 2.57 | 29 | 1.14 | |

Alanine aminotransferase = ALT; IU/L; Aspartate aminotransferase = AST; IU/L; Alkaline phosphatase = ALP; IU/L; Total Bilirubin = TBIL; μmol/L; Total Protein = PROT; g/L; Creatinine = CREA; umol/L; Urea nitrogen = UREA; mmol/L; Glucose = GLU; mmol/L; Total Cholesterol = CHOL; mmol/L; Chloride = Cl; mmol/L; Potassium = K; mmol/L; Sodium = Na; mmol/L; Albumin = ALB; g/L; Triglycerides = TRIG; mmol/L; Calcium = Ca; mmol/L; Phosphorus = P; mmol/L; Globulin (calculated) = GLOB; g/L; Ratio Albumin/Globulin = A/G (calculated)

TABLE 62
14-day Intravenous Toxicity Study in Rat;
Food Consumption by Animal Over Time.

| Food Consumption | Ani- | | |
|---|---|---|---|
| (g)/Animal/Day | mals no | Week 1 | Week 2 |
| Male - group 1 (control) | 1 | 25.7 | 26.7 |
| | 2 | | |
| | 3 | | |
| | 4 | | |
| | 5 | | |
| Male - group 2 (EV) | 1 | 27.9 | 28.6 |
| | 2 | | |
| | 3 | | |
| | 4 | | |
| | 5 | | |
| Female - group 1 (control) | 1 | 18.3 | 18.1 |
| | 2 | | |
| | 3 | | |
| | 4 | | |
| | 5 | | |
| Female - group 2 (EV) | 1 | 18.5 | 19.4 |
| | 2 | | |
| | 3 | | |
| | 4 | | |
| | 5 | | |

TABLE 63
14-day Intravenous Toxicity Study in Rat; Organ Weight by Animal.

| Organ | Animal | | | | | | | | | | | |
|---|---|---|---|---|---|---|---|---|---|---|---|---|
| Weight | nº | Spleen | Liver | Kidneys | Adrenals | Testes | Epididymides | Thymus | Heart | Brain | Ovaries | Uterus |
| Male- | 1 | 0.608 | 8.87 | 2.38 | 0.0537 | 3.13 | 0.662 | 0.562 | 1.36 | 2.06 | | |
| group 1 | 2 | 0.671 | 9.23 | 2.15 | 0.0512 | 3.06 | 0.659 | 0.673 | 1.32 | 1.93 | | |
| (control) | 3 | 0.53 | 7.48 | 2.4 | 0.038 | 2.96 | 0.6 | 0.596 | 1.46 | 2.08 | | |
| | 4 | 0.596 | 7.32 | 1.98 | 0.0528 | 2.87 | 0.645 | 0.733 | 1.09 | 1.99 | | |
| | 5 | 0.519 | 9.15 | 2.57 | 0.0474 | 3.16 | 0.686 | 0.547 | 1.15 | 1.97 | | |
| Male- | 1 | 0.541 | 8.56 | 2.3 | 0.0447 | 3.26 | 0.735 | 0.465 | 1.15 | 1.95 | | |
| group 2 | 2 | 0.588 | 8.22 | 2.37 | 0.0508 | 3.1 | 0.695 | 0.719 | 1.19 | 2.06 | | |
| (EV) | 3 | 0.839 | 10.7 | 2.68 | 0.0572 | 2.91 | 0.562 | 0.685 | 1.43 | 2.12 | | |
| | 4 | 0.65 | 9.14 | 2.43 | 0.0493 | 3.44 | 0.633 | 0.642 | 1.45 | 2.11 | | |
| | 5 | 0.554 | 8.19 | 2.17 | 0.0406 | 3.03 | 0.639 | 0.553 | 1.37 | 1.86 | | |
| Female- | 1 | 0.494 | 6.67 | 1.52 | 0.0542 | | | 0.532 | 1.006 | 1.88 | 0.1401 | 0.387 |
| group 1 | 2 | 0.4 | 5.82 | 1.49 | 0.0618 | | | 0.352 | 0.963 | 1.83 | 0.1034 | 0.321 |
| (control) | 3 | 0.453 | 6.78 | 1.64 | 0.0536 | | | 0.431 | 0.798 | 1.84 | 0.126 | 0.464 |
| | 4 | 0.581 | 7.33 | 1.91 | 0.0824 | | | 0.672 | 0.867 | 1.93 | 0.1281 | 0.384 |
| | 5 | 0.431 | 7.02 | 1.88 | 0.0608 | | | 0.417 | 0.97 | 1.84 | 0.1175 | 0.388 |
| Female- | 1 | 0.363 | 5.66 | 1.42 | 0.0534 | | | 0.406 | 0.726 | 1.9 | 0.0872 | 0.385 |
| group 2 | 2 | 0.483 | 7.27 | 1.61 | 0.0583 | | | 0.569 | 0.953 | 1.86 | 0.1236 | 0.731 |
| (EV) | 3 | 0.52 | 7.94 | 1.59 | 0.0503 | | | 0.713 | 1.147 | 1.72 | 0.0652 | 0.367 |
| | 4 | 0.576 | 7.07 | 1.59 | 0.067 | | | 0.503 | 0.923 | 1.82 | 0.1025 | 0.562 |
| | 5 | 0.567 | 7.28 | 1.75 | 0.079 | | | 0.524 | 0.863 | 1.89 | 0.16 | 0.377 |

TABLE 64
14-day Intravenous Toxicity Study in Rat; Urinalysis by Animal.

| | | | | | | | | Blood: | Leukocytes: | |
|---|---|---|---|---|---|---|---|---|---|---|
| | | | | | | | | Red | White | |
| | | | | | | | | Blood | Blood | |
| | Animal | pH | Proteins | Glucose | Ketones | Urobilinogen | | Cell | Cell | Specific |
| Urinalysis | nº | value | (g/L) | (g/L) | (g/L) | (mg/dL) | Bilirubin | Count/μL | Count/μL | Gravity |
| Male- | 1 | 6.5 | 0.3 | 0 | 0.15 | 0.2 | 0 | 0 | 0 | 1.02 |
| group 1 | 2 | 7 | 0 | 0 | 0.15 | 0.2 | 0 | 0 | 0 | 1.02 |
| (control) | 3 | 7 | 0.3 | 0 | 0.15 | 0.2 | 0 | 0 | 0 | 1.02 |
| | 4 | 7 | 0 | 0 | 0 | 0.2 | 0 | 0 | 0 | 1.01 |
| | 5 | 7 | 0 | 0 | 0.15 | 0.2 | 0 | 0 | 0 | 1.015 |
| Male- | 1 | 6 | 0 | 0 | 0.15 | 0.2 | 0 | 0 | 0 | 1.02 |
| group 2 | 2 | 7.5 | 0 | 0 | 0.15 | 0.2 | 0 | 0 | 0 | 1.015 |
| (EV) | 3 | 7 | 0 | 0 | 0 | 0.2 | 0 | 0 | 0 | 1.02 |
| | 4 | 6.5 | 0 | 0 | 0 | 0.2 | 0 | 0 | 0 | 1.015 |
| | 5 | 7 | 0 | 0 | 0 | 0.2 | 0 | 0 | 0 | 1.015 |
| Female- | 1 | 6 | 0 | 0 | 0 | 0.2 | 0 | 0 | 0 | 1.02 |
| group 1 | 2 | 6.5 | 0 | 0 | 0 | 0.2 | 0 | 0 | 0 | 1.02 |
| (control) | 3 | 6.5 | 0 | 0 | 0 | 0.2 | 0 | 0 | 0 | 1.02 |
| | 4 | 6.5 | 0 | 0 | 0 | 0.2 | 0 | 0 | 0 | 1.02 |
| | 5 | 6.5 | 0 | 0 | 0 | 0.2 | 0 | 0 | 0 | 1.02 |
| Female- | 1 | 6.5 | 0 | 0 | 0 | 0.2 | 0 | 0 | 0 | 1.02 |
| group 2 | 2 | 6.5 | 0 | 0 | 0 | 0.2 | 0 | 0 | 0 | 1.025 |
| (EV) | 3 | | | | | | | | | |
| | 4 | 7.5 | | 0 | 0 | 0.2 | 0 | 0 | 0 | 1.015 |
| | 5 | 6 | 0 | 0 | 0 | 0.2 | 0 | 0 | 0 | 1.025 |

TABLE 65
14-day Tumorgenicity Study in Nude Mice; Body Weight by Animal Over Time.

| Body | Animal | D1 | D4 | D7 | | | | | | | | | | | | |
|---|---|---|---|---|---|---|---|---|---|---|---|---|---|---|---|---|
| Weight (g) | nº | Randomization | Predose | Dosing | D14 | D21 | D28 | D35 | D42 | D49 | D56 | D63 | D70 | D77 | D84 | D90 |
| Male- | 1 | 18.6 | 20.0 | 21.1 | | | | | | | | | | | | |
| group 1 | 2 | 19.2 | 20.9 | 22.3 | | | | | | | | | | | | |
| (HeLa) | 3 | 17.6 | 18.6 | 20.6 | | | | | | | | | | | | |
| | 4 | 14.0 | 15.0 | 17.3 | | | | | | | | | | | | |
| | 5 | 16.6 | 17.4 | 19.8 | | | | | | | | | | | | |
| Female- | 1 | 14.3 | 14.8 | 16.1 | | | | | | | | | | | | |
| group 1 | 2 | 15.2 | 15.8 | 17.3 | | | | | | | | | | | | |
| (HeLa) | 3 | 14.5 | 15.7 | 16.1 | | | | | | | | | | | | |
| | 4 | 13.9 | 14.9 | 16.2 | | | | | | | | | | | | |
| | 5 | 16.0 | 16.7 | 18.2 | | | | | | | | | | | | |
| Male- | 1 | 15.6 | 17.3 | 18.9 | 19.8 | 20.8 | 21.5 | 21.8 | 22.6 | 23.3 | 23.2 | 23.7 | 23.9 | 24.3 | 24.5 | 23.7 |
| group 2 | 2 | 18.4 | 19.8 | 20.7 | 21.5 | 22.6 | 23.3 | 23.6 | 23.5 | 24.7 | 24.8 | 24.8 | 25.1 | 25.4 | 25.6 | 26.1 |
| (PBS) | 3 | 18.7 | 20.4 | 22.2 | 22.3 | 23.1 | 24.1 | 23.9 | 24.0 | 24.6 | 24.9 | 24.9 | 24.6 | 25.0 | 26.2 | 26.1 |
| | 4 | 17.3 | 18.0 | 20.8 | 22.3 | 22.8 | 23.3 | 23.8 | 23.5 | 25.0 | 25.5 | 24.4 | 25.3 | 25.8 | 25.8 | 26.1 |
| | 5 | 17.0 | 18.0 | 20.7 | 21.4 | 22.0 | 22.1 | 22.7 | 22.7 | 24.2 | 23.8 | 24.4 | 25.2 | 25.5 | 25.7 | 25.8 |
| Female- | 1 | 15 | 14.8 | 16.4 | 17.7 | 18.4 | 18.9 | 20.3 | 19.7 | 21.3 | 21.1 | 21.5 | 21.8 | 22.4 | 21.8 | 22.5 |
| group 2 | 2 | 14.3 | 16.2 | 17.6 | 17.8 | 19.1 | 19.3 | 20.5 | 21.2 | 20.8 | 21.1 | 21.9 | 21.7 | 22.1 | 21.9 | 20.9 |
| (PBS) | 3 | 13.8 | 14.4 | 15.8 | 16.9 | 17.6 | 18.1 | 18.2 | 19 | 19.4 | 19.2 | 19.8 | 19.8 | 20.1 | 20.1 | 21.1 |
| | 4 | 15.4 | 15.7 | 16.1 | 18.5 | 19.2 | 19.5 | 20.8 | 20.9 | 20.8 | 21.6 | 21.8 | 22.2 | 22.5 | 23.0 | 23.3 |
| | 5 | 14.4 | 15.2 | 17.7 | 17.9 | 18.9 | 20.1 | 20.3 | 20.6 | 20.7 | 21.6 | 21.4 | 21.9 | 22.0 | 22.4 | 22.6 |
| Male- | 1 | 18.1 | 19.1 | 20.6 | 20.6 | 21.5 | 21.5 | 22.0 | 22.9 | 23.6 | 23.7 | 23.8 | 23.5 | 24.8 | 24.2 | 24.8 |
| group 3 | 2 | 16.5 | 17.8 | 19.9 | 21.2 | 21.7 | 22.5 | 23.6 | 23.9 | 25.1 | 24.6 | 25.2 | 25.4 | 26.2 | 26.0 | 26.3 |
| (EV) | 3 | 19.3 | 20.4 | 21.6 | 21.2 | 22.4 | 23.1 | 23.2 | 23.8 | 23.6 | 24.2 | 24.9 | 24.0 | 24.8 | 25.4\| | 25.4 |
| | 4 | 18.3 | 19.4 | 21.3 | 22.1 | 23.0 | 23.5 | 24.3 | 24.9 | 25.1 | 25.4 | 25.3 | 25.6 | 26.4 | 26.4 | 27.2 |
| | 5 | 17.0 | 19.0 | 21.5 | 21.1 | 23.0 | 23.3 | 23.7 | 24.7 | 24.9 | 24.6 | 25.1 | 25.5 | 26.6 | 26.7 | 27.4 |
| Female- | 1 | 15.3 | 15.6 | 17.8 | 18.4 | 18.8 | 19.9 | 21.3 | 21.2 | 21.4 | 22.2 | 22.4 | 22.3 | 22.3 | 22.5 | 23.1 |
| group 3 | 2 | 14.8 | 15.6 | 17.5 | 18.1 | 19.1 | 18.9 | 20.7 | 20.6 | 21.3 | 21.4 | 21.5 | 20.9 | 21.7 | 21.8 | 22.2 |
| (EV) | 3 | 13.4 | 14.3 | 16.5 | 17.6 | 18.7 | 19.4 | 19.9 | 20.0 | 20.2 | 20.9 | 21.3 | 21.5 | 21.5 | 21.9 | 21.7 |
| | 4 | 14.4 | 15.3 | 16.5 | 17.6 | 18.2 | 18.4 | 19.5 | 19.9 | 20.4 | 20.4 | 20.9 | 20.7 | 20.9 | 20.6 | 21.5 |
| | 5 | 14.2 | 15.8 | 17.5 | 18.1 | 19.0 | 19.6 | 20.5 | 20.6 | 22.0 | 21.9 | 22.1 | 22.3 | 22.6 | 22.5 | 22.9 |

TABLE 66
14-day Tumorgenicity Study in Nude Mice; Hematology by Animal.

| Hema- | Animal | | | | | | | | | | | | |
|---|---|---|---|---|---|---|---|---|---|---|---|---|---|
| tology | nº | WBC | RBC | HGB | HCT | THR | NEUT | EOSI | BASO | LYMP | MONO | LUC | RET |
| Male- | 1 | | | | | | | | | | | | |
| group 1 | 2 | | | | | | | | | | | | |
| (HeLa) | 3 | 2.5 | 10.1 | 16.4 | 52 | 1053 | 1.21 | 0.13 | 0.02 | 1.1 | 0.06 | 0.01 | 345 |
| | 4 | | | | | | | | | | | | |
| | 5 | 4.2 | 10.9 | 17.4 | 55 | 1187 | 1.81 | 0.11 | 0.01 | 2 | 0.17 | 0.04 | 392 |
| Female- | 1 | | | | | | | | | | | | |
| group 1 | 2 | 3.5 | 10.1 | 16.0 | 49 | 1042 | 1.55 | 0.07 | 0.0 | 1.8 | 0.04 | 0.02 | 183 |
| (HeLa) | 3 | 3.5 | 10.0 | 16.5 | 51 | 1132 | 1.30 | 0.06 | 0.01 | 2.1 | 0.03 | 0.04 | 301 |
| | 4 | | | | | | | | | | | | |
| | 5 | | | | | | | | | | | | |
| Male- | 1 | 6.9 | 10.2 | 15.9 | 50 | 1219 | 3.04 | 0.25 | 0.01 | 3.3 | 0.24 | 0.03 | 222 |
| group 2 | 2 | 2.3 | 10.1 | 15.9 | 51 | 1181 | 1.13 | 0.14 | 0.00 | 1 | 0.03 | 0.00 | 335 |
| (PBS) | 3 | 3.5 | 10.4 | 16.4 | 51 | 974 | 1.79 | 0.15 | 0.01 | 1.5 | 0.07 | 0.00 | 307 |
| | 4 | 2.3 | 10.2 | 15.8 | 51 | 1226 | 1.03 | 0.05 | 0.00 | 1.2 | 0.03 | 0.00 | 310 |
| | 5 | 3.3 | 10.6 | 16.6 | 52 | 1340 | 1.64 | 0.06 | 0.00 | 1.5 | 0.05 | 0.01 | 299 |
| Female- | 1 | | | | | | | | | | | | |
| group 2 | 2 | 8.9 | 10.8 | 17.1 | 54 | 1255 | 4.31 | 0.12 | 0.05 | 4.3 | 0.1 | 0.03 | 320 |
| (PBS) | 3 | 3.8 | 10.6 | 16.9 | 53 | 1222 | 2.59 | 0.09 | 0.01 | 1.1 | 0.02 | 0.01 | 321 |
| | 4 | 5.1 | 10.0 | 16.2 | 50 | 1070 | 3.18 | 0.1 | 0.01 | 1.7 | 0.13 | 0.01 | 198 |
| | 5 | 2.9 | 9.9 | 15.7 | 50 | 1239 | 1.95 | 0.06 | 0.00 | 0.9 | 0.02 | 0.00 | 365 |
| Male- | 1 | 2.4 | 10.1 | 15.8 | 51 | 1244 | 0.92 | 0.13 | 0.00 | 1.3 | 0.04 | 0.00 | 245 |
| group 3 | 2 | 1.9 | 10.3 | 15.9 | 50 | 1359 | 1.12 | 0.09 | 0.00 | 0.6 | 0.02 | 0.00 | 33 |
| (EV) | 3 | 2.1 | 10.8 | 16.4 | 52 | 1449 | 1.36 | 0.04 | 0.00 | 0.7 | 0.01 | 0.00 | 282 |
| | 4 | 2.3 | 9.8 | 15.6 | 50 | 1391 | 1.5 | 0.05 | 0.00 | 0.7 | 0.02 | 0.02 | 351 |
| | 5 | 2.2 | 10.8 | 16.5 | 53 | 1270 | 1.07 | 0.23 | 0.01 | 0.8 | 0.03 | 0.02 | 404 |
| Female- | 1 | 2.0 | 10.4 | 16.2 | 51 | 1171 | 1.59 | 0.06 | 0.01 | 0.4 | 0.01 | 0.00 | 310 |
| group 3 | 2 | 1.6 | 9.8 | 15.8 | 49 | 1079 | 1.17 | 0.06 | 0.00 | 0.3 | 0.02 | 0.00 | 345 |
| (EV) | 3 | | | | | | | | | | | | |
| | 4 | 3.2 | 8.7 | 13.6 | 44 | 776 | 2.60 | 0.06 | 0.00 | 0.4 | 0.07 | 0.00 | 266 |
| | 5 | 1.7 | 9.6 | 15.5 | 50 | 1372 | 1.16 | 0.02 | 0.00 | 0.5 | 0.00 | 0.01 | 361 |

White blood cell count = WBC; Giga/L; Red blood cell count = RBC; Tera/L; Haemoglobin = HGB; g/100 mL; Haematocrit = HCT; %; Thrombocyte count = THR; Giga/L; Neutrophil count = NEUT; Giga/L; Eosinophil count = EOSI; Giga/L; Basophil count = BASO; Giga/L; Lymphocyte count = LYMP; Giga/L; Monocyte count = MONO; Giga/L; Large unstained cell count = LUC; Giga/L; Reticulocytes = RET; Giga/L

TABLE 67
14-day Tumorgenicity Study in Nude Mice;
Clinical Chemistry by Animal.

| Clinical | Animal | | | | | | | | | | |
|---|---|---|---|---|---|---|---|---|---|---|---|
| Chemistry | nº | ALT | AST | ALP | TBIL | CREA | UREA | Cl | K | Na | ALB |
| Male- | 1 | 38 | 76 | | 1.82 | 15 | 9.6 | | | | 23 |
| group 1 | 2 | 137 | 375 | | | 21 | 10.3 | 113.4 | 6.23 | 144 | 20 |
| (HeLa) | 3 | 103 | 247 | | 0.82 | 19 | 10.2 | | | | |
| | 4 | 147 | | | | 18 | 12.0 | | | | |
| | 5 | 70 | | | | 15 | 9.8 | | | | |
| Female- | 1 | 61 | 156 | 126 | 1.48 | 11 | 8 | 112.8 | 4.85 | 146 | 22 |
| group 1 | 2 | | | | | | | | | | |
| (HeLa) | 3 | 67 | 152 | | | 12 | 12.7 | | | | |
| | 4 | | | | | | | | | | |
| | 5 | 116 | | | | 17 | 8.7 | | | | |
| Male- | 1 | 52 | 178 | 66 | 3.01 | 19 | 10.3 | 119.1 | 5.96 | 141 | 28 |
| group 2 | 2 | 39 | 99 | 83 | 2.04 | 20 | 8.7 | 117.3 | 3.7 | 143 | 25 |
| (PBS) | 3 | 96 | 133 | 86 | 1.43 | 22 | 9.0 | 122.3 | 5.03 | 152 | 27 |
| | 4 | 67 | 166 | 91 | 1.31 | 23 | 8.8 | 117.5 | 4.52 | 146 | 24 |
| | 5 | 122 | 142 | 108 | 2.35 | 23 | 9.2 | 116.7 | 4.77 | 143 | 25 |
| Female- | 1 | 75 | 181 | 97 | 3.67 | 14 | 14.2 | 119.9 | 4.57 | 141 | 26 |
| group 2 | 2 | 72 | | | | 9 | 9.2 | | | | |
| (PBS) | 3 | 109 | 207 | | 1.01 | | 8.9 | | | | |
| | 4 | 73 | 98 | 93 | 1.48 | 17 | 9.7 | 116.1 | 4.21 | 141 | 25 |
| | 5 | 228 | 304 | | 2.33 | | | | | | |
| Male- | 1 | 43 | 82 | 82 | 1.15 | 22 | 8.6 | 117.9 | 4.39 | 144 | 24 |
| group 3 | 2 | 97 | 115 | 94 | 1.41 | 19 | 8.1 | 119.7 | 4.3 | 145 | 25 |
| (EV) | 3 | 102 | 285 | 100 | 2.31 | 18 | 8.9 | 121.1 | 4.55 | 146 | 25 |
| | 4 | 31 | 85 | 77 | 1.96 | 21 | 9.5 | 116.0 | 4.23 | 144 | 22 |
| | 5 | 57 | 143 | 70 | 0.92 | 19 | 10.9 | 116.2 | 4.83 | 143 | 25 |
| Female- | 1 | 68 | 297 | | 1.33 | | 11.9 | | | | |
| group 3 | 2 | 110 | 454 | 91 | 2.30 | 15 | 13.6 | 121.6 | 4.07 | 142 | 24 |
| (EV) | 3 | | | | | | | | | | |
| | 4 | 112 | 438 | 99 | 3.32 | 15 | 22 | 119.5 | 4.48 | 143 | 21 |
| | 5 | 92 | 356 | | 4.72 | | 18.5 | | | | |

Alanine aminotransferase = ALT; IU/L; Aspartate aminotransferase = AST; IU/L; Alkaline phosphatase = ALP; IU/L; Total Bilirubin = TBIL; μmol/L; Creatinine = CREA; μmol/L; Urea nitrogen = UREA; mmol/L; Chloride = Cl; mmol/L; Potassium = K; mmol/L; Sodium = Na; mmol/L; Albumin = ALB; g/L

TABLE 68
14-day Tumorgenicity Study in Nude Mice; Organ Weight by Animal.

| Organ | Animal | | | | | |
|---|---|---|---|---|---|---|
| Weight (g) | nº | Brain | Heart | Kidneys | Liver | Spleen |
| Male- | 1 | 0.35 | 0.166 | 0.37 | 1.29 | 0.07 |
| group 1 | 2 | 0.38 | 0.137 | 0.38 | 1.22 | 0.104 |
| (HeLa) | 3 | 0.37 | 0.166 | 0.45 | 1.56 | 0.092 |
| | 4 | 0.34 | 0.098 | 0.38 | 1.28 | |
| | 5 | 0.33 | 0.139 | 0.43 | 1.46 | 0.108 |
| Female- | 1 | 0.37 | 0.145 | 0.33 | 1.15 | 0.99 |
| group 1 | 2 | 0.37 | 0.112 | 0.3 | 1.16 | 0.84 |
| (HeLa) | 3 | 0.37 | 0.15 | 0.3 | 1.23 | 0.89 |
| | 4 | 0.04 | 0.1 | 0.27 | 1.05 | 0.69 |
| | 5 | 0.37 | 0.154 | 0.33 | 1.43 | 0.088 |
| Male- | 1 | 0.35 | 0.167 | 0.4 | 1.22 | 0.07 |
| group 2 | 2 | 39 | 0.147 | 0.46 | 1.27 | 0.075 |
| (PBS) | 3 | 0.38 | 0.16 | 0.47 | 1.3 | 0.088 |
| | 4 | 0.38 | 0.143 | 0.48 | 1.21 | 0.089 |
| | 5 | 0.37 | 0.151 | 0.46 | 1.27 | 0.084 |
| Female- | 1 | 0.38 | 0.142 | 0.32 | 0.95 | 0.071 |
| group 2 | 2 | 0.37 | 0.12 | 0.31 | 0.95 | 0.095 |
| (PBS) | 3 | 0.36 | 0.113 | 0.3 | 0.95 | 0.06 |
| | 4 | 0.4 | 0.127 | 0.35 | 1.01 | 0.071 |
| | 5 | 0.36 | 0.155 | 0.3 | 0.88 | 0.084 |
| Male- | 1 | 0.38 | 0.181 | 0.41 | 1.21 | 0.066 |
| group 3 | 2 | 0.37 | 0.159 | 0.43 | 1.22 | 0.093 |
| (EV) | 3 | 0.37 | 0.125 | 0.41 | 1.1 | 0.064 |
| | 4 | 0.38 | 0.19 | 0.45 | 1.18 | 0.072 |
| | 5 | 0.36 | 0.15 | 0.44 | 1.3 | 0.122 |
| Female- | 1 | 0.38 | 0.135 | 0.34 | 0.96 | 0.077 |
| group 3 | 2 | 0.39 | 0.116 | 0.31 | 0.93 | 0.065 |
| (EV) | 3 | 0.38 | 0.132 | 0.31 | 0.97 | 0.066 |
| | 4 | 0.38 | 0.138 | 0.35 | 1.04 | 0.073 |
| | 5 | 0.4 | 0.125 | 0.32 | 0.99 | 0.071 |

### Example 40

### CTC1-EV Release Panel

A summary of the panel used to evaluate and release *9 (Test 27) is given in TABLE 69.

TABLE 69
A subset of the testing panel used to evaluate *9 (Test 27) and
in-process samples leading to the final product, *9 (Test 27).

| QC Release | Specification |
|---|---|
| Secretome - Particle size distribution | Between 20 and 200 nm |
| Secretome - Particle concentration | >125.10e9 EV/ml |
| Secretome - Extractable volume | 2 ml +/− 5% |
| Secretome - Uniformity by MACSPlex ®, MFI CD9 | 85 to 115% of the mean value |
| Secretome - Sterility | Negative at D10 |
| Secretome - Mycoplasma | <10 CFU/ml |
| Secretome - Endotoxin | <2 EU/ml |
| Cells - Adventitious agent viral testing | No viral contaminant detected |
| (spent media) | |
| Secretome - Residual DNA, characterisation | No known oncogenic mutations detected |
| Secretome - Appearance: Clarity and degree of | Clear and no visible particles |
| opalescence of liquids; and visible particles | |
| (particulate contamination) | |
| Secretome - Appearance: Degree of coloration of liquids | Colorless |
| Secretome - Integrity | Package integrity |
| Secretome - pH | 7.20-7.50 |
| Secretome - EV marker identity after TFF | MFI CD9 > 10 |
| | MFI CD63 > 10 |
| | MFI CD81 >10 |
| Secretome - Potency in vitro | Increased proliferation of HUVEC >20% |
| Secretome - Immunogenicity in vitro: Activation of | Lack of increased secretion of IL-2 and |
| allogeneic PBMC assessed by secretion of IL-2 and IFNg | IFNg compared with the control |
| Secretome - Immunogenicity in vitro: Degranulation of | Lack of expression of CD107a compared |
| Natural Killer cells | with the control |

### Example 41

### Additional Characterization of CTC1-EV in Process and Final Formulation Samples

Additional characterization was performed on samples *1 though *9 from Test Example 25, Test Example 26 and Test Example 27. Analyses performed and results obtained are shown TABLE 70.

TABLE 70
Characterisation of CTC1-EV in-process and final formulation.

| | | | Test results | | | | | | |
|---|---|---|---|---|---|---|---|---|---|
| | | | *1 | | | | | | |
| | | | (Test 26) | | | | | | |
| | | | (D + 0 | | | | | | |
| | | | cells. | | | *4 | | | |
| | | | final | | | (Test 26) | *5 | | |
| | | | poole | | | (D + 5 | (Test 26) | *8 | *9 |
| | | | d cell | | | Cond- | (Cond- | (Test 27) | (Test 27) |
| | | | suspe | | | itioned | itioned | (Final | (Final |
| | | | nsion | *2 | | media | media | formula- | formula- |
| | | | prior | (Test 26) | *3 | before | after | tion before | tion after |
| | | Specific- | to | (D + 3 | (Test 26) | clarifica- | clarifica- | sterilizing | sterilizing |
| Test | Method | ation | seeding) | material) | (D + 5 cells) | tion) | tion) | filtration) | filtration) |
| Cells- | Nucleo | Inform- | 8.8E+09 | 1.1B+10 | 7.9E+09 | | | | |
| Number | Counter ® | ative | | (harvest | (harvested | | | | |
| of Viable | NC-200TM | | | ed from | from | | | | |
| Cells | (DAPI/ | | | T75) | CS10s | | | | |
| | AO | | | | and T75) | | | | |
| | staining) | | | | | | | | |
| | (EP | | | | | | | | |
| | 2.7.29) | | | | | | | | |
| Cells-% | Nucleo | >60% | 84% | 94% | 92% | | | | |
| Viability | Counter ® | for D + 0; | | | | | | | |
| | NC-200TM | and | | | | | | | |
| | (DAPI/ | >70% | | | | | | | |
| | AO | for | | | | | | | |
| | staining) | D + 3 | | | | | | | |
| | (EP | and | | | | | | | |
| | 2.7.29) | D + 5 | | | | | | | |
| Sterility | Aerobic + | Negative | Negative | Negative | | | Negative | Negative | Negative |
| | anaerobic | at D10 | | (spent | | | | | |
| | bacterial | | | media, | | | | | |
| | cultures | | | CS10) | | | | | |
| | (EP 2.6.1 | | | | | | | | |
| | (2.6.27) | | | | | | | | |
| Sterility | Membrane | Negative | | Negative | | | Negative | | |
| | filtration | at D7 | | (spent | | | | | |
| | (EP 2.6.1) | | | media, | | | | | |
| | | | | CS10) | | | | | |
| Myco- | qPCR | <10 | | <10 | | <10 | | <10 | <10 |
| plasma | (EP 2.6.7) | CFU/ml | | CFU/ml | | CFU/ml | | CFU/ml | CFU/ml |
| | | | | (spent | | | | | |
| | | | | media, | | | | | |
| | | | | CS10) | | | | | |
| Endo- | Kinetic | <2 | | <2 | | <2 | | <2 | <2 |
| toxin | chromo- | EU/ml | | EU/ml | | EU/ml | | EU/ml | EU/ml |
| | genic LAL | | | (spent | | | | | |
| | (EP | | | media, | | | | | |
| | 2.6.14) | | | CS10) | | | | | |
| Cells- | Convent- | 46 XX, | | 46 XX, | 46 XX, | | | | |
| Karyo- | ional | no | | no | no | | | | |
| type | Cytogenct | abnorma | | abnorm- | abnorm- | | | | |
| | ICS | lities | | alities | alities | | | | |
| | | | | (harvest- | (harvest- | | | | |
| | | | | ed from | ed from | | | | |
| | | | | T25) | T25) | | | | |
| Advent- | F-PERT | No | | | | | No reverse | | |
| itious agent | assay for | reverse | | | | | transcriptase | | |
| testing | the | transcrip | | | | | activity | | |
| (Bovine, | detection | tase | | | | | | | |
| porcino | of reverso | activity | | | | | | | |
| virus | transcrip- | | | | | | | | |
| detection; | tase activity | | | | | | | | |
| retrovirus | (ICHQSA | | | | | | | | |
| testing) | ICHQSD, | | | | | | | | |
| | (EP 5.2.3 | | | | | | | | |
| | and EP | | | | | | | | |
| | 5.2.12)) | | | | | | | | |
| | Analysis | No virus | | | | | No virus | | |
| | by thin | observed | | | | | observed | | |
| | section | | | | | | | | |
| | Transm- | | | | | | | | |
| | ission | | | | | | | | |
| | Electron | | | | | | | | |
| | Micro- | | | | | | | | |
| | scopy for | | | | | | | | |
| | bulk | | | | | | | | |
| | product | | | | | | | | |
| | (quanti- | | | | | | | | |
| | tative) | | | | | | | | |
| | (ICHQSA, | | | | | | | | |
| | (EP 5.2.3 | | | | | | | | |
| | and EP | | | | | | | | |
| | 5.2.12) | | | | | | | | |
| | Detection | No viral | | | No viral | | | | |
| | of Bovine | contamin- | | | contaminant | | | | |
| | & Porcine | ant | | | detected | | | | |
| | contam- | detected | | | | | | | |
| | inant using | | | | | | | | |
| | Vero, BT | | | | | | | | |
| | & ST cell | | | | | | | | |
| | lines | | | | | | | | |
| | (ICHQ5A, | | | | | | | | |
| | (EP 5.2.3 | | | | | | | | |
| | and EP | | | | | | | | |
| | 5.2.12)) | | | | | | | | |
| Secre- | BCA | Inform- | | | | 1.5 | 1.6 mg/ml | 1.0 mg/ml | 0.8 mg/ml |
| tome- | assay, | ative | | | | mg/ml | | | |
| Protein | method 4 | | | | | | | | |
| concen- | (EP 2.5.33) | | | | | | | | |
| tration | | | | | | | | | |
| Secre- | Mass | Inform- | | | | | | | |
| tome- | Spectrom | ative | | | | | | | |
| Protein | etry | | | | | | | | |
| profile | | | | | | | | | |
| content | | | | | | | | | |
| Secre- | GRP94 | Inform- | | | | | 17 ng/ml | | 122 ng/ml |
| tome- | assays | ative | | | | | | | |
| Host | | | | | | | | | |
| cell | | | | | | | | | |
| protein | | | | | | | | | |
| assess- | | | | | | | | | |
| ment | | | | | | | | | |
| Secre- | qPCR | Inform- | | | | | Y | Y | 2.2 μg/ml |
| tome- | (EP 2.6.35) | ative | | | | | | | (>80 basepair |
| Residual | | | | | | | | | fragments) |
| DNA, | | | | | | | | | 1.3 μg/ml |
| quantif- | | | | | | | | | (>200 basepair |
| ication | | | | | | | | | fragments) |
| | | | | | | | | | 0.9 μg/ml |
| | | | | | | | | | (<200 basepair |
| | | | | | | | | | fragments) |
| Secre- | Immun- | <0.4 | | | | <0.4 | | | |
| tome- | oassay | mg/ml | | | | mg/ml | | | |
| residual | | | | | | | | | |
| Anti- | | | | | | | | | |
| biotics | | | | | | | | | |
| Cells- | Flow | Inform- | Y | Y | Y | | | | |
| Identity | Cytometry | ative | | (harvest- | (harvest- | | | | |
| | (EP 2.7.24) | | | ed from | ed from | | | | |
| | | | | T75) | CS10s) | | | | |
| Cells- | RNA | <0.37 | | Below | Below | | | | |
| Transcrip- | sequenc- | copies/ | | detect- | detect- | | | | |
| tome | ing & | μL | | able limits | able limits | | | | |
| | ddPCR | (corresp- | | (i.e., not | (i.e., not | | | | |
| | | onding | | detect- | detect- | | | | |
| | | to <2% | | able) | able) | | | | |
| | | of | | | | | | | |
| | | residual | | | | | | | |
| | | iPSC) | | | | | | | |
| Cells- | Micro- | Inform- | | Y | Y | | | | |
| Morph- | scopy | ative | | (as | (as | | | | |
| ology | | | | viewed | viewed | | | | |
| | | | | on T75 | on T75 | | | | |
| | | | | and | and | | | | |
| | | | | CS10) | CS10s) | | | | |
| Secre- | Flow | MFI | | | | | | MFI | MFI |
| tome- | Cytom- | CD9 > 10 | | | | | | CD9 = 32 | CD9 = 27 |
| EV | etry: CD9/ | MFI | | | | | | MFI | MFI |
| marker | CD63/ | CD63 > | | | | | | CD63 = 132 | CD63 = 132 |
| identity | CD81 EV | 10 | | | | | | MFI | MFI |
| after- | surface | MFI | | | | | | CD81 = 155 | CD81 = 140 |
| TFF | markers | CD81 > | | | | | | | |
| | by | 10 | | | | | | | |
| | MACSPleX ® | | | | | | | | |
| | (Miltenyi) | | | | | | | | |
| | for 13 μL | | | | | | | | |
| | of sample | | | | | | | | |
| | (EP | | | | | | | | |
| | 2.7.24) | | | | | | | | |
| Secre- | Nano- | Inform- | | | | Y | Y | Y | Y |
| tome- | particle | ative | | | | | | | |
| Particle | Tracking | | | | | | | | |
| concen- | Analysis | | | | | | | | |
| tration | (NanoSig | | | | | | | | |
| | ht) | | | | | | | | |
| Secre- | | Between | | | | Y | Y | Y | Y |
| tome- | | 20 and | | | | | | | |
| Particle | | 200 nm | | | | | | | |
| size | | | | | | | | | |
| distribu- | | | | | | | | | |
| tion | | | | | | | | | |
| Secre- | Correlation | >125 × | | | | Y | Y | 201 × 109 | 196 × 109 |
| tome- | NTA vs | 109 | | | | | | EV/ml | EV/ml |
| Particle | MACSPlex | EV/ml | | | | | | | |
| concen- | | | | | | | | | |
| tration | | | | | | | | | |
| Secre- | EP 2.9.17 | 2 ml +/− | | | | | | | 1.96 (+/− |
| tome- | | 5% | | | | | | | 0.06) |
| Extrac- | | | | | | | | | |
| table | | | | | | | | | |
| volume | | | | | | | | | |
| Secre- | EP 2.9.6 | 85 to | | | | | | | 85 to |
| tome- | | 115% of | | | | | | | 115% of |
| Uniform- | | the mean | | | | | | | the mean |
| ity by | | value | | | | | | | value |
| MACSPIex ®, | | | | | | | | | |
| MFI | | | | | | | | | |
| CD9 | | | | | | | | | |
| Secre- | GLP | No | | | | | | | Y |
| tome- | animal | safety | | | | | | | |
| in vivo | studics | concerns | | | | | | | |
| Secre- | Clarity | Clear | | | | | | | Clear |
| tome- | and | and no | | | | | | | and no |
| Appear- | degree of | visible | | | | | | | visible |
| ance | opales- | particles | | | | | | | particles |
| | cence of | | | | | | | | |
| | liquids; | | | | | | | | |
| | and | | | | | | | | |
| | visibles | | | | | | | | |
| | particles | | | | | | | | |
| | (particulate | | | | | | | | |
| | contamin- | | | | | | | | |
| | ation) | | | | | | | | |
| | (EP 2.2.1 | | | | | | | | |
| | and | | | | | | | | |
| | 2.9.20) | | | | | | | | |
| Secre- | Degree of | colorless | | | | | | | Colorless |
| tome- | coloration | | | | | | | | |
| Appear- | of liquids | | | | | | | | |
| ance | (EP 2.2.2) | | | | | | | | |
| Secre- | EP 5.1.1 | Package | | | | | | | Package |
| tome- | and USP | integrity | | | | | | | integrity |
| Integrity | 1207 | | | | | | | | |
| Secre- | Potent- | 7.20- | | | | | | | 7.48 |
| tome- | iometric | 7.50 | | | | | | | |
| pH | determin- | | | | | | | | |
| | ation | | | | | | | | |
| | (EP 2.2.3) | | | | | | | | |
| Secre- | RNA | Inform- | | | | | | | Y |
| tome- | sequen- | ative | | | | | | | |
| RNA | cing | | | | | | | | |
| profilo | | | | | | | | | |
| Secre- | HUVEC | Increased | | | | | | | 47% |
| tome- | prolifer- | prolif- | | | | | | | |
| Potency | ation | eration > | | | | | | | |
| in vitro | | 20% | | | | | | | |
| Secret | Activation | Lack of | | | | | | | Lack of |
| ome- | of | increased | | | | | | | increased |
| Immunog | allogencic | secretion | | | | | | | secretion |
| enicity in | PBMC | of IL-2 | | | | | | | of IL-2 |
| vitro | assessed | and | | | | | | | and IFNg |
| | by | IFNg | | | | | | | compared |
| | secretion | compared | | | | | | | with |
| | of IL-2 | with | | | | | | | the control |
| | and IFNg | the | | | | | | | |
| | (ICH S8) | control | | | | | | | |
| | Degranula | Lack of | | | | | | | Lack of |
| | tion of | expre- | | | | | | | expression |
| | Natural | ssion of | | | | | | | of CD107a |
| | Killer | CD107a | | | | | | | compared |
| | cells | compared | | | | | | | with |
| | (ICH S8) | with | | | | | | | the control |
| | | the | | | | | | | |
| | | control | | | | | | | |

### Use of the MACSPlex Exosome Kit to Determine EV Concentrations.

As depicted TABLE 70, one of the metrics used to assess CTC1-EV containing secretomes was the secretome particle concentration as determined by the correlation of Nanoparticle Tracking Analysis (NTA) and MACSPlex (labeled “Correlation NTA vs MACSPlex” in TABLE 70). This is a novel method for evaluating EV concentration which was established as follows.

NTA is considered one of the standard methods for the determination of particle concentration. The NanoSight is an instrument which can perform NTA on EV containing samples. However, NTA lacks the robustness and reproducibility needed for a qualified assay to be used as a QC release assay. A qualified assay to assess EV concentration was required as a QC release assay. Preliminary work suggested that there may be a correlation between the NTA, and the MACSPlex Exosome kit flow cytometry method probing for one of the EV tetraspanin markers. Since there are standard methods that can be applied to qualifying flow cytometry-based methods for QC release assays, such a correlation could potentially be exploited to develop a qualifiable EV quantification assay. Since CTC1-EV contain CD9, this tetraspanin marker was selected as the target tetraspanin for this assay development. It should be noted that not all EV from all cell sources express CD9. For other EV types, it should be determined whether CD9, CD63 or CD81 are best suited as the tetraspanin target for the MACSPlex Exosome kit analysis, and then the same experiments as below can be repeated to determine the correlation factor for the EV type of interest between NTA and MACSPlex Exosome Kit MFI results.

To explore whether the MFI for CD9 by the MACSPlex Exosome kit (“CD9 MFI”) was well correlated to the concentration of particles as measured by the NanoSight, 120 samples and/or individual sample dilutions of CTC1-EV were analyzed by NTA and for CD9 MFI. The data find that the concentration of particles, as measured by the NTA, is indeed correlated to the CD9 MFI for CTC1-EV. The correlation that was found is depicted in FIG. 98A.

Next, the linearity of the CD9 MFI was determined. For this determination, various input volumes of *9 (Test 27) were used. As depicted in FIG. 98B, an excellent linearity was found, with an R² value of 0.9757 for this dataset. This dataset also enabled the establishment of a minimum linear range of 4 to 100 in CD9 MFI, which is equivalent to a range of particle concentrations by NTA from 3.4×10⁸ particles/mL to 8.6×10⁹ particles/mL.

### Additional HUVEC Scratch Wound Healing Assay

Further to this testing, an aliquot of clarified conditioned media was obtained from both Test Example 25 and Test Example 26, and ultracentrifuged to produce samples *5a.uc (Test 25) and *5b.uc (Test 26) illustrated in FIG. 90 and FIG. 96 for Example 19. These samples were evaluated by HUVEC Scratch Wound Healing Assay, essentially as described in Example 4, where a 1× dose was calculated to represent the sEV-enriched secretome produced by 150,000 mother cells. Mock-EV controls were generated by UC from comparable virgin media controls as previously described and dosed into the assay as previously described. These CTC1-EV samples dramatically stimulated HUVEC Scratch Wound Healing, as illustrated in FIG. 91.

The results show that for all of the doses tested, both *5a.uc (Test 25) and *5b.uc (Test 26) have the ability to improve HUVEC scratch wound healing. This indicates that the conditioned media from which the *5a.uc (Test 25) and *5b.uc (Test 26) have been generated, which was the same lot of conditioned media that was subjected to TFF as part of the process to produce the CTC1-EV Final Formulation (*9 (Test 27)), contained components which have the ability to activate the scratch wound healing ability of endothelial cells, such as HUVECs.

There is some indication of a dose-response in the three doses analyzed for the two samples analyzed in this assay. The strongest effects were seen for the 3× dose of (*5a.uc (Test 25)) and the 3× dose of *5b.uc (Test 26), which had an effect of 49% compared to the positive control and 50% compared to the positive control, respectively. When compared to the highest effects seen in the mock-EV controls (highest effect observed was in 2× dose, which was 0.043, or 4.3%), this is a more than an 11-fold increase in HUVEC scratch wound healing for *5a.uc (Test 25) and *5b.uc (Test 25)).

### Example 43

### CTC1-EV Stability Testing

Three stability panels were designed to evaluate *9(Test 27) whose preparation is described in detail in Example 19. The first panel tracks the stability of frozen *9 (Test 27) stored at −80 °C (TABLE 71). The second tracks *9(Test 27) stability at room temperature after a vial has been thawed (TABLE 72), and the third tracks *9 (Test 27) stability at room temperature after thawing and diluting in 0.9% NaCl (TABLE 73) The parameters selected to monitor stability are given in TABLE 71, TABLE 72, and TABLE 73. The stability testing results for *9 (Test 27) are also given in TABLE 71, TABLE 72, and TABLE 73.

TABLE 71
Stability testing of *9 (Test 27) in frozen storage at −80 °C (between −65 °C and −85 °C).

| CTC1-EV (*9 (Test 27)) | Months | | | | | | | | | | | |
|---|---|---|---|---|---|---|---|---|---|---|---|---|
| Stability in frozen storage | 0 | 1 | 2 | 7 | 12 | 18 | 21 | 24 | 27 | 30 | 33 | 36 |

Quantity

| | | | | | | | | | | | | |
|---|---|---|---|---|---|---|---|---|---|---|---|---|
| Particle concentration | 174 | 209 | 189 | 185 | 225 | 223 | 214 | tbd | tbd | tbd | tbd | tbd |
| (10 °p/mL) (by Macsplex | | | | | | | | | | | | |
| exosome kit, Miltenyi ®) | | | | | | | | | | | | |

Identity

| | | | | | | | | | | | | |
|---|---|---|---|---|---|---|---|---|---|---|---|---|
| Particle size (mode, nm) | 166 | 126 | 111 | 133 | 121 | 115 | 110 | tbd | tbd | tbd | tbd | tbd |
| CD9 expression | 50.6 | 60.8 | 55 | 53.9 | 65.4 | 65.0 | 62.7 | tbd | tbd | tbd | tbd | tbd |
| (for 25 μL of sample) | | | | | | | | | | | | |
| (specification: MFI > 10) | | | | | | | | | | | | |
| CD63 expression | 25 | 271 | 302 | 230 | 260 | 209 | 199 | tbd | tbd | tbd | tbd | tbd |
| (for 25 μL of sample) | | | | | | | | | | | | |
| (specification: MFI > 10) | | | | | | | | | | | | |
| CD81 expression | 267 | 281 | 294 | 231 | 310 | 249 | 237 | tbd | tbd | tbd | tbd | tbd |
| (for 25 μL of sample) | | | | | | | | | | | | |
| (specification: MFI > 10) | | | | | | | | | | | | |

In vitro function

| | | | | | | | | | | | | |
|---|---|---|---|---|---|---|---|---|---|---|---|---|
| HUVEC proliferation | ND | ND | ND | 46% | 51% | 32% | 50% | tbd | tbd | tbd | tbd | tbd |
| (specification: > | | | | | | | | | | | | |
| 20% over controls) | | | | | | | | | | | | |

Safety

| | | | | | | | | | | | | |
|---|---|---|---|---|---|---|---|---|---|---|---|---|
| Sterility | negative | negative | negative | negative | negative | negative | negative | tbd | tbd | tbd | tbd | tbd |
| (specification: | | | | | | | | | | | | |
| negative at 10 days) | | | | | | | | | | | | |

TBD: to be determined (in progress); ND: Not done

TABLE 72
Stability testing of *9 (Test 27) at room temperature
(between 18 °C and −25 °C) for 48 hours.

| | | | | |
|---|---|---|---|---|
| CTC1-EV (*9(Test 27) | | | | |
| Stability at Room | Reference | At thaw | 24 h post- | 48 h post- |
| Temperature | (CoA) | (=0 h) | thaw | thaw |

Quantity

| | | | | |
|---|---|---|---|---|
| Particle concentration | 204 | 202 | 201 | 212 |
| (10⁹ p/mL) (by Macsplex | | | | |
| exosome kit, Miltenyi ®) | | | | |

Identity

| | | | | |
|---|---|---|---|---|
| CD9 expression | 56.9 | 58.7 | 58.5 | 61.9 |
| (for 25 μL of sample) | | | | |
| (specification: MFI > 10) | | | | |
| CD63 expression | 257 | 263 | 274 | 273 |
| (for 25 μL of sample) | | | | |
| (specification: MFI > 10) | | | | |
| CD81 expression | 267 | 276 | 289 | 284 |
| (for 25 μL of sample) | | | | |
| (specification: MFI > 10) | | | | |

TABLE 73
Stability testing of *9 (Test 27) after dilution in NaCl at room
temperature (between 18 °C and −25 °C) for 4 hours.

| | | | | |
|---|---|---|---|---|
| CTC1-EV (*9 (Test 27)) | | | Diluted | Diluted |
| Stability in 0.9% NaCl, | Reference | Thawed, | 1:6.25, | 1:12.5, |
| Room Temperature | (CoA) | not diluted | 4 H | 4 H |

Quantity

| | | | | |
|---|---|---|---|---|
| Particle concentration | 204 | 209 | 206 | 199 |
| (10⁹ p/mL) (normalized to | | | | |
| the dilution factor) | | | | |
| (by Macsplex exosome kit, | | | | |
| Miltenyi ®) | | | | |

Identity

| | | | | |
|---|---|---|---|---|
| CD9 expression | 56.9 | 60.8 | 46.1 | 22.3 |
| (for 25 μL of sample) | | | | |
| (specification: MFI > 10) | | | | |
| CD63 expression | 257 | 266 | 234 | 158 |
| (for 25 μL of sample) | | | | |
| (specification: MFI > 10) | | | | |
| CD81 expression | 267 | 268 | 229 | 166 |
| (for 25 μL of sample) | | | | |
| (specification: MFI > 10) | | | | |

Additional CTC1-EV were tested for stability. Results are detailed in TABLE 74 through TABLE 78 (five tables).

TABLE 74
Stability by NTA.

| EV NTA | | | | | | | | | | | | | | |
|---|---|---|---|---|---|---|---|---|---|---|---|---|---|---|
| in 10e9 | Months | | | | | | | | | | | | | |
| EV/mL | 0 | 1 | 2 | 4 | 6 | 7 | 8 | 12 | 14 | 18 | 22 | 24 | 27 | 30 |
| Final | | 110 | | 145 | 255 | 215 | | | | | | | | 300 |
| formulation, | | | | | | | | | | | | | | |
| Test 17 | | | | | | | | | | | | | | |
| *7, sample | 100 | | 210 | 135 | | | | | | | | | 200 | |
| a (Test 20) | | | | | | | | | | | | | | |
| *7, sample | 120 | 270 | | | | | | | 150 | | | 130 | | |
| a (Test 22) | | | | | | | | | | | | | | |
| *9 (Test 27) | 285 | 101 | 61 | | | | 65 | 51 | | 135 | 113 | | | |

TABLE 75
Stability by Mascplex.

| EV Macsplex | | | | | | | | | | | | | | |
|---|---|---|---|---|---|---|---|---|---|---|---|---|---|---|
| in 10e9 | Months | | | | | | | | | | | | | |
| EV/mL | 0 | 1 | 2 | 4 | 6 | 7 | 8 | 12 | 14 | 18 | 22 | 24 | 27 | 30 |
| Final | | 146 | | 140 | 132 | 170 | | | | | | | | 210 |
| formulation, | | | | | | | | | | | | | | |
| Test 17 | | | | | | | | | | | | | | |
| *7, sample | 132 | | 137 | 150 | | | | | | | | | 173 | |
| a (Test 20) | | | | | | | | | | | | | | |
| *7, sample | 134 | 158 | | 141 | | | 165 | | 175 | | 172 | 166 | | |
| a (Test 22) | | | | | | | | | | | | | | |
| *9 (Test 27) | 173 | 204 | 198 | | | | 180 | 225 | | 223 | 214 | | | |

TABLE 76
Stability by CD9 MFI.

| CD9 | | | | | | | | | | | | | | |
|---|---|---|---|---|---|---|---|---|---|---|---|---|---|---|
| MFI/μl | 0 | 1 | 2 | 4 | 6 | 7 | 8 | 12 | 14 | 18 | 22 | 24 | 27 | 30 |
| Final | | | | 1.63 | 1.53 | 1.98 | | | | | | | | 2.44 |
| formulation, | | 1.7007 | | 090 | 770 | 038 | | | | | | | | 635 |
| Test 17 | | 9914 | | 329 | 881 | 256 | | | | | | | | 493 |
| *7, | 1.53 | | | 1.74 | | | | | | | | | 2.01 | |
| sample a | 770 | | 1.5959 | 739 | | | | | | | | | 533 | |
| (Test 20) | 881 | | 5536 | 638 | | | | | | | | | 049 | |
| *7, | 1.56 | | | 1.64 | | | 1.92 | | 2.03 | | 2.00 | 1.933 | | |
| sample a | 100 | .84059 | | 255 | | | 213 | | 862 | | 368 | 785 | | |
| (Test 22) | 743 | 085 | | 26 | | | 602 | | 911 | | 118 | 33 | | |
| | 2.01 | | | | | | 2.09 | 2.62 | | 2.59 | 2.49 | | | |
| *9 (Test | 533 | .37645 | 30656 | | | | 687 | 109 | | 779 | 295 | | | |
| 27) | 049 | 908 | 322 | | | | 566 | 457 | | 595 | 217 | | | |

TABLE 77
Stability by CD9 MFI.

| CD9 | | | | | | | | | | | | | | |
|---|---|---|---|---|---|---|---|---|---|---|---|---|---|---|
| MFI/25 μl | 0 | 1 | 2 | 4 | 6 | 7 | 8 | 12 | 14 | 18 | 22 | 24 | 27 | 30 |
| Final | | 42.5 | | 40.7 | 38.4 | 49.5 | | | | | | | | 61.1 |
| formulation, | | 1997 | | 7258 | 4272 | 0956 | | | | | | | | 5887 |
| Test 17 | | 86 | | 22 | 03 | 41 | | | | | | | | 33 |
| *7, sample | 38.4 | | 39.8 | 43.6 | | | | | | | | | 50.3 | |
| a (Test 20) | 4272 | | 9888 | 8490 | | | | | | | | | 8326 | |
| | 03 | | 4 | 95 | | | | | | | | | 23 | |
| *7, sample | 39.0 | 46.0 | | 41.0 | | | 48.0 | | 50.9 | | 50.0 | 48.3 | | |
| a (Test 22) | 2518 | 1477 | | 6381 | | | 5340 | | 6572 | | 9202 | 4463 | | |
| | 58 | 13 | | 49 | | | 04 | | 77 | | 95 | 32 | | |
| *9 (Test | 50.3 | 59.4 | 57.6 | | | | 52.4 | 65.5 | | 64.9 | 62.3 | | | |
| 27) | 8326 | 1147 | 6408 | | | | 2189 | 2736 | | 4489 | 2380 | | | |
| | 23 | 69 | 05 | | | | 14 | 42 | | 88 | 42 | | | |

TABLE 78
Percent evolution of various CTC1-EV lots.

| % | | | | | | | | | | | | | | |
|---|---|---|---|---|---|---|---|---|---|---|---|---|---|---|
| Evolution | 0 | 1 | 2 | 4 | 6 | 7 | 8 | 12 | 14 | 18 | 22 | 24 | 27 | 30 |
| Final | | 1 | | 0.95 | 0.90 | 1.16 | | | | | | | | 1.43 |
| formulation, | | | | 890 | 4109 | 4383 | | | | | | | | 835 |
| Test 17 | | | | 411 | 59 | 56 | | | | | | | | 616 |
| *7, sample | | | 1.03 | 1.13 | | | | | | | | | 1.31 | |
| a (Test 20) | 1 | | 7878 | 636 | | | | | | | | | 0606 | |
| | | | 79 | 364 | | | | | | | | | 06 | |
| *7, sample | | 1.17 | | 1.05 | | | 1.23 | | 1.30 | | 1.28 | 1.23 | | |
| a (Test 22) | 1 | 9104 | | 223 | | | 1343 | | 5970 | | 3582 | 8805 | | |
| | | 48 | | 881 | | | 28 | | 15 | | 09 | 97 | | |
| *9 (Test | | 1.17 | 1.14 | | | | 1.04 | 1.30 | | 1.28 | 1.23 | | | |
| 27) | 1 | 9190 | 4508 | | | | 0462 | 0578 | | 9017 | 6994 | | | |
| | | 75 | 67 | | | | 43 | 03 | | 34 | 22 | | | |

The results are depicted in FIGS. 93A and 93B. FIG. 93A and FIG. 93B show the particle size distribution and particle concentration data generated by Nanosight nanoparticle tracking analysis (NTA) on the *9 (Test 27) samples at a timepoint close to manufacture (“To”) and on other vials of *9 (Test 27) which had been maintained in −80 degree Celsius storage for the following amount of time: 1 month, 2 months, 6 months or 1 year. At each timepoint, a vial of *9 (Test 27). *9 (Test 27) is thawed and evaluated by NTA. The particle size distribution is illustrated as a spectrum (graphs found in the third row of data from the top). Each spectrum in the figure represents the average and standard deviation of five spectra collected by the NTA for that sample. This is called a merged spectrum. If multiple technical replicates were performed resulting in merged spectra for a given timepoint, a representative merged spectrum is shown in the figure. The average mean particle size (“Average mean”), the average mode particle size (“Average mode”), and the average particle concentration (“Average concentration”) are given in the fourth, fifth and sixth rows of FIG. 93A and FIG. 93B from the top, respectively. The “Average mean”, “Average mode” and “Average concentration” data presented in rows 4, 5 and 6, respectively, are the average values of technical replicates performed at that timepoint.

For the five Timepoints analyzed, the Average mean did not change more than 40% of the Average mean at T₀. The Average mean measured at the 1-year Timepoint did not change from the Average mean measured at the To timepoint by more than 30%. For the five timepoints analyzed, the Average mean was always in the range of 100 nm to 175 nm.

For the five timepoints analyzed, the Average mode did not change more than 15% of the Average mode at T₀. The Average mode measured at the 1-year timepoint did not change from the Average mean measured at the T0 timepoint by more than 15%. For the five timepoints analyzed, the Average mode was always in the range of 80 nm to 150 nm.

For the five timepoints analyzed, the Average concentration did not change more than 85% of the Average concentration at To. The Average concentration measured at the 1 year timepoint did not change from the Average concentration measured at the TO timepoint by more than 85%. For the five timepoints analyzed, the Average concentration was always in the range of 5×10¹⁰ particles/mL to 3×10¹¹ particles/mL.

### Example 44

### In Vitro Analysis of the Stability of

### CTC1-EV Potency in a HUVEC Survival Assay

The functionality and potency of CTC1-EV as a function of storage time was further analyzed by the HUVEC Stress Assay, as described in Example 24. Briefly, HUVEC cells were cultured in serum-containing medium on 0.1% gelatin-coated 96-well plates until confluence. At confluence, the media was changed to a serum depleted (poor) media with or without staurosporine (0.01 uM concentration) for one hour. The staurosporine was then washed out by multiple media exchanges. The cells were then incubated with poor media or poor media supplemented with 5×10⁹ particles of *9 (Test 27) for 20 hours. Cells were counted using the Cell Counting Kit-8 as described in Example 34. The positive control is the Poor media without staurosporine. The results were normalised to the negative control (Poor media with staurosporine). Vials of these *9 (Test 27) were analyzed soon after production (DO) an again after 1 month, 6 months and 1 year of frozen storage (in −80 °C freezers). The results are depicted in FIG. 94. Data are shown as cell fold change over the negative control (Poor media with staurosporine). Results show that the material retained potency in this assay for at least 1 year.

### Example 45

### Second Example of Optimization of Filter Cut-Off Size for Purifying EV/Secretomes

Aliquots of conditioned media (MC) from an “Aggregate vesiculation”, a “Fresh CPC Plated Vesiculation” and a “Thawed CPC Plated Vesiculation” from the examples illustrated in FIG. 2 were used to test the impact of filter MWCO (molecular weight cut off) on particle concentration, particle size distribution, and biological function as assessed by an H9c2 viability assay. The MWCOs tested were 3 kDa, 30 kDa, and 100 kDa. For each test, 15 mL of conditioned media were filtered through an Amicon Ultra-15, PLTK, membrane Ultracel-PL (membrane=regenerated cellulose) as directed by the manufacturer. The retentates were collected in PBS, aliquoted and frozen at −80 °C until analysis. Matching virgin media underwent the same process.

### Nanosight Results:

In the samples tested, the particle concentrations were similar between 3 kDa and 30 kDa retentates. The concentration of particles was significantly reduced in the 100 kDa retentates. The results are shown in TABLE 79.

TABLE 79
Particles concentrations by nanosight.

| | 3 kDa | 30 kDa | 100 kDa |
|---|---|---|---|
| UF MC | Concentration: | Concentration: | Concentration: |
| HYPERFLASK | 61 × 10\hat{ }6 p/ul | 76 × 10\hat{ }6 p/ul | 46 × 10\hat{ }6 p/ul |
| Thawed CPC, Plated | | | |
| UF MC | Concentration: | Concentration: | Concentration: |
| HYPERFLASK | 85 × 10\hat{ }6 p/ul | 86 × 10\hat{ }6 p/ul | 28 × 10\hat{ }6 p/ul |
| Fresh CPC, plated | | | |

### H9c2 Results:

The retentates were assessed by H9c2 cell viability assays essentially as described in Example 4. 60 μL of each UF retentate preparation was used for the H9c2 assay.

A Poor media control was H9c2 cells cultured in the serum-free medium alone. To analyze if a test material has the capacity to improve H9c2 survival in a serum-deprivation stress condition, the test material was added to the serum-free medium (a test condition) and the H9c2 were cultured in the test condition. The results of a test condition are the number of H9c2 cells remaining in culture at the end of the assay. The results of technical replicates were averaged, and the averaged results were normalized to the Virgin Media 100 kDa test condition.

The results are illustrated in FIG. 95. For the Thawed CPC plated Vesiculation test conditions (first three bars on the left in the figure), all three MWCO test conditions improved H9c2 cell survival in a stress condition. The 30 kDa retentate improved the H9c2 cell survival by more than 2.2 fold over the matched Virgin media UF Retentate 30 kDa retentate test condition.

Some of the in vitro effects of the secretome on H9c2 survival were lost after filtering out material<100 kDa. The 30 kDa size was therefore selected.

### Example 46

### Additional Characterization of CTC1-EV Using ONi Nanoimager

Single particle tetraspanin marker expression profiles for *9 (Test 27) were generated using the ONi Nanoimager. The ONi is a d-STORM capable super-resolution fluorescence microscope. CTC1-EV were assayed using the EV Profiler Kit according to the manufacturer's directions and the resulting chip was imaged on the ONi microscope using 488, 560, and 640 lasers. The principle of this kit is that CTC1-EV are immobilized onto a chip and then are probed for tetraspanin marker expression. The ONi software identifies clusters of fluorescence (i.e., extracellular vesicles) and then determines the relative abundance of each cluster sub-type. A cluster-sub-type is defined as having a distinct combination of one, two or three of the canonical EV tetraspanins, CD9, CD63 and CD81. Single positive (“SP”), double positive (“DP”) and triple positive (“TP”) subtypes are possible. SP are positive for one and only one of the three tetraspanins. DP are positive for two and only two of the three tetraspanins. TP are positive for all three of the tetrapanins. For example, “CD9_SP” is positive for CD9, but negative for CD81 and negative for CD63. This can also be described as CD9+/CD63−/CD81−. There are three possible single positive cluster sub-types (CD9_SP; CD63_SP; CD81_SP). There are three possible double positive cluster sub-types (CD63/CD9_DP; CD81/CD9_DP; CD81/CD63_DP). There is only one possible TP cluster sub-type (CD81/CD63/CD9_TP). The order of the tetraspanins in a cluster sub-type name is meaningless and arbitrary and can be rearranged without changing the meaning. For example, CD81/CD9_DP and CD9/CD81_DP are referring to the same cluster sub-type. FIG. 99 shows a representative cluster from *9 (Test 27) visualized by ONi that is CD81/CD63/CD9_TP. The CD9 signal, CD81 signal and CD63 signal are shown individually and as an overlay, confirming the presence of each of the three markers in a single cluster. FIG. 99B indicates the relative abundance of each cluster sub-type in the *9 (Test 27) as detected by ONi super-resolution microscopy (shown as a % under each bar). The absolute counts are graphed in the figure and given above each bar.

### Example 47

### Summary of CTC1-EV Features

Molecular and functional features of CTC1-EV are summarized in FIG. 100A-D.

### Example 48

### Clinical Trail Design

The clinical trial design is described below and depicted in FIG. 101

### Main Inclusion Criteria:

Age between 18 to 80 years.
Dilated cardiomyopathy defined by a dilated LV with a reduced EF≤40% on echocardiography and/or CMR imaging, unexplained by pressure or volume overload (severe arterial hypertension or significant valve disease), coronary artery disease (as assessed by coronary angiography) or a systemic disease.
NYHA Class III in spite of optimal heart failure maximally tolerated guideline-directed medical therapy, including cardiac resynchronization if needed, without other treatment options.
Plasma level of B-type natriuretic peptide (BNP)>150 μg/mL or, N-terminal pro-BNP (NT-proBNP)≥400 μg/mL.

### Dose:

Cohort 1: 20×10⁹ particles/kg for each infusion, with a total of 3 infusions, for a cumulative dose of 60×10⁹ particles/kg.
Cohort 2, in the absence of safety issues in Cohort 1: 40×10⁹ particles/kg for each infusion, with a total of 3 infusions, for a cumulative dose of 120×10⁹ particles/kg.
Treatment schedule: 3 infusions, 21 days between infusions (day 0, day 21 and day 42).
Duration of treatment: 42 days.

### Route of Administration:

Intravenous infusions.

### Administration Procedure:

Infusions of the IMP (diluted in 0.9% NaCl solution to a final 50 mL-volume per infusion) via a peripheral vein by means of a flow rate controlled syringe pump over 45-60 minutes, in an Intensive Care Unit.

### Additional Precautions:

Bolus injection of corticosteroids and antihistaminic agents 1 hour before the IMP infusion.
Before each infusion, screening for antibodies against the HLA expressed by the secretome-producing cells and if antibodies are detected at a Mean Fluorescence Intensity≥5000, the next planned infusion will be cancelled.

### Brief Summary:

The goal of this clinical trial is to assess the safety and efficacy of three intravenous injections of the extracellular vesicle-enriched secretome of cardiovascular progenitor cells in severely symptomatic patients with drug-refractory left ventricular (LV) dysfunction secondary to non-ischemic dilated cardiomyopathy. The main questions it aims to answer are:

Are these repeated injections safe and well tolerated?

Do they improve cardiac function and, if yes, to what extent?

Condition or Disease: Heart Failure with Reduced Ejection Fraction

Intervention/treatment: Biological; extracellular vesicle-enriched secretome of cardiovascular progenitor cells differentiated from induced pluripotent stem cells.

### DETAILED DESCRIPTION

The overall objective of this study is to assess the safety and efficacy of repeated intravenous injections of the secretome of cardiovascular progenitor cells in severely symptomatic patients with drug-refractory left ventricular (LV) dysfunction secondary to non-ischemic dilated cardiomyopathy.

The rationale and design of this trial are based on three main assumptions:

The tissue-repair capacity of transplanted cells can be duplicated by the delivery of the extracellular vesicles (EV) that they secrete.

The greatest therapeutic efficacy seems to be achieved by using secreting cells that are committed to the same lineage as those of the tissue to be repaired, hence, the use of cardiovascular progenitor cells as the source of the EV-enriched secretome.

Leveraging the benefits of cells, or their secreted products, by repeated administrations requires a non-invasive approach, which highlights the potential interest of the intravenous approach.

### Experimental: Treated Group

A maximum of 12 patients will be included in the study following a dose-escalating design:
Cohort 1 (4 patients) will receive 20×10E9 particles/kg for each infusion, with a total of 3 infusions, for a cumulative dose of 60×10E9 particles/kg;
Cohort 2: in the absence of safety issues in Cohort 1, 8 patients will receive 40×10E9 particles/kg for each infusion, with a total of 3 infusions, for a cumulative dose of 120×10E9 particles/kg.

### Intervention/Treatment:

Biological: Extracellular vesicle-enriched secretome of cardiovascular progenitor cells differentiated from induced pluripotent stem cells

Repeated (×3) intravenous infusions of the extracellular vesicle-enriched secretome of cardiovascular progenitor cells (differentiated from human induced pluripotent stem cells)

### Primary Outcome Measures:

Serious Adverse Events. Time Frame: 10 weeks after the onset of treatment: 6 weeks of treatment and 4 weeks of follow-up after the last IMP infusion.

Number of any potentially Serious Adverse Events (SAEs)/Reactions attributed to the experimental treatment: death (cardiovascular or of any cause), hospitalization for worsening heart failure, acute coronary syndrome (including myocardial infarction), sustained atrial and ventricular arrhythmias, ischemic stroke, immune-allergic or infectious reactions to the intravenous infusions of the IMP, and any other potential adverse effects detected and corroborated by clinical presentation, laboratory investigations and image analysis.

### Secondary Outcome Measures:

Validation of the bioactivity of the EV-enriched secretome by proliferation of human vascular endothelial cells. Time Frame: 12 months.

Bioactivity of the IMP (potency tests) assessed by proliferation of human vascular endothelial cells assessed by BrdU (>20% relative to the control).

Validation of the bioactivity of the EV-enriched secretome by activation of allogeneic peripheral blood mononuclear cells. Time Frame: 12 months.

Bioactivity of the IMP (potency tests) assessed by activation of allogeneic peripheral blood mononuclear cells assessed by the secretion of IL-2 and IFNγ (lack of increased secretion compared with the control).

Validation of the bioactivity of the EV-enriched secretome. Time Frame: 12 months.

Bioactivity of the IMP (potency tests) assessed by degranulation of Natural Killer cells assessed by the expression of CD107 (compared with a negative control).

Assessment of the effects of the IMP on immune and inflammatory responses at 3 weeks after the onset of the treatment. Time Frame: 3 weeks after the onset of the treatment.

Detection of donor-specific antibodies before the second secretome infusion.

Assessment of the effects of the IMP on immune and inflammatory responses at 6 weeks after the onset of the treatment. Time Frame: 6 weeks after the onset of the treatment.

Detection of donor-specific antibodies before the third secretome infusion.

Assessment of the effects of the IMP on immune and inflammatory responses at 10 weeks after the onset of the treatment. Time Frame: 10 weeks after the onset of the treatment.

Detection of donor-specific antibodies at 28 days following the last secretome infusion.

Assessment of the effects of the IMP on immune and inflammatory responses at 6 months after the last secretome infusion. Time Frame: 6 months after the last secretome infusion.

Detection of donor-specific antibodies at 6 months following the last secretome infusion if DSA are detected at the 28 days post-treatment study point at MFI≥5000.

Inflammatory response to IMP infusions. Time Frame: 28 days, 6 and 12 months following the third infusion.

Assessment of blood levels of interleukins, C-Reactive Protein and immune cells.

Monitoring for Major Cardiovascular Adverse Events (MACE). Time Frame: 28 days following the last IMP infusion and subsequently until 1 year after the end of treatment.

MACE including cardiac death, rehospitalization for heart failure, acute coronary syndromes, ischemic stroke and ventricular arrhythmias during the 1-year follow-up.

Changes in V function assessed by NYHA at 28 days after the end of the treatment. Time Frame: 28 days after the end of the treatment.

New York Heart Association (NYHA) functional class.

Changes in LV function assessed by NYHA at 6 months after the end of the treatment. Time Frame: 6 months after the end of the treatment.

New York Heart Association (NYHA) functional class.

Changes in LV function assessed by NYHA at 12 months after the end of the treatment. Time Frame: 12 months after the end of the treatment.

New York Heart Association (NYHA) functional class.

Changes in LV function assessed by Minnesota Living With Heart Failure questionnaire at 6 months after the end of the treatment. Time Frame: 6 months after the end of the treatment.

Quality of life assessed by Minnesota Living With Heart Failure questionnaire.

Changes in LV function assessed by Minnesota Living With Heart Failure questionnaire at 12 months after the end of the treatment. Time Frame: 12 months after the end of the treatment.

Quality of life assessed by Minnesota Living With Heart Failure questionnaire.

Changes in LV function assessed by LV ejection fraction at 28 days after the end of the treatment. Time Frame: 28 days after the end of the treatment.

Measurements of LV ejection fraction (EF %) by Doppler-echocardiography.

Changes in LV function assessed by LV ejection fraction at 6 months after the end of the treatment. Time Frame: 6 months after the end of the treatment.

Measurements of LV ejection fraction (EF %) by Doppler-echocardiography.

Changes in LV function assessed by LV ejection fraction at 12 months after the end of the treatment. Time Frame: 12 months after the end of the treatment.

Measurements of LV ejection fraction (EF %) by Doppler-echocardiography.

Changes in LV function assessed by LV Volumes at 28 days after the end of the treatment. Time Frame: 28 days after the end of the treatment.

LV Volumes ml/m2 by Doppler-echocardiography.

Changes in LV function assessed by LV Volumes at 6 months after the end of the treatment. Time Frame: 6 months after the end of the treatment.

LV Volumes ml/m2 by Doppler-echocardiography.

Changes in LV function assessed by LV Volumes at 12 months after the end of the treatment. Time Frame: 12 months after the end of the treatment.

LV Volumes ml/m2 by Doppler-echocardiography.

Changes in LV function assessed by LV global longitudinal strain at 28 days after the end of the treatment. Time Frame: 28 days after the end of the treatment.

LV global longitudinal strain (%) by Doppler-echocardiography.

Changes in LV function assessed by LV global longitudinal strain at 6 months after the end of the treatment. Time Frame: 6 months after the end of the treatment.

LV global longitudinal strain (%) by Doppler-echocardiography.

Changes in LV function assessed by LV global longitudinal strain at 12 months after the end of the treatment. Time Frame: 12 months after the end of the treatment.

LV global longitudinal strain (%) by Doppler-echocardiography.

Changes in LV function assessed by LV ejection fraction (%) by Cardiac Magnetic Resonance at 6 months after the end of the treatment. Time Frame: 6 months after the end of the treatment.

Measurements of LV ejection fraction (%) by Cardiac Magnetic Resonance.

Changes in LV function assessed by LV ejection fraction (%) by Cardiac Magnetic Resonance at 12 months after the end of the treatment. Time Frame: 12 months after the end of the treatment.

Measurements of LV ejection fraction (%) by Cardiac Magnetic Resonance.

Changes in LV function assessed by LV volumes (ml/m2) by Cardiac Magnetic Resonance at 6 months after the end of the treatment. Time Frame: 6 months after the end of the treatment.

LV volumes (ml/m2) by Cardiac Magnetic Resonance (CMR).

Changes in LV function Changes in LV function assessed by LV volumes (ml/m2) by Cardiac Magnetic Resonance at 12 months after the end of the treatment. Time Frame: 12 months after the end of the treatment.

LV volumes (ml/m2) by Cardiac Magnetic Resonance (CMR).

Changes in LV function assessed by the presence/extent of myocardial late-enhancement at 6 months after the end of the treatment. Time Frame: 6 months after the end of the treatment.

Presence/extent of myocardial late-enhancement after gadolinium administration, in the absence of contra-indication, by Cardiac Magnetic Resonance.

Changes in LV function assessed by the presence/extent of myocardial late-enhancement at 12 months after the end of the treatment. Time Frame: 12 months after the end of the treatment.

Presence/extent of myocardial late-enhancement after gadolinium administration, in the absence of contra-indication, by Cardiac Magnetic Resonance.

Changes in LV function assessed by maximum oxygen consumption at 6 months after the end of the treatment. Time Frame: 6 months after the end of the treatment.

Maximum oxygen consumption at exercise (mL/min/kg).

Changes in LV function assessed by maximum oxygen consumption at 12 months after the end of the treatment. Time Frame: 12 months after the end of the treatment.

Maximum oxygen consumption at exercise (mL/min/kg).

Changes in LV function assessed by Natriuretic peptide plasma levels at 28 days after the end of the treatment. Time Frame: 28 days after the end of the treatment.

Natriuretic peptide plasma levels (BNP or NT-ProBNP in μg/mL).

Changes in LV function assessed by Natriuretic peptide plasma levels at 6 months after the end of the treatment. Time Frame: 6 months after the end of the treatment.

Natriuretic peptide plasma levels (BNP or NT-ProBNP in μg/mL).

Changes in LV function assessed by Natriuretic peptide plasma levels at 12 months after the end of the treatment. Time Frame: 12 months after the end of the treatment.

Natriuretic peptide plasma levels (BNP or NT-ProBNP in μg/mL).

Serious Adverse Events Time Frame: 12 months.

Number of any potentially Serious Adverse Events (T-SAEs)/Reactions attributed to the experimental treatment (primary endpoint) up to 12 months.

### Criteria

### Inclusion Criteria:

Aged between 18 to 80 years;

Signed written informed consent;

French Social Security affiliation;

Dilated cardiomyopathy defined by a dilated LV with a reduced EF≤40% on echocardiography and/or CMR imaging, unexplained by pressure or volume overload (severe arterial hypertension or significant valve disease), coronary artery disease (as assessed by coronary angiography) or a systemic disease; in case of chemotherapy-induced cardiomyopathy, patients should have a period of at least two years of clinical cancer-free state* and a low estimated likelihood of recurrence (≤30% at 5 years), as determined by an oncologist, based on tumor type, response to therapy, and negative metastatic work-up at the time of diagnosis (*exceptions to this are carcinoma in situ or fully resected basal and squamous cell cancer of the skin);

NYHA Class III in spite of optimal heart failure maximally tolerated guideline-directed medical therapy, including cardiac resynchronization if needed, without other treatment options;

Plasma level of B-type natriuretic peptide (BNP)>150 μg/mL or, N-terminal pro-BNP (NT-proBNP)≥400 μg/mL;

For child-bearing aged women, efficient contraception such as combined (estrogen and progestogen containing) hormonal contraception or progestogen-only hormonal contraception associated with inhibition of ovulation and for men efficient contraception such as condom, during treatment and until the end of the relevant systemic exposure, i.e. until 3 months after the end of treatment.

### Exclusion Criteria:

Implantation of a cardiac resynchronisation therapy device or an ICD unit during the preceding 3 months;

End-stage heart failure with reduced EF (HFrEF) defined as patients with American College of Cardiology Foundation/American Heart Association (ACCF/AHA) stage D (candidates for specialized interventions, including heart transplantation and mechanical assistance) or terminal HF (advanced HF with poor response to all forms of treatment, frequent hospitalizations and life expectancy<12 months);

Patients treated with inotropic agents during the 1 month period prior to inclusion;

Acute heart failure (regardless of the cause);

Heart failure caused by cardiac valve disease, untreated hypertension or documented coronary artery disease with lesions which could explain the cardiomyopathy;

Cardiomyopathy due to a reversible cause e.g., endocrine disease, alcohol or drug abuse, myocarditis, Tako-Tsubo, or arrhythmias;

Cardiomyopathy due a syndromic/systemic disease (e.g., Duchenne's muscular dystrophy, immune/inflammatory/infiltrative disorders [amyloidosis, hemochromatosis]);

If post-chemotherapy cardiomyopathy: a history of radiation therapy AND evidence of constrictive physiology; a baseline computerized tomography scan or CMR showing new tumor or suspicious lymphadenopathy raising concern of malignancy; a trastuzumab treatment within the last 3 months;

Previous cardiac surgery;

Recent stroke (within the last 3 months);

Documented presence of a known LV thrombus, aortic dissection, or aortic aneurysm;

Uncontrolled ventricular tachycardia defined by sustained ventricular tachycardia, including electrical storm and incessant ventricular tachycardia with no response to antiarrhythmic medication; Internal Cardioverter Defibrillator firing in the 30 days prior to the first infusion;

History of drug-induced allergic reactions or allergy of any type having required treatment;

Contraindication to corticosteroids or anti-histaminic agents;

Contraindication to gadoterate meglumine if it will be used with CMR;

Hematological disease: anaemia (haematocrit<25%), leukopenia (leucocytes<2,500/μL) orthrombocytopenia (thrombocytes<100,000/μL); myeloproliferative disorders, myelodysplastic syndrome, acute or chronic leukaemia, and plasma cell dyscrasias (multiple myeloma);

Coagulopathy not due to a reversible cause;

Diminished functional capacity for other reasons such as: Chronic Obstructive Pulmonary Disease (COPD) with Forced Expiratory Volume (FEV)<1 L/min, moderate to severe claudication or morbid obesity;

Diabetes with poorly controlled blood glucose levels and/or evidence of proliferative retinopathy;

Dialysis-dependent renal insufficiency;

Autoimmune disorders or current immunosuppressive therapy;

History of organ transplant or cell-based treatment;

Serum positivity for HIV, hepatitis BsAg, or viremic hepatitis C;

Female patient who is pregnant, nursing, or of child-bearing potential and not using effective birth control;

Active infection;

Known allergy to aminoglycosides;

Patient under legal protection (guardianship);

Participation in another interventional trial;

Life expectancy less than one year.

Contraindication to 18FDG-PETscan.

### Example 49

First-In-Man Treatment of Non-Ischemic Cardiomyopathy by Repeated Intravenous Delivery of the EV-Enriched Secretome from Cardiovascular Progenitor Cells (CTC1-EV)

The SECRET-HF trial (NCT05774509) is a Phase I, open-label, single-centre trial, which assesses the effects of three intravenous infusions of the extracellular vesicle (EV)-enriched secretome of cardiovascular progenitor cells (CPC), three weeks apart, in severely symptomatic patients with drug-refractory left ventricular (LV) dysfunction secondary to non-ischemic dilated cardiomyopathy. The trial has been developed on the basis of three major observations in animal models: (1) the functional benefits of stem cells, intramyocardially transplanted, commonly outlast their limited engraftment time, suggesting that they act primarily through their secretome, which contains soluble factors and EV, that may stimulate endogenous tissue repair, see Garbernand and Lee (Cell Stem Cell, 2013; 12:689-698)); (2) the best outcomes seem to come from EV derived from immature cells phenotypically close to those of the tissue to repair, see Khan et al. (Stem Cell Rev and Rep, 2022; 18:1143-1167), hence the choice of CPC as the EV donors; (3) this secretome still retains its cardio-reparative properties when given intravenously, see Lee et al. (Pharmaceutics, 2023; 15:325) and Desgres et al. (Front Cardiovasc Med, 2023; 10:1206279), enabling convenient repeated administrations.

Briefly, human induced pluripotent stem cells (iPSC) were reprogrammed from the somatic cells of a healthy donor. They were differentiated to CPC at the FUJIFILM Cellular Dynamics, Inc. Innovation Facility for Advanced Cell Therapy (Madison, WI) according to current Good Manufacturing Practices (cGMP). CPC underwent extensive quality control testing (ex. viability, identity, purity, sterility, genomic integrity, adventitious agent screening) after which the cryopreserved cells were shipped to the MEARY Cell and Gene Therapy Center, Assistance Publique-Höpitaux de Paris, France, where they were thawed, quality-tested (ex. viability, identity, sterility) and expanded in a “vesiculation” medium. The conditioned medium, was filtered, enriched for EV and concentrated by tangential flow filtration using a molecular weight cut-off of 30 kDa, which retained the small EV fraction and large secreted proteins. The retentate was filter-sterilised and aliquoted into 2-mL glass vials which were stored at −80 °C. Samples of the final clinical-grade drug product were quality-tested (ex. number and size of particles, EV marker identity, sterility, in vitro potency (cell proliferation assay), in vitro immunogenicity, stability, and evaluated for toxicology and oncogenicity in vivo (GLP animal studies; treated with 10-fold higher dose than the anticipated maximum human dose). The Ethics Review Board of Île-de-France V approved the trial that was authorized by the French Regulatory Agency (Agence nationale de wsécurité du médicament et des produits de santé, ANSM) on Feb. 2, 2023 (EUDRACT: 2022-001844-75).

The primary outcome measure of the trial is the number of any potentially serious adverse events (SAEs) or reactions recorded after each infusion or up to one year thereafter. A maximum of 12 patients are planned with dose escalation using the Bayesian optimal interval (BOIN) design with 4 patients receiving three infusions of 20×10⁹ particles/kg (the equivalent of the secretome recovered from 1 million cells per kg per infusion), and, in the absence of dose-limiting toxicity (DLT), a second cohort of 8 patients receiving three infusions of 40×10⁹ particles/kg.

This 59-year-old male was followed for a non-ischemic cardiomyopathy with a pathogenic variant of tropomyosin. At the time of referral, he was in New York Heart Association (NYHA) class III, despite an optimal guideline-directed medical therapy. He had been previously implanted with an automatic internal defibrillator. Echocardiography showed LV end-diastolic (LVEDV) and end-systolic (LVESV) volumes of 220 mL and 164 mL, respectively, with an ejection fraction (EF) of 25%. The peak VO₂ was 13.4 mL/min/kg. The three secretome infusions (20×10⁹ particles/kg each, three weeks apart) were performed through an intravenous peripheral line in the Intensive Care Unit (Hôpital Europden Georges Pompidou, APHP, Paris, France), under continuous monitoring of heart rate, blood pressure and oxygen saturation. Each infusion was preceded by an intravenous premedication of anti-histaminic drug and methylprednisolone. All infusions were well tolerated without SAEs, allowing for rapid discharge. The C-Reactive Protein decreased from 15.6 mg/L from before the first infusion to 1.9 mg/L after the third one. Eleven weeks after the first infusion, he was in NYHA Class II without significant changes in echo parameters, with reduced need for diuretics (from 240 mg to 160 mg). Donor-specific antibody (DSA) testing, repeated after each infusion and 28 days post-treatment, showed no allogeneic-immunization against the clinical product.

### Discussion

Multiple studies spanning a wide variety of preclinical disease models have shown that the benefits of transplanting cells can be duplicated by the administration of their secretome alone, including for heart conditions. See Kervadec et al. (The Journal of Heart and Lung Transplantation, 2016; 35:795-807). This case report illustrates the feasibility of manufacturing a cell-derived secretome under cGMP conditions and at Phase I clinical manufacturing scale, and supports the short-term safety of repeated intravenous delivery of the product and, more specifically, the lack of immune-inflammatory response or allogeneic immunisation subsequent to repeated doses. This supports the idea that, in contrast to allogeneic pluripotent stem cell therapy, secreted EV are not immunogenic. See Lima et al. (Cardiovascular Research, 2021; 117:292-307). The stability of this secretome product after cryo-storage enables its off-the-shelf use, which is a significant advantage of a secretome product over a cellular product, as discussed elsewhere. Furthermore, while mesenchymal stromal cell-derived EV-enriched secretomes have been shown to be effective in preclinical studies, see Kou et al. (Cell Death Dis, 2022; 13:580), the choice of using an human iPSC-derived mother cell type overcomes the challenge of donor to donor reproducibility.

The mechanisms by which intravenously delivered EV could be cardio-protective remain unclear in view of their preferential biodistribution in the liver, spleen and lungs. However, the consistent observation that an intravenous delivery route can be functionally effective in heart, see Desgres et al. (Front Cardiovasc Med, 2023; 10:1206279), brain and kidney models, see Lee et al. (Pharmaceutics, 2023; 15:325), has led to the idea that infused EV could rewire endogenous immune cells, circulating and in peripheral organs, to take on a reparative phenotype. See Savitz and Cox (Nat Rev Neurol, 2022). These cells could travel to the heart to carry out the reparative processes they were instructed to do by the therapeutic vesicles, including the mitigation of inflammation, see Viola et al. (IJMS, 2021; 22:7831), which is a hallmark of cardiac failure. See Adamo et al. (Nat Rev Cardiol, 2020; 17:269-285).

Lengthy table referenced here
US20260345503A1-20261105-T00001
Please refer to the end of the specification for access instructions.

Lengthy table referenced here
US20260345503A1-20261105-T00002
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260345503A1). An electronic copy of the table will also be available from the USPTO upon request and payment of the fee set forth in 37 CFR 1.19(b)(3).

What is claimed is:

1. A method for generating a secretome, said method comprising:
(a) culturing one or more progenitor cells in a first serum-free culture medium, wherein said first serum-free culture medium comprises basal medium, human serum albumin, and one or more growth factors;
(b) removing said first serum-free culture medium from said one or more progenitor cells;
(c) culturing said one or more progenitor cells in a second serum-free culture medium, wherein said second serum-free culture medium comprises basal medium, but does not comprise human serum albumin or growth factors; and
(d) recovering the second serum-free culture medium after the culturing of step (c), to thereby obtain conditioned medium comprising the secretome of the one or more progenitor cells.

2. The method of claim 1, wherein one of said one or more growth factors is fibroblast growth factor 2 (FGF-2).

3. The method of claim 1 or 2, wherein said first and second serum-free media are supplemented with a carbohydrate source.

4. The method of claim 3, wherein said carbohydrate source is glucose.

5. The method of any one of claims 1-4, wherein said first and second serum-free media are supplemented with an antibiotic.

6. The method of claim 5, wherein said antibiotic is gentamicin.

7. The method of any one of claims 1-6, wherein said first serum-free media further comprises one or more selected from the group consisting of: glutamine; biotin; DL alpha tocopherol acetate; DL alpha-tocopherol; vitamin A; catalase; insulin; transferrin; superoxide dismutase; corticosterone; D-galactose; ethanolamine, glutathione; L-carnitine; linoleic acid; progesterone; putrescine; sodium selenite; triodo-I-thyronine; an amino acid; sodium pyruvate; lipoic acid; vitamin B12; nucleosides; and ascorbic acid.

8. The method of any one of claims 1-7, wherein said basal medium is a Minimum Essential Medium (MEM).

9. The method of claim 8, wherein said MEM is a-MEM.

10. The method of any one of claims 1-9, wherein the culturing of step (a) is for 6-96 hours.

11. The method of claim 10, wherein the culturing of step (a) is for 12-96 hours.

12. The method of claim 11, wherein the culturing of step (a) is for 36-84 hours.

13. The method of claim 12, wherein the culturing of step (a) is for about 72 hours.

14. The method of any one of claims 1-13, wherein the culturing of step (c) is for 6-96 hours.

15. The method of claim 14, wherein the culturing of step (c) is for 12-72 hours.

16. The method of claim 15, wherein the culturing of step (c) is for 36-60 hours.

17. The method of claim 16, wherein the culturing of step (c) is for about 48 hours.

18. The method of claim 14, wherein the last 12-36 hours of the culturing of step (c) is conducted under hypoxic conditions.

19. The method of claim 18, wherein said culture conditions comprise culturing in an atmosphere having 1-21% oxygen.

20. The method of any one of claims 1-19, wherein after step (b), but before step (c), said one or more progenitor cells are washed.

21. The method of any one of claims 1-20, wherein said one or more progenitor cells comprise progenitor cells selected from the group consisting of cardiomyocyte progenitor cells, cardiac progenitor cells, vascular progenitor cells and cardiovascular progenitor cells.

22. The method of any one of claims 1-21, wherein said one or more progenitor cells are obtained from induced pluripotent stem cells (iPSCs).

23. The method of any one of claims 1-4 and 7-22, wherein said first and second serum-free media do not contain an antibiotic.

24. The method of any one of claims 1-23, wherein the culturing in one or more of steps (a) and (c) is two-dimensional cell culture.

25. The method of claim 24, wherein said two-dimensional cell culture comprises culturing said one or more progenitor cells on a surface of a culture vessel.

26. The method of claim 25, wherein said culture vessel surface is coated with a substance to promote cell adhesion.

27. The method of claim 26, wherein said substance to promote cell adhesion is vitronectin or fibronectin.

28. The method of any one of claims 1-23, wherein the culturing in one or more of steps (a) and (c) is three-dimensional cell culture.

29. The method of claim 28, wherein the three-dimensional cell culture comprises culturing cell aggregates in suspension in a bioreactor, spinner flask, or stirred culture vessel, or comprises culturing cells in a microcarrier culture system.

30. The method of any one of claims 1-29, wherein said method further comprises pre-clearing the medium recovered in step (d) by centrifugation, filtration, or a combination of centrifugation and filtration.

31. The method of any one of claims 1-30, wherein said method further comprises optionally freezing the medium recovered in step (d).

32. The method of any one of claims 1-31, wherein said one or more progenitor cells cultured in step (a) have previously been frozen.

33. The method of any one of claims 1-32, wherein said method further comprises concentrating and/or enriching for a small extracellular vesicle-enriched fraction (sEV) from the medium recovered in step (d).

34. The method of claim 33, wherein said sEV is concentrated and/or enriched from the recovered medium by at least one process selected from the group consisting of ultracentrifugation, filtration, ultrafiltration, tangential flow filtration, size exclusion chromatography, and affinity capture.

35. The method of claim 33, wherein said enriching enriches for extracellular vesicles that have one or more of the following characteristics: (a) are CD63⁺, CD81⁺ and/or CD9⁻; (b) are between 50-200 nm in diameter; (c) are positive for one or more of CD49e, ROR1, SSEA-4, MSCP, CD146, CD41b, CD24, CD44, CD236, CD133/1, CD29 and CD142; and/or (d) are negative for one or more of CD19, CD4, CD209, HLA-ABC, CD62P, CD42a and CD69.

36. The method of claim 33, wherein said sEV comprises one or more of exosomes, microparticles, extracellular vesicles and secreted proteins.

37. A secretome-containing composition obtained by the method of any one of claims 1-32.

38. An sEV-containing composition obtained by the method of any one of claims 33-36.

39. A method for producing a therapeutic composition suitable for administration to a patient, said method comprising producing a secretome-containing composition according to the method of any one of claims 1-32.

40. The method of claim 39, wherein said method further comprises purifying, concentrating, isolating, and/or enriching, said secretome-containing composition by one or more purification, concentration, isolation, and/or enrichment, steps.

41. The method of claim 39, wherein said method further comprises adding a pharmaceutically acceptable excipient or carrier to the secretome-containing composition.

42. A method for producing a therapeutic composition suitable for administration to a patient, said method comprising producing an sEV-containing composition according to the method of any one of claims 33-36.

43. The method of claim 42, wherein said method further comprises purifying, concentrating, isolating, and/or enriching, said sEV-containing composition by one or more purification, concentration, isolation, and/or enrichment, steps.

44. The method of claim 42, wherein said method further comprises adding a pharmaceutically acceptable excipient or carrier to the sEV-containing composition.

45. A therapeutic composition comprising the secretome-containing composition of claim 37, and a pharmaceutically acceptable excipient or carrier.

46. A therapeutic composition comprising the sEV-containing composition of claim 38, and a pharmaceutically acceptable excipient or carrier.

47. A secretome-containing composition obtained by the method of claim 1, wherein said one or more progenitor cells comprise progenitor cells selected from the group consisting of cardiomyocyte progenitor cells, cardiac progenitor cells, vascular progenitor cells and cardiovascular progenitor cells.

48. An sEV-containing composition obtained by the method of claim 33, wherein said one or more progenitor cells comprise progenitor cells selected from the group consisting of cardiomyocyte progenitor cells, cardiac progenitor cells, vascular progenitor cells, and cardiovascular progenitor cells.

49. A therapeutic composition comprising the secretome-containing composition of claim 47 and a pharmaceutically acceptable excipient or carrier.

50. A therapeutic composition comprising the composition of claim 48 and a pharmaceutically acceptable excipient or carrier.

51. A method for treating acute myocardial infarction, chemotherapy-induced cardiomyopathy, heart failure, myocarditis, ischemic cardiomyopathy, cardiomyopathy, ventricular dysfunction, atrial dysfunction, or arrhythmia in a subject in need thereof, comprising administering to the subject the therapeutic composition of claim 49 or 50.

52. A method for improving angiogenesis, comprising administering to a subject in need thereof the therapeutic composition of claim 49 or 50.

53. A method for improving cardiac performance, comprising administering to a subject in need thereof the therapeutic composition of claim 49 or 50.

54. The method of claim 11, wherein the culturing of step (a) is for 60-84 hours.

55. The method of claim 14, wherein the last 12-36 hours of the culturing of step (c) is conducted under normoxic conditions.

56. The method of claim 55, wherein said normoxic conditions comprise culturing in an atmosphere containing 20-21% oxygen.

57. The method of claim 29, wherein the bioreactor is a vertical wheel bioreactor.

58. The method of claim 39, wherein said method further comprises cryopreserving, freezing, or lyophilizing, said secretome-containing composition.

59. The method of claim 42, wherein said method further comprises cryopreserving, freezing, or lyophilizing, said sEV-containing composition.

60. The method of claim 2, wherein said first serum-free media comprises 0.1-10 μg/mL FGF-2.

61. The method of claim 60, wherein said first serum-free media comprises 0.5-5 μg/mL FGF-2.

62. The method of claim 61, wherein said first serum-free media comprises 0.5-2.5 μg/mL FGF-2.

63. The method of claim 62, wherein said first serum-free media comprises about 1 μg/mL FGF-2.

64. The method of any of claims 1-36, 39-44 and 54-63, wherein said method is Good Manufacturing Practices (GMP)-ready.

65. The secretome-containing composition of claim 37, wherein said composition is GMP-ready.

66. The sEV-containing composition of claim 38, wherein said composition is GMP-ready.

67. The method of claim 14, wherein the last 12-36 hours of the culturing of step (c) is conducted under normoxic conditions.

68. The method of claim 67, wherein said normoxic conditions comprises culturing in an atmosphere containing between 20-21% of oxygen.

69. The method of claim 30, wherein said pre-clearing comprises at least three filtration steps.

70. The method of claim 34, wherein the separation of said sEV from the recovered medium comprises tangential flow filtration.

71. The secretome-containing composition of claim 37 comprising trehalose and L-histidine.

72. The sEV-containing composition of claim 3 comprising trehalose and L-histidine.

73. The secretome-containing composition of claim 37 or 65, wherein said composition is able to promote wound scratch healing in an in vitro wound scratch healing assay, and/or is able to promote cardiomyocyte viability in an in vitro cardiomyocyte viability assay.

74. The sEV-containing composition of claim 38 or 66, wherein said composition is able to promote wound scratch healing in an in vitro wound scratch healing assay, and/or is able to promote cardiomyocyte viability in an in vitro cardiomyocyte viability assay.

75. The secretome-containing composition of claim 37 or 65, wherein said composition is at least one of the following: a composition that has been enriched for extracellular vesicles having a diameter of between about 50-200 nm or between 50-200 nm, preferably having a diameter of between about 50-150 nm or between 50-150 nm; a composition that is substantially free or free of whole cells; and/or a composition that is substantially free of one or more culture medium components.

76. The sEV-containing composition of claim 38 or 66, wherein said composition is at least one of the following: a composition that has been enriched for extracellular vesicles having a diameter of between about 50-200 nm or between 50-200 nm, preferably having a diameter of between about 50-150 nm or between 50-150 nm; a composition that is substantially free or free of whole cells; and/or a composition that is substantially free of one or more culture medium components.

77. The method of claim 51, wherein the heart failure is acute heart failure, chronic heart failure, ischemic heart failure, non-ischemic heart failure, heart failure with ventricular dilation, heart failure without ventricular dilation, heart failure with reduced left ventricular ejection fraction, or heart failure with preserved left ventricular ejection fraction.

78. The method of claim 77, wherein the heart failure is selected from the group consisting of ischemic heart disease, cardiomyopathy, myocarditis, hypertrophic cardiomyopathy, diastolic hypertrophic cardiomyopathy, dilated cardiomyopathy, and post-chemotherapy induced heart failure.

79. The secretome-containing composition of claim 37 or 65, wherein said composition is able to promote cardiomyocyte viability in an in vitro chemotherapy-induced cardiomyopathy viability assay.

80. The sEV-containing composition of claim 38 or 66, wherein said composition is able to promote cardiomyocyte viability in an in vitro chemotherapy-induced cardiomyopathy viability assay.

81. The secretome-containing composition of claim 79, wherein in said in vitro chemotherapy-induced cardiomyopathy viability assay, said chemotherapy is an anthracycline.

82. The secretome-containing composition of claim 81, wherein said anthracycline is doxorubicin.

83. The sEV-containing composition of claim 80, wherein in said in vitro chemotherapy-induced cardiomyopathy viability assay, said chemotherapy is an anthracycline.

84. The sEV-containing composition of claim 83, wherein said anthracycline is doxorubicin.

85. The method according to claim 51, wherein said chemotherapy-induced cardiomyopathy is caused by an anthracycline.

86. The method according to claim 85, wherein said anthracycline is doxorubicin.

87. A method of maintaining physiological heart volume in a subject by administering to said subject the therapeutic composition of any one of claims 45, 46, 49 and 50.

88. The method of any one of claims 51-53 and 87, wherein said method maintains Left Ventricular End Systolic Volume (LVESV) within 15% of the pre-treatment LVESV.

89. The method of any one of claims 51-53 and 87, wherein said method maintains LVEDV within 2% of the pre-treatment volume.

90. The method of any one of claims 51-53 and 87, wherein the method prevents progressive post-ischemic heart failure.

91. The method of any one of claims 51-53 and 87, wherein the method improves endothelial cell survival, health and function in said subject.

92. The method of any one of claims 51-53 and 87, wherein said method reduces fibrosis in stimulated cardiac fibroblasts.

93. The method of claim 92, wherein said method reduces the expression of the pro-fibrotic marker, POSTN, in TGF-β1-stimulated cardiac fibroblasts to level prior to stimulation with TGF-β1 or below.

94. The method of any one of claims 51-53 and 87, wherein the method does not induce an allogeneic inflammatory response in a subject.

95. The method of any one of claims 51-53 and 87, wherein said method does not induce an allogeneic peripheral blood mononuclear cell (PBMC) activation.

96. The method of any one of claims 51-53 and 87, wherein said method does not induce a significant increase in the percentage of IFNg or IL-2 expressing PBMCs.

97. The method of any one of claims 51-53 and 87, wherein the method does not induce allogeneic natural killer (NK) cell degranulation.

98. The method of any one of claims 51-53 and 87, wherein the method does not induce a significant increase in the percentage of CD107 expressing NK cells.

99. A method of improving heart function in a patient experiencing heart failure by administering the therapeutic composition of any one of claims 45, 46, 49 and 50.

100. The method of claim 99, wherein the method improves survival of stressed cardiomyocyte cells.

101. The method of claim 99, wherein the method improves one or more of the seeding, survival, viability and proliferation of stressed endothelial cells in vitro.

102. The method of claim 99, wherein the method improves cell migration and/or wound healing capabilities in stressed endothelial cells.

103. The method of any one of claims 99-102, wherein the method improves wound healing in said subject.

104. The method of any of one of claims 99-103, wherein the method reduces signs of fibrosis in fibroblast cells of said subject.

105. The method of claim 104, wherein the fibroblasts are activated with TGF-β1.

106. The method of any one of claims 99-105, wherein the method does not stimulate allogeneic human PBMC activation.

107. The method of any of claims 99-106, wherein the method does not induce NK degranulation of allogeneic human NK cells.

108. The method of any of claims 99-107, wherein the composition is non-toxic in mice and rats at a dose of 4×10¹¹ particles/kg.

109. The method of any of claims 99-108, wherein the composition is not tumorigenic in mice at dose of 4×10¹¹ particles/kg.

110. The method of any of claims 99-109, wherein the composition does not contain DNA fragments ranging from 179 to 742 pb, at concentrations in the μg/mL range.

111. The method of any of claims 51-53 and 87-110, wherein said therapeutic composition is administered as an intravenous infusion, direct cardiac injection or is administered intra-arterially.

112. The method of claim 111, wherein the therapeutic composition is administered at a dose containing secretome obtained from 0.1 to 10 million cells per kg weight of said subject per administration.

113. The method of claim 111, wherein the therapeutic composition is administered at a dose containing secretome obtained from 0.5 to 5 million cells per kg weight of said subject per administration.

114. The method of claim 111, wherein the therapeutic composition is administered at a dose containing secretome obtained from 1 to 3 million cells per kg weight of said subject.

115. The method of claim 111, wherein the therapeutic composition is administered at a dose containing secretome obtained from 1 to 2 million cells per kg weight of said subject.

116. The method of claim 111, wherein said therapeutic composition is administered at a dose containing from 1×10⁹ to 60×10⁹ particles, as measured by Nanoparticle Tracking Analysis (NTA), per kg weight of the subject.

117. The method of claim 111, wherein said therapeutic composition is administered at a dose containing from 10×10⁹ to 60×10⁹ particles, as measured by NTA, per kg weight of the subject.

118. The method of claim 111, wherein said therapeutic composition is administered at a dose containing from 10×10⁹ to 40×10⁹ particles, as measured by NTA, per kg weight of the subject.

119. The method of claim 111, wherein said therapeutic composition is administered at a dose containing from 20×10⁹ to 40×10⁹ particles, as measured by NTA, per kg weight of the subject.

120. The method of claim 111, wherein said therapeutic composition is administered at a cumulative daily dose containing from 20×10⁹ to 200×10⁹ particles, as measured by NTA, per kg weight of the subject.

121. The method of claim 111, wherein said therapeutic composition is administered at a cumulative daily dose containing from 30×10⁹ to 100×10⁹ particles, as measured by NTA, per kg weight of the subject.

122. The method of claim 111, wherein said therapeutic composition is administered at a cumulative daily dose containing 60×10⁹ particles, as measured by NTA, per kg weight of the subject.

123. The method of claim 111, wherein said therapeutic composition is administered at a cumulative daily dose containing 40×10⁹ particles, as measured by NTA, per kg weight of the subject.

124. The method of any one of claims 111-123, wherein said composition is administered 1 to 10 times per day.

125. The method of any one of 111-123, wherein said composition is administered 3 to 6 times per day.

126. The method of any one of 111-123, wherein said composition is administered 1 to 5 times per day.

127. The method of any one of 111-123, wherein said composition is administered 3 times per day.

128. The method of any one of 111-123, wherein said composition is administered 2 times per day.

129. The method of any one of claims 51-53 and 87-128, wherein the duration of treatment is 60 days or less.

130. The method of claim 129, wherein the duration of treatment is from 5 to 50 days.

131. The method of claim 129, wherein the duration of treatment is from 10 to 50 days.

132. The method of claim 129, wherein the duration of treatment is from 20 to 45 days.

133. The method of claim 129, wherein the duration of treatment is 42 days.

134. The method of any one of claims 111-133, wherein said therapeutic composition is administered every day.

135. The method of any one of claims 111-133, wherein said therapeutic composition is administered every other day.

136. The method of any one of claims 111-133, wherein said therapeutic composition is administered at a frequency of from every day to every 30 days.

137. The method of any one of claims 111-133, wherein said therapeutic composition is administered at a frequency of from every 7 days to every 21 days.

138. The method of any one of claims 111-133, wherein said therapeutic composition is administered every 21 days.

139. The method of any one of claims 111-138, wherein said therapeutic composition is formulated in a solution comprising one or more pharmaceutically acceptable excipient.

140. A secretome-containing composition obtained by the method of any one of claims 1-32, wherein said secretome-containing composition comprises extracellular vesicles secreted from said progenitor cells.

141. The secretome-containing composition of claim 140, wherein said extracellular vesicles comprise one or more miR selected from hsa-miR-302a-5p, hsa-miR-16-5p, hsa-miR-93-5p, hsa-miR-126-3p, hsa-miR-148a-3p, hsa-miR-21-5p, hsa-miR-20a-5p, hsa-miR-143-3p, hsa-miR-335-5p, hsa-miR-218-5p, hsa-miR-101-3p, hsa-miR-302d-3p, hsa-miR-25-3p, hsa-miR-126-5p, hsa-miR-423-5p, hsa-miR-532-5p, hsa-miR-1246, hsa-miR-302a-3p, hsa-miR-20b-5p, hsa-miR-148b-3p, hsa-miR-34a-5p, hsa-miR-1-3p, hsa-miR-191-5p, hsa-miR-26b-5p, hsa-miR-151a-3p, hsa-miR-103a-3p/107, hsa-miR-660-5p, hsa-miR-320a-3p/320b/320c/320d/320e, hsa-miR-130a-3p, hsa-miR-19b-3p, hsa-miR-27a-3p/27b-3p, hsa-miR-186-5p, hsa-miR-26a-5p, hsa-miR-125b-5p, hsa-miR-7-5p, hsa-miR-24-3, hsa-miR-483-5p, hsa-miR-99b-5p, hsa-miR-205-5p, and hsa-miR-302b-3p.

142. The secretome-containing composition of claim 140, wherein said extracellular vesicles comprise one or more miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p.

143. The secretome-containing composition of claim 140, wherein said extracellular vesicles comprise at least five miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p.

144. The secretome-containing composition of any one of claims 141-143, wherein an expression level of the one or more miR ranges from −5 to +5 units.

145. A secretome-containing composition comprising a secretome from progenitor cells, said secretome comprising extracellular vesicles secreted from said progenitor cells.

146. The secretome-containing composition of claim 145, wherein said progenitor cells are cardiovascular progenitor cells.

147. The secretome-containing composition of claim 145 or 146, wherein said extracellular vesicles comprise one or more miR selected from hsa-miR-302a-5p, hsa-miR-16-5p, hsa-miR-93-5p, hsa-miR-126-3p, hsa-miR-148a-3p, hsa-miR-21-5p, hsa-miR-20a-5p, hsa-miR-143-3p, hsa-miR-335-5p, hsa-miR-218-5p, hsa-miR-101-3p, hsa-miR-302d-3p, hsa-miR-25-3p, hsa-miR-126-5p, hsa-miR-423-5p, hsa-miR-532-5p, hsa-miR-1246, hsa-miR-302a-3p, hsa-miR-20b-5p, hsa-miR-148b-3p, hsa-miR-34a-5p, hsa-miR-1-3p, hsa-miR-191-5p, hsa-miR-26b-5p, hsa-miR-151a-3p, hsa-miR-103a-3p/107, hsa-miR-660-5p, hsa-miR-320a-3p/320b/320c/320d/320e, hsa-miR-130a-3p, hsa-miR-19b-3p, hsa-miR-27a-3p/27b-3p, hsa-miR-186-5p, hsa-miR-26a-5p, hsa-miR-125b-5p, hsa-miR-7-5p, hsa-miR-24-3, hsa-miR-483-5p, hsa-miR-99b-5p, hsa-miR-205-5p, and hsa-miR-302b-3p.

148. The secretome-containing composition of claim 145 or 146, wherein said extracellular vesicles comprise one or more miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p.

149. The secretome-containing composition of claim 145 or 146, wherein said extracellular vesicles comprise at least five miR selected from hsa-miR-1-5p, hsa-miR-11401, hsa-miR-1263-3p, hsa-miR-3085-3p, hsa-miR-3161-5p, hsa-miR-3678-3p, hsa-miR-3942-5p, hsa-miR-4652-5p, hsa-miR-4758-5p, hsa-miR-4760-5p, hsa-miR-4779-3p, hsa-miR-508-5p, hsa-miR-548ad-3p, hsa-miR-5580-5p, hsa-miR-559-5p, hsa-miR-6791-5p, hsa-miR-6889-5p, and hsa-miR-96-3p.

150. The secretome-containing composition of any one of claims 147-149, wherein an expression level of the one or more miR ranges from −5 to +5 units.


SEQUENCE LISTING (14 sequences)

SEQ ID NO: 1 - DNA, 24 bp, synthetic construct
  source 1..24: mol_type = other DNA
gcgagtggatgccgcctttaactg

SEQ ID NO: 2 - DNA, 24 bp, synthetic construct
  source 1..24: mol_type = other DNA
gtccacgacggcatccaggttatc

SEQ ID NO: 3 - DNA, 24 bp, synthetic construct
  source 1..24: mol_type = other DNA
ggagaaacggtgcgattcacatat

SEQ ID NO: 4 - DNA, 24 bp, synthetic construct
  source 1..24: mol_type = other DNA
agagcatttttgtcccgtatcaga

SEQ ID NO: 5 - DNA, 22 bp, synthetic construct
  source 1..22: mol_type = other DNA
tacaaaaattagccgggcgtgg

SEQ ID NO: 6 - DNA, 22 bp, synthetic construct
  source 1..22: mol_type = other DNA
gttcaagcgattctcctgcctc

SEQ ID NO: 7 - DNA, 25 bp, synthetic construct
  source 1..25: mol_type = other DNA
cgcgcctgtaatcccagctactcgg

SEQ ID NO: 8 - DNA, 22 bp, synthetic construct
  source 1..22: mol_type = other DNA
ctcacgcctgtaatcccagcac

SEQ ID NO: 9 - DNA, 22 bp, synthetic construct
  source 1..22: mol_type = other DNA
atctcggctcactgcaacctcc

SEQ ID NO: 10 - DNA, 26 bp, synthetic construct
  source 1..26: mol_type = other DNA
cgagaccagcccggccaacacggtga

SEQ ID NO: 11 - DNA, 25 bp, synthetic construct
  source 1..25: mol_type = other DNA
aagtccctgccctttgtacacaccg

SEQ ID NO: 12 - DNA, 20 bp, synthetic construct
  source 1..20: mol_type = other DNA
gcaattattccccatgaacg

SEQ ID NO: 13 - DNA, 20 bp, synthetic construct
  source 1..20: mol_type = other DNA
aacaggtctgtgatgccctt

SEQ ID NO: 14 - DNA, 20 bp, synthetic construct
  source 1..20: mol_type = other DNA
ggcctcactaaaccatccaa